(19) United States
(12) Patent Application Publication
(10) Pub. No.: US 2026/0137091 A1
(43) Pub. Date: May 21, 2026

(54) NEW PEPTIDES AND THE USE OF SAME FOR MODULATING ACCUMULATION OF A PROTEIN

(71) Applicants: UNIVERSITÉ TOULOUSE III - PAUL SABATIER, Toulouse Cedex 9 (FR); CENTRE NATIONAL DE LA RECHERCHE SCIENTIFIQUE, Paris (FR)

(72) Inventors: Jean-Philippe COMBIER, CASTANET-TOLOSAN (FR); Bruno GUILLOTIN, CASTANET-TOLOSAN (FR); Dominique LAURESSERGUES, CASTANET-TOLOSAN (FR)

(73) Assignees: UNIVERSITÉ TOULOUSE III - PAUL SABATIER, Toulouse Cedex 9 (FR); CENTRE NATIONAL DE LA RECHERCHE SCIENTIFIQUE, Paris (FR)

(21) Appl. No.: 19/122,509

(22) PCT Filed: Oct. 19, 2023
(86) PCT No.: PCT/EP2023/079155

§ 371 Date: Apr. 18, 2025

(30) Foreign Application Priority Data
Oct. 19, 2022 (FR) FR2210832

(51) Int. Cl. A01N 63/50; A01C 1/06; A01P 13/00; C07K 7/06

(52) U.S. Cl. CPC A01N 63/50; A01C 1/06; A01P 13/00; C07K 7/06

(57) ABSTRACT

New peptides (cPEPs) having a size from 4 to 70 amino acid residues and a method for preparing the cPEPs. Also, a method for modulating the accumulation of specific proteins in plant cells by introducing the cPEPs into the plant cells. The cPEPs may be introduced to either promote the development of a plant or slow down or prevent the development of a plant.

### FIELD OF THE INVENTION

The present invention relates to new peptides (cPEPs), a method for their preparation, and their use for modulating the accumulation of specific proteins.

### PRIOR ART

Generally, peptides are short sequences of between 2 and around 100 amino acids. They are often highly active molecules, such as hormones or venom compounds.

In plants, peptides fulfil numerous biological functions, such as development or defence mechanisms. As peptide detection is relatively difficult, only a limited number of peptides have been identified, probably underestimating the quantity and role of peptides in these organisms.

Most of the peptides characterised in plants probably result from the degradation of functional proteins. However, it has been shown that primary transcripts of plant microRNAs (miRs) actually contain small open reading frames (miORFs) encoding regulatory peptides, called miPEPs (Lauressergues D et al. Primary transcripts of microRNAs encode regulatory peptides. Nature. 2015 Apr. 2; 520(7545):90-3). The miPEPs are produced at the same site as the miRs from which they originate and enhance transcription of the corresponding pri-miRs. The activity of a miPEP is highly specific to the corresponding miR, making it possible to up-regulate the chosen miR without affecting the expression of other miRs. The use of miPEPs can modulate the expression of a gene if it is regulated by a miR, itself regulated by a miPEP (WO 2015/063431).

### BRIEF OVERVIEW

In this context, the present invention provides a universal and readily exploitable means of specifically modulating the accumulation of a selected protein in a plant using a non-naturally occurring peptide, i.e. a peptide that is not naturally produced by the plant.

One aspect of the invention is to propose a method for preparing and determining a “cPEP” peptide capable of modulating the accumulation (expression) of a specific protein in a plant cell. A second aspect of the invention is to propose a method for modulating the accumulation of a protein in a plant using a cPEP. A third aspect of the invention is to propose the use of a cPEP to modulate the accumulation of a protein in a plant. A fourth aspect of the invention is to propose a method for promoting, slowing down or preventing the development of a plant. A fifth aspect of the invention is to propose cPEP peptides for modulating the accumulation of a protein in a plant. Other complementary aspects of the invention concern a nucleic acid encoding a cPEP, compositions comprising a cPEP and modified or transgenic plants comprising a cPEP.

### DETAILED DESCRIPTION

In a first aspect, the invention relates to a method for preparing and determining a cPEP, said cPEP:
having a size from 4 to 70 amino acids, in particular from 4 to 41 amino acids;
being capable of modulating the accumulation of a protein in a plant cell; and
not being capable of modulating the accumulation of the mRNA encoding said protein,
   said method comprising:
a. a step for determining the nucleic acid sequence of the pre-messenger RNA (pre-mRNA) encoding said protein;
b. a step for determining within this pre-mRNA one of the nucleic acid sequences deemed to be non-coding;
c. a step for determining a fragment of this nucleic acid sequence deemed to be non-coding,
said fragment having a size of 3n nucleotides, n being from 4 to 70, in particular n being from 4 to 41;
d. a step for producing the peptide encoded by said fragment; and
e. a comparison step:
   between the accumulation of said protein in a plant cell in the presence of said peptide and the accumulation of said protein in a plant cell of the same type in the absence of said peptide; and/or
   between the phenotype of a plant in the presence of said peptide and the phenotype of a plant of the same type in the absence of said peptide,
wherein:
   a difference in the amount of said protein in the presence of said peptide compared to the amount of said protein in the absence of said peptide; and/or
   a difference in the phenotype in the presence of said peptide compared with the phenotype in the absence of said peptide,
indicates that said peptide is a cPEP capable of modulating the accumulation of said protein in a plant cell.

The present invention is based on the Inventors' unexpected observation that it is possible to specifically modulate the accumulation of a protein using a particular peptide that is not produced naturally, the sequence of which corresponds to the (artificial) translation of a fragment of the pre-messenger RNA (pre-mRNA) encoding the said protein, the said fragment being chosen from one of the nucleic acid sequences deemed to be non-coding of the said pre-mRNA.

In this invention, the term “cPEP” (complementary peptide) refers to a peptide capable of specifically modulating the accumulation of a protein when introduced into a plant cell.

According to the invention, a cPEP is not naturally present in a plant cell. This means that the plant cell contains the cPEP information but does not contain the nucleic sequence capable of enabling its expression. Only the peptide sequence of the cPEP can be deduced from the sequence of the pre-messenger RNA (pre-mRNA) encoding the protein, the accumulation of which is to be modulated.

A cPEP is only present in a plant cell once it has been introduced in the form of a peptide or in the form of a nucleic acid encoding said peptide.

The specificity of the cPEP in relation to a target protein (a target gene) is determined by its amino acid sequence. The sequence of a cPEP corresponds to the in silico (artificial) translation into amino acids of a fragment of the pre-messenger RNA (pre-mRNA) encoding the said protein, the said fragment being chosen from one of the nucleic acid sequences deemed to be non-coding of the said pre-mRNA. In particular, said fragment is smaller in size than the naturally translated nucleic acid sequence.

The peptide sequence of a cPEP can therefore be determined from a fragment of the pre-messenger RNA (pre-mRNA) encoding the said protein by applying, from the first nucleotide of the said fragment, the genetic code assigning a specific amino acid to each triplet of nucleotides (AUC=Isoleucine, ACA=Threonine, etc.).

In the invention, the fragment of the pre-mRNA used to determine the cPEP sequence can be selected from the three existing reading frames on the pre-mRNA sequence. In other words, a fragment can be selected from reading frames +1, +2 or +3. In this respect, it is possible that all three reading frames contain the information of a cPEP, just as it is possible that only one of the three reading frames (the +1, the +2 or the +3) or two of the three reading frames (the +1 and the +2, the +1 and the +3, or the +2 and the +3) contain the information of a cPEP.

In the invention, the term “reading frame” refers to the grouping of nucleotides making up a nucleic acid sequence into consecutive triplets (or codons), which follow one another without interruption or overlap.

Generally speaking, cPEPs have a size from 4 to 70 amino acids, in particular from 4 to 41 amino acids, in particular a size from 5 to 40 amino acids, from 7 to 20 amino acids or more particularly a size from 8 to 15 amino acids. Consequently, the sequence of a cPEP corresponds to the translation of a fragment comprising from 4 to 70 triplets of nucleotides, in particular comprising from 4 to 41 triplets of nucleotides, in particular a fragment comprising from 5 to 40 triplets of nucleotides, from 7 to 20 triplets of nucleotides or more particularly a fragment comprising from 8 to 15 triplets of nucleotides.

In other words, the sequence of a cPEP corresponds to the translation into amino acids of a fragment of “3n” nucleotides of the pre-mRNA of the target protein, n being from 4 to 70, in particular n being from 4 to 70, in particular from 4 to 41, in particular from 5 to 40, from 7 to 20 or more particularly from 8 to 15.

For example, if n is equal to 5, the cPEP has a size of 5 amino acids and corresponds to the translation of a fragment of 15 (=3×5) nucleotides. For example, if n is equal to 40, the cPEP is 40 amino acids in size and corresponds to the translation of a fragment of 120 (=3×40) nucleotides. For example, if n is equal to 70, the cPEP is 70 amino acids in size and corresponds to the translation of a fragment of 210 (=3×70) nucleotides. And so on.

The cPEPs are 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids, and correspond respectively to the translation of fragments of 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, 204, 207 or 210 nucleotides.

In one embodiment, the invention relates to the method for preparing and determining a cPEP as described above, wherein said fragment has a size of 3n nucleotides, n being comprised:
from 4 to 41;
from 5 to 40;
from 7 to 20; or
from 8 to 15.

In one embodiment, the invention relates to the method for preparing and determining a cPEP as described above, wherein said peptide has a size selected from: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 and 70 amino acids.

In one embodiment, the invention relates to the method for preparing and determining a cPEP as described above, wherein said cPEP has a size smaller than that of said protein (i.e. the protein, the accumulation of which is modulated by the cPEP).

As indicated above, cPEPs have the ability to specifically modulate the accumulation of a protein without affecting the accumulation of the corresponding mRNA. In other words, the addition of a cPEP to a plant cell does not modify the quantity of mRNA used to express the protein that it has the capacity to regulate, but only the quantity of the said protein.

According to the invention, the term “protein” refers to a sequence of amino acids whose information is encoded by a gene present in the genome of a plant cell. By “gene” is therefore meant, in particular, the nucleic acid sequence necessary for the synthesis of the said protein. A gene also includes more than the nucleotides encoding the amino acid sequence of the protein. For example, a gene includes the DNA sequences required to synthesise a pre-messenger (pre-mRNA), which is then matured by the cellular machinery into a messenger RNA (mRNA). This can then be translated into a protein via the ribosomes.

In view of the above, it is clear that pre-messenger RNA (pre-mRNA) has not undergone splicing and is likely to contain introns, whereas mature messenger RNA (mRNA) may have undergone splicing and contains only exons.

To prepare a cPEP capable of modulating the accumulation of a protein, a fragment of the pre-messenger RNA (pre-mRNA) encoding said protein must be translated to obtain the sequence of said cPEP, which fragment is chosen from a nucleic acid sequence of the pre-mRNA known to be non-coding (e.g. introns, 5′-UTR part or 3′-UTR part).

In the invention, “modulation” of the accumulation of a protein designates either an increase in the accumulation of the said protein (i.e. an increase in the quantity of protein in the plant cell), or a decrease in the accumulation of the said protein (i.e. a decrease in the quantity of protein in the plant cell). In other words, one embodiment of the invention relates to the method for preparing and determining a cPEP as described above, wherein said modulation of the accumulation of said protein induced by said cPEP is:
a reduction in the accumulation of the said protein; or
an increase in the accumulation of the said protein.

The increase and decrease in the accumulation of said protein can be measured and monitored using methods well known to those skilled in the art, such as coupling the protein to a marker using specific expression cassettes, or a Western blot.

In one embodiment, the invention relates to the method for preparing and determining a cPEP as described above, wherein in step e., the amount of protein in the presence of said peptide is greater than the amount of protein in the absence of said peptide. In other words, in the presence of a cPEP promoting increased protein accumulation, translation of the corresponding mRNA is increased, leading to greater production of the protein without altering the amount of said mRNA.

In another embodiment, the invention relates to the method for preparing and determining a cPEP as described above, wherein in step e., the amount of protein in the presence of said peptide is less than the amount of protein in the absence of said peptide. In other words, in the presence of a cPEP favouring a reduction in the accumulation of the protein, the translation of the corresponding mRNA is reduced (inhibited), which leads to a lower production of the protein without the quantity of said mRNA being modified.

In the invention, as the fragment of the pre-mRNA encoding said cPEP is located within one of the nucleic acid sequences of the pre-mRNA deemed non-coding, this means on the one hand that these nucleic acid sequences deemed non-coding are not naturally translated in said plant cell. On the other hand, it also means that the fragment of the pre-mRNA encoding said cPEP is not naturally translated in said plant cell. The existence of a cPEP within the said plant cell is therefore artificial and is the result of human action. To achieve this, it is possible either to artificially introduce said cPEP as such, or to introduce an expression cassette comprising the nucleic acid sequence encoding said cPEP and the means of expressing it in said plant cell.

In the invention, a region of the pre-mRNA that is not naturally translated, also referred to as “non-coding”, corresponds to a region of the pre-mRNA that does not encode any part of the functional protein of the gene, i.e. the protein encoded by the main open reading frame, the accumulation of which is to be modulated.

In the invention, the terms “open reading frame” and “ORF” are equivalent and can be used interchangeably. They correspond to a sequence of nucleotides (nucleic acids) in a DNA or RNA molecule that can potentially encode a peptide or protein: the said open reading frame begins with a START codon (the START codon generally encoding a methionine), followed by a series of codons (each codon encoding an amino acid), and ends with a STOP codon (the STOP codon not being translated).

In a non-limiting way, a non-coding region of a pre-mRNA, i.e. one of the nucleic acid sequences considered to be non-coding, corresponds to:
the 5′UTR region, i.e. the untranslated region located upstream of the main ORF;
the 3′UTR region, i.e. the untranslated region located downstream of the main ORF; or
an intron, i.e. a region of the pre-mRNA deleted by splicing and absent from the mature mRNA.

In one embodiment, the invention relates to the method for preparing and determining a cPEP as described above, one of said nucleic acid sequences deemed to be non-coding being:
the 5′UTR region;
the 3′UTR region; or
an intron.

In another embodiment, the invention relates to the method for preparing and determining a cPEP as described above, one of said nucleic acid sequences deemed to be non-coding being the 5′UTR region or the 3′UTR region. In another embodiment, the invention relates to the method for preparing and determining a cPEP as described above, one of said nucleic acid sequences deemed non-coding being the 5′UTR region. In another embodiment, the invention relates to the method for preparing and determining a cPEP as described above, one of said nucleic acid sequences deemed non-coding being the 3′UTR region. In another embodiment, the invention relates to the method for preparing and determining a cPEP as described above, one of said nucleic acid sequences deemed to be non-coding being an intron.

In the invention, the sequence of a cPEP is determined by (artificially) translating a fragment of the pre-mRNA of the protein, the accumulation of which is to be modulated, said fragment being chosen from the non-coding nucleic acid sequence. Also, the same non-coding nucleic acid sequence in the said plant cell can give different cPEPs depending on the fragment of the pre-mRNA chosen. Furthermore, this same fragment of pre-mRNA can also give different cPEPs depending on the reading frame used to translate it (artificially), i.e. depending on the grouping of the nucleotides of the sequence into consecutive triplets. As already mentioned, translation can be performed in three different reading frames, potentially leading to three different cPEPs.

According to the invention, the “+1” reading frame corresponds to the reading frame determined by the initiation codon of the protein, i.e. the START codon of the open reading frame naturally used for translation of the mRNA, which serves as a reference for identifying the +1 reading frame of the pre-mRNA. In other words, in the case of a fragment of pre-mRNA corresponding to a non-coding region and artificially translated according to the +1 reading frame, the latter corresponds to that used for translation of the mRNA.

The “+2” and “+3” reading frames correspond to shifted reading frames. According to the invention, the “+2” reading frame corresponds to the reading frame shifted by one nucleotide at 3′ (or by two nucleotides at 5′) relative to the open +1 reading frame. According to the invention, the “+3” reading frame corresponds to the reading frame shifted by two nucleotides at 3′ (or one nucleotide at 5′) relative to the +1 open reading frame.

In one embodiment, the invention relates to the method for preparing and determining a cPEP as described above, wherein said fragment is devoid of:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the method for preparing and determining a cPEP as described above, wherein said fragment is devoid of:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

It is therefore understood that a cPEP, according to the above embodiments, comprises either an AUG codon (and no STOP codon), or a STOP codon (and no AUG codon), or neither of these two elements. In the present case, it is up to the person skilled in the art to add, if necessary, the missing element or elements to enable, in a non-limiting manner, either the in vitro production of a cPEP by means, for example, of a microorganism and then its use (in a composition, for example), or the introduction of its sequence and the means of expressing it via a vector into a plant cell or a plant.

In view of the foregoing, it is understood that in another embodiment, the invention relates to the method for preparing and determining a cPEP as described above, said cPEP:
having a size from 4 to 70 amino acids, in particular from 4 to 41 amino acids;
being capable of modulating the accumulation of a protein in a plant cell; and
not being capable of modulating the accumulation of the mRNA encoding said protein,
   said method comprising:
a. a step for determining the nucleic acid sequence of the pre-messenger RNA (pre-mRNA) encoding said protein;
b. a step for determining within this pre-mRNA one of the nucleic acid sequences deemed to be non-coding;
c. a step for determining a non-naturally translated fragment of this nucleic acid sequence deemed to be non-coding, said non-naturally translated fragment having a size of 3n nucleotides, n being from 4 to 70, in particular n being from 4 to 41;
d. a step for producing the peptide encoded by said fragment; and
e. a comparison step:
   between the accumulation of said protein in a plant cell in the presence of said peptide and the accumulation of said protein in a plant cell of the same type in the absence of said peptide; and/or
   between the phenotype of a plant in the presence of said peptide and the phenotype of a plant of the same type in the absence of said peptide,
   wherein:
   a difference in the amount of said protein in the presence of said peptide compared to the amount of said protein in the absence of said peptide; and/or
   a difference in the phenotype in the presence of said peptide compared with the phenotype in the absence of said peptide, indicates that said peptide is a cPEP capable of modulating the accumulation of said protein in a plant cell.

In one embodiment, the invention relates to the method for preparing and determining a cPEP as described above, wherein said fragment comprises:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention therefore relates to the method for preparing and determining a cPEP as described above, wherein said fragment comprises an AUG initiator codon encoding a methionine initiator and lacks a STOP codon chosen from the codons: UAG, UGA and UAA. The invention also relates to the method for preparing and determining a cPEP as described above, wherein said fragment lacks an initiator codon AUG encoding a methionine initiator and comprises a STOP codon selected from codons: UAG, UGA and UAA.

In one embodiment, the invention relates to the method for preparing and determining a cPEP as described above, wherein said fragment comprises:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

Although a cPEP, according to the methods described above, comprises an AUG codon and/or a STOP codon, it should be remembered that the peptide resulting from the in silico translation of the fragment of the pre-mRNA of the protein, the accumulation of which is to be modulated does not exist naturally in a plant cell or a plant.

In one embodiment, the invention relates to the method for preparing and determining a cPEP as described above, wherein the translation of the pre-mRNA fragment is carried out in the same reading frame as the open reading frame of the nucleic acid sequence naturally translated in the said plant cell. In other words, the invention relates to the method for preparing and determining a cPEP as described above, wherein the translation of the fragment of the pre-mRNA is carried out in the reading frame determined by the initiation codon of the open reading frame of the said protein.

In one embodiment, the invention relates to the method for preparing and determining a cPEP as described above, wherein the translation of the fragment of the pre-mRNA is carried out in a reading frame different from the open reading frame of the nucleic acid sequence naturally translated in the said plant cell. In particular, the invention relates to the method for preparing and determining a cPEP as described above, wherein the translation of the pre-mRNA fragment is performed in a reading frame shifted by one nucleotide at 3′ (or by two nucleotides at 5′) relative to the open reading frame of the nucleic acid sequence naturally translated in said plant cell. In particular, the invention also relates to the method for preparing and determining a cPEP as described above, wherein the translation of the pre-mRNA fragment is carried out in a reading frame shifted by two nucleotides 3′ (or one nucleotide 5′) with respect to the open reading frame of the nucleic acid sequence naturally translated in the said plant cell. In other words, the invention relates to the method for preparing and determining a cPEP as described above, wherein the translation of the pre-mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in said plant cell.

In one embodiment, the invention relates to the method for preparing and determining a cPEP as described above, wherein said cPEP is a hydrophobic peptide or a hydrophilic peptide. In particular, the invention relates to the method for preparing and determining a cPEP as described above, wherein said cPEP is a hydrophobic peptide. By “hydrophobic peptide” is meant a peptide, the amino acid sequence of which comprises more than 50% hydrophobic amino acids. More than 50%” means that the amino acid sequence comprises more than 55%, more than 60%, more than 65%, more than 70%, more than 75% or more than 80% hydrophobic amino acids. By “more than 50%” is also meant that the amino acid sequence comprises at least 51%, at least 56%, at least 61%, at least 66%, at least 71%, at least 76% or at least 81% hydrophobic amino acids. By “hydrophobic amino acids” is meant amino acids selected from: alanine (Ala/A), isoleucine (Ile/I), leucine (Leu/L), methionine (Met/M), phenylalanine (Phe/F), tryptophan (Trp/W), tyrosine (Tyr/Y) and valine (Val/V).

In particular, the invention also relates to the method for preparing and determining a cPEP as described above, wherein said cPEP is a hydrophilic peptide. By “hydrophilic peptide” is meant a peptide, the amino acid sequence of which comprises more than 50% hydrophilic amino acids. More than 50% means that the amino acid sequence comprises more than 55%, more than 60%, more than 65%, more than 70%, more than 75% or more than 80% hydrophilic amino acids. By “more than 50%” is also meant that the amino acid sequence comprises at least 51%, at least 56%, at least 61%, at least 66%, at least 71%, at least 76% or at least 81% hydrophilic amino acids. By “hydrophilic amino acids” is meant amino acids selected from: aspartic acid (Asp/D), glutamic acid (Glu/E), arginine (Arg/R), asparagine (Asn/N), glutamine (Gln/Q), histidine (His/H), lysine (Lys/K), serine (Ser/S) and threonine (Thr/T).

A cPEP, the sequence of which corresponds to the translation of a fragment located in a non-coding region of the pre-mRNA has a different sequence to that of a fragment of the protein naturally encoded by the said pre-mRNA.

According to the invention, the sequence of a cPEP is determined by translating a fragment of the pre-mRNA. The same fragment of this pre-mRNA can therefore give different cPEPs depending on the reading frame used for translation (+1, +2 or +3), i.e. depending on how the nucleotides in the sequence are grouped into consecutive triplets.

In a particular embodiment, the invention relates to a method for preparing and determining a cPEP, said cPEP:
having a size from 4 to 70 amino acids, in particular from 4 to 71 amino acids;
being capable of modulating the accumulation of a protein in a plant cell; and
not being capable of modulating the accumulation of the mRNA encoding said protein,
   said method comprising:
a. a step for determining the nucleic acid sequence of the pre-messenger RNA (pre-mRNA) encoding said protein;
b. a step for determining within this pre-mRNA one of the nucleic acid sequences comprising two contiguous parts:
   a part located within a nucleic acid sequence known to be non-coding (i.e. not naturally translated, e.g. intron), and
   a part located within a nucleic acid sequence considered to be coding (i.e. naturally translated, e.g. exon);
c. a step for determining within this nucleic acid sequence comprising two contiguous parts of a fragment thereof, said fragment having a size of 3n nucleotides capable of being translated via the genetic code into a peptide, n being from 4 to 70, in particular n being from 4 to 41;
d. a step for producing said peptide; and
e. a comparison step:
   between the accumulation of said protein in a plant cell in the presence of said peptide and the accumulation of said protein in a plant cell of the same type in the absence of said peptide; and/or
   between the phenotype of a plant in the presence of said peptide and the phenotype of a plant of the same type in the absence of said peptide,
wherein:
   a difference in the amount of said protein in the presence of said peptide compared to the amount of said protein in the absence of said peptide; and/or
      a difference in the phenotype in the presence of said peptide compared with the phenotype in the absence of said peptide,
indicates that said peptide is a cPEP capable of modulating the accumulation of said protein in a plant cell.

According to the invention, a cPEP can be produced by any type of means accessible to the person skilled in the art.

A cPEP can be produced either by synthesis or by recombinant expression in homologous or heterologous systems. The cPEP thus produced can then be introduced into a cell to modulate the accumulation of a target protein.

It is also possible to produce a cPEP directly in the plant cell containing the target protein, by artificially introducing a nucleic acid (such as an expression vector) encoding said cPEP.

In one embodiment, the invention relates to the method for preparing and determining a cPEP as described above, wherein, in step d., the said peptide is produced by peptide synthesis or by recombinant expression.

In one embodiment, the invention relates to the method for preparing and determining a cPEP as described above, wherein, in step d., said peptide is produced using a nucleic acid encoding said peptide introduced into a cell.

In one embodiment, the invention relates to the method for preparing and determining a cPEP as described above, wherein, in step e., the production of said peptide is carried out using a nucleic acid encoding said peptide introduced into said plant cell or into said plant.

In one embodiment, the invention relates to a method for preparing and determining a cPEP as described above, wherein, in step e., said peptide is brought into contact with said plant cell or in said plant.

In one embodiment, the invention relates to the method for preparing and determining a cPEP as described above, wherein, in step e., said peptide is present in said plant cell or in said plant following expression of a nucleic acid encoding said peptide in said plant cell or in said plant.

In view of the foregoing, it is understood that another embodiment of the invention relates to the method for preparing and determining a cPEP as described above, wherein, in step e., the presence of said peptide in said plant cell or in said plant results:
the introduction of a nucleic acid sequence encoding said peptide and comprising the means for expressing it; or
the introduction of an amino acid sequence corresponding to the said peptide.

A cPEP can be used to modulate the accumulation of a protein present naturally (i.e. endogenously) or not (i.e. exogenously) in said plant cell or in said plant.

A “protein naturally present in a plant cell or plant” is an endogenous protein encoded by a gene present in the genome of the plant cell or plant without the need for direct or indirect intervention by a human being.

A “protein which is not naturally present in a plant cell or in a plant” corresponds to an exogenous protein encoded by a nucleic acid sequence present on the genome of the plant cell or the plant which required the intervention of a human being and the use of means known to the person skilled in the art. Such a nucleic acid sequence may come from the same plant species or from another plant species.

In one embodiment, the invention relates to the method for preparing and determining a cPEP as described above, wherein said protein is of endogenous origin in said plant cells or plants used in step e.

In one embodiment, the invention relates to the method for preparing and determining a cPEP as described above, wherein said protein is of exogenous origin in said plant cells or said plants used in step e., said plant cells or said plants used in step e. then comprising a nucleic acid sequence allowing expression of said protein.

In one embodiment, the invention relates to the method for preparing and determining a cPEP as described above, wherein the accumulation of the said protein is determined using a technique chosen from: Western blot, measurement of enzymatic activity, mass spectrometry and translational fusion. In particular, the invention relates to the method for preparing and determining a cPEP as described above, wherein the accumulation of the said protein is determined using a Western blot.

Surprisingly, the Inventors have found that the use of cPEPs makes it possible to modify the phenotypes of a plant that are visible on a macroscopic scale. It is therefore entirely possible to use cPEPs to confirm (or deny) that the peptide determined in steps a., b. and c., and possibly produced in step d., is a cPEP (or not). This is also made possible by the so-called phenotypic comparison alternative implemented in step e. For example, if the peptide determined on the pre-mRNA of a protein involved in the size of the stem of a plant causes an increase, or a decrease, in the size of the stem of a plant treated with the latter compared with an untreated plant, this means that the said peptide is a cPEP capable of modulating the accumulation of the said protein in the size of the stem.

In the invention, the term “plant” refers generally to:
a set of plant cells organised in all or part of a plant, whatever its stage of development (including the plant in the form of a seed or young shoot);
one or more plant organs (e.g. leaves, roots, stems, flowers);
to one or more plant cells; or
a cluster of plant cells (e.g. a callus).

In the invention, the term “phenotype” designates, in a non-limiting manner, macroscopically visible characteristics such as the number of lateral roots, the number of leaves, the size of the stem, the duration of flowering and resistance to stress. In one embodiment, the invention therefore relates to the method for preparing and determining a cPEP as described above, wherein said protein is involved in at least one plant phenotype chosen from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

According to the invention, a protein is “involved in a phenotype” if a change in its accumulation is associated with a change in the said phenotype. In other words, a protein is involved in a phenotype if it is involved in the character(s) corresponding to the said phenotype.

In view of the foregoing, it is understood that an object of the invention is the method for preparing and determining a cPEP as described above, wherein the phenotype observed in step e. is chosen from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

In one embodiment, the invention relates to the method for preparing and determining a cPEP as described above, said cPEP:
having a size from 4 to 70 amino acids, in particular from 4 to 41 amino acids;
being capable of modulating the accumulation of a protein in a plant cell; and
not being capable of modulating the accumulation of the mRNA encoding said protein,
   and wherein said plant cell (i.e. the one wherein it is desired to modulate the accumulation of a protein) belongs to: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vignifera (grapevine) and Zea mays (maize).

In one embodiment, the invention relates to the method for preparing and determining a cPEP as described above, wherein said plant cells or said plants used in step e. belong to: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vignifera (grapevine) and Zea mays (maize).

In one embodiment, the invention relates to the method for preparing and determining a cPEP as described above, wherein said plant cell (i.e. the one wherein it is desired to modulate the accumulation of a protein) is an algal cell.

In one embodiment, the invention relates to the method for preparing and determining a cPEP as described above, wherein said plant cells or said plants used in step e belong to an alga.

In one embodiment, the invention relates to the method for preparing and determining a cPEP as described above, wherein said protein is encoded by a gene selected from: Aae15 (Acyl-activating enzyme 15), Aae16 (AMP-dependent synthetase and ligase family protein), Abcg11 (White-brown complex-like protein), Abdcg34 (ABC transporter G family member 34), Acc1 (Acetyl-CoA Carboxylase), Agb1 (GTP binding protein beta 1), Als (Acetolactate synthase (chloroplastic)), Anac076 (NAC domain-containing protein 76), Apg9 (Autophagy 9), Arlb1 (GTP-binding protein 1), Arr1 (Two-component response regulator ARRI), Arr5 (Two-component response regulator ARR5), Arr6 (Two-component response regulator ARR6), At59 (Pectate lyase family protein), Bak1 (Brassinosteroid insensitive 1-associated receptor kinase 1), Bccp1 (Acetyl-CoA Carboxylase (chloroplastic) subunit 1), Bccp2 (Acetyl-CoA Carboxylase (chloroplastic) subunit 2), Bri1 (Brassinosteroid insensitive 1), Bzo2h3 (bZIP transcription factor family protein), Cesa6 (Cellulose synthase A catalytic subunit 6), Cipk3 (CBL-interacting protein kinase 3), Cks1 (Cyclin-dependent kinases regulatory subunit 1), Cobl8 (COBRA-like protein 8 precursor), Coi1 (Coronatine-insensitive protein 1), Cpk3 (Calcium-dependent protein kinase 3), Crk34 (Cysteine-rich receptor-like protein kinase 34), Cyp705a18 (Cytochrome P450, family 705, subfamily A, polypeptide 18), Cyp71b26 (Cytochrome P450, family 71, subfamily B, polypeptide 26), Cyp78a8 (Cytochrome P450, family 78, subfamily A, polypeptide 8), Cyp97b3 (Cytochrome P450, family 97, subfamily B, polypeptide 3), Dcl1 (Endoribonuclease Dicer homolog 1), Dur3 (Urea-proton symporter DUR3), Ein2 (Ethylene-insensitive protein 2), Emb175 (Pentatricopeptide repeat-containing protein), Emb2726 (Elongation factor Ts family protein), Emb9 (Dihydrofolate synthetase), Epsps (5-enolpyruvylshikimate-3-phosphate (chloroplastic)), Fnr1 (Ferredoxin-NADP[+]-oxidoreductase 1), Fve (Transducin family protein/WD-40 repeat family protein), Ga2ox7 (Gibberellin 2-beta-dioxygenase 7), Gapc (Glyceraldehyde-3-phosphate dehydrogenase), Gcn2 (ABC transporter family protein), Gdi2 (Guanosine nucleotide diphosphate dissociation inhibitor 2), Gln2 (Glutamine synthetase (chloroplastic)), Gsl3 (Callose synthase 2), Hag5 (Histone acetyltransferase of the MYST family 2), Hda18 (Histone deacetylase 18), Hexol (Beta-hexosaminidase 1), Hppd (4-hydroxyphenyl-pyruvate-dioxygenase), Hsl1 (B3 domain-containing transcription repressor VAL2), Iaa31 (Indole-3-acetic acid inducible 31), Iqd28 (IQ-domain 28), Jac1 (J-domain protein required for chloroplast accumulation response 1), Jar1 (Jasmonoyl-L-amino acid synthetase), Kp1 (Kinesin-like protein 1), Lrx2 (Leucine-rich repeat/extensin 2), Mapkkk3 (Mitogen-activated protein kinase kinase kinase 3), Mapkkk5 (Mitogen-activated protein kinase kinase kinase 5), Mfp2 (Multifunctional protein 2), Mrb1 (Transmembrane protein, putative (DUF3537)), Nsp1 (Nodulation signaling pathway 1), Pds (Phytoene desaturase (chloroplastic)), Pen3 (Phosphatidylinositol 3,4,5-trisphosphate 3-phosphatase and protein-tyrosine-phosphatase), Phyb (Phytochrome B), Pif3 (Phytochrome interacting factor 3), Pizza (Brassinosteroid-related acyltransferase 1), Ppox1 (Protoporphyrinogen oxidase (chloroplastic) 1), Ppox2 (Protoporphyrinogen oxidase (chloroplastic) 2), Prp39 (Tetratricopeptide repeat (TPR)-like superfamily protein), PsbA (Photosystem II DI protein), Pskr1 (Phytosulfokin receptor 1), Rd21 (Granulin repeat cysteine protease family protein), Ring1 (RING/U-box superfamily protein), Ros1 (DNA glycosylase/AP lyase ROS1), Rpt4a (26S proteasome regulatory subunit 10B homolog A), Sfr6 (Mediator of RNA polymerase II transcription subunit 16), Shr (Protein SHORT-ROOT), Shy2 (Auxin-responsive protein IAA3), Sk1 (EIN2-like protein, nramp transporter), Sps1 (Sucrose phosphate synthase 2F), Spt (Transcription factor SPATULA), Stn8 (Serine/threonine-protein kinase), Tap46 (PP2A regulatory subunit TAP46), Topp6 (Serine/threonine-protein phosphatase PP1 isozyme 7), TubB6 (Tubulin), TubB8 (Tubulin), Uba1a (RNA-binding (RRM/RBD/RNP motifs) family protein), Vim3 (E3 ubiquitin-protein ligase), Sgr1 (Magnesium dechelatase), Abi5 (Abscisic acid (ABA)-insensitive 5), Hsp101 (Heat shock protein 101), Rh10 (ATP-dependent RNA helicase) and Wus (WUSCHEL).

In particular, the invention relates to the method for preparing and determining a cPEP as described above, wherein said protein is encoded by a gene selected from: Cpk3, Dcl1 and Nsp1. In particular, the invention relates to the method for preparing and determining a cPEP as described above, wherein said protein is encoded by the Cpk3 gene. In particular, the invention relates to the method for preparing and determining a cPEP as described above, wherein the said protein is encoded by the Dcl1 gene. In particular, the invention relates to the method for preparing and determining a cPEP as described above, wherein the said protein is encoded by the Nsp1 gene.

The genes Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexol, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Sk1, Sps1, Spt, Stn8, Tap46, Topp6, TubB6, TubB8, Uba1a, Vim3, Sgr1, Abi5, Hsp101, Rh10 and Wus refer to the proteins indicated in brackets. Of course, the invention also relates to homologous and/or similar genes that may bear different names. For example, in A. thaliana the Gsl3 gene encoding callose synthase 2 is also called Cals2.

In one embodiment, the invention relates to the method for preparing and determining a cPEP as described above, wherein said protein is encoded by an ORF comprising a nucleic acid sequence having at least 80% identity, preferably at least 90% identity, with a sequence chosen from the sequences: SEQ ID NO: 1 (ORF of the Aae15 protein, A. thaliana), SEQ ID NO: 2 (ORF of the Aae16 protein, A. thaliana), SEQ ID NO: 3 (ORF of the abcg11 protein, A. thaliana), SEQ ID NO: 4 (ORF of the Abdcg34 protein, A. thaliana), SEQ ID NO: 5 (ORF of the Acc1 protein, A. thaliana), SEQ ID NO: 6 (ORF of the Agb1 protein, A. thaliana), SEQ ID NO: 7 (ORF of the Als protein, A. thaliana), SEQ ID NO: 8 (ORF of the Anac076 protein, A. thaliana), SEQ ID NO: 9 (ORF of the Apg9 protein, A. thaliana), SEQ ID NO: 10 (ORF of the Arlb1 protein, A. thaliana), SEQ ID NO: 11 (ORF of Arr1 protein, A. thaliana), SEQ ID NO: 12 (ORF of Arr5 protein, A. thaliana), SEQ ID NO: 13 (ORF of Arr6 protein, A. thaliana), SEQ ID NO: 14 (ORF of At59 protein, A. thaliana), SEQ ID NO: 15 (ORF of Bak1 protein, A. thaliana), SEQ ID NO: 16 (ORF of the Beep1 protein, A. thaliana), SEQ ID NO: 17 (ORF of the Bccp2 protein, A. thaliana), SEQ ID NO: 18 (ORF of the Bri1 protein, A. thaliana), SEQ ID NO: 19 (ORF of the Bzo2h3 protein, A. thaliana), SEQ ID NO: 20 (ORF of the Cesa6 protein, A. thaliana), SEQ ID NO: 21 (ORF of the Cipk3 protein, A. thaliana), SEQ ID NO: 22 (ORF of the Cks1 protein, A. thaliana), SEQ ID NO: 23 (ORF of the Cobl8 protein, A. thaliana), SEQ ID NO: 24 (ORF of Coi1 protein, A. thaliana), SEQ ID NO: 25 (ORF of Coi1 protein, A. thaliana), SEQ ID NO: 26 (ORF of Cpk3 protein, A. thaliana), SEQ ID NO: 27 (ORF of Cpk3 protein, A. hypochondriacus), SEQ ID NO: 28 (ORF of Cpk3 protein, B. distachyon), SEQ ID NO: 29 (ORF of Cpk3 protein, B. distachyon), SEQ ID NO: 30 (ORF of Cpk3 protein, G. max), SEQ ID NO: 31 (ORF of Cpk3 protein, G. max), SEQ ID NO: 32 (ORF of Cpk3 protein, G. max), SEQ ID NO: 33 (ORF of Cpk3 protein, G. max), SEQ ID NO: 34 (ORF of Cpk3 protein, O. sativa), SEQ ID NO: 35 (ORF of Cpk3 protein, O. sativa), SEQ ID NO: 36 (ORF of Cpk3 protein, S. lycopersicum), SEQ ID NO: 37 (ORF of Cpk3 protein, Z. mays), SEQ ID NO: 38 (ORF of Cpk3 protein, Z. mays), SEQ ID NO: 39 (ORF of Cpk3 protein, Z. mays), SEQ ID NO: 40 (ORF of Cpk3 protein, B. rapa), SEQ ID NO: 41 (ORF of Cpk3 protein, B. rapa), SEQ ID NO: 42 (ORF of Cpk3 protein, H. vulgare), SEQ ID NO: 43 (ORF of Cpk3 protein, H. vulgare), SEQ ID NO: 44 (ORF of Cpk3 protein, S. tuberosum), SEQ ID NO: 45 (ORF of Cpk3 protein, A. palmeri), SEQ ID NO: 46 (ORF of Cpk3 protein, M. truncatula), SEQ ID NO: 47 (ORF of Cpk3 protein, M. truncatula), SEQ ID NO: 48 (ORF of Cpk3 protein, T. aestivum), SEQ ID NO: 49 (ORF of Cpk3 protein, T. aestivum), SEQ ID NO: 50 (ORF of Cpk3 protein, T. aestivum), SEQ ID NO: 51 (ORF of Cpk3 protein, T. aestivum), SEQ ID NO: 52 (ORF of Cpk3 protein, L. perenne), SEQ ID NO: 53 (ORF of Cpk3 protein, L. perenne), SEQ ID NO: 54 (ORF of Cpk3 protein, L. perenne), SEQ ID NO: 55 (ORF of protein Cpk3, L. perenne), SEQ ID NO: 56 (ORF of protein Crk34, A. thaliana), SEQ ID NO: 57 (ORF of protein Cyp705a18, A. thaliana), SEQ ID NO: 58 (ORF of protein Cyp71b26, A. thaliana), SEQ ID NO: 59 (ORF of protein Cyp78a8, A. thaliana), SEQ ID NO: 60 (ORF of Cyp97b3 protein, A. thaliana), SEQ ID NO: 61 (ORF of Dell protein, A. thaliana), SEQ ID NO: 62 (ORF of Dell protein, A. thaliana), SEQ ID NO: 63 (ORF of Dell protein, A. hypochondriacus), SEQ ID NO: 64 (ORF of Dcl1 protein, B. distachyon), SEQ ID NO: 65 (ORF of Dcl1 protein, G. max), SEQ ID NO: 66 (ORF of Dcl1 protein, G. max), SEQ ID NO: 67 (ORF of Dcl1 protein, O. sativa), SEQ ID NO: 68 (ORF of Dcl1 protein, S. lycopersicum), SEQ ID NO: 69 (ORF of Dcl1 protein, Z. mays), SEQ ID NO: 70 (ORF of Dcl1 protein, B. rapa), SEQ ID NO: 71 (ORF of Dcl1 protein, H. vulgare), SEQ ID NO: 72 (ORF of Dcl1 protein, S. tuberosum), SEQ ID NO: 73 (ORF of Dcl1 protein, M. truncatula), SEQ ID NO: 74 (ORF of Dcl1 protein, T. aestivum), SEQ ID NO: 75 (ORF of Dcl1 protein, T. aestivum), SEQ ID NO: 76 (ORF of the Dcl1 protein, T. aestivum), SEQ ID NO: 77 (ORF of the Dcl1 protein, T. aestivum), SEQ ID NO: 78 (ORF of the Dcl1 protein, L. perenne), SEQ ID NO: 79 (ORF of the Dcl1 protein, L. perenne), SEQ ID NO: 80 (ORF of the Dur3 protein, A. thaliana), SEQ ID NO: 81 (ORF of the Ein2 protein, A. thaliana), SEQ ID NO: 82 (ORF of Emb175 protein, A. thaliana), SEQ ID NO: 83 (ORF of Emb2726 protein, A. thaliana), SEQ ID NO: 84 (ORF of Emb9 protein, A. thaliana), SEQ ID NO: 85 (ORF of Epsps protein, A. thaliana), SEQ ID NO: 86 (ORF of Fnr1 protein, A. thaliana), SEQ ID NO: 87 (ORF of the Fve protein, A. thaliana), SEQ ID NO: 88 (ORF of the Ga2ox7 protein, A. thaliana), SEQ ID NO: 89 (ORF of the Gapc protein, N. benthamiana), SEQ ID NO: 90 (ORF of the Gcn2 protein, A. thaliana), SEQ ID NO: 91 (ORF of the Gdi2 protein, A. thaliana), SEQ ID NO: 92 (ORF of Gln2 protein, A. thaliana), SEQ ID NO: 93 (ORF of Gs13 protein, A. thaliana), SEQ ID NO: 94 (ORF of Hag5 protein, A. thaliana), SEQ ID NO: 95 (ORF of the Hda18 protein, A. thaliana), SEQ ID NO: 96 (ORF of the Hexol protein, A. thaliana), SEQ ID NO: 97 (ORF of the Hppd protein, A. thaliana), SEQ ID NO: 98 (ORF of the Hsl1 protein, A. thaliana), SEQ ID NO: 99 (ORF of the Iaa31 protein, A. thaliana), SEQ ID NO: 100 (ORF of the Iqd28 protein, A. thaliana), SEQ ID NO: 101 (ORF of the Jac1 protein, A. thaliana), SEQ ID NO: 102 (ORF of the Jar1 protein, A. thaliana), SEQ ID NO: 103 (ORF of the Kp1 protein, A. thaliana), SEQ ID NO: 104 (ORF of the Lrx2 protein, A. thaliana), SEQ ID NO: 105 (ORF of the Mapkkk3 protein, A. thaliana), SEQ ID NO: 106 (ORF of the Mapkkk5 protein, A. thaliana), SEQ ID NO: 107 (ORF of the Mfp2 protein, A. thaliana), SEQ ID NO: 108 (ORF of the Mrb1 protein, A. thaliana), SEQ ID NO: 109 (ORF of the Nsp1 protein, M. truncatula), SEQ ID NO: 110 (ORF of the Nsp1 protein, A. thaliana), SEQ ID NO: 111 (ORF of the Nsp1 protein, B. distachyon), SEQ ID NO: 112 (ORF of the Nsp1 protein, G. max), SEQ ID NO: 113 (ORF of the Nsp1 protein, G. max), SEQ ID NO: 114 (ORF of the Nsp1 protein, O. sativa), SEQ ID NO: 115 (ORF of the Nsp1 protein, S. lycopersicum), SEQ ID NO: 116 (ORF of the Nsp1 protein, S. lycopersicum), SEQ ID NO: 117 (ORF of the Nsp1 protein, Z. mays), SEQ ID NO: 118 (ORF of the Nsp1 protein, Z. mays), SEQ ID NO: 119 (ORF of Nsp1 protein, Z. mays), SEQ ID NO: 120 (ORF of Nsp1 protein, Z. mays), SEQ ID NO: 121 (ORF of Nsp1 protein, B. rapa), SEQ ID NO: 122 (ORF of Nsp1 protein, H. vulgare), SEQ ID NO: 123 (ORF of Nsp1 protein, H. vulgare), SEQ ID NO: 124 (ORF of Nsp1 protein, H. vulgare), SEQ ID NO: 125 (ORF of the Nsp1 protein, H. vulgare), SEQ ID NO: 126 (ORF of the Nsp1 protein, H. vulgare), SEQ ID NO: 127 (ORF of the Nsp1 protein, H. vulgare), SEQ ID NO: 128 (ORF of the Nsp1 protein, H. vulgare), SEQ ID NO: 129 (ORF of the Nsp1 protein, S. tuberosum), SEQ ID NO: 130 (ORF of the Nsp1 protein, S. tuberosum), SEQ ID NO: 131 (ORF of the Nsp1 protein, T. aestivum), SEQ ID NO: 132 (ORF of the Nsp1 protein, T. aestivum), SEQ ID NO: 133 (ORF of the Nsp1 protein, L. perenne), SEQ ID NO: 134 (ORF of the Nsp1 protein, L. perenne), SEQ ID NO: 135 (ORF of the Pds protein, A. thaliana), SEQ ID NO: 136 (ORF of the Pen3 protein, A. thaliana), SEQ ID NO: 137 (ORF of the Phyb protein, A. thaliana), SEQ ID NO: 138 (ORF of the Pif3 protein, A. thaliana), SEQ ID NO: 139 (ORF of the Pizza protein, A. thaliana), SEQ ID NO: 140 (ORF of the Ppox1 protein, A. thaliana), SEQ ID NO: 141 (ORF of the Ppox2 protein, A. thaliana), SEQ ID NO: 142 (ORF of the Prp39 protein, A. thaliana), SEQ ID NO: 143 (ORF of the PsbA protein, A. thaliana), SEQ ID NO: 144 (ORF of the Pskr1 protein, A. thaliana), SEQ ID NO: 145 (ORF of the Rd21 protein, A. thaliana), SEQ ID NO: 146 (ORF of the Ring1 protein, A. thaliana), SEQ ID NO: 147 (ORF of the Ros1 protein, A. thaliana), SEQ ID NO: 148 (ORF of the Rpt4a protein, A. thaliana), SEQ ID NO: 149 (ORF of the Sfr6 protein, A. thaliana), SEQ ID NO: 150 (ORF of the Shr protein, A. thaliana), SEQ ID NO: 151 (ORF of the Shy2 protein, A. thaliana), SEQ ID NO: 152 (ORF of the Sk1 protein, M. truncatula), SEQ ID NO: 153 (ORF of the Sps1 protein, A. thaliana), SEQ ID NO: 154 (ORF of the Spt protein, A. thaliana), SEQ ID NO: 155 (ORF of the Stn8 protein, A. thaliana), SEQ ID NO: 156 (ORF of the Tap46 protein, A. thaliana), SEQ ID NO: 157 (ORF of the Topp6 protein, A. thaliana), SEQ ID NO: 158 (ORF of the TubB6 protein, A. thaliana), SEQ ID NO: 159 (ORF of the TubB8 protein, A. thaliana), SEQ ID NO: 160 (ORF of the Uba1a protein, A. thaliana), SEQ ID NO: 161 (ORF of the Vim3 protein, A. thaliana), SEQ ID NO: 171 (ORF of the Sgr1 protein, A. thaliana), SEQ ID NO: 172 (ORF of the Abi5 protein, A. thaliana), SEQ ID NO: 173 (ORF of the Hsp101 protein, A. thaliana), SEQ ID NO: 174 (ORF of the Rh10 protein, M. truncatula) and SEQ ID NO: 175 (ORF of the Wus protein, A. thaliana).

In one embodiment, the invention relates to the method for preparing and determining a cPEP as described above, wherein said protein is encoded by an ORF comprising a nucleic acid sequence having at least 80% identity, preferably at least 90% identity, with a sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method for preparing and determining a cPEP as described above, wherein the said protein is encoded by a gene comprising a nucleic acid sequence having at least 80% identity, preferably at least 90% identity, with a sequence chosen from the sequences: SEQ ID NOs: 26 to 55 (Cpk3). In particular, the invention relates to the method for preparing and determining a cPEP as described above, wherein said protein is encoded by a gene comprising a nucleic acid sequence having at least 80% identity, preferably at least 90% identity, with a sequence chosen from the sequences: SEQ ID NOs: 61 to 79 (Dcl1). In particular, the invention relates to the method for preparing and determining a cPEP as described above, wherein said protein is encoded by a gene comprising a nucleic acid sequence having at least 80% identity, preferably at least 90% identity, with a sequence chosen from the sequences: SEQ ID NOs: 109 to 134 (Nsp1). “Percentage identity” between two nucleic acid (or amino acid) sequences refers to the percentage of identical nucleotides (or amino acid residues) between the two sequences to be compared, obtained after the best alignment. This percentage is purely statistical and the differences between the two sequences are randomly distributed over the entire length of the sequences. The best alignment (or optimal alignment) is the alignment for which the percentage of identity between the two sequences to be compared, as calculated below, is the highest. Sequence comparisons between two nucleic acid (or amino acid) sequences are traditionally performed by comparing these sequences after they have been optimally aligned, said comparison being performed by segment or by comparison window to identify and compare local regions of sequence similarity. The optimal alignment of sequences for comparison can be carried out manually or using algorithms and software available to those skilled in the art, for example, the BLAST platform or the MatGat program (Campanella, Bitincka and Smalley, 2003).

The percentage of identity between two sequences is determined by comparing these two optimally aligned sequences by comparison window wherein the region of the sequence to be compared may comprise additions or deletions relative to the reference sequence for optimal alignment between these two sequences. The percentage identity is calculated by determining the number of identical positions for which the nucleotide (or amino acid) is identical between the two sequences, dividing this number of identical positions by the total number of positions in the comparison window and multiplying the result obtained by 100.

For the purposes of the invention, it is understood that sequences exhibiting “at least 80% identity” with a reference sequence may in particular exhibit at least 80%, 81%, 82%, 83%, 84%, 85%, 86%, 87%, 88%, 89%, 90%, 91%, 92%, 93%, 94%, 95%, 96%, 97%, 98%, 99% or 100% identity with said reference sequence.

In one embodiment, the invention relates to the method for preparing and determining a cPEP as described above, wherein the said protein is encoded by a gene comprising a nucleic acid sequence chosen from the sequences: SEQ ID NOs: 1 to 161 and 171 to 175. In one embodiment, the invention also relates to the method for preparing and determining a cPEP as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method for preparing and determining a cPEP as described above, wherein said protein is encoded by a gene comprising one selected from the sequences: SEQ ID NOs: 26 to 55 (Cpk3). In particular, the invention relates to the method for preparing and determining a cPEP as described above, wherein said protein is encoded by a gene comprising one selected from the sequences: SEQ ID NOs: 61 to 79 (Dcl1). In particular, the invention relates to the method for preparing and determining a cPEP as described above, wherein said protein is encoded by a gene comprising one selected from the sequences: SEQ ID NOs: 109 to 134 (Nsp1).

In another embodiment, the invention relates to the method for preparing and determining a cPEP as described above, wherein the sequence of said peptide is chosen from the sequences: SEQ ID NO: 162 (NSP1-5′UTR-11), SEQ ID NO: 163 (NSP1-5′UTR-5) and SEQ ID NO: 164 (NSP1-3′UTR).

In a second aspect, the invention relates to a cPEP as obtained by implementing the method as described above. According to this same aspect, the invention also covers a cPEP, of 4 to 70 amino acids, in particular of 4 to 41 amino acids, the amino acid sequence of which corresponds to the translation via the genetic code of a fragment of a nucleic acid sequence deemed to be non-coding present on a pre-mRNA of a protein, said cPEP being capable of modulating the accumulation of said protein in a plant cell and not being capable of modulating the accumulation of the mRNA encoding said protein.

In one embodiment, the invention relates to cPEP as previously described, said fragment having a size of 3n nucleotides, n being comprised:
from 4 to 41;
from 5 to 40;
from 7 to 20; or
from 8 to 15.

In other words, the invention relates to the cPEP as described above, said cPEP comprising 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

In particular, the invention relates to the cPEP as described above, said cPEP comprising from 5 to 40 amino acids. In particular, the invention relates to the cPEP as described above, said cPEP comprising from 7 to 20 amino acids. In particular, the invention also relates to the cPEP as described above, said cPEP comprising from 8 to 15 amino acids.

In one embodiment, the invention relates to the cPEP as described above, wherein the size of said cPEP is smaller than that of said protein.

In the invention, the nucleic acid sequence carrying the cPEP peptide information is chosen from a region that is not translated naturally in the said plant cell. Non-limitingly, it corresponds to:
the 5′UTR region, i.e. the untranslated region located upstream of the main ORF;
the 3′UTR region, i.e. the untranslated region located downstream of the main ORF; or
an intron, i.e. a region of the pre-mRNA deleted by splicing and absent from the mature mRNA.

In one embodiment, the invention relates to the cPEP as described above, said nucleic acid sequence deemed non-coding being:
the 5′UTR region;
the 3′UTR region; or
an intron.

In another embodiment, the invention relates to the cPEP as described above, one of said nucleic acid sequences deemed non-coding being the 5′UTR region or the 3′UTR region. In another embodiment, the invention relates to the cPEP as described above, one of said nucleic acid sequences deemed non-coding being the 5′UTR region. In another embodiment, the invention relates to the cPEP as described above, one of said nucleic acid sequences deemed non-coding being the 3′UTR region. In another embodiment, the invention relates to the cPEP as described above, one of said nucleic acid sequences deemed to be non-coding being an intron.

In one embodiment, the invention relates to the cPEP as described above, said fragment lacking:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the cPEP as described above, said fragment lacking:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the cPEP as described above, said fragment comprising:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention therefore relates to the cPEP as described above, said fragment comprising an AUG initiator codon encoding a methionine initiator and lacking a STOP codon chosen from the codons: UAG, UGA and UAA. The invention also relates to the cPEP as described above, said fragment lacking an initiator codon AUG encoding a methionine initiator and comprising a STOP codon chosen from codons: UAG, UGA and UAA.

In one embodiment, the invention relates to the cPEP as described above, said fragment comprising:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the cPEP as described above, wherein the translation of the pre-mRNA fragment is carried out in the same reading frame as the open reading frame of the nucleic acid sequence naturally translated in said plant cell. In other words, the invention relates to the cPEP as described above, wherein the translation of the pre-mRNA fragment is carried out in the reading frame determined by the initiation codon of the open reading frame of the said protein.

In one embodiment, the invention relates to the cPEP as described above, wherein the translation of the pre-mRNA fragment is carried out in a reading frame different from the open reading frame of the nucleic acid sequence naturally translated in said plant cell. In particular, the invention relates to the cPEP as described above, wherein the translation of the pre-mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the cPEP as described above, wherein the translation of the pre-mRNA fragment is carried out in a reading frame shifted by two nucleotides 3′ (or one nucleotide 5′) with respect to the open reading frame of the nucleic acid sequence naturally translated in said plant cell. In other words, the invention relates to the cPEP as described above, wherein the translation of the pre-mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in said plant cell.

In one embodiment, the invention relates to a cPEP, of 4 to 70 amino acids, in particular of 4 to 41 amino acids, the amino acid sequence of which corresponds to the translation via the genetic code of a fragment of a nucleic acid sequence of a pre-mRNA of a protein, said nucleic acid sequence comprising two contiguous parts:
a part located within a nucleic acid sequence known to be non-coding (i.e. not naturally translated, e.g. intron); and
a part located within a nucleic acid sequence considered to be coding (i.e. naturally translated, e.g. exon),
   said fragment having a size of 3n nucleotides, n being from 4 to 70, in particular n being from 4 to 41, and said cPEP being capable of modulating the accumulation of said protein in a plant cell and not being capable of modulating the accumulation of the mRNA encoding said protein.

In one embodiment, the invention relates to cPEP as previously described, wherein said cPEP is capable of increasing the accumulation of said protein in said plant cell.

In one embodiment, the invention relates to the cPEP as described above, wherein said cPEP is capable of decreasing the accumulation of said protein in said plant cell.

In one embodiment, the invention relates to the cPEP as described above, wherein said cPEP is a synthetic peptide.

In one embodiment, the invention relates to the cPEP as described above, wherein said cPEP is an isolated peptide.

In one embodiment, the invention relates to cPEP as described previously, wherein said cPEP is a recombinant peptide.

In one embodiment, the invention relates to the cPEP as described above, said cPEP being a hydrophobic peptide or a hydrophilic peptide.

In one embodiment, the invention relates to the cPEP as described above, wherein said protein is naturally present in said plant cell.

In one embodiment, the invention relates to the cPEP as described above, wherein said protein is not naturally present in said plant cell. In particular, the invention relates to the cPEP as described above, wherein said protein is encoded by a transgene artificially introduced into said plant cell. In particular, the invention relates to the cPEP as described above, wherein said protein is encoded by a vector artificially introduced into said plant cell.

In one embodiment, the invention relates to the cPEP as described above, wherein said plant cell belongs to: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (trefoil), Medicago sativa (alfalfa), Medicago truncatula (lucerne), Nicotiana benthamiana (tobacco), Oryza sativa (rice), Pisum sativum (pea), Raphanus sativus, Solanum lycopersicum (tomato), Solanum melongena (aubergine), Solanum tuberosum (potato), Thellungiella halophila, Theobroma cacao, Triticum spp. (wheat), Vitis vignifera (grapevine) and Zea mays (maize).

In one embodiment, the invention relates to the cPEP as described above, wherein said plant cell is an algal cell.

In one embodiment, the invention relates to the cPEP as described above, wherein said protein is encoded by a gene selected from: Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexol, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Sk1, Sps1, Spt, Stn8, Tap46, Topp6, TubB6, TubB8, Uba1a, Vim3, Sgr1, Abi5, Hsp101, Rh10 and Wus.

In particular, the invention relates to the cPEP as described above, wherein the said protein is encoded by a gene chosen from the following genes: Cpk3, Dcl1 and Nsp1. In particular, the invention relates to the cPEP as described above, wherein the said protein is encoded by the Cpk3 gene. In particular, the invention relates to the cPEP as described above, wherein the said protein is encoded by the Dcl1 gene. In particular, the invention relates to the cPEP as described above, wherein said protein is encoded by the Nsp1 gene.

In one embodiment, the invention relates to the cPEP as described above, wherein said protein is encoded by a gene comprising a nucleic acid sequence having at least 80% identity, preferably at least 90% identity, with a sequence chosen from the sequences: SEQ ID NOs: 1 to 161 and 171 to 175.

In particular, the invention relates to the cPEP as described above, wherein the said protein is encoded by a gene comprising a nucleic acid sequence having at least 80% identity, preferably at least 90% identity, with a sequence chosen from the sequences: SEQ ID NOs: 26 to 55 (Cpk3), SEQ ID NOs: 61 to 79 (Dcl1) and SEQ ID NOs: 109 to 134 (Nsp1). In particular, the invention also relates to the cPEP as described above, wherein the said protein is encoded by a gene comprising a nucleic acid sequence having at least 80% identity, preferably at least 90% identity, with a sequence chosen from the sequences: SEQ ID NOs: 26 to 55 (Cpk3). In particular, the invention also relates to the cPEP as described above, wherein the said protein is encoded by a gene comprising a nucleic acid sequence having at least 80% identity, preferably at least 90% identity, with a sequence chosen from the sequences: SEQ ID NOs: 61 to 79 (Dcl1). In particular, the invention also relates to the cPEP as described above, wherein the said protein is encoded by a gene comprising a nucleic acid sequence exhibiting at least 80% identity, preferably at least 90% identity, with a sequence chosen from the sequences: SEQ ID NOs: 109 to 134 (Nsp1).

In one embodiment, the invention relates to the cPEP as described above, wherein the said protein is encoded by a gene comprising a nucleic acid sequence chosen from the sequences: SEQ ID NOs: 1 to 161 and 171 to 175. The invention relates in particular to the cPEP as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the cPEP as described above, wherein said protein is encoded by a gene comprising one selected from the sequences: SEQ ID NOs: 26 to 55 (Cpk3). In particular, the invention relates to the cPEP as described above, wherein said protein is encoded by a gene comprising one selected from the sequences: SEQ ID NOs: 61 to 79 (Dcl1). In particular, the invention relates to the cPEP as described above, wherein said protein is encoded by a gene comprising one selected from the sequences: SEQ ID NOs: 109 to 134 (Nsp1).

In another embodiment, the invention relates to the cPEP as described above, wherein the sequence of said peptide is chosen from the sequences: SEQ ID NOs: 162 to 164.

In one embodiment, the invention relates to the cPEP as described above, wherein said protein is involved in at least one plant phenotype selected from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

In the invention, a cPEP can be fused or linked to one or more molecules that facilitate entry of the cPEP into the cell. These molecules include penetrating peptides (Numata, K., et al. Library screening of cell-penetrating peptide for BY-2 cells, leaves of Arabidopsis, tobacco, tomato, poplar, and rice callus. Sci Rep 8, 10966 (2018)) and palmitic acid. Penetrating peptide” (hereinafter CPP) refers to small peptides that penetrate cellular lipid bilayers or destabilise cell membranes. CPPs can be classified into three groups: cationic, amphipathic and hydrophobic. In particular:
Cationic CPPs contain many positively charged amino acids, such as lysine (Lys) and arginine (Arg);
amphipathic CPPs are generally composed of an alternating sequence of polar and non-polar amino acids; and
hydrophobic CPPs are composed of non-polar amino acids with relatively low net charges.

In one embodiment, the invention relates to the cPEP as described above, said cPEP being fused to a peptide facilitating its entry into the plant cell. In particular, the invention relates to the cPEP as described above, said cPEP being fused to a penetrating peptide.

In one embodiment, the invention relates to the cPEP as described above, said cPEP being fused at the N-terminus or at the C-terminus with said peptide facilitating its entry into the plant cell. In particular, the invention relates to the cPEP as described above, said cPEP being fused at the N-terminus or at the C-terminus with said penetrating peptide.

In one embodiment, the invention relates to the cPEP as described above, said cPEP being fused with:
TAT peptide (SEQ ID NO: 170);
penetratin;
a polyhistidine peptide (in particular a peptide with at least 4 histidine residues); or
a polyarginine peptide (in particular a peptide with 4 arginine residues).

In one embodiment, the invention relates to the cPEP as described above, said cPEP being linked to one or more palmitic acid molecules.

In one embodiment, the invention relates to the cPEP as described above, said cPEP being linked at the N-terminus or at the C-terminus to one or more palmitic acid molecules.

On this point, it should be noted that the quantity of cPEP required to modulate the accumulation of a protein may vary depending on whether or not the cPEP is modified with one of the molecules facilitating its cellular penetration.

In a third aspect, the invention relates to a nucleic acid encoding a cPEP as described above. According to this same aspect, the invention also relates to a nucleic acid of 3n nucleotides, which nucleic acid corresponds to a fragment of a nucleic acid sequence deemed to be non-coding present on a pre-mRNA of a protein.

In one embodiment, the invention relates to cPEP nucleic acid as described above, said fragment lacking:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to cPEP nucleic acid as described above, said fragment lacking:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to cPEP nucleic acid as described above, said fragment comprising:
an AUG initiator codon encoding a methionine initiator; or
a STOP codon chosen from: UAG, UGA and UAA,
   and wherein said fragment is selected from:
either in the same reading frame as the open reading frame encoding the said protein;
or in a reading frame shifted by one or two nucleotides relative to the open reading frame encoding said protein.

In one embodiment, the invention therefore relates to the nucleic acid as described above, said fragment comprising an AUG initiator codon encoding a methionine initiator and lacking a STOP codon chosen from the codons: UAG, UGA and UAA. The invention also relates to the nucleic acid as described above, the said fragment lacking an initiator codon AUG encoding a methionine initiator and comprising a STOP codon chosen from the codons: UAG, UGA and UAA.

In one embodiment, the invention relates to cPEP nucleic acid as described above, said fragment comprising:
an AUG initiator codon encoding a methionine initiator; and
a STOP codon chosen from: UAG, UGA and UAA,
   and wherein said fragment is selected from:
either in the same reading frame as the open reading frame encoding the said protein;
or in a reading frame shifted by one or two nucleotides relative to the open reading frame encoding said protein.

In one embodiment, the invention relates to the cPEP nucleic acid as described above, wherein the translation of the pre-mRNA fragment is carried out in the same reading frame as the open reading frame of the nucleic acid sequence naturally translated in said plant cell. In other words, the invention relates to the cPEP nucleic acid as described above, wherein the translation of the fragment of the pre-mRNA is carried out in the reading frame determined by the initiation codon of the open reading frame of the said protein.

In one embodiment, the invention relates to the cPEP nucleic acid as described above, wherein the translation of the pre-mRNA fragment is carried out in a reading frame different from the open reading frame of the nucleic acid sequence naturally translated in said plant cell. In particular, the invention relates to the cPEP nucleic acid as described above, wherein the translation of the pre-mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the cPEP nucleic acid as described above, wherein the translation of the pre-mRNA fragment is carried out in a reading frame shifted by two nucleotides 3′ (or one nucleotide 5′) with respect to the open reading frame of the nucleic acid sequence naturally translated in said plant cell. In other words, the invention relates to the cPEP nucleic acid as described above, wherein the translation of the pre-mRNA fragment is carried out in a reading frame shifted by one (or two nucleotides at 5′) or two nucleotides at 3′ (or one nucleotide at 5′) with respect to the initiation codon of the open reading frame of the nucleic acid sequence naturally translated in said plant cell.

In one embodiment, the invention relates to a nucleic acid of 3n nucleotides, which nucleic acid corresponds to a fragment of a nucleic acid sequence of a pre-mRNA of a protein, said nucleic acid sequence comprising two contiguous parts:
a part located within a nucleic acid sequence known to be non-coding (i.e. not naturally translated, e.g. intron); and
a part located within a nucleic acid sequence considered to be coding (i.e. naturally translated, e.g. exon).

In particular, the invention relates to nucleic acid as described above, where n is:
from 4 to 70;
from 4 to 41;
from 5 to 40;
from 7 to 20; or
from 8 to 15.

In another aspect, the invention relates to a composition comprising a cPEP as described above as active substance.

In another embodiment, the invention relates to a composition comprising a cPEP as active substance, said cPEP:
having a size from 4 to 70 amino acids, in particular from 4 to 41 amino acids, the amino acid sequence of which corresponds to the translation via the genetic code of a fragment of a nucleic acid sequence deemed to be non-coding present on a pre-mRNA of a protein, said fragment having a size of 3n nucleotides, n being from 4 to 70, in particular n being from 4 to 41; and
being capable of modulating the accumulation of said protein in the plant cell and not being capable of modulating the accumulation of the mRNA encoding said protein.

In one embodiment, the invention relates to the composition as described above, wherein said fragment is devoid of:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the composition as described above, wherein said fragment is devoid of:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the composition as previously described, wherein said fragment comprises:
an AUG initiator codon encoding a methionine initiator; or
a STOP codon chosen from: UAG, UGA and UAA,
   and wherein said fragment is selected from:
either in the same reading frame as the open reading frame encoding the said protein;
or in a reading frame shifted by one or two nucleotides relative to the open reading frame encoding said protein.

In one embodiment, the invention therefore relates to the composition as described above, wherein the said fragment comprises an AUG initiator codon encoding a methionine initiator and lacks a STOP codon chosen from the codons: UAG, UGA and UAA. The invention also relates to the composition as described above, wherein the said fragment lacks an initiator codon AUG encoding a methionine initiator and comprises a STOP codon chosen from codons: UAG, UGA and UAA.

In one embodiment, the invention relates to the composition as previously described, wherein said fragment comprises:
an AUG initiator codon encoding a methionine initiator; and
a STOP codon chosen from: UAG, UGA and UAA,
   and wherein said fragment is selected from:
either in the same reading frame as the open reading frame encoding the said protein;
or in a reading frame shifted by one or two nucleotides relative to the open reading frame encoding said protein.

In one embodiment, the invention relates to the composition as described above, wherein the translation of the pre-mRNA fragment is carried out in the same reading frame as the open reading frame of the nucleic acid sequence naturally translated in the said plant cell. In other words, the invention relates to the composition as described above, wherein the translation of the pre-mRNA fragment is carried out in the reading frame determined by the initiation codon of the open reading frame of the said protein.

In one embodiment, the invention relates to the composition as described above, wherein the translation of the pre-mRNA fragment is carried out in a reading frame different from the open reading frame of the nucleic acid sequence naturally translated in the said plant cell. In particular, the invention relates to the composition as described above, wherein the translation of the pre-mRNA fragment is carried out in a reading frame shifted by one nucleotide at 3′ (or by two nucleotides at 5′) with respect to the open reading frame of the nucleic acid sequence naturally translated in the said plant cell. In particular, the invention also relates to the composition as described above, wherein the translation of the pre-mRNA fragment is carried out in a reading frame shifted by two nucleotides 3′ (or one nucleotide 5′) with respect to the open reading frame of the nucleic acid sequence naturally translated in the said plant cell. In other words, the invention relates to the composition as described above, wherein the translation of the pre-mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in the said plant cell.

In one embodiment, the invention relates to a composition comprising a cPEP as active substance, said cPEP:
having a size from 4 to 70 amino acids, in particular from 4 to 41 amino acids, the amino acid sequence of which corresponds to the translation via the genetic code of a fragment of a nucleic acid sequence of a pre-mRNA of a protein, said fragment having a size of 3n nucleotides, n being from 4 to 70, in particular n being from 4 to 41, and said nucleic acid sequence comprising two contiguous parts:
   a part located within a nucleic acid sequence known to be non-coding (i.e. not naturally translated, e.g. intron); and
   a part located within a nucleic acid sequence known to be coding (i.e. naturally translated, e.g. exon); and
being capable of modulating the accumulation of said protein in the plant cell and not being capable of modulating the accumulation of the mRNA encoding said protein.

In one embodiment, the invention relates to the composition as described above, wherein the said cPEP is at a concentration from 10⁻⁹ M to 10⁻³ M. On this point, it should be noted on the one hand that the composition of the invention does not exist in the natural state, and this is all the more true as such a concentration of cPEP cannot exist within a plant cell. Furthermore, by “concentration from 10⁻⁹ M to 10⁻³ M”, is meant that the concentration of cPEP can be from 10⁻⁹ to 10⁻⁴ M, from 10⁻⁸ to 10⁻⁴ M, from 10⁻⁹ to 10⁻⁵ M, from 10⁻⁸ to 10⁻⁵ M, or from 5 μM to 500 μM, from 30 μM to 70 μM, or even 50 μM.

In particular, the invention relates to the composition as described above, wherein the said cPEP is at a concentration from 10⁻⁹ to 10⁻⁴ M, from 10⁻⁸ to 10⁻⁴ M, from 10⁻⁹ to 10⁻⁵ M or from 10⁻⁸ to 10⁻⁵ M. In particular, the invention relates to the composition as described above, wherein the said cPEP is at a concentration from 5 μM to 500 μM or from 30 μM to 70 μM.

In particular, the invention relates to the composition as described above, wherein the said cPEP is at a concentration of 50 μM. Alternatively, this concentration may be 10⁻⁹ M, 10⁻⁸ M, 10⁻⁷ M, 10⁻⁶ M, 10⁻⁵ M or 10⁻⁴ M.

In view of the above, it is understood that the invention also relates to the composition as described above comprising a cPEP as active substance, said cPEP:
having a size from 4 to 70 amino acids, in particular from 4 to 41 amino acids, the amino acid sequence of which corresponds to the translation via the genetic code of a fragment of a nucleic acid sequence deemed to be non-coding present on a pre-mRNA of a protein;
being capable of modulating the accumulation of said protein in the plant cell and not being capable of modulating the accumulation of the mRNA encoding said protein; and
being in particular at a concentration ranging from 5 μM to 500 μM or from 30 μM to 70 μM, or being in particular at a concentration of 50 μM.

It should be noted that “composition comprising a cPEP” means that the composition of the invention comprises at least one cPEP. In other words, a mixture of cPEPs is conceivable, said cPEPs being able to target the same protein or several proteins depending on the nucleic acid fragment from which they are derived. In this respect, the concentrations mentioned above relate either to the mixture of cPEPs as such, or to each of the cPEPs in the said mixture, the said cPEPs being able to be at the same concentration or being able to be at different concentrations from those mentioned above.

In one embodiment, the invention relates to the composition as described above, said composition being a phytopharmaceutical composition, a herbicidal composition or a coating composition, in particular said coating composition further comprising at least one fixing agent.

In particular, the invention relates to the composition as described above, said composition being a phytopharmaceutical composition. In particular, the invention relates to the composition as described above, the said composition being a herbicidal composition. In particular, the invention relates to the composition as described above, said composition being a coating composition. Preferably, the invention relates to the composition as described above, the said composition being a coating composition additionally comprising at least one fixing agent.

In one embodiment, the invention relates to the composition as described above, said composition further comprising at least one solvent. Preferably, the said solvent is chosen from: acetone, acetonitrile, acetic acid, formic acid, dimethyl adipate, benzyl acetate, bi-butyl carbonate, dimethyl sulphoxide (DMSO), water, dimethyl glutarate, ammonium hydroxide, iso-butanol, iso-propanol, diethyl hexyl lactate, light aromatic naphtha solvent, heavy aromatic naphtha solvent, diethyl succinate and mixtures thereof (e.g. mixture [water; acetic acid]).g. mixture [water; acetic acid]; [acetonitrile; acetic acid], [water, acetonitrile; acetic acid], [water; DMSO], [water; acetonitrile] or [water; ammonium hydroxide]).

The solubility properties of cPEPs are determined in particular by their amino acid composition. Hydrophilic cPEPs can be solubilised and packaged in aqueous solutions, such as water. Hydrophobic cPEPs can be solubilised and packaged in solvents, such as organic solvents.

For treatment of plants with cPEPs, the organic solvents are non-toxic for the plants in small quantities, i.e. they have no deleterious effect on the development of the plant. By way of example, the organic solvents may be those mentioned above and in particular selected from acetonitrile and acetic acid.

As mentioned above, cPEPs can also be solubilised and packaged in solvent mixtures such as, for example, an organic solvent mixture [acetonitrile; acetic acid], a mixture [water; DMSO] in a volume:volume ratio from 99:1 to 1:99, a mixture [water; acetonitrile] in a volume:volume ratio from 99:1 to 1:99 or a mixture [water; ammonium hydroxide]:volume ratio from 99:1 to 1:99, a [water; acetonitrile] mixture in a volume:volume ratio from 99:1 to 1:99 or a [water; ammonium hydroxide] mixture in a volume:volume ratio from 99:1 to 99.9:0.1. The cPEPs can also be solubilised in a solution comprising 50% acetonitrile, 10% acetic acid and 40% water (v/v/v).

In one embodiment, the invention relates to the composition as described above, said composition further comprising at least one diluent.

In one embodiment, the invention relates to the composition as described above, said composition further comprising at least one adjuvant.

In one embodiment, the invention relates to the composition as described above, said composition further comprising at least one fixing agent.

By “fixing agent” is meant a chemical or natural agent which enables the composition of the invention to adhere to a plant seed so as to coat the said plant seed. It also means a substance that makes it possible to apply and hold the active substance(s) on the seed. Available fixing agents include carboxymethyl cellulose (CMC) and gum arabic. In addition, and without limitation, a fixing agent can include organic solvents, water, dispersants, emulsifiers, surfactants, wetting agents and dyes.

In one embodiment, the invention relates to the composition as described above, said composition further comprising at least one plant nutrient. In particular, the invention relates to the composition as described above, the said composition additionally comprising at least one fixing agent and at least one plant nutrient.

By “plant nutrient” is meant an element assimilated by the plant to enable it to develop. By no means restrictive, a plant nutrient can be chosen from: nitrogen, phosphorus, potassium, calcium, magnesium, sulphur, manganese, iron, copper, boron, zinc, molybdenum and mixtures thereof.

In view of the foregoing, it is understood that another aspect of the invention relates to a coated seed comprising a plant seed, said plant seed being coated with a coating composition as previously described.

The coating can be produced using methods conventionally used in the food industry and can be obtained by using a material capable of disintegrating in a solvent or in the earth, such as a binder or clay.

According to the invention, the coating can be used to confer particular properties on a seed in combination with a cPEP, such as improved growth or resistance to certain biotic or abiotic stresses.

In one embodiment, the invention relates to the coated seed as described above, wherein said plant seed belongs to: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (trefoil), Medicago sativa (alfalfa), Medicago truncatula (lucerne), Nicotiana benthamiana (tobacco), Oryza sativa (rice), Pisum sativum (pea), Raphanus sativus, Solanum lycopersicum (tomato), Solanum melongena (aubergine), Solanum tuberosum (potato), Thellungiella halophila, Theobroma cacao, Triticum spp. (wheat), Vitis vignifera (grapevine) and Zea mays (maize).

In one embodiment, the invention relates to coated seed as described above, said seed being treated by dipping in a cPEP-containing composition. During dipping, the seed is then totally or partially immersed in a composition containing a cPEP.

In another aspect, the invention relates to a use of a cPEP as a plant protection agent to modulate the accumulation of a protein in a plant cell, said cPEP having a size from 4 to 70 amino acids, in particular from 4 to 41 amino acids, the amino acid sequence of which corresponds to the translation via the genetic code of a fragment of a nucleic acid sequence deemed to be non-coding present on a pre-mRNA of a protein, said cPEP being capable of modulating the accumulation of said protein in the plant cell and not being capable of modulating the accumulation of the mRNA encoding said protein.

In one embodiment, the invention relates to the use of a cPEP as described above, wherein said fragment lacks:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the use of a cPEP as described above, wherein said fragment lacks:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the use of a cPEP as described above, wherein said fragment comprises:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention therefore relates to the use of a cPEP as described above, wherein said fragment comprises an AUG initiator codon encoding a methionine initiator and lacks a STOP codon selected from the codons: UAG, UGA and UAA. The invention also relates to the use of a cPEP as described above, wherein said fragment lacks an initiator codon AUG encoding a methionine initiator and comprises a STOP codon selected from codons: UAG, UGA and UAA.

In one embodiment, the invention relates to the use of a cPEP as described above, wherein said fragment comprises:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the use of a cPEP as described above, wherein the translation of the pre-mRNA fragment is carried out in the same reading frame as the open reading frame of the nucleic acid sequence naturally translated in said plant cell. In other words, the invention relates to the use of a cPEP as described above, wherein the translation of the pre-mRNA fragment is carried out in the reading frame determined by the initiation codon of the open reading frame of the said protein.

In one embodiment, the invention relates to the use of a cPEP as described above, wherein the translation of the pre-mRNA fragment is carried out in a reading frame different from the open reading frame of the nucleic acid sequence naturally translated in said plant cell. In particular, the invention relates to the use of a cPEP as described above, wherein the translation of the pre-mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the use of a cPEP as described above, wherein the translation of the pre-mRNA fragment is carried out in a reading frame shifted by two nucleotides 3′ (or one nucleotide 5′) with respect to the open reading frame of the nucleic acid sequence naturally translated in said plant cell. In other words, the invention relates to the use of a cPEP as described above, wherein the translation of the pre-mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in said plant cell.

In one embodiment, the invention relates to the use of a cPEP as a plant protection agent to modulate the accumulation of a protein in a plant cell, said cPEP having a size from 4 to 70 amino acids, in particular from 4 to 41 amino acids, the amino acid sequence of which corresponds to the translation via the genetic code of a fragment of a nucleic acid sequence of a pre-mRNA of a protein, said fragment having a size of 3n nucleotides, n being from 4 to 70, in particular n being from 4 to 41, and said nucleic acid sequence comprising two contiguous parts:
a part located within a nucleic acid sequence known to be non-coding (i.e. not naturally translated, e.g. intron); and
a part located within a nucleic acid sequence considered to be coding (i.e. naturally translated, e.g. exon);
   said cPEP being capable of modulating the accumulation of said protein in the plant cell and not being capable of modulating the accumulation of the mRNA encoding said protein.

In one embodiment, the invention relates to the use of a cPEP as described above to increase the accumulation of said protein in the plant cell. The presence of the cPEP causes the amount of said protein in the treated plant cell to be greater than that in an untreated plant cell.

In one embodiment, the invention relates to the use of a cPEP as described above to decrease (inhibit) the accumulation of said protein in the plant cell. The presence of the cPEP causes the amount of said protein in the treated plant cell to be less than that in an untreated plant cell.

In one embodiment, the invention relates to the use of a cPEP as described above, said nucleic acid sequence deemed non-coding (i.e. not naturally translated) being:
the 5′UTR region;
the 3′UTR region; or
an intron.

In one embodiment, the invention relates to the use of a cPEP as described above, wherein said cPEP is produced outside said plant cell prior to being introduced into said plant cell.

In one embodiment, the invention relates to the use of a cPEP as described above, wherein said cPEP is a synthetic peptide.

In one embodiment, the invention relates to the use of a cPEP as described above, wherein said cPEP is an isolated peptide.

In one embodiment, the invention relates to the use of a cPEP as described above, wherein said cPEP is a recombinant peptide.

In one embodiment, the invention relates to the use of a cPEP as described above, wherein said cPEP is a hydrophobic peptide or a hydrophilic peptide.

In one embodiment, the invention relates to the use of a cPEP as described above, wherein said cPEP is introduced into said plant cell in the form of a nucleic acid encoding said cPEP. In particular, the invention relates to the use of a cPEP as described above, wherein said cPEP is introduced into said plant cell in the form of a nucleic acid encoding said cPEP and comprising the means for expressing it.

In one embodiment, the invention relates to the use of a cPEP as described above, wherein said protein is naturally present in said plant cell.

In one embodiment, the invention relates to the use of a cPEP as described above, wherein said protein is not naturally present in said plant cell. In particular, the invention relates to the use of a cPEP as described above, wherein said protein is encoded by a transgene artificially introduced into said plant cell. In particular, the invention relates to the use of a cPEP as described above, wherein said protein is encoded by a vector artificially introduced into said plant cell.

In one embodiment, the invention relates to the use of a cPEP as described above, wherein the accumulation of said protein is determined via the implementation of a technique selected from: Western blot, measurement of enzymatic activity, mass spectrometry and translational fusion. In particular, the invention relates to the use of a cPEP as described above, wherein the accumulation of said protein is determined via the implementation of a Western blot.

In one embodiment, the invention relates to the use of a cPEP as described above, wherein said cPEP has a size from 4 to 41 amino acids, from 5 to 40 amino acids, from 7 to 20 amino acids or more particularly a size from 8 to 15 amino acids. In particular, said cPEP has a size of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

In one embodiment, the invention relates to the use of a cPEP as described above, wherein said plant cell belongs to: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (trefoil), Medicago sativa (alfalfa), Medicago truncatula (lucerne), Nicotiana benthamiana (tobacco), Oryza sativa (rice), Pisum sativum (pea), Raphanus sativus, Solanum lycopersicum (tomato), Solanum melongena (aubergine), Solanum tuberosum (potato), Thellungiella halophila, Theobroma cacao, Triticum spp. (wheat), Vitis vignifera (grapevine) and Zea mays (maize).

In particular, the invention relates to the use of a cPEP as described above, wherein said plant cell is an algal cell.

In one embodiment, the invention relates to the use of a cPEP as described above, wherein said protein is encoded by a gene selected from: Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexol, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Sk1, Sps1, Spt, Stn8, Tap46, Topp6, TubB6, TubB8, Uba1a, Vim3, Sgr1, Abi5, Hsp101, Rh10 and Wus.

In view of the above, it is understood that in another embodiment, the invention relates to the use of a cPEP as described above, said cPEP having a size from 4 to 70 amino acids, in particular from 4 to 41 amino acids, the amino acid sequence of which corresponds to the translation via the genetic code of a non-naturally translated fragment of a nucleic acid sequence deemed to be non-coding present on a pre-mRNA of a protein, said cPEP being capable of modulating the accumulation of said protein in the plant cell and not being capable of modulating the accumulation of the mRNA encoding said protein, said protein being encoded by a gene selected from: Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexol, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Sk1, Sps1, Spt, Stn8, Tap46, Topp6, TubB6, TubB8, Uba1a, Vim3, Sgr1, Abi5, Hsp101, Rh10 and Wus.

In one embodiment, the invention relates to the use of a cPEP as described above, wherein said protein is encoded by an ORF comprising a nucleic acid sequence having at least 80% identity, preferably at least 90% identity, with a sequence selected from the sequences: SEQ ID NOs: 1 to 161 and 171 to 175.

In one embodiment, the invention relates to the use of a cPEP as described above, wherein said protein is encoded by an ORF comprising a nucleic acid sequence having at least 80% identity, preferably at least 90% identity, with a sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the use of a cPEP as described above, wherein the said protein is encoded by a gene comprising a nucleic acid sequence having at least 80% identity, preferably at least 90% identity, with a sequence chosen from the sequences: SEQ ID NOs: 26 to 55 (Cpk3). In particular, the invention relates to the use of a cPEP as described above, wherein said protein is encoded by a gene comprising a nucleic acid sequence having at least 80% identity, preferably at least 90% identity, with a sequence selected from the sequences: SEQ ID NOs: 61 to 79 (Dcl1). In particular, the invention relates to the use of a cPEP as described above, wherein said protein is encoded by a gene comprising a nucleic acid sequence having at least 80% identity, preferably at least 90% identity, with a sequence selected from the sequences: SEQ ID NOs: 109 to 134 (Nsp1).

In one embodiment, the invention relates to the use of a cPEP as described above, wherein said protein is encoded by a gene comprising a nucleic acid sequence selected from the sequences: SEQ ID NOs: 1 to 161 and 171 to 175. In one embodiment, the invention also relates to the use of a cPEP as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the use of a cPEP as described above, wherein said protein is encoded by a gene comprising one selected from the sequences: SEQ ID NOs: 26 to 55 (Cpk3). In particular, the invention relates to the use of a cPEP as described above, wherein said protein is encoded by a gene comprising one selected from the sequences: SEQ ID NOs: 61 to 79 (Dcl1). In particular, the invention relates to the use of a cPEP as described above, wherein said protein is encoded by a gene comprising one selected from the sequences: SEQ ID NOs: 109 to 134 (Nsp1).

In another embodiment, the invention relates to the use of a cPEP as described above, wherein said cPEP is selected from the sequences: SEQ ID NOs: 162 to 164.

In another embodiment, the invention relates to the use of a cPEP as described above, wherein said protein is involved in at least one plant phenotype selected from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

In another embodiment, the invention concerns the use of a cPEP as described above, to modulate the accumulation of a recombinant protein, the nucleic acid sequence of which encoding it corresponds to the fusion of the nucleic acid sequences of two distinct genes.

In particular, the coding sequence of at least one of the two genes is that of a reporter gene, for example a gene encoding a fluorescent protein (such as GFP) or a protein enabling the plant to resist a compound.

In one embodiment, the invention relates to the use of a cPEP as described above, to modulate the accumulation of a recombinant protein, the nucleic acid sequence of which encoding it corresponds to the fusion:
a nucleic acid sequence known to be non-coding for a first gene; and
of a nucleic acid sequence coding for a second gene,
   the sequence of said cPEP corresponding to the translation via the genetic code of a fragment of the nucleic acid sequence deemed non-coding of the first gene.

In another aspect, the invention relates to a method for modulating the accumulation of a protein in a plant cell comprising a step for introducing:
a cPEP; or
of a nucleic acid encoding said cPEP and the means of expressing it,
   in said plant cell, the introduction of said cPEP resulting in a modulation of the quantity of said protein in said plant cell,
   said cPEP having a size from 4 to 70 amino acids, in particular from 4 to 41 amino acids, the amino acid sequence of which corresponds to the translation via the genetic code of a fragment of a nucleic acid sequence deemed to be non-coding present on a pre-mRNA of a protein,
   said cPEP being capable of modulating the accumulation of said protein in the plant cell and not being capable of modulating the accumulation of the mRNA encoding said protein.

In one embodiment, the invention relates to the method as described above, wherein said fragment is devoid of:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the method as described above, wherein said fragment is devoid of:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the method as described above, wherein said fragment comprises:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention therefore relates to the method as described above, wherein said fragment comprises an AUG initiator codon encoding a methionine initiator and lacks a STOP codon chosen from the codons: UAG, UGA and UAA. The invention also relates to the method as described above, wherein said fragment lacks an initiator codon AUG encoding a methionine initiator and comprises a STOP codon selected from codons: UAG, UGA and UAA.

In one embodiment, the invention relates to the method as described above, wherein said fragment comprises:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the method as described above, wherein the translation of the pre-mRNA fragment is carried out in the same reading frame as the open reading frame of the nucleic acid sequence naturally translated in said plant cell. In other words, the invention relates to the method as described above, wherein the translation of the pre-mRNA fragment is carried out in the reading frame determined by the initiation codon of the open reading frame of the said protein.

In one embodiment, the invention relates to the method as described above, wherein the translation of the pre-mRNA fragment is carried out in a reading frame different from the open reading frame of the nucleic acid sequence naturally translated in said plant cell. In particular, the invention relates to the method as described above, wherein the translation of the pre-mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the method as described above, wherein the translation of the pre-mRNA fragment is carried out in a reading frame shifted by two nucleotides 3′ (or one nucleotide 5′) with respect to the open reading frame of the nucleic acid sequence naturally translated in said plant cell. In other words, the invention relates to the method as described above, wherein the translation of the pre-mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in said plant cell.

In one embodiment, the invention relates to a method for modulating the accumulation of a protein in a plant cell comprising a step for introducing:
a cPEP; or
of a nucleic acid encoding said cPEP and the means of expressing it,
   in said plant cell, the introduction of said cPEP resulting in a modulation of the quantity of said protein in said plant cell, said cPEP having a size from 4 to 70 amino acids, in particular from 4 to 41 amino acids, the amino acid sequence of which corresponds to the translation via the genetic code of a fragment of a nucleic acid sequence of a pre-mRNA of a protein, said fragment having a size of 3n nucleotides, n being from 4 to 70, in particular n being from 4 to 41, and said nucleic acid sequence comprising two contiguous parts:
a part located within a nucleic acid sequence known to be non-coding (i.e. not naturally translated, e.g. intron); and
a part located within a nucleic acid sequence considered to be coding (i.e. naturally translated, e.g. exon);
   said cPEP being capable of modulating the accumulation of said protein in the plant cell and not being capable of modulating the accumulation of the mRNA encoding said protein.

In one embodiment, the invention relates to the method as described above, said method allowing:
promote the development of a plant; or
slow down or prevent the development of a plant.

In particular, the invention relates to the method as described above, said method making it possible to promote the development of a plant. In particular, the invention relates to the method as described above, said method making it possible to slow down or prevent the development of a plant.

In one embodiment, the invention relates to the method as described above for increasing the accumulation of said protein in the plant cell. The presence of cPEP causes the amount of said protein in the treated plant cell to be greater than that in an untreated plant cell.

In one embodiment, the invention relates to the method as described above for decreasing (inhibiting) the accumulation of said protein in the plant cell. The presence of cPEP means that the quantity of said protein in the treated plant cell is less than that in an untreated plant cell.

In one embodiment, the invention relates to the method as described above, said nucleic acid sequence deemed non-coding (i.e. not naturally translated) being:
the 5′UTR region;
the 3′UTR region; or
an intron.

In one embodiment, the invention relates to the method as described above, wherein said cPEP is produced outside said plant cell before being introduced into said plant cell.

In one embodiment, the invention relates to the method as described above, wherein said cPEP is a synthetic peptide.

In one embodiment, the invention relates to the method as described above, wherein said cPEP is an isolated peptide.

In one embodiment, the invention relates to the method as described above, wherein said cPEP is a recombinant peptide.

In one embodiment, the invention relates to the method as described above, wherein said cPEP being a hydrophobic peptide or a hydrophilic peptide.

In one embodiment, the invention relates to the method as previously described, wherein said cPEP is introduced into said plant cell in the form of a nucleic acid encoding said cPEP. In particular, the invention relates to the method as described above, wherein said cPEP is introduced into said plant cell in the form of a nucleic acid encoding said cPEP and comprising the means for expressing it.

In one embodiment, the invention relates to the method as described above, wherein said protein is naturally present in said plant cell.

In one embodiment, the invention relates to the method as described above, wherein said protein is not naturally present in said plant cell. In particular, the invention relates to the method as described above, wherein said protein is encoded by a transgene artificially introduced into said plant cell. In particular, the invention relates to the method as described above, wherein said protein is encoded by a vector artificially introduced into said plant cell.

In one embodiment, the invention relates to the method as described above, wherein the accumulation of the said protein is determined using a technique selected from: Western blot, measurement of enzyme activity, mass spectrometry and translational fusion. In particular, the invention relates to the method as described above, wherein the accumulation of the said protein is determined using a Western blot.

In one embodiment, the invention relates to the method as described above, wherein said cPEP has a size from 4 to 41 amino acids, from 5 to 40 amino acids, from 7 to 20 amino acids or more particularly a size from 8 to 15 amino acids. In particular, said cPEP has a size of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

In one embodiment, the invention relates to the method as described above, wherein said plant cell or said plant belongs to: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (trefoil), Medicago sativa (alfalfa), Medicago truncatula (lucerne), Nicotiana benthamiana (tobacco), Oryza sativa (rice), Pisum sativum (pea), Raphanus sativus, Solanum lycopersicum (tomato), Solanum melongena (aubergine), Solanum tuberosum (potato), Thellungiella halophila, Theobroma cacao, Triticum spp. (wheat), Vitis vignifera (grapevine) and Zea mays (maize).

In particular, the invention relates to the method as described above, wherein said plant cell is an algal cell.

In one embodiment, the invention relates to the method as described above, wherein said protein is encoded by a gene selected from: Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexol, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Sk1, Sps1, Spt, Stn8, Tap46, Topp6, TubB6, TubB8, Uba1a, Vim3, Sgr1, Abi5, Hsp101, Rh10 and Wus.

In view of the foregoing, it is understood that in another embodiment, the invention relates to the method as described above comprising a step for introducing:
a cPEP; or
of a nucleic acid encoding said cPEP and the means of expressing it,
   in said plant cell, the introduction of said cPEP resulting in a modulation of the quantity of said protein in said plant cell,
   said cPEP having a size from 4 to 70 amino acids, in particular from 4 to 41 amino acids, the amino acid sequence of which corresponds to the translation via the genetic code of a non-naturally translated fragment of a nucleic acid sequence deemed to be non-coding present on a pre-mRNA of a protein,
   said cPEP being capable of modulating the accumulation of said protein in the plant cell and not being capable of modulating the accumulation of the mRNA encoding said protein,
   said protein being encoded by a gene selected from: Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexol, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Sk1, Sps1, Spt, Stn8, Tap46, Topp6, TubB6, TubB8, Uba1a, Vim3, Sgr1, Abi5, Hsp101, Rh10 and Wus.

In one embodiment, the invention relates to the method as described above, wherein said protein is encoded by an ORF comprising a nucleic acid sequence having at least 80% identity, preferably at least 90% identity, with a sequence chosen from the sequences: SEQ ID NOs: 1 to 161 and 171 to 175.

In one embodiment, the invention relates to the method as described above, wherein said protein is encoded by an ORF comprising a nucleic acid sequence having at least 80% identity, preferably at least 90% identity, with a sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method as described above, wherein the said protein is encoded by a gene comprising a nucleic acid sequence having at least 80% identity, preferably at least 90% identity, with a sequence chosen from the sequences: SEQ ID NOs: 26 to 55 (Cpk3). In particular, the invention relates to the method as described above, wherein said protein is encoded by a gene comprising a nucleic acid sequence having at least 80% identity, preferably at least 90% identity, with a sequence chosen from the sequences: SEQ ID NOs: 61 to 79 (Dcl1). In particular, the invention relates to the method as described above, wherein said protein is encoded by a gene comprising a nucleic acid sequence having at least 80% identity, preferably at least 90% identity, with a sequence chosen from the sequences: SEQ ID NOs: 109 to 134 (Nsp1).

In one embodiment, the invention relates to the method as described above, wherein said protein is encoded by a gene comprising a nucleic acid sequence chosen from the sequences: SEQ ID NOs: 1 to 161 and 171 to 175. In one embodiment, the invention also relates to the method as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method as described above, wherein said protein is encoded by a gene comprising one selected from the sequences: SEQ ID NOs: 26 to 55 (Cpk3). In particular, the invention relates to the method as described above, wherein said protein is encoded by a gene comprising one selected from the sequences: SEQ ID NOs: 61 to 79 (Dcl1). In particular, the invention relates to the method as described above, wherein the said protein is encoded by a gene comprising one selected from the sequences: SEQ ID NOs: 109 to 134 (Nsp1).

In another embodiment, the invention relates to the method as described above, wherein said cPEP is chosen from the sequences: SEQ ID NOs: 162 to 164.

In another embodiment, the invention relates to the method as described above, wherein said protein is involved in at least one plant phenotype selected from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and duration of juvenile phase;
duration of flowering;
resistance to biotic stress;
resistance to abiotic stress; and
the number of cells.

In one embodiment, the invention relates to the method as described above, wherein the introduction of said cPEP leads to earlier bolting in said plant.

In one embodiment, the invention relates to the method as described above, wherein the introduction of said cPEP results in earlier flowering in said plant.

In one embodiment, the invention relates to the method as described above, wherein introduction of said cPEP results in an increase in stem size in said plant.

In one embodiment, the invention relates to the method as described above, wherein the introduction of said cPEP results in earlier stem growth in said plant.

The Inventors have unexpectedly found that it is possible to apply a cPEP directly to the plant, e.g. using the composition of the invention (see above) comprising a cPEP, to modulate the accumulation of a target protein in the plant, which indicates that the cPEP is taken up by the plant.

Therefore, in one embodiment, the invention relates to the method as described above, wherein said cPEP is introduced into said plant:
by watering, by spraying or by adding a fertiliser, a potting soil, a culture substrate or a support in contact with the plant, the said cPEP being administered to the plant in particular in the form of a composition comprising from 10⁻⁹ M to 10⁻⁴ M of the said cPEP;
by watering, soaking, spraying or by adding a fertiliser, a potting soil, a growing substrate or a support in contact with the plant, the said cPEP being administered in particular to a seed or a seedling in the form of a composition comprising from 10⁻⁹ M to 10⁻⁴ M of the said cPEP; or
by means of a nucleic acid encoding said cPEP and comprising the means for expressing said cPEP, said nucleic acid being artificially introduced into the plant.

In one embodiment, the invention relates to the method as defined above, wherein said cPEP is artificially introduced externally into the plant, preferably by watering, spraying or by the addition of a fertiliser, potting soil, growing substrate or inert support.

In one embodiment, the invention relates to the method as defined above, wherein said cPEP is introduced by watering.

In one embodiment, the invention relates to the method as defined above, wherein said cPEP is introduced by spraying.

In one embodiment, the invention relates to the method as defined above, wherein said cPEP is introduced by the addition of a fertiliser.

In one embodiment, the invention relates to the method as defined above, wherein the plant is treated with a composition comprising from 10⁻⁹ M to 10⁻⁴ M of said cPEP, or comprising in particular 10⁻⁹ M, 10⁻⁸ M, 10⁻⁷ M, 10⁻⁶ M, 10⁻⁵ M or 10⁻⁴ M of said cPEP. Preferably, the compositions have a concentration of 10⁻⁸ M to 10⁻⁵ M for application to the plant by watering or spraying.

In addition, more or less concentrated compositions can be used to treat the plant with cPEP. For example, and without limitation, more concentrated compositions comprising from 10⁻¹ M to 10⁻³ M, or comprising in particular 10⁻² M of cPEP, can be used in the case where the cPEP artificially introduced externally is administered to the plant by spreading.

In another aspect, the invention concerns a modified plant containing a cPEP, which “modified plant” corresponds to a plant into which a cPEP has been artificially introduced, in particular by watering, spraying or via a fertiliser.

In one embodiment, the invention relates to the plant modified comprising a cPEP introduced artificially by an exogenous route as described above, said cPEP having a size from 4 to 70 amino acids, in particular from 4 to 41 amino acids, the amino acid sequence of which corresponds to the translation via the genetic code of a fragment of a nucleic acid sequence deemed to be non-coding present on a pre-mRNA of a protein, said cPEP being capable of modulating the accumulation of said protein in the plant cell and not being capable of modulating the accumulation of the mRNA encoding said protein.

In one embodiment, the invention relates to the modified plant as described above, wherein said fragment lacks:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the modified plant as described above, wherein said fragment lacks:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the modified plant as described above, wherein said fragment comprises:
an AUG initiator codon encoding a methionine initiator; or
a STOP codon chosen from: UAG, UGA and UAA,
   and wherein said fragment is selected from:
either in the same reading frame as the open reading frame encoding the said protein;
or in a reading frame shifted by one or two nucleotides relative to the open reading frame encoding said protein.

In one embodiment, the invention therefore relates to the modified plant as described above, wherein said fragment comprises an AUG initiator codon encoding a methionine initiator and lacks a STOP codon chosen from the codons: UAG, UGA and UAA. The invention also relates to the modified plant as described above, wherein said fragment lacks an initiator codon AUG encoding a methionine initiator and comprises a STOP codon selected from codons: UAG, UGA and UAA.

In one embodiment, the invention relates to the modified plant as described above, wherein said fragment comprises:
an AUG initiator codon encoding a methionine initiator; and
a STOP codon chosen from: UAG, UGA and UAA,
   and wherein said fragment is selected from:
either in the same reading frame as the open reading frame encoding the said protein;
or in a reading frame shifted by one or two nucleotides relative to the open reading frame encoding said protein.

In one embodiment, the invention relates to the modified plant as described above, wherein the translation of the pre-mRNA fragment is carried out in the same reading frame as the open reading frame of the nucleic acid sequence naturally translated in said plant cell. In other words, the invention relates to the modified plant as described above, wherein the translation of the fragment of the pre-mRNA is carried out in the reading frame determined by the initiation codon of the open reading frame of the said protein.

In one embodiment, the invention relates to the modified plant as described above, wherein the translation of the pre-mRNA fragment is carried out in a reading frame different from the open reading frame of the nucleic acid sequence naturally translated in said plant cell. In particular, the invention relates to the modified plant as described above, wherein the translation of the pre-mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the modified plant as described above, wherein the translation of the pre-mRNA fragment is carried out in a reading frame shifted by two nucleotides 3′ (or one nucleotide 5′) with respect to the open reading frame of the nucleic acid sequence naturally translated in said plant cell. In other words, the invention relates to the modified plant as described above, wherein the translation of the pre-mRNA fragment is carried out in a reading frame shifted by one (or two nucleotides at 5′) or two nucleotides at 3′ (or one nucleotide at 5′) with respect to the initiation codon of the open reading frame of the nucleic acid sequence naturally translated in said plant cell.

In one embodiment, the invention relates to a modified plant comprising an artificially exogenously introduced cPEP, said cPEP having a size from 4 to 70 amino acids, in particular from 4 to 41 amino acids, the amino acid sequence of which corresponds to the translation via the genetic code of a fragment of a nucleic acid sequence of a pre-mRNA of a protein, the said fragment having a size of 3n nucleotides, n being from 4 to 70, in particular n being from 4 to 41, and the said nucleic acid sequence comprising two contiguous parts:
a part located within a nucleic acid sequence known to be non-coding (i.e. not naturally translated, e.g. intron); and
a part located within a nucleic acid sequence considered to be coding (i.e. naturally translated, e.g. exon);
   said cPEP being capable of modulating the accumulation of said protein in the plant cell and not being capable of modulating the accumulation of the mRNA encoding said protein.

In one embodiment, the invention relates to the modified plant as described above, said plant being: Alopecurus myosuroides, Amaranthus hypochondriacus, Amaranthus palmeri, Amaranthus tuberculatus, Arabidopsis halleri, Arabidopsis lyrata, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (trefoil), Medicago sativa (alfalfa), Medicago truncatula (lucerne), Nicotiana benthamiana (tobacco), Oryza sativa (rice), Pisum sativum (pea), Raphanus sativus, Solanum lycopersicum (tomato), Solanum melongena (aubergine), Solanum tuberosum (potato), Thellungiella halophila, Theobroma cacao, Triticum spp. (wheat), Vitis vignifera (grapevine) and Zea mays (maize).

In another aspect, the invention concerns a transgenic plant comprising a nucleic acid encoding a cPEP and the means of expressing it,
said cPEP having a size from 4 to 70 amino acids, in particular from 4 to 41 amino acids, the amino acid sequence of which corresponds to the translation via the genetic code of a fragment of a nucleic acid sequence deemed to be non-coding present on a pre-mRNA of a protein,
said cPEP being capable of modulating the accumulation of said protein in the plant cell and not being capable of modulating the accumulation of the mRNA encoding said protein.

In one embodiment, the invention relates to the transgenic plant as described above, wherein said fragment lacks:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the transgenic plant as described above, wherein said fragment lacks:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the transgenic plant as described above, wherein said fragment comprises:
an AUG initiator codon encoding a methionine initiator; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention therefore relates to the transgenic plant as described above, wherein said fragment comprises an AUG initiator codon encoding a methionine initiator and lacks a STOP codon chosen from the codons: UAG, UGA and UAA. The invention also relates to the transgenic plant as described above, wherein the said fragment lacks an AUG initiator codon encoding a methionine initiator and comprises a STOP codon chosen from the codons: UAG, UGA and UAA.

In one embodiment, the invention relates to the transgenic plant as described above, wherein said fragment comprises:
an AUG initiator codon encoding a methionine initiator; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the transgenic plant as described above, wherein the translation of the pre-mRNA fragment is carried out in the same reading frame as the open reading frame of the nucleic acid sequence naturally translated in said plant cell. In other words, the invention relates to the transgenic plant as described above, wherein the translation of the fragment of the pre-mRNA is carried out in the reading frame determined by the initiation codon of the open reading frame of the said protein.

In one embodiment, the invention relates to the transgenic plant as described above, wherein the translation of the pre-mRNA fragment is carried out in a reading frame different from the open reading frame of the nucleic acid sequence naturally translated in said plant cell. In particular, the invention relates to the transgenic plant as described above, wherein the translation of the pre-mRNA fragment is carried out in a reading frame shifted by one nucleotide at 3′ (or by two nucleotides at 5′) with respect to the open reading frame of the nucleic acid sequence naturally translated in the said plant cell. In particular, the invention also relates to the transgenic plant as described above, wherein the translation of the pre-mRNA fragment is carried out in a reading frame shifted by two nucleotides 3′ (or one nucleotide 5′) with respect to the open reading frame of the nucleic acid sequence naturally translated in said plant cell. In other words, the invention relates to the transgenic plant as described above, wherein the translation of the pre-mRNA fragment is carried out in a reading frame shifted by one (or two nucleotides at 5′) or two nucleotides at 3′ (or one nucleotide at 5′) with respect to the initiation codon of the open reading frame of the nucleic acid sequence naturally translated in the said plant cell.

In one embodiment, the invention relates to a transgenic plant comprising a nucleic acid encoding a cPEP and the means for expressing it, said cPEP having a size from 4 to 70 amino acids, in particular from 4 to 41 amino acids, the amino acid sequence of which corresponds to the translation via the genetic code of a fragment of a nucleic acid sequence of a pre-mRNA of a protein, the said fragment having a size of 3n nucleotides, n being from 4 to 70, in particular n being from 4 to 41, and the said nucleic acid sequence comprising two contiguous parts:
a part located within a nucleic acid sequence known to be non-coding (i.e. not naturally translated, e.g. intron); and
a part located within a nucleic acid sequence considered to be coding (i.e. naturally translated, e.g. exon);
   said cPEP being capable of modulating the accumulation of said protein in the plant cell and not being capable of modulating the accumulation of the mRNA encoding said protein.

In one embodiment, the invention relates to the transgenic plant as defined above, wherein the sequence encoding said cPEP is shorter than the sequence of the pre-mRNA encoding said protein.

In one embodiment, the invention relates to the transgenic plant as described above, said plant being: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vignifera (grapevine) and Zea mays (maize).

In one embodiment, the invention relates to the transgenic plant as described above, wherein expression of said cPEP is placed under the control of a strong promoter, preferably a constitutive strong promoter such as the 35S promoter.

In any event, it should be noted that the various aspects of the invention, like the various embodiments thereof, are interdependent. They may therefore be combined to obtain aspects and/or preferred embodiments of the invention not explicitly described. This also applies to all the definitions provided in this description, which apply to all aspects of the invention and its embodiments.

In addition, the present invention is illustrated by, but not limited to, the following Figures and Examples.

### LIST OF FIGURES

FIG. 1 is a schematic representation illustrating the first steps of the method for preparing and determining a cPEP. In particular, the steps for determining one of the nucleic acid sequences within a protein pre-mRNA that is considered to be non-coding, from which the peptide to be tested (i.e. the potential cPEP) is identified, are illustrated. SEQ ID NOs: 165 to 168 are provided as examples only.

FIG. 2 is a schematic representation of the NSP1-GUS construct expressed in the transformed Medicago truncatula root.

The NSP1 promoter region, the NSP1 CDS, the β-glucuronidase gene (GUS) and the NSP1 3′UTR region are indicated on the sequence of the construct. The cPEPs peptides are positioned above the regions that were used to construct each of the cPEPs.

FIG. 3 shows the effect of cPEP NSP1-5′UTR-10 on the accumulation of the NSP1-GUS fusion protein in Medicago truncatula.

The y-axis represents the quantification of GUS activity in the roots of an NSP1-GUS fusion in response to treatment for 5 days with 0.1 μM of a cPEP targeting the 5′UTR region of the NSP1 gene. Error bars represent SEM, asterisks indicate a significant difference between test condition and control according to Student's t-test (n=30, p<0.05).

FIG. 4 shows the effect of cPEP NSP1-3′UTR-10 on the accumulation of the NSP1-GUS fusion protein in Medicago truncatula.

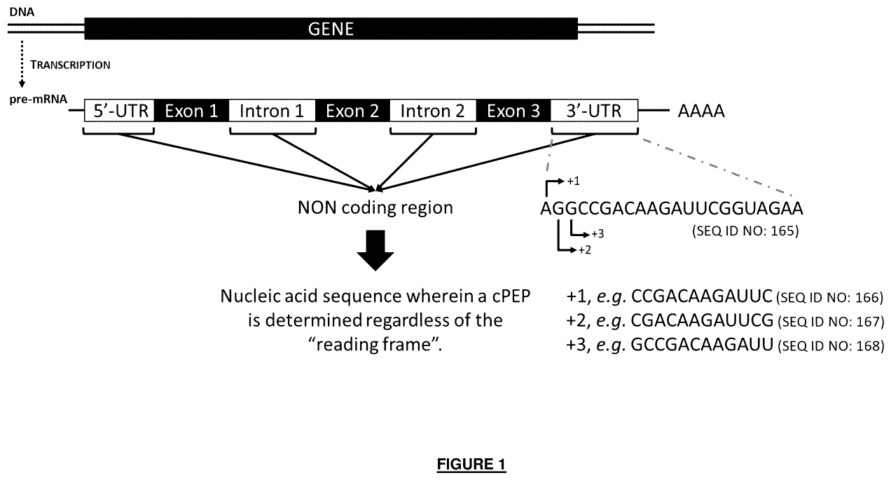

The y-axis represents the quantification of GUS activity in the roots of an NSP1-GUS fusion in response to treatment for 5 days with 0.1 μM of a cPEP targeting the 5′UTR region of the NSP1 gene. Error bars represent SEM, asterisks indicate a significant difference between test condition and control according to Student's t-test (n=30, p<0.05).

### EXAMPLES

### Materials & Methods

1. Preparation of cPEPs

The NSP1-5′UTR-5 cPEP sequence SEQ ID NO: 163 (5 amino acids) was obtained by translating a 15 nucleotide fragment located in the 5′UTR part of the NSP1 pre-mRNA (SEQ ID NO: 169) in the +3 reading frame relative to the initiator codon of the nsp1 protein.

The NSP1-5′UTR-11 cPEP sequence SEQ ID NO: 162 (11 amino acids) was obtained by translating a 30 nucleotide fragment located in the 5′UTR part of the NSP1 pre-mRNA (SEQ ID NO: 169) in the +3 reading frame relative to the initiator codon of the nsp1 protein.

The sequence SEQ ID NO: 164 of the NSP1-3′UTR cPEP (10 amino acids) was obtained by translating a 30 nucleotide fragment located in the 3′UTR part of the NSP1 pre-mRNA (SEQ ID NO: 169) in the +2 reading frame relative to the initiator codon of the nsp1 protein.

Each peptide was synthesised (Smartox Biotech) and diluted to 10 mM in water.

### 2. Construction of the Plasmid

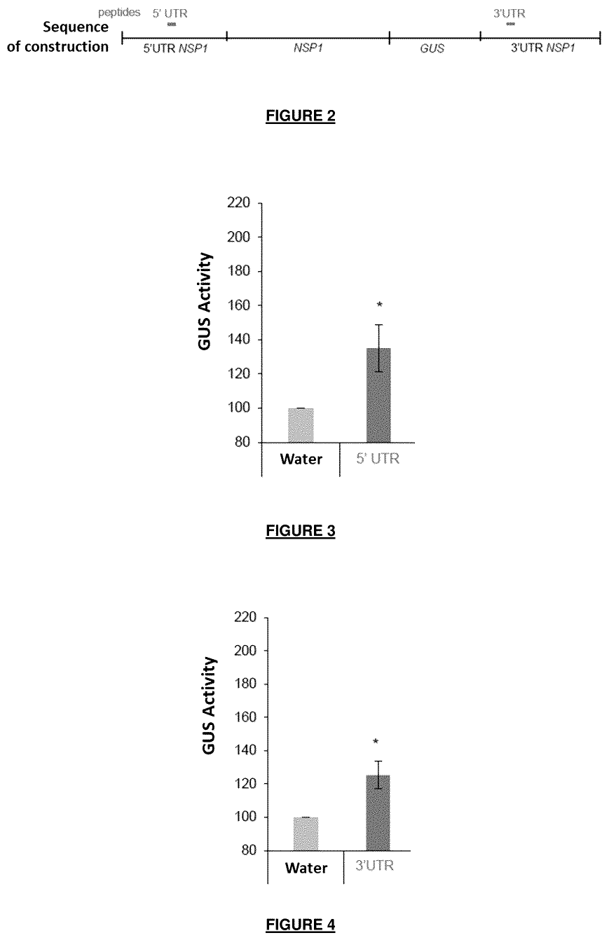

NSP1::GUS fusion was achieved, using a modified pCambia vector (Lauressergues et al., Nature, 520: 90-3, 2015), by cloning 3 kb of NSP1 promoter, the NSP1 gene coding sequence, the GUS protein coding sequence and 3 kb of the NSP1 gene downstream sequence (FIG. 2).

### 3. Plant Methoding

Root transformation of M. truncatula was carried out using the method described in Boisson-Dernier et al, Mol Plant Microbe Interact, 14(6): 695-700, 2001.

### 4. Measurement of Gene Expression

Plants were treated by spraying with a solution containing a low concentration of cPEPs (from 0.1 μM to 10 μM) or with water.

The accumulation of the protein targeted by cPEP was then measured in each plant by quantifying GUS activity or by quantifying protein activity by Western blot (Blizquez M., Quantitative GUS activity assay in intact plant tissue, Cold Spring Harbor Laboratory Press, Cold Spring Harbor, NY, USA, 2002).

### Results

The cPEPs targeting the 5′UTR (SEQ ID NO: 162) and 3′UTR (SEQ ID NO: 164) non-coding regions of the NSP1 gene induced an increase in the accumulation of the NSP1 protein in Medicago truncatula, regardless of the reading frame used to construct the cPEP and regardless of the position of the fragment used to construct the cPEP (FIGS. 3 and 4).

1-11. (canceled)

12. A composition comprising a cPEP as an active substance, said cPEP:
having a size from 4 to 70 amino acids, the amino acid sequence of which corresponds to the translation via the genetic code of a fragment of a nucleic acid sequence deemed to be non-coding present on a pre-mRNA of a protein; and
being capable of modulating the accumulation of said protein in the plant cell and not being capable of modulating the accumulation of the mRNA encoding said protein.

13. The composition according to claim 12, said cPEP having a size from 4 to 41 amino acids.

14. The composition according to claim 12, said protein being encoded by an ORF comprising a nucleic acid sequence having at least 80% identity with a sequence chosen from the sequences: SEQ ID NO: 1 (ORF of the Aae15 protein, A. thaliana), SEQ ID NO: 2 (ORF of the Aae16 protein, A. thaliana), SEQ ID NO: 3 (ORF of the abcg11 protein, A. thaliana), SEQ ID NO: 4 (ORF of the Abdcg34 protein, A. thaliana), SEQ ID NO: 5 (ORF of the Acc1 protein, A. thaliana), SEQ ID NO: 6 (ORF of the Agb1 protein, A. thaliana), SEQ ID NO: 7 (ORF of the A1s protein, A. thaliana), SEQ ID NO: 8 (ORF of the Anac076 protein, A. thaliana), SEQ ID NO: 9 (ORF of the Apg9 protein, A. thaliana), SEQ ID NO: 10 (ORF of the Arlb1 protein, A. thaliana), SEQ ID NO: 11 (ORF of Arr1 protein, A. thaliana), SEQ ID NO: 12 (ORF of Arr5 protein, A. thaliana), SEQ ID NO: 13 (ORF of Arr6 protein, A. thaliana), SEQ ID NO: 14 (ORF of At59 protein, A. thaliana), SEQ ID NO: 15 (ORF of Bak1 protein, A. thaliana), SEQ ID NO: 16 (ORF of the Bccp1 protein, A. thaliana), SEQ ID NO: 17 (ORF of the Bccp2 protein, A. thaliana), SEQ ID NO: 18 (ORF of the Bri1 protein, A. thaliana), SEQ ID NO: 19 (ORF of the Bzo2h3 protein, A. thaliana), SEQ ID NO: 20 (ORF of the Cesa6 protein, A. thaliana), SEQ ID NO: 21 (ORF of the Cipk3 protein, A. thaliana), SEQ ID NO: 22 (ORF of the Cks1 protein, A. thaliana), SEQ ID NO: 23 (ORF of the Cobl8 protein, A. thaliana), SEQ ID NO: 24 (ORF of Coi1 protein, A. thaliana), SEQ ID NO: 25 (ORF of Coi1 protein, A. thaliana), SEQ ID NO: 26 (ORF of Cpk3 protein, A. thaliana), SEQ ID NO: 27 (ORF of Cpk3 protein, A. hypochondriacus), SEQ ID NO: 28 (ORF of Cpk3 protein, B. distachyon), SEQ ID NO: 29 (ORF of Cpk3 protein, B. distachyon), SEQ ID NO: 30 (ORF of Cpk3 protein, G. max), SEQ ID NO: 31 (ORF of Cpk3 protein, G. max), SEQ ID NO: 32 (ORF of Cpk3 protein, G. max), SEQ ID NO: 33 (ORF of Cpk3 protein, G. max), SEQ ID NO: 34 (ORF of Cpk3 protein, O. sativa), SEQ ID NO: 35 (ORF of Cpk3 protein, O. sativa), SEQ ID NO: 36 (ORF of Cpk3 protein, S. lycopersicum), SEQ ID NO: 37 (ORF of Cpk3 protein, Z. mays), SEQ ID NO: 38 (ORF of Cpk3 protein, Z. mays), SEQ ID NO: 39 (ORF of Cpk3 protein, Z. mays), SEQ ID NO: 40 (ORF of Cpk3 protein, B. rapa), SEQ ID NO: 41 (ORF of Cpk3 protein, B. rapa), SEQ ID NO: 42 (ORF of Cpk3 protein, H. vulgare), SEQ ID NO: 43 (ORF of Cpk3 protein, H. vulgare), SEQ ID NO: 44 (ORF of Cpk3 protein, S. tuberosum), SEQ ID NO: 45 (ORF of Cpk3 protein, A. palmeri), SEQ ID NO: 46 (ORF of Cpk3 protein, M. truncatula), SEQ ID NO: 47 (ORF of Cpk3 protein, M. truncatula), SEQ ID NO: 48 (ORF of Cpk3 protein, T. aestivum), SEQ ID NO: 49 (ORF of Cpk3 protein, T. aestivum), SEQ ID NO: 50 (ORF of Cpk3 protein, T. aestivum), SEQ ID NO: 51 (ORF of Cpk3 protein, T. aestivum), SEQ ID NO: 52 (ORF of Cpk3 protein, L. perenne), SEQ ID NO: 53 (ORF of Cpk3 protein, L. perenne), SEQ ID NO: 54 (ORF of Cpk3 protein, L. perenne), SEQ ID NO: 55 (ORF of protein Cpk3, L. perenne), SEQ ID NO: 56 (ORF of protein Crk34, A. thaliana), SEQ ID NO: 57 (ORF of protein Cyp705a18, A. thaliana), SEQ ID NO: 58 (ORF of protein Cyp71b26, A. thaliana), SEQ ID NO: 59 (ORF of protein Cyp78a8, A. thaliana), SEQ ID NO: 60 (ORF of Cyp97b3 protein, A. thaliana), SEQ ID NO: 61 (ORF of Dcl1 protein, A. thaliana), SEQ ID NO: 62 (ORF of Dcl1 protein, A. thaliana), SEQ ID NO: 63 (ORF of Dcl1 protein, A. hypochondriacus), SEQ ID NO: 64 (ORF of Dcl1 protein, B. distachyon), SEQ ID NO: 65 (ORF of Dcl1 protein, G. max), SEQ ID NO: 66 (ORF of Dcl1 protein, G. max), SEQ ID NO: 67 (ORF of Dcl1 protein, O. sativa), SEQ ID NO: 68 (ORF of Dcl1 protein, S. lycopersicum), SEQ ID NO: 69 (ORF of Dcl1 protein, Z. mays), SEQ ID NO: 70 (ORF of Dcl1 protein, B. rapa), SEQ ID NO: 71 (ORF of Dcl1 protein, H. vulgare), SEQ ID NO: 72 (ORF of Dcl1 protein, S. tuberosum), SEQ ID NO: 73 (ORF of Dcl1 protein, M. truncatula), SEQ ID NO: 74 (ORF of Dcl1 protein, T. aestivum), SEQ ID NO: 75 (ORF of Dcl1 protein, T. aestivum), SEQ ID NO: 76 (ORF of the Dcl1 protein, T. aestivum), SEQ ID NO: 77 (ORF of the Dcl1 protein, T. aestivum), SEQ ID NO: 78 (ORF of the Dcl1 protein, L. perenne), SEQ ID NO: 79 (ORF of the Dcl1 protein, L. perenne), SEQ ID NO: 80 (ORF of the Dur3 protein, A. thaliana), SEQ ID NO: 81 (ORF of the Ein2 protein, A. thaliana), SEQ ID NO: 82 (ORF of Emb175 protein, A. thaliana), SEQ ID NO: 83 (ORF of Emb2726 protein, A. thaliana), SEQ ID NO: 84 (ORF of Emb9 protein, A. thaliana), SEQ ID NO: 85 (ORF of Epsps protein, A. thaliana), SEQ ID NO: 86 (ORF of Fnr1 protein, A. thaliana), SEQ ID NO: 87 (ORF of the Fve protein, A. thaliana), SEQ ID NO: 88 (ORF of the Ga2ox7 protein, A. thaliana), SEQ ID NO: 89 (ORF of the Gapc protein, N. benthamiana), SEQ ID NO: 90 (ORF of the Gcn2 protein, A. thaliana), SEQ ID NO: 91 (ORF of the Gdi2 protein, A. thaliana), SEQ ID NO: 92 (ORF of Gln2 protein, A. thaliana), SEQ ID NO: 93 (ORF of Gs13 protein, A. thaliana), SEQ ID NO: 94 (ORF of Hag5 protein, A. thaliana), SEQ ID NO: 95 (ORF of the Hda18 protein, A. thaliana), SEQ ID NO: 96 (ORF of the Hexol protein, A. thaliana), SEQ ID NO: 97 (ORF of the Hppd protein, A. thaliana), SEQ ID NO: 98 (ORF of the Hsl1 protein, A. thaliana), SEQ ID NO: 99 (ORF of the Iaa31 protein, A. thaliana), SEQ ID NO: 100 (ORF of the Iqd28 protein, A. thaliana), SEQ ID NO: 101 (ORF of the Jac1 protein, A. thaliana), SEQ ID NO: 102 (ORF of the Jar1 protein, A. thaliana), SEQ ID NO: 103 (ORF of the Kp1 protein, A. thaliana), SEQ ID NO: 104 (ORF of the Lrx2 protein, A. thaliana), SEQ ID NO: 105 (ORF of the Mapkkk3 protein, A. thaliana), SEQ ID NO: 106 (ORF of the Mapkkk5 protein, A. thaliana), SEQ ID NO: 107 (ORF of the Mfp2 protein, A. thaliana), SEQ ID NO: 108 (ORF of the Mrb1 protein, A. thaliana), SEQ ID NO: 109 (ORF of the Nsp1 protein, M. truncatula), SEQ ID NO: 110 (ORF of the Nsp1 protein, A. thaliana), SEQ ID NO: 111 (ORF of the Nsp1 protein, B. distachyon), SEQ ID NO: 112 (ORF of the Nsp1 protein, G. max), SEQ ID NO: 113 (ORF of the Nsp1 protein, G. max), SEQ ID NO: 114 (ORF of the Nsp1 protein, O. sativa), SEQ ID NO: 115 (ORF of the Nsp1 protein, S. lycopersicum), SEQ ID NO: 116 (ORF of the Nsp1 protein, S. lycopersicum), SEQ ID NO: 117 (ORF of the Nsp1 protein, Z. mays), SEQ ID NO: 118 (ORF of the Nsp1 protein, Z. mays), SEQ ID NO: 119 (ORF of Nsp1 protein, Z. mays), SEQ ID NO: 120 (ORF of Nsp1 protein, Z. mays), SEQ ID NO: 121 (ORF of Nsp1 protein, B. rapa), SEQ ID NO: 122 (ORF of Nsp1 protein, H. vulgare), SEQ ID NO: 123 (ORF of Nsp1 protein, H. vulgare), SEQ ID NO: 124 (ORF of Nsp1 protein, H. vulgare), SEQ ID NO: 125 (ORF of the Nsp1 protein, H. vulgare), SEQ ID NO: 126 (ORF of the Nsp1 protein, H. vulgare), SEQ ID NO: 127 (ORF of the Nsp1 protein, H. vulgare), SEQ ID NO: 128 (ORF of the Nsp1 protein, H. vulgare), SEQ ID NO: 129 (ORF of the Nsp1 protein, S. tuberosum), SEQ ID NO: 130 (ORF of the Nsp1 protein, S. tuberosum), SEQ ID NO: 131 (ORF of the Nsp1 protein, T. aestivum), SEQ ID NO: 132 (ORF of the Nsp1 protein, T. aestivum), SEQ ID NO: 133 (ORF of the Nsp1 protein, L. perenne), SEQ ID NO: 134 (ORF of the Nsp1 protein, L. perenne), SEQ ID NO: 135 (ORF of the Pds protein, A. thaliana), SEQ ID NO: 136 (ORF of the Pen3 protein, A. thaliana), SEQ ID NO: 137 (ORF of the Phyb protein, A. thaliana), SEQ ID NO: 138 (ORF of the Pif3 protein, A. thaliana), SEQ ID NO: 139 (ORF of the Pizza protein, A. thaliana), SEQ ID NO: 140 (ORF of the Ppox1 protein, A. thaliana), SEQ ID NO: 141 (ORF of the Ppox2 protein, A. thaliana), SEQ ID NO: 142 (ORF of the Prp39 protein, A. thaliana), SEQ ID NO: 143 (ORF of the PsbA protein, A. thaliana), SEQ ID NO: 144 (ORF of the Pskr1 protein, A. thaliana), SEQ ID NO: 145 (ORF of the Rd2l protein, A. thaliana), SEQ ID NO: 146 (ORF of the Ring1 protein, A. thaliana), SEQ ID NO: 147 (ORF of the Ros1 protein, A. thaliana), SEQ ID NO: 148 (ORF of the Rpt4a protein, A. thaliana), SEQ ID NO: 149 (ORF of the Sfr6 protein, A. thaliana), SEQ ID NO: 150 (ORF of the Shr protein, A. thaliana), SEQ ID NO: 151 (ORF of the Shy2 protein, A. thaliana), SEQ ID NO: 152 (ORF of the Sk1 protein, M. truncatula), SEQ ID NO: 153 (ORF of the Sps1 protein, A. thaliana), SEQ ID NO: 154 (ORF of the Spt protein, A. thaliana), SEQ ID NO: 155 (ORF of the Stn8 protein, A. thaliana), SEQ ID NO: 156 (ORF of the Tap46 protein, A. thaliana), SEQ ID NO: 157 (ORF of the Topp6 protein, A. thaliana), SEQ ID NO: 158 (ORF of the TubB6 protein, A. thaliana), SEQ ID NO: 159 (ORF of the TubB8 protein, A. thaliana), SEQ ID NO: 160 (ORF of the Uba1a protein, A. thaliana), SEQ ID NO: 161 (ORF of the Vim3 protein, A. thaliana), SEQ ID NO: 171 (ORF of the Sgr1 protein, A. thaliana), SEQ ID NO: 172 (ORF of the Abi5 protein, A. thaliana), SEQ ID NO: 173 (ORF of the Hsp101 protein, A. thaliana), SEQ ID NO: 174 (ORF of the Rh10 protein, M. truncatula) and SEQ ID NO: 175 (ORF of the Wus protein, A. thaliana).

15. The composition according to claim 12, wherein the sequence of said cPEP is chosen from the sequences: SEQ ID NO: 162 (NSP1-5′UTR-11), SEQ ID NO: 163 (NSP1-5′UTR-5) and SEQ ID NO: 164 (NSP1-3′UTR).

16. The composition according to claim 12, said cPEP being at a concentration from 5 μM to 500 μM or from 30 μM to 70 μM, or at a concentration of 50 μM.

17. The composition according to claim 12, said composition being a phytopharmaceutical composition, a herbicidal composition or a coating composition.

18. The composition according to claim 12, said composition being a coating composition further comprising at least one fixing agent.

19. The composition according to claim 12, said composition being a herbicidal to decrease the growth of weeds by targeting a Amaranthus and Brassicaceae species.

20. A method for modulating accumulation of a protein in a plant cell comprising introducing into said plant cell a cPEP as a plant protection agent,
said cPEP having a size from 4 to 70 amino acids, the amino acid sequence of which corresponds to the translation via the genetic code of a fragment of a nucleic acid sequence deemed to be non-coding present on a pre-mRNA of a protein, and
said cPEP being capable of modulating the accumulation of said protein in the plant cell and not being capable of modulating the accumulation of the mRNA encoding said protein.

21. The method according to claim 20, said cPEP having a size from 4 to 41 amino acids.

22. The method according to claim 20, said protein being encoded by an ORF comprising a nucleic acid sequence having at least 80% identity with a sequence chosen from the sequences: SEQ ID NOs: 1 to 161 and 171 to 175.

23. The method according to claim 20, wherein the sequence of said cPEP is chosen from the sequences: SEQ ID NOs: 162 to 164.

24. A method for modulating the accumulation of a protein in a plant cell comprising a step for introducing:
a cPEP; or
of a nucleic acid encoding said cPEP and the means of expressing it,
in said plant cell, the introduction of said cPEP resulting in a modulation of the quantity of said protein in said plant cell,
said cPEP having a size from 4 to 70 amino acids, the amino acid sequence of which corresponds to the translation via the genetic code of a fragment of a nucleic acid sequence deemed to be non-coding present on a pre-mRNA of a protein, and
said cPEP being capable of modulating the accumulation of said protein in the plant cell and not being capable of modulating the accumulation of the mRNA encoding said protein.

25. The method according to claim 24, said cPEP having a size from 4 to 41 amino acids.

26. The method according to claim 24, said protein being encoded by an ORF comprising a nucleic acid sequence having at least 80% identity with a sequence chosen from the sequences: SEQ ID NOs: 1 to 161 and 171 to 175.

27. The method according to claim 24, wherein the sequence of said cPEP is chosen from the sequences: SEQ ID NOs: 162 to 164.

28. The method according to claim 24, said method allowing:
promote the development of a plant; or
slow down or prevent the development of a plant; or
promoting the stress tolerance of a plant.

29. The method according to claim 24, said method modulating the accumulation of a protein in a plant cell chosen from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize)

30. The method according to claim 24, said method modulating the accumulation of a protein in a plant cell chosen from: Brassica napus, Brassica oleracea, Brassica rapa, Camelina sativa, Carica papaya, Glycine max, Gossypium raimondii, Gossypium spp., Hordeum vulgare, Lollium spp., Lotus japonicus, Medicago sativa, Medicago truncatula, Nicotiana benthamiana, Oryza sativa, Pisum sativum, Raphanus sativus, Solanum lycopersicum, Solanum melongena, Solanum tuberosum, Theobroma cacao, Triticum spp., Vitis vinifera and Zea mays.


SEQUENCE LISTING (175 sequences)

SEQ ID NO: 1 - DNA, 2184 bp, Arabidopsis thaliana
  misc_feature 1..2184: note = ORF of protein Aae15
  source 1..2184: mol_type = unassigned DNA
atgcaaattcgtctgaaacctgattattccttcttcatcgcctcttcctcaacgatggcttcgacttcttctttgggaccttctacactactctcttacggttctccttctcgtcagtttcctgattttgggttcagattgatttcgggtcacgaaagtgttcgaattccatcattccggcgatttcgggttcactgcgagtcaaaggaaaaagaagtgaagccgtcttctccatttcttgaaagctcctcgttttcgggagatgctgctttgcgctctagtgaatggaaggctgttcctgatatttggagatcatctgcagaaaagtatggtgatagagttgcattggtggatccttatcatgatcctcctttgaaactgacgtacaagcagttggaacaagaaattttggactttgctgagggcttacgagttcttggagtgaaagcagatgagaagattgcactttttgctgataactcctgccgatggcttgtttcagatcaaggtataatggccacaggggcagtcaatgttgtcagaggatctaggtcctctgttgaagagttactgcagatataccgtcattctgaaagcgtagccattgttgtggataatcctgagtttttcaaccgcattgctgagtcatttacgtcaaaggcatctctgagatttttgatacttctctggggtgagaaatcatcactggtcacacaggggatgcagattccagtttacagttatgcagaaattataaaccaaggacaggagagtcgtgcaaaattatcagcatctaatgataccaggagctatagaaatcaattcatcgattcagatgatacagctgcaattatgtataccagtggtaccacgggaaatccaaaaggcgttatgcttacacatcggaatctcttacaccagataaaacatttatccaaatatgtacctgctcaagctggggataaatttctaagcatgctaccatcatggcatgcctatgaacgtgctagtgaatacttcatattcacttgtggagttgagcaaatgtatacatctataagatacttaaaggatgatctaaagcggtaccaaccgaactatattgtgtccgttcctctagtatatgagacactttacagtgggattcaaaagcaaatttctgcaagttctgctggccgtaaatttctagcacttacattgatcaaagtcagtatggcatatatggagatgaaaaggatatatgagggtatgtgtctgacaaaagagcaaaagcctccaatgtatattgttgcttttgtggattggttgtgggcgagagtaattgctgccttgttgtggccattacatatgttggccaaaaagcttatctacaagaaaattcattcgtctattgggatatcgaaggctggtattagcggaggtggtagtttacccattcatgttgacaagttttttgaggccatcggtgtgattctacaaaatggttatggtttgacagagacctcacctgttgtctgtgcacggacacttagctgcaatgttcttggctcagctgggcatccaatgcatggtacagaattcaaaattgtagatcctgagactaataatgtactccctcctggttcaaagggcattatcaaagtcagaggtccacaggttatgaagggttattataagaatccatcgactacaaagcaagttctaaatgagagtggatggttcaatacaggagacaccggttggattgctcctcatcactcaaaagggcggagtcgccactgtggaggtgtcattgttcttgaaggccgtgcaaaagacacaattgtactttccacaggtgaaaatgtggaaccgttggagattgaagaagccgccatgagaagcagggtgattgaacaaattgttgttattggacaggaccgacgtcgccttggagctatcattatcccaaacaaagaggaagcacaaagagtagatcctgaaacatccaaagaaacactaaagagcttggtctaccaagaactgagaaaatggacatcagaatgttcgtttcaagtcggaccagtcttgatcgtcgacgaccctttcacgatagacaacgggttaatgacaccaactatgaagattagacgggacatggtcgtggctaaatacaaagaggagattgatcaactctacagttaa

SEQ ID NO: 2 - DNA, 2169 bp, Arabidopsis thaliana
  misc_feature 1..2169: note = ORF of protein Aae16
  source 1..2169: mol_type = unassigned DNA
atggcttcaacgtctctcggagcttcgattctcgtttctcactgctcctcagctcctgaatttcaagtttctgggatgagattggtgtttggttacaaggcttttggctgcagaacttcacgcaggggatttcgagttcgctgcgaatccaagattcaggagaaggagttaaggcggtgttcgccattcttagaacgcttatcattgccaagggaggctgctttgagctctaatgaatggaagtctgttcctgatatttggagatcatctgtggagaagtacggtgacagagttgcggtggtagatccgtatcatgacccgccttctacattcacgtacagacagttggaacaagaaatcttggactttgttgagggtttacgagtcgttggagtgaaagcagacgagaagattgcactttttgctgataactcctgtcgatggcttgttgcggatcaaggtataatggccacaggagcagtcaatgttgttagaggatccagatcgtctgtggaagagttattgcagatatactgtcattcggaaagtgtagcccttgttgtggataaccctgagtttttcaatcgcattgcggagtcattttcttacaaggcagctccaaaatttgtgattcttctctggggggaaaaatcgtcgttggttacagcgggtaggcacacaccagtctatagttacaacgaaattaaaaagtttggacaagagagacgtgcaaaatttgcaagatctaatgattctgggaagtatgaatatgaatacatcgatccagatgatatagccacaattatgtataccagtggaaccacaggaaatccaaaaggtgttatgctcacacatcagaatttgttacaccagataagaaacttgtccgattttgtgcctgcggaagctggggaaagatttctgagtatgttgccatcatggcatgcttatgaacgggcttgtgaatacttcatatttacatgtggagttgagcaaaagtatacgtctataagattcttaaaggatgatctcaagcgttatcaaccacactatcttatttcagttcctttagtatatgagacactctacagcgggattcaaaagcaaatttctgcaagctcccctgctcgtaaatttttggcacttacattgatcaaagtcagcctggcatatacggaaatgaaaagagtttatgagggtctttgtttgacaaaaaatcaaaagcctccaatgtatattgtttcgttggtggattggttgtgggcgagagtagttgcatttttcctatggccattgcatatgttggctgaaaagcttgtacacagaaaaattcgttcgtctattgggataacaaaggctggtgttagtggaggtggtagtttacctatgcatgttgacaagttttttgaggccatcggtgtgaatgtacaaaatgggtatggtttgacagaaacctcaccggttgtctctgcgcggaggcttaggtgtaacgttcttggctcagttgggcatcctattaaagatacagagttcaaaattgtagatcatgagactggtactgttcttccacctggttcaaagggcattgtcaaagtcagaggcccaccggtgatgaaaggttactacaagaatccactggccaccaagcaggttatagatgacgatggatggttcaatactggagatatgggttggattactcctcagcactcaacaggacggagtcgtagctgtggaggtgtcatcgttcttgaagggcgtgccaaggacactatcgtgctttccacaggtgaaaatgtggaaccattggagattgaagaagcggccatgagaagcaatttgattcaacaaatagtggttattggacaggatcaacgccgccttggagctattgttatcccaaacaaagaagcagcagaaggagcagcaaagcagaaaatttcacctgtagattctgaagtcaatgaacttagcaaggagacgataacaagcatggtctatgaagaactaaggaaatggacgtcacaatgctcgttccaagttggaccagttctgatcgtggatgaaccattcacgatagacaacggtttaatgacaccgacaatgaaaataagacgggacaaggtggttgatcaatacaagaatgaaatagaaagactctacaagtag

SEQ ID NO: 3 - DNA, 2112 bp, Arabidopsis thaliana
  misc_feature 1..2112: note = ORF of protein Abcg11
  source 1..2112: mol_type = unassigned DNA
atggagatagaagcaagcagacaacaaaccaccgtaccggtttcagtcggcggtgggaattttccggttggtgggttaagtccgttgagtgaagctatatggagagagaaagctccaacggagtttgtcggagacgtgtcagcgaggcttacgtggcaagatctgacggtgatggtgactatgggagatggtgagactcaaaatgttcttgaaggtcttaccggttacgctgaaccgggatctctcacggctctcatgggcccttccggctccgggaaatctactatgctcgacgcattagctagccgcctcgctgctaacgcttttctctccggcaccgttcttcttaacggccgcaaaaccaaactctcctttggaacagctgcatatgtgacccaagacgataacttgattggtacgttaactgtgagagagacgatttggtattcagcgagagttcgtcttccggacaagatgttacggtcggagaagcgtgctctagtggaaaggacaataatagagatgggtcttcaggattgtgcagatactgtcataggaaattggcatttgcgtggaattagtggtggagagaagagaagagtcagtattgctcttgagattttaatgagacctcgtttgctgtttctcgatgagccaacaagtggtcttgatagtgcttctgctttctttgtgactcagactttgcgtgctttatcacgagatggaaggacggtgatagcatcgatccaccaaccgagcagtgaggtttttgagttgtttgatcggttgtatctgctttctggaggcaaaactgtttactttggtcaagcttcagatgcatatgagttctttgcacaagctgggtttccatgtcctgcgttaaggaacccttctgatcatttcctgagatgcataaactcagactttgacaaagttagagccactttgaaaggttctatgaagctaaggtttgaagcaagcgatgatccgttggaaaagattaccacagccgaagctattagactattggttgactattaccacacttctgattactactacacagcaaaggctaaggttgaggagatatcccaatttaaagggactattcttgactccggaggcagccaggctagtttccttctgcagacctacactttaacaaagcgatcattcatcaacatgtcaagggactttggttattactggctcagacttctaatttacatcttggtcacagtctgcattggaaccatctatctgaacgttgggactagctacagcgccattctggcacggggctcatgtgcgtctttcgtctttggctttgtaacattcatgtcaattggtggatttccttcgttcgttgaagacatgaaggtcttccaaagagagagactaaacgggcactatggagtagctgcgtttgtgatagccaacacattatctgcaacaccattcctgatcatgataactttcatctccgggacaatctgttacttcatggtgggtctgcatccaggattcactcactacctcttctttgttctctgcctctatgcaagtgtcaccgttgtggagagcttaatgatggcaattgctagtattgttcccaacttcctcatgggtatcatcatcggagctggaatccaggggatcttcatgctggtttctggattcttcaggcttccaaatgacatcccaaaacctttctggcgttacccaatgtcctacatcagcttccacttctgggctctacaaggtcagtaccagaatgatctgagaggcttgacgtttgacagccaagggagtgctttcaagattccgggtgaatatgttcttgagaacgtctttcagatcgacttgcatcgatcaaaatggattaacctcagtgtgattcttagcatgattatcatctaccgcatcatcttcttcatcatgattaagaccaacgaggatgttactccttgggtcagaggttacatagcacgcagaaggatgaagcagaagaatggaactcagaacaccacagttgcaccagatggtcttacccagtctccttctctgagaaattacattgctacacgaacagatggagctcgcagatggtag

SEQ ID NO: 4 - DNA, 4362 bp, Arabidopsis thaliana
  misc_feature 1..4362: note = ORF of protein Abdcg34
  source 1..4362: mol_type = unassigned DNA
atgttaggacgagatgaagatctggtccgaacaatgagcgggagaggaagtttaggctccacaagtcaccggagtttagccggagctgcttcaaaaagcttcagagatgtgtttgctcccccaacggacgacgtgtttggtcggagcgatagacgagaagaagatgacgtggagctccgatgggcggcgcttgagagattaccgacttacgatcggttaagaaaaggtatgttgccgcaaacgatggtcaacggtaagattggacttgaagatgttgacgtcacgaatcttgctcctaaggagaagaaacatcttatggaaatgattctgaaattcgttgaagaagataacgaaaagtttcttcggcgattgagagaaagaactgacagagtgggaatcgaagttccgaaaattgaagtaaggtatgagaatctttcagtggaaggagatgttcgtagtgcaagcagagctcttcctactctcttcaacgtcactttgaatacaatcgagagtattcttggattattccacctgcttccatctaagaagaggaagattgagattcttaaagatataagtggcattatcaaaccatcaaggatgactttattacttggtccacctagttcagggaaaacaactttgttacaagctttagctgggaagcttgatgacactctccagatgtctgggaggataacttactgcggtcatgagtttcgtgagtttgttcctcagaagacttgtgcttacattagtcagcatgaccttcactttggagaaatgactgtgcgagagtcattggacttttcgggacgatgtttaggtgttgggactcggtaccagcttttgactgagctctcaaggagggagagagaagctggaattaagcctgaccctgagattgatgcatttatgaaatccattgctatatctggccaagaaactagtttggttacagactatgtacttaagttacttggtctggacatttgtgctgacacacttgttggagatgtgatgagacgaggtatttctggtggacagcggaaacgtctaacaacaggagagatgttggttggaccagcaacagctcttttcatggatgaaatatcaacagggttagacagttccacaacattccaaatttgcaagttcatgaggcaactagttcatatcgcggatgtcacaatggtcatttcgcttctacaacctgcgccagagacatttgagcttttcgacgacattatcctactctcagagggccaaattgtctaccaggggtcacgagacaacgttcttgagttctttgagtacatgggtttcaaatgtcctgaaaggaaaggtattgcagattttctgcaagaagttacgtctaagaaggaccaagaacagtattggaacaggagagaacatccttacagctatgtatcagtgcatgacttctcaagcggcttcaattcttttcacgcagggcaacaacttgcttcagaattcagggttccttatgacaaagcaaaaactcatcctgcagcactagtgacacaaaagtatggtatatcaaacaaggatctattcaaggcatgctttgatagagaatggcttcttatgaaacgtaactcctttgtgtacgtgttcaagaccgtccagataaccatcatgtctttgattgccatgacggtttattttcggacagaaatgcatgtcggcactgtgcaagatggtcaaaagttttatggtgctctctttttcagcttgatcaatctaatgtttaatggaatggctgaactagccttcacagtgatgaggcttccggttttcttcaagcagagggacttcttgttctatcctccatgggctttcgcattacccggttttcttctcaagattccgttatctcttattgaatcagttatatggatcgctcttacatactacactattggttttgctccttctgctgccaggttcttccggcagttgctagcatacttctgtgtgaatcagatggcactttctttatttagattccttggagcccttggaagaacagaagtcatcgccaactctggcgggacattagcattgctagtcgtatttgttcttggaggctttattatttctaaagatgacatcccgtcgtggctgacttggtgctattatacatcacctatgatgtatggacagactgcattagttataaatgaatttttggatgagcgatggggcagcccaaacaatgatacacgcatcaacgcgaaaacagttggagaagtcttgctgaagagcagaggcttctttacagaaccatattggttttggatctgtattggggcgctacttgggtttactgtgttgttcaacttctgttacataatagccttgatgtatttgaaccctcttggtaactccaaagctactacagttgtggaagaaggtaaagacaaacacaaagggagtcacagtggaacaggaggttctgtagtggaactcactagtacttcaagtcatggaccaaagaaaggaatggttttacctttccaaccactttctctagcattcaacaatgtgaactactacgtggatatgcctgcagaaatgaaggctcaaggagttgaaggagatcgacttcagttactaagagacgttggtggagctttcaggcctggcgtattgacagcattggtcggtgttagtggtgcgggtaagactaccttaatggatgtcttggccggtaggaaaacaggaggctatgtagaagggagtattaacatatctggttacccaaagaaccaagcaacatttgctagagtcagtggttactgtgagcaaaatgatatccattctccacatgttaccgtttatgaatcccttatctattcagcatggcttcgtctttccgccgatatagataccaaaacacgagagatgtttgtggaggaagtgatggagttggtggagcttaaacctcttagaaactctatagttggtcttcctggtgtagatggtctttcaacagaacagaggaagaggcttactatagcagttgaattggtagctaatccatcaataatcttcatggacgagccaacatctggtcttgatgcaagagctgctgccattgttatgcgtactgttaggaatactgttgatacaggaagaactgttgtttgtacaattcaccaacctagcatcgacattttcgaatccttcgatgagttgctgttgatgaaacgaggaggacaagttatatatgctggaacactaggacatcactcacaaaaactcgttgaatactttgaggctattgaaggggttccaaagatcaaggacggatacaatcctgcgacgtggatgcttgacgtcactactccttcaatggagtcacaaatgagcgtggactttgctcaaatattcgttaactcctctgttaatcggagaaatcaggaactcatcaaagagctgagtactccaccaccgggatcgaatgatctctacttccgaaccaagtacgcgcaaccattttctactcaaaccaaagcttgcttctggaaaatgtattggtcaaactggagatatcctcagtacaatgccattcggtttctcatgacagtagtcattggtgtcttgtttggtctacttttctggcaaacaggaactaaaatagagaaagagcaagacctgaataatttctttggagccatgtatgctgctgtattgttcctcggtgccaccaacgctgcaacagttcaacccgcagttgctattgagcgaacggttttctaccgcgaaaaagccgctggaatgtactccgccattccctatgcaatttctcaggtagcagtagaaatcatgtacaacactatacaaaccggagtttacacacttatcctttactcgatgatcggatacgactggactgtggtcaaattcttctggttctactactacatgttaacatgcttcgtctacttcaccctatacggtatgatgcttgtcgccttgacaccaaactatcaaatagctggaatctgcttgtccttcttcctcagtttctggaatcttttctccggtttcctcatccctagaccgcaaataccaatatggtggagatggtactattgggcatcacctgtggcttggacattgtatggaatcatcacatctcaagtaggagacagggattcgattgtgcacatcactggtgttggagatatgagtcttaaaacgttgctcaaaaatggattcgggttcgattatgacttcttaccagttgtagccgttgtccacatcgcctggatcttgatctttctctttgcctttgcttatggtatcaagttcctcaacttccaaagaaggtga

SEQ ID NO: 5 - DNA, 6765 bp, Arabidopsis thaliana
  misc_feature 1..6765: note = ORF of protein Acc1
  source 1..6765: mol_type = unassigned DNA
atggctggctcggttaacgggaatcatagtgctgtaggacctggtataaattatgagacggtgtctcaagtggatgagttctgtaaagcacttagagggaaaaggccgatccatagtattttgatagctaacaatggaatggcggctgtgaagtttatacgtagtgtcagaacatgggcttatgaaacatttggtacggaaaaagccatattgttggtggggatggcaacccctgaagacatgcggatcaatgcggagcatatcagaatcgctgatcagtttgttgaggttcccggaggaaccaacaataacaattatgctaacgttcagctgattgtggagatggctgaagtaacacgcgtggatgcagtttggcctggttggggtcatgcatctgaaaaccccgaattacctgatgccctagatgcaaaaggaatcatatttcttggtcctccagcatcttcaatggcagcactgggagataagattggttcttcgttgattgcacaagctgctgatgtacccactctgccatggagtggttcccatgttaaaatacctcctaatagcaacttggtaaccatcccagaggagatctaccggcaagcatgtgtctacacaactgaagaagcgattgctagctgtcaagttgtcggttacccagcaatgatcaaagcatcgtggggtggtggtggtaaaggaatcaggaaggttcataatgatgatgaggttagggctctattcaagcaagttcagggtgaggtcccaggctcaccaatattcataatgaaggttgcgtcacagagtcggcatctagaggtccagctgctctgtgacaagcatggaaatgtttcagctctgcatagccgtgattgtagcgtccagagaagacatcaaaagatcatagaggagggtccaattactgtggctccgccagaaactgtcaagaaacttgaacaagcagctagaaggttggctaagagtgttaactatgttggagctgctactgttgagtatctctacagtatggacactggggagtactacttcttagagcttaaccctcgcttacaggttgagcatcctgtcactgagtggattgccgagataaatcttcctgctgcccaagttgctgtggggatgggaattcctctctggcaaatccctgagataagacggttctatggaatagaacatggtggaggttatgattcttggcgaaaaacatctgttgtagccttcccttttgattttgataaagctcaatctataaggccaaaaggtcattgtgtggctgtacgtgtgacaagtgaggatcctgatgacgggttcaaaccaaccagcggtagagttcaggagttgagttttaagagcaagccaaatgtgtgggcgtacttctctgtcaagtctggtggaggcatccacgagttctcggattcccagtttggacatgtttttgcatttggggaatccagagccctggcgatagcgaatatggttcttgggctaaaagaaattcagatccgtggagaaattaggactaacgttgactacacgatcgaccttttacatgcttctgattaccgtgataacaaaattcacactggttggttggatagtaggattgctatgcgggtcagagctgagaggcctccatggtatctctctgttgtcggcggagctctctataaagcatcagcgaccagtgctgctgtggtttcagattacgttggttatctggagaaggggcaaatccctccaaagcatatatctcttgtacattctcaagtgtctctgaatattgaaggaagtaaatatacgattgatgtagtccggggtggatcaggaacctacaggctaagaatgaacaagtcagaagtggtagcagaaatacacactctacgtgatggaggtctgttgatgcagttggatggcaaaagccatgtgatatatgcagaggaagaagctgcaggaactcgtcttctcattgatggaagaacttgtttgctacagaatgaccacgatccatcaaagttaatggctgagacaccgtgcaagttgatgaggtatttgatttccgacaacagcaatattgacgctgatacgccttatgccgaagttgaggtcatgaagatgtgcatgccacttctttcacctgcttcaggagttatccattttaaaatgtctgaaggacaagccatgcaggctggtgaacttatagccaatcttgatcttgatgatccttctgctgtaagaaaggccgaacccttccatggaagtttcccaagattagggcttccaactgcaatatccggtagagttcatcagagatgtgccgcaacattaaatgctgcacgcatgattcttgctggctatgagcataaagtagatgaggttgttcaagacttacttaattgccttgatagccctgaactcccatttcttcagtggcaagagtgctttgcagttctggcgacacgactacctaaaaatctcaggaacatgctagaatcaaagtatagggaatttgagagtatttccagaaactctttgaccaccgatttccctgccaaacttttaaaaggcattcttgaggcacatttatcttcttgtgatgagaaagagagaggtgcccttgaaaggctcattgaaccattgatgagccttgcaaaatcttatgaaggtggtagagaaagtcatgcccgtgttattgttcattctctctttgaagaatatctatcagtagaagaattattcaatgataacatgctggctgatgttatagaacgcatgcgtcagctatacaagaaagatctgttgaaaattgtggatatagtgctctcacaccagggcataaaaaacaaaaacaaactcgttctccggctcatggagcagcttgtttaccctaatcctgctgcttacagagataaacttattcgattctcaacacttaaccatactaactactctgagttggcgctcaaggcgagtcaattacttgaacagaccaaactaagtgagcttcgttcaaacattgctagaagcctttcagagttagaaatgtttacagaggacggagaaaatatggatactcccaagaggaaaagtgccattaatgaaagaatagaagatcttgtaagcgcatctttagctgttgaagacgctctcgtgggactatttgaccatagcgatcacacacttcaaagacgggttgttgagacttatattcgcagattataccagccctacgtcgttaaagatagcgtgaggatgcagtggcaccgttctggtcttcttgcttcctgggagttcctagaggagcatatggaaagaaaaaacattggcttagacgatcccgacacatctgaaaaaggattggttgagaagcgtagtaagagaaaatggggggctatggttataatcaaatctttgcagtttcttccaagtataataagtgcagcattgagagaaacaaagcacaacgactatgaaactgccggagctcctttatctggcaatatgatgcacattgctattgtgggcatcaacaaccagatgagtctgcttcaggacagtggggatgaagaccaagctcaggaaagagtaaacaagttggccaaaattcttaaagaggaagaagtgagttcaagcctctgttctgccggtgttggtgtaatcagctgtataattcagcgagatgaaggacgaacacccatgagacattctttccattggtcgttggagaaacagtattatgtagaagagccgttgctgcgtcatcttgaacctcctctgtccatttaccttgagttggataagctgaaaggatactcaaatatacaatatacgccttctcgagatcgtcaatggcatctgtatactgttacagacaagccagtgccaatcaagaggatgttcctgagatctcttgttcgacaggctacaatgaacgatggatttatattgcagcaagggcaggataagcagcttagccaaacactgatctccatggcgtttacgtcgaaatgtgttctgaggtctttgatggatgccatggaggaactggaactgaatgcccataatgctgcaatgaaaccagatcacgcacatatgtttctttgcatattgcgtgagcagcagatagatgatcttgtgcctttccccaggagagttgaagtgaatgcggaggatgaagaaactacagttgaaatgatcttagaagaagcagcacgagagatacatagatctgttggagtgagaatgcataggttgggcgtgtgcgagtgggaagtgcggctgtggttggtgtcctctggactggcatgtggtgcttggagggttgtggttgcaaacgtgacaggccgtacatgcactgtccacatataccgagaagttgaaactcctggaagaaacagtttaatctaccactcaataaccaagaagggacctttgcatgaaacaccaatcagtgatcaatataagcccctgggatatctcgacaggcaacgtttagcagcaaggaggagtaacactacttattgctatgacttcccgttggcatttgggacagccttggaactgttgtgggcatcacaacacccaggagttaagaaaccatataaggatactctgatcaatgttaaagagcttgtattctcaaaaccagaaggttcttcgggtacatctctagatctggttgaaagaccacccggtctcaacgactttggaatggttgcctggtgcctagatatgtcgaccccagagtttcctatggggcggaaacttctcgtgattgcgaatgatgtcaccttcaaagctggttcttttggtcctagagaggacgcgtttttccttgctgttactgaactcgcttgtgccaagaagcttcccttgatttacttggcagcaaattctggtgcccgacttggggttgctgaagaagtcaaagcctgcttcaaagttggatggtcggatgaaatttcccctgagaatggttttcagtatatatacctaagccctgaagaccacgaaaggattggatcatctgtcattgcccatgaagtaaagctctctagtggggaaactaggtgggtgattgatacgatcgttggcaaagaagatggtattggtgtagagaacttaacaggaagtggggccatagcgggtgcttactcaaaggcatacaatgaaacttttactttaacctttgttagtggaagaacggttggaattggtgcttatcttgcccgcctaggtatgcggtgcatacagagacttgatcagccgatcatcttgactggcttctctacactcaacaagttacttgggcgtgaggtctatagctctcacatgcaactgggtggcccgaaaatcatgggcacaaatggtgttgttcatcttacagtctcagatgatcttgaaggcgtatcagcaattctcaactggctcagctacattcctgcttacgtgggtggtcctcttcctgttcttgcccctttagatccaccggagagaattgtggagtatgtcccagagaactcttgcgacccacgagcggctatagctggggtcaaagacaataccggtaaatggcttggaggtatctttgataaaaatagtttcattgagactcttgaaggctgggcaaggacggtagtgactggtagagccaagctcgggggaatacccgttggagttgttgcagttgagacacagactgtcatgcagatcatcccagccgatcctggacagcttgactctcatgaaagagtggttccgcaagcagggcaagtctggtttcctgattcagcggccaagactgctcaagcgcttatggatttcaaccgggaagagcttccattgtttatcctagcgaactggagagggttttcaggtgggcagagagatcttttcgaaggaatacttcaggcaggttcaactatagtagaaaatctgagaacctatcgtcagccagtgtttgtgtacatcccaatgatgggagagctgcgcggtggagcgtgggttgttgttgacagccagataaattcggattatgttgaaatgtatgctgatgaaacagctcgtggaaatgtgcttgagccagaagggacaatagagataaaatttagaacaaaagagctattagagtgcatgggaaggttggaccagaagctaatcagtctgaaagcaaaactgcaagatgccaagcaaagcgaggcctatgcaaacatcgagcttctccagcaacagattaaagcccgagagaaacagcttttaccagtttatatccaaatcgccaccaaatttgcagaacttcatgacacttccatgagaatggctgcaaagggagtgatcaaaagtgttgtggaatggagcggctcgcggtccttcttctacaaaaagctcaataggagaatcgctgagagctctcttgtgaaaaacgtaagagaagcatctggagacaacttagcatataaatcttcaatgcgtctgattcaggattggttctgcaactctgatattgcaaaggggaaagaagaagcttggacagacgaccaagtgttctttacatggaaggacaatgttagtaactacgagttgaagctgagcgagttgagagcgcagaaactactgaaccaacttgcagagattgggaattcctcagatttgcaagctctgccacaaggacttgctaatcttctaaacaaggtggagccgtcgaaaagagaagagctggtggctgctattcgaaaggtcttgggttga

SEQ ID NO: 6 - DNA, 1134 bp, Arabidopsis thaliana
  misc_feature 1..1134: note = ORF of protein Agb1
  source 1..1134: mol_type = unassigned DNA
atgtctgtctccgagctcaaagaacgccacgccgtcgctacggagaccgttaataacctccgtgaccagcttagacagagacgcctccagctcctcgataccgatgtggcgaggtattcagcggcgcaaggacgtactcgggtgagcttcggagcaacggatctggtttgttgtcgtactcttcagggacacaccggaaaggtttattcattagattggacaccggagaggaaccggattgtcagtgcatctcaagatgggagattaatcgtgtggaatgctctaacgagtcagaaaactcatgctattaaactcccttgtgcatgggttatgacatgtgctttctctccaaatggtcagtcggttgcgtgtggtggattagacagtgtatgttctatctttagccttagctcaacggcggacaaggatggaactgtaccggtttcaagaatgctcactggtcacaggggatatgtttcgtgctgtcagtatgtcccaaatgaggatgcccaccttatcaccagttcaggtgatcaaacttgtatcttatgggatgtaactactggtctcaaaacttctgtttttggcggtgaatttcagtctggacatactgctgatgtactaagcgtctcaatcagtggatcaaacccaaactggtttatatctggttcatgcgattccacagcacggttgtgggacactcgtgctgcaagccgagcagtgcgtacctttcatggtcacgagggagatgttaatacggtcaagttctttccggatgggtatagatttgggactggatcagacgatggaacatgcaggctgtatgacataaggactggtcaccaactccaggtctatcagccacatggtgatggtgagaacggacctgtcacctccattgcattctctgtgtcagggagacttcttttcgctggctatgcgagcaacaacacttgctacgtttgggataccctcttgggagaggttgtattggatttgggattacagcaggattcacacaggaatagaataagctgtttggggttgtcagcagatggaagtgccttgtgtacaggaagttgggattcaaatctaaagatatgggcgtttggaggacacaggagagtgatttga

SEQ ID NO: 7 - DNA, 2013 bp, Arabidopsis thaliana
  misc_feature 1..2013: note = ORF of protein Als
  source 1..2013: mol_type = unassigned DNA
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatactga

SEQ ID NO: 8 - DNA, 1134 bp, Arabidopsis thaliana
  misc_feature 1..1134: note = ORF of protein Anac076
  source 1..1134: mol_type = unassigned DNA
atggaatcggtggatcaatcatgtagtgttcctccgggattcagattccatccaacagatgaagagctcgttggttactatttaaggaaaaaagttgcatcgcaaaagatcgatcttgatgtcataagagatattgatctctacagaatcgaaccatgggatttacaagagagctgccgaatcggatatgaggaaagaaatgaatggtatttcttcagccacaaagataagaaatatccaacaggaacaagaacaaacagagcgaccatggctgggttttggaaagccacgggccgagacaaggctgtttacgacaagtcaaaactgattggtatgagaaaaacacttgtgttctacaaaggaagagcccctaatggccaaaaaaccgattggatcatgcatgaataccggctagagtcagatgagaatgcacctcctcaggaagaagggtgggtggtttgtagagcgttcaagaaaaagccaatgaccgggcaagccaagaacacagaaacttggagctcaagttacttttacgacgaattaccgagtggagtacgctcagttacggagcctcttaactacgtatctaagcagaaacaaaacgtttttgcacaagatttaatgttcaagcaagaactagaaggatcagatatcggtttaaacttcatccactgcgatcaattcattcaacttccgcagcttgaaagcccttcactccctcttaccaaaagaccagtgagcttgacgtcaattacatcattggagaagaataaaaatatctacaaaagacatttaatagaagaggatgtgagcttcaatgcgctaataagtagtggaaataaagataagaagaagaagaaaacatcagtgatgacgacggattggagagcactcgataaatttgttgcttctcaacttatgagccaagaagatggagtttcgggttttggaggtcatcatgaagaagataacaataaaatcggtcattacaataacgaagagagcaataacaagggatcagtagagactgcttcttccacgttattgagtgatagagaagaagagaacagattcatcagtggattattgtgttccaacttggactatgacttatatagggatttacatgtttga

SEQ ID NO: 9 - DNA, 2601 bp, Arabidopsis thaliana
  misc_feature 1..2601: note = ORF of protein Apg9
  source 1..2601: mol_type = unassigned DNA
atgatgagcagtgggcataagggtccaaatgtacgtaacttttttaaatggcaacgtggtgaatcatcatcgtctttgactactggattgcttcataatgagtcacacgagattgagctatctaactacggaggaataccaagtcctggtagtgagagcccatcgggacttcttaatggagagagtttaaatgttcaaccaattgctgatttggatctttttgtcgaaaggctctacagctactacagagacaaaggactttggtgtattatagtaaagtgggctgttgagcttctaagtctgggcttcatcatatgcttctctgggtttttcttgctgtatgttgattggaatggtcttcagaatgcaaaatgtggaatggatgcagttgaatcaggaactaaaccatgtgatcttgtcaaagaagctatccatccgcatcctttatcccctttcacgctcacaactgctataattgttggatatttggccctcttctctgtgtactggctcttctgcttcttaaggttttttgcccagttgaaagatactttggactttcggcacttttattacaacaatctccatgtcacagacaatgaaatccttactatgccatgggcaacagtccttgagaaggttgttcagttacaaagctcacaatgcctttgtgtggttaaggatctttctgctcatgatatggttatgcgccttatgcggaaagaaaactacttgattggaatgctcaacaaaggtttgctttctttcccaatttcccattggatccctggtgctggtccagcagtcaaatccgcaccagatgggacacagtatcaccttgtattgacaaagacccttgagtggacccttaactggtgcatattgcagagcatgtttgactgcaattttcgtgttagaagggactttgtttcaaatcctacaacactgaagaaacgactatttgtggttgggcttgctatgcttctgctgtcaccgtttcttgtcatctttatgttggtgtatttattcctaaggcatgctgaacagttctacaatcatccaagtactgcatcttctcgaagatggtccaatttgtcaaagtggctcttcagggaattcaatgaggttgaccatttattcaagcatcggattaatagcagtgtagtacatgcatccgaatatctaaagcaatttccctcgcctatcatctccattattgcaaagtttgtctcgtttgtttctggcggctttgctgctgtcttaatcatcattgcgtttcttgaagaatctctacttgagggacatatattcggccgcaatctgttctggtatgccgctgtttttggaacaattacagctattagtagagctgccatatcggatgaacttttggtcctcgacccagtaggaactatgtctttggtggtccaaaacactcattatatgcctaagagatggcgtggaaaggagaacaaggatgatgtccgcttggagttagagactctgtttcagtatactggaatgatgctattggaggagatagcttcaatcttcatcacaccatttttgctgatgtttgtcgtgccaaagcgggtggatgacatcttgcaattcattaaggatttcacagttgatattgaaggtgtaggccatgtttgcagctttagtgctttctactttgagaaccatggaaatatcaaatatggttcaccacataatgcaactcgtcgtgagcagagaagctcacaagggaaaatggagaaatcattcttgagtttccagagtagttatccttcttgggagtcagattctcttgggaagcagtttctgtcaaatctcagaactttccgggaccgaaagcttcatgaaataaacacaagacactcatctcctagtagggcgtggcgagaaagcactaatacccctgctttatacagagacattccaagaaatccgcttgcgagcggcaatcacactgattcaatgtggctgattgatccagatcagaggaatcatccttatcttcttgactggtattacacttctcaggctcacaacagaactgatcacccaatagagagagcaaatgaaatcttaaccgcaaatcagaacgctacagattgttggcctcctgatttgggaatccgtggtgaagatagcagagatctgctcaacatggaggcaagcacatcaggtcagtttttcagagagagcattctgcgccatgaccaacccgagggagaggatagttatgggagtcagcatcctcttgatggtaggaaccagtggtggggaagaggcaaccactctcagatcagcacagctcatcccgcaaccaccaatagcttcatcgagccacctgattttataaaccgctacacagcaggaaacttattagacaactcttggagcagaagaagcatagaagaagaagatgaagaagaagaagaattggattgggaagagaatgcaagaagaaatttgtcgagaactacattcatggacgataacgatatcgaagctgggattgatctccatttcgacgatgtatatagttcaagacctcaagaaacttcaacatcaagcaccacattacggtga

SEQ ID NO: 10 - DNA, 618 bp, Arabidopsis thaliana
  misc_feature 1..618: note = ORF of protein Arlb1
  source 1..618: mol_type = unassigned DNA
atgttttctcttatgtctggactatggagttacatgtttagcaagactgagtttaatgtgcttattcttggtatcgacaaagctgggaagacgacatttttggagaaactgaagacgatatactcaatctcggaaggtcttcctcatgatcgaattgttcctactgtggggcttaatattggtcgcattgaagtctccaatgctaaaattgtgttctgggatctgggaggtcagcctggtttgcggtctatttgggagaaatactatgaagaagcacacgcgttgatatatttgattgatgcagcttgcccaacccgctttgaagattctaaatctgctttagaaaaagccctgcggcatgaggatttgcaaggagcccctcttttaatattggcaaacaagcaagacctcacaaatgctgtatcggctgaggaactggaccgataccttgatttaaagaaattggatgaaagagtgtacatgtttgaagcagtttctggatatgatgggcgaggaatcaaagaaagtatagaatggctggttggagtaatggagaaaagcaagagaaccgaatcattaagagcccgtgcagggtacactcctgtgccaaattcatag

SEQ ID NO: 11 - DNA, 2073 bp, Arabidopsis thaliana
  misc_feature 1..2073: note = ORF of protein Arr1
  source 1..2073: mol_type = unassigned DNA
atgatgaatccgagtcacggaagaggactcggatcggctggtgggtccagctccggtagaaatcaaggaggtggtggtgagaccgtcgtcgagatgtttccttctggtcttcgagttcttgtcgttgacgatgacccaacttgtctcatgatcttagagaggatgcttaggacttgtctttacgaagtaacgaaatgcaacagagcagagatggcattgtctctgctccggaagaacaaacatggattcgatatagtaatcagtgatgttcatatgcctgacatggacggtttcaagcttcttgagcatgttggtctagagatggacttacctgttatcatgatgtctgcggatgattcaaagagtgtggttctaaagggagtaacgcacggtgcggttgattaccttatcaagcctgtacgtatggaggcacttaagaacatatggcagcatgtagttaggaagaggagaagtgaatggagtgtaccggaacattctgggagcattgaggagactggcgagagacagcagcagcaacatagaggaggtggtggtggtgcagctgtttctggtggagaggatgcggtggatgataactcatcctcggttaacgaaggtaacaattggaggagcagttcacggaagaggaaagacgaggaaggagaagagcaaggagatgataaggacgaagatgcgtcgaatttgaagaaaccgcgtgtcgtctggtctgttgaattgcatcagcagtttgttgctgctgttaatcagctcggcgttgagaaggcggttcctaaaaagatcttagagctgatgaatgttcctggtctaacccgagaaaacgtagcaagtcacctccagaaataccggatatatctaagacggcttggaggggtatcgcagcaccaaggcaatcttaacaactcgtttatgacgggtcaggatgcgagcttcggacctctttcgacattgaatgggtttgatcttcaagcactagccgtcacaggtcagttacctgcacagagtcttgcacagcttcaagccgctggtttaggccggcctgcgatggtctctaagtcaggtttgccggtttcctccattgtggatgagagaagcatcttcagctttgacaacacgaaaacaagatttggagaagggcttgggcatcacgggcaacaaccccaacagcaaccacagatgaacttacttcacggtgtccccacgggtttacaacagcagcttcctatgggtaatcgaatgagtattcaacaacagattgctgctgttcgagctggaaatagtgttcaaaacaacggaatgctgatgcctctagcgggtcagcagtctttgcctcggggaccaccgcctatgctaacctcttcgcaatcatccatcaggcagccgatgttatcaaaccgcatttccgagagaagtggtttctctggaaggaacaatatccccgagagcagcagagtgttaccgacaagttacactaatctcacaacacaacactcatcaagctcgatgccttataacaacttccaaccagaacttcccgtgaacagtttcccgctggcaagtgcaccagggatatcagtaccggttcggaaagccacttcttaccaggaagaggttaacagctccgaagcgggtttcactacgccgagctacgacatgttcaccaccagacagaatgattgggatctgaggaatattggaatagcctttgactcacatcaggactcagaatccgctgcgttttccgcttcagaagcctactcttcttcgtccatgtcaagacacaacacgacagttgcagccaccgagcatggccgaaaccaccagcagccaccatcgggaatggtacagcaccatcaggtttatgcagacggaaacggtggttcagtgagggtgaaatcagagagagtggctacggatacagcaacaatggcgtttcacgagcagtatagtaatcaagaagatcttatgagcgcacttcttaagcaggaagggattgcaccggttgatggcgaattcgactttgacgcatactccatcgataacattccggtttga

SEQ ID NO: 12 - DNA, 555 bp, Arabidopsis thaliana
  misc_feature 1..555: note = ORF of protein Arr5
  source 1..555: mol_type = unassigned DNA
atggctgaggttttgcgtcccgagatgttagatatctctaacgacacttcttcattagcatcaccgaaacttcttcatgttcttgctgttgatgatagtatggttgatcggaagttcatcgagcggttactcagagtctcatcttgtaaagttactgttgtcgatagtgcgacaagagctttacaatatcttggattagatggagagaataatagttcggttggatttgaggatctgaagattaatttgataatgacggattactctatgcctgggatgactggatatgaactactgaagaagatcaaagaatcatcagctttcagagaaataccagttgtgattatgtcttcagagaacatcttgcctcgtatcgatagatgtcttgaagaaggagctgaagatttcttattgaagcctgtgaaattagctgatgtgaagagattaagagattctttaatgaaagctgaggaaagagcttttaagaatattatgcacaagagagagcttgaagctaatgatatctactcgcagctaaaacgcgcaaagatctga

SEQ ID NO: 13 - DNA, 561 bp, Arabidopsis thaliana
  misc_feature 1..561: note = ORF of protein Arr6
  source 1..561: mol_type = unassigned DNA
atggctgaagttatgctaccgaggaagatggagattctcaaccattcttcaaagtttggatcaccggatcctcttcatgttcttgccgtcgacgacagtcacgttgatcgtaaattcatcgagcgtttgctcagagtatcttcttgcaaagttactgttgttgatagtgcaacaagagctctccaataccttggtttggatgttgaggaaaaatcagtcggttttgaggatttgaaggttaatttaattatgactgattactcaatgcccggtatgactggatatgaactcttgaagaagatcaaagaatcctcagcttttagagaagtaccggtggtaattatgtcctccgagaacattttgcctcgtattgatagatgtcttgaagaaggggctgaagatttcttactgaagccggtaaaactctcggatgtaaaaagattaagagattctctaatgaaggttgaagatttgtctttcacaaagagtattcagaagagagagctagaaacagagaatgtctaccctgttcactcgcagctcaaacgcgcaaagatctga

SEQ ID NO: 14 - DNA, 1380 bp, Arabidopsis thaliana
  misc_feature 1..1380: note = ORF of protein At59
  source 1..1380: mol_type = unassigned DNA
atggcagcagctttcttgaatcttggcggctatgttttcgttttcttctcttcgttcttagctatagtggccccacaggttcgaggcaatgttgctgtctttgatagctactggacgcaacgacaaagtgatgcattgaaacaaaccatcggctcttacgatcctcatccacttaacgtcaccaaccattttaattaccatgtcaatatagcagtggatgcatcggaatcaaggaacgatacaaggagagagcttactcaggtgagaagtggtcgtaaaacgcacaaaagcagcggaaaatgcctagcttataacccgatagacaactgctggcgatgcgatcgtaactgggctaataaccggaagaaactagccgactgtgtgctcggattcggccggagaaccaccggaggaaaagacggaccgatatacgtcgtcaaagacgcgtctgacaacgatcttatcaaccctaaaccaggaacgttaaggcatgcagtgactcgagacggaccgctttggatcatattcgcaagaagcatgatcataaaactgcaacaagaactgatgataacgagcgacaaaacaatcgatggaagaggagctagggtttacatcatggaaggtgctggattaacgttgcaatttgtgaataatgtgatcattcacaatatttatgtcaagcacattgttcctgggaacggtgggttgatcagagactctgaggctcacatagggcttaggacaaaaagtgatggtgatggaatcagtttgtttggagcaaccaatatttggatcgaccatgtctcaatgacaagatgtgcagatggtatgatcgatgctattgacggctccactgctgtaactatttccaacagtcatttcaccgaccaccaagaggtgatgttgtttggggcgagggacgaacacgtgatcgacaagaaaatgcagataacggttgcgtttaaccacttcggtaagcgattggaacagagaatgcctcgttgcagatacggaacgatacatgtggtgaacaatgactacacacattgggaaatgtatgcaattggtgggaacatgaaccctacaattattagtcaaggcaatcgtttcattgctcctcctaatgaagaagccaaacagattacaaaaagagagtacacaccatacggtgaatggaaatcttggaattggcaatcagaaggagattactttctcaacggagcttattttgtgcaatccggaaaagcaaatgcgtggagctctaaaccgaaaaccccgctcccaaacaagtttacaattcgacctaaaccgggaacgatggtccggaaacttaccatggatgcaggagtacttggctgcaaacttggtgaagcttgctga

SEQ ID NO: 15 - DNA, 1848 bp, Arabidopsis thaliana
  misc_feature 1..1848: note = (ORF of protein Bak1
  source 1..1848: mol_type = unassigned DNA
atggaacgaagattaatgatcccttgcttcttttggttgattctcgttttggatttggttctcagagtctcgggcaacgccgaaggtgatgctctaagtgcactgaaaaacagtttagccgaccctaataaggtgcttcaaagttgggatgctactcttgttactccatgtacatggtttcatgttacttgcaatagcgacaatagtgttacacgtgttgaccttgggaatgcaaatctatctggacagctcgtaatgcaacttggtcagcttccaaacttgcagtacttggagctttatagcaataacattactgggacaatcccagaacagcttggaaatctgacggaattggtgagcttggatctttacttgaacaatttaagcgggcctattccatcaactctcggccgacttaagaaactccgtttcttgcgtcttaataacaatagcttatctggagaaattccaaggtctttgactgctgtcctgacgctacaagttctggatctctcaaacaatcctctcaccggagatattcctgttaatggttccttttcacttttcactccaatcagttttgccaacaccaagttgactccccttcctgcatctccaccgcctcctatctctcctacaccgccatcacctgcagggagtaatagaattactggagcgattgcgggaggagttgctgcaggtgctgcacttctatttgctgttccggccattgcactagcttggtggcgaaggaaaaagccgcaggaccacttctttgatgtaccagctgaagaggacccagaagttcatttaggacaactgaagaggttttcattgcgtgaactacaagttgcttcggataattttagcaacaagaacatattgggtagaggtggttttggtaaagtttataaaggacggttagctgatggtactttagtggccgttaaaaggctaaaagaggagcgcacccaaggtggcgaactgcagttccagacagaggttgagatgattagtatggcggttcacagaaacttgcttcggcttcgtggattttgcatgactccaaccgaaagattgcttgtttatccctacatggctaatggaagtgttgcctcctgtttaagagaacgtcccgagtcccagccaccacttgattggccaaagagacagcgtattgcgttgggatctgcaagagggcttgcgtatttacatgatcattgcgacccaaagattattcatcgagatgtgaaagctgcaaatattttgttggatgaagagtttgaagccgtggttggggattttggacttgcaaaactcatggactacaaagacacacatgtgacaaccgcagtgcgtgggacaattggtcatatagcccctgagtacctttccactggaaaatcatcagagaaaaccgatgtctttgggtatggagtcatgcttcttgagcttatcactggacaaagggcttttgatcttgctcgcctcgcgaatgatgatgatgtcatgttactagactgggtgaaagggttgttaaaagagaagaaattggaagcactagtagatgttgatcttcagggtaattacaaagacgaagaagtggagcagctaatccaagtggctttactctgcactcagagttcaccaatggaaagacccaaaatgtctgaagttgtaagaatgcttgaaggagatggtttagctgagagatgggaagagtggcaaaaggaggaaatgttcagacaagatttcaactacccaacccaccatccagccgtgtctggctggatcattggcgattccacttcccagatcgaaaacgaatacccctcgggtccaagataa

SEQ ID NO: 16 - DNA, 843 bp, Arabidopsis thaliana
  source 1..843: mol_type = unassigned DNA
atggcgtcttcgtcgttctcagtcacatctccagctgctgctgcttccgtctatgcagtcactcaaacctcctcgcacttcccaatccaaaaccgctctcgcagagtttctttccgtctctctgctaagcccaagcttcgctttctctccaagcctagtcgcagtagctaccctgtggtgaaagcacaatctaacaaggttagtactggtgcatcatcaaatgctgccaaagttgatgggccatcatcagctgaaggaaaggagaaaaactcattgaaggagtcgtctgcttcttctcctgaattagctacagaagagtctatttctgagttccttacccaagtaacaactcttgtcaagcttgtggattcaagagacattgttgagttgcagttgaaacaactcgactgtgaactagtcattcgaaaaaaagaagccttacctcaacctcaagctcctgcatcttatgttatgatgcagcaaccaaatcaaccatcttatgcccagcaaatggctcctcctgctgcacctgctgctgccgcaccagccccttctacgccagcctctctgcctccaccatccccacctactccagccaaatcgtcacttcctactgttaaaagccccatggctggcacattctaccgtagtcctgcacctggtgaaccaccctttattaaggttggagacaaagtgcagaaggggcaagttctatgcattgttgaagccatgaagttaatgaatgaaatagagtctgaccataccggaaccgtagtcgatattgtcgcagaagacggcaagcctgtcagcctcgacactcctctgtttgtggttcaaccgtag

SEQ ID NO: 17 - DNA, 768 bp, Arabidopsis thaliana
  misc_feature 1..768: note = ORF of protein Bccp2
  source 1..768: mol_type = unassigned DNA
atggcgtcattgtcggtaccttgcgtcaagatctgtgctctgaacaggcgggtcggatctctccctggaatctctactcaaagatggcagccgcaacccaatgggatctcctttccctccgatgtttctcagaatcattctgcattctggaggttgcgtgcaacaactaatgaggttgtttctaactccactccaatgactaatggtgggtatatgaacggaaaagcgaaaaccaatgttcctgaacccgccgagctctctgaatttatggctaaagtctctggtcttcttaagcttgtggattcaaaagacatagtggaacttgaactaaagcagctcgactgtgagatcgttattcgaaagaaggaagctttacagcaagctgtaccaccagctccagtttatcactcaatgcctcctgtaatggcagacttttcaatgcctccagctcaaccagtggctcttcctccttctcctactcctacctcaacgcctgcaacagcaaaaccaacatccgccccatcctcgtctcatcctccactcaagagtcccatggctggtactttctatagatctcctggacccggtgaacccccttttgtaaaggttggagataaggtacagaagggtcaaattgtttgtattatcgaagctatgaaactgatgaacgagattgaggctgagaagtcaggaaccatcatggaactactggctgaagatggaaaaccagtcagcgttgacacgcctctttttgtcatcgcaccttga

SEQ ID NO: 18 - DNA, 3591 bp, Arabidopsis thaliana
  misc_feature 1..3591: note = ORF of protein Bri1
  source 1..3591: mol_type = unassigned DNA
atgaagactttttcaagcttctttctctctgtaacaactctcttcttcttctccttcttttctctttcatttcaagcttcaccatctcagtctttatacagagaaatccatcagcttataagcttcaaagacgttcttcctgacaagaatcttctcccagactggtcttccaacaaaaacccgtgtactttcgatggcgttacttgcagagacgacaaagttacttcgattgatctcagctccaagcctctcaacgtcggattcagtgccgtgtcctcgtctctcctgtctctcaccggattagagtctctgtttctctcaaactcacacatcaatggctccgtttctggcttcaagtgctctgcttctttaaccagcttggatctatctagaaactctctttcgggtcctgtaacgactctaacaagccttggttcttgctccggtctgaagtttcttaacgtctcttccaatacacttgattttcccgggaaagtttcaggtgggttgaagctaaacagcttggaagttctggatctttctgcgaattcaatctccggtgctaacgtcgttggttgggttctctccgatgggtgtggagagttgaaacatttagcgattagcggaaacaaaatcagtggagacgtcgatgtttctcgctgcgtgaatctcgagtttctcgatgtttcctccaacaatttctccactgggattcctttcctcggagattgctctgctctgcaacatcttgacatctccgggaacaaattatccggcgatttctcccgtgctatctctacttgcacagagctcaagttgttgaacatctctagtaaccaattcgtcggaccaatccctccgctaccgcttaaaagtctccaatacctctctctggccgagaacaaattcaccggcgagatccctgactttctctccggcgcgtgtgatacactcactggtctcgatctctctggaaatcatttctacggtgcggttcctccattcttcggttcatgttctcttctcgaatcactcgcgttgtcgagtaacaacttctctggcgagttaccgatggatacgttgttgaagatgagaggactcaaagtacttgatctgtctttcaacgagttttccggcgaattaccggaatctctgacgaatctatccgcttcgttgctaacgttagatctcagctccaacaatttctccggtccgattctcccaaatctctgccagaaccctaaaaacactctgcaggagctttaccttcagaacaatggcttcaccgggaagattccaccgactttaagcaactgttctgagctggtttcgcttcacttgagcttcaattacctctccgggacaatcccttcgagcttaggctctctatcgaagcttcgagatctgaaactatggctgaatatgttagaaggagagatccctcaggagctcatgtatgtcaagaccttagagactctgatcctcgacttcaacgatttaaccggtgaaatcccttccggtttaagtaactgtaccaatcttaactggatttctctgtcgaataaccggttaaccggtgagattccgaaatggattggccggttagagaatctcgctatcctcaagctaagcaacaattcattctccgggaacattccggctgagctcggcgactgcagaagcttaatctggcttgatctcaacaccaatctcttcaatggaacgattccggcggcgatgtttaaacaatccgggaaaatcgctgccaatttcatcgccggtaagaggtacgtttatatcaaaaacgatgggatgaagaaagagtgtcatggagctggtaatttacttgagtttcaaggaatcagatccgaacaattaaaccggctttcaacgaggaacccttgtaatatcactagcagagtctatggaggtcacacttcgccgacgtttgataacaatggttcgatgatgtttctggacatgtcttacaacatgttgtctggatacataccgaaggagattggttcgatgccttatctgtttattctcaatttgggtcataacgatatctctggttcgattcctgatgaggtaggtgatctaagaggtttaaacattcttgatctttcaagcaataagctcgatgggaggattcctcaggctatgtcagctcttactatgcttacggaaatcgatttgtcgaataataatttgtctggtccgattcctgagatgggtcagtttgagacttttccaccggctaagttcttgaacaatcctggtctctgtggttatcctcttccgcggtgtgatccttcaaatgcagacggttatgctcatcatcagagatctcatggaaggagaccagcgtcccttgctggtagtgtggcgatgggattgttgttctcttttgtgtgtatatttgggctgatccttgttggtagagagatgaggaagagacggagaaagaaagaggcggagttggagatgtatgcggaaggacatggaaactctggcgatagaactgctaacaacaccaattggaagctgactggtgtgaaagaagccttgagtatcaatcttgctgctttcgagaagccattgcggaagctcacgtttgcggatcttcttcaggctaccaatggtttccataatgatagtctgattggttctggtgggtttggagatgtttacaaagcgattttgaaagatggaagcgcggtggctatcaagaaactgattcatgttagcggtcaaggtgatagagagttcatggcggagatggaaaccattgggaagatcaaacatcgaaatcttgtgcctcttcttggttattgcaaagttggagacgagcggcttcttgtgtatgagtttatgaagtatggaagtttagaagatgttttgcacgaccccaagaaagctggggtgaaactaaactggtccacacggcggaagattgcgataggatcagctagagggcttgctttccttcaccacaactgcagtccgcatatcatccacagagacatgaaatccagtaatgtgttgcttgatgagaatttggaagctcgggtttcagattttggcatggcgaggctgatgagtgcgatggatacgcatttaagcgtcagtacattagctggtacaccgggttacgttcctccagagtattaccaaagtttcaggtgttcaacaaaaggagacgtttatagttacggtgtggtcttactcgagctactcacgggtaaacggccaacggattcaccggattttggagataacaaccttgttggatgggtgaaacagcacgcaaaactgcggattagcgatgtgtttgaccccgagcttatgaaggaagatccagcattagagatcgaacttttacaacatttaaaagttgcggttgcgtgtttggatgatcgggcttggagacgaccgacaatggtacaagtcatggccatgtttaaggagatacaagccgggtcagggatagattcacagtcaacgatcagatcaatagaggatggagggttcagtacaatagagatggttgatatgagtataaaagaagttcctgaaggaaaattatga

SEQ ID NO: 19 - DNA, 753 bp, Arabidopsis thaliana
  misc_feature 1..753: note = ORF of protein Bzo2h3
  source 1..753: mol_type = unassigned DNA
atggaaaaagttttctccgacgaagaaatctccggtaaccatcactggtcggttaatggaatgacgtcgttgaatcgcagtgcttccgaatgggcattcaatcgtttcatacaagaatcctccgccgctgcagacgacggagaatctacgacggcgtgtggtgtttccgtctcctctcctcctaatgttcctgtagattcagaggaatacagagcatttctcaagagtaaacttaatcttgcttgtgctgctgtcgccatgaaaaggggaactttcatcaaacctcaggatacttctggtagatctgacaatggtggagccaatgaatcagaacaagcctctcttgcttcttccaaagctacaccaatgatgagcagtgctataacaagtggatctgagctctctggtgatgaagaagaagctgatggtgaaactaatatgaaccctactaatgttaaacgcgttaaaaggatgctctctaatagagaatcagctagacggtccagaagaagaaagcaagcacacttgagtgagctagagacacaagtttcacagcttcgtgtagagaattcaaaactcatgaagggtctcactgatgtaactcaaacattcaatgatgcatctgtagaaaacagagttttaaaagccaatattgagacactacgagcaaaggtaaaatactttcttttatctctctgtctaccaaaactcgctattgaagcatgtttgtttttggcaggtgaaaatggctga

SEQ ID NO: 20 - DNA, 3255 bp, Arabidopsis thaliana
  misc_feature 1..3255: note = ORF of protein Cesa6
  source 1..3255: mol_type = unassigned DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 21 - DNA, 1356 bp, Arabidopsis thaliana
  misc_feature 1..1356: note = ORF of protein Cipk3
  source 1..1356: mol_type = unassigned DNA
atgttgatccccaacaaaaaattaagagaaatgaatcggagacagcaagtgaaacgtagagtaggtaaatatgaagttggaagaacaattggagaaggaacgtttgctaaagttaagtttgctagaaactctgaaactggagaacctgttgctctcaagattcttgataaagagaaagttctcaagcataaaatggctgaacagattagaagagagatagctactatgaagttgataaaacatccaaatgttgttcaattatatgaggtgatggcaagtaagacgaaaatatttatcatcttggagtatgttacaggaggagaactctttgataagattgtaaatgatgggcggatgaaagaagatgaggcgcggagatatttccaacagcttatacatgctgtggactactgtcatagcagaggggtctaccatagagacctcaagcctgaaaatttactattggactcctatggaaacctcaagatctcagattttggattaagtgctttgtcccaacaagtcagggatgatggactcttgcatacatcgtgtggaacaccaaactacgttgctcctgaggttctcaatgatagaggctatgatggagcaacagctgacatgtggtcatgcggtgttgtactctatgtcctgcttgcaggttacttaccttttgatgattctaatctaatgaatctttataaaaaaatatcatctggtgaattcaactgtcctccgtggctctcactcggtgccatgaaactcatcactagaatcttagatccgaatccgatgactcgtgtaacaccgcaagaggttttcgaagatgaatggttcaagaaagattacaagccacctgttttcgaggagagggatgattcaaacatggacgatattgatgctgttttcaaggactctgaggaacatcttgttactgagaagagagaagaacagccagcggcgatcaatgccttcgagatcatttcaatgtcaaggggacttaacctagagaatctgtttgatccagaacaggaatttaagagggaaacaaggataacattgagaggaggcgcgaatgagatcatcgagaagatagaagaagctgcaaagcctctcggtttcgatgttcaaaagaagaactacaagatgaggcttgagaatgtgaaggctggaagaaaggggaatctcaatgtagcgacagagatattccaagtagcgccaagtcttcatatggttcaagtatcgaagtcgaaaggagacactctcgaatttcacaagttctataagaagctctctaattctctggagcaagtagtctggacgaataacgaagttaagaaagaaacagcaaagtga

SEQ ID NO: 22 - DNA, 264 bp, Arabidopsis thaliana
  misc_feature 1..264: note = ORF of protein Cks1
  source 1..264: mol_type = unassigned DNA
atgggtcagatccaatactccgagaaatacttcgatgacactttcgaatacaggcacgtcgttcttcctcctgaagtcgctaaacttcttccaaagaatcgtcttctctccgaaaacgaatggcgagcgataggagtgcagcaaagccgcggatgggtacattacgcggttcatcgacctgagccgcacataatgctattcaggaggcctcttaactatcagcagcagcaggagaatcaagctcagaacatgcttgttaagtga

SEQ ID NO: 23 - DNA, 1962 bp, Arabidopsis thaliana
  misc_feature 1..1962: note = ORF of protein Cobl8
  source 1..1962: mol_type = unassigned DNA
atgggtttaactcgaaatttcattttatggattcttttgtcatctctcttcacggcgattcaactaacgtcgtctcaacgcaacactccaccaccacgctctaaagacgccgatctctgcaacggcgtgttcgtttcctacacttacttaaccggtaccaagattaaaccaaacgacacgaagaaccagccgtaccggttcgaatcagagatcacggtcctaaacaacggccgcgacgagcttaaatcgtggcaagtctttgttaaattcgcccaccgcgagattcttgtttcagcgaccaacgctgttctctctgacggttctagcctcccggctagcgttgagaacgggacaatcttcgccgggtttccatcggcggatttgaagacggcgatcatgacggccggcgacgtaactcagatggaagcacgtgttgagctcgtgggtactcagtttggagttgctccgccgagtgttccgcttccgaagaacatcactcttgttaatgatggatggtcttgtcctaaacccactcaacaaggtagtaatgttctgcaagtatgttgcacaccgaatccgaacattacaacaagcaaaattggtcaaaagttccttcctaggcaagaaggagatctaaccataatgtacgatgtgaccagagcatatcagtcgagttactcagcgcaggtcactatcgagaatcataacctgcttggtcggcttgacaattgggatctaagttttatgtggatgaaagatgagtttctcttctcaaccaaaggagcttatccaagtgttgtcgattcgtcggattgcatcactggtcctcaagcaaaatactataaagatcttgacttctctaatgtcatgagctgtgcaagaaggccacacatcatagacctacccctgactaagtataacgataccaatgtcgggcgtatcccatactgctgcagaaatggaactattctgccacggtccatggatccagagaagtctaaatcggtttttcaaatagaagtttacaaaatgcctcctgatctcaacatctcctctataacacctccccagagctggcagattaaaggtaaccttaacccggattacaaatgtggccctcctcttcgtgtcagctcaagccagttccctgatacaagcggtttgccttcaaacaagagtgcatttgctagctggcaagtcgtctgcaacatcactcaaccaactcctcctaaatgctgcgtctcattctcttcttatttcaatgactcagtaatcccgtgtaagacctgcgcttgtggaggttgctctagcgacagagtggcgcgtacttgcagcacaacttctccagctcttcctcttccatatcaggctcttctaattccatttgacaaccgaaccaaacttaccaacgcttgggcagtgttaaaaaaccggaaagtcccggacccattaccttgtggagataactgtggagtcagcataaactggcacctagctacagactatcgaggcggatggactgctcgagtcacactcttcaactggggtgatactgatttcgtggattggtttaccgcggttgaactgagaaatgctgcacccggttttcagaaagcgtactcgtttaacggaagtatcatcgcggtcaacgggaagaacacaactgttttaatggaagggctccctggactgaactatctccttgcagagaaagacggaaaaaacccgagtgaagactttcggatacccgggaaacagcagtcggttatctccttcaccaagaaactgactcctggaatcaaagttggttccaaagatgggtttcccaccaaagtactcttcaacggccaggaatgttcactcccttctgtactacccacgagcaacagtcacaggaaacatgtatccacctttcttctcatcctgacaccatttttggctctactgtttcttcggatttaa

SEQ ID NO: 24 - DNA, 1779 bp, Arabidopsis thaliana
  misc_feature 1..1779: note = ORF of protein Coi1
  source 1..1779: mol_type = unassigned DNA
atggaggatcctgatatcaagaggtgtaaattgagctgcgtcgcgacggttgatgatgtcatcgagcaagtcatgacctatataactgacccgaaagatcgcgattcggcttctttggtgtgtcggagatggttcaagattgattccgagacgagagagcatgtgactatggcgctttgctacactgcgacgcctgatcgtcttagccgtcgattcccgaacttgaggtcgctcaagcttaaaggcaagcctagagcagctatgtttaatctgatccctgagaactggggaggttatgttactccttgggttactgagatttctaacaaccttaggcagctcaaatcggtgcacttccgacggatgattgtcagtgacttagatctagatcgtttagctaaagctagagcagatgatcttgagactttgaagctagacaagtgttctggttttactactgatggacttttgagcatcgttacacactgcaggaaaataaaaactttgttaatggaagagagttcttttagtgaaaaggatggtaagtggcttcatgagcttgctcagcacaacacatctcttgaggttttaaacttctacatgacggagtttgccaaaatcagtcccaaagacttggaaaccatagctagaaattgccgctctctggtatctgtgaaggtcggtgactttgagattttggaactagttgggttctttaaggctgcagctaatcttgaagaattttgtggtggctccttgaatgaggatattggaatgcctgagaagtacatgaatctggtttttccccgaaaactatgtcggcttggtctctcttacatgggacctaatgaaatgccaatactatttccattcgcggcccaaatccgaaagctggatttgctttatgcattgctagaaactgaagaccattgtacgcttatccaaaagtgtcctaatttggaagttctcgagacaaggaatgtaatcggagataggggtctagaggtccttgcacagtactgtaagcagttgaagcggctgaggattgaacgcggtgcagatgaacaaggaatggaggacgaagaaggcttagtctcacaaagaggattaatcgctttggctcagggctgccaggagctagaatacatggcggtgtatgtctcagatataactaacgaatctcttgaaagcataggcacatatctgaaaaacctctgtgacttccgccttgtcttactcgaccgggaagaaaggattacagatctgccactggacaacggagtccgatctcttttgattggatgcaagaaactcagacgatttgcattctatctgagacaaggcggcttaaccgacttgggcttaagctacatcggacagtacagtccaaacgtgagatggatgctgctgggttacgtaggtgaatcagatgaaggtttaatggaattctcaagaggctgtccaaatctacagaagctagagatgagaggttgttgcttcagtgagcgagcaatcgctgcagcggttacaaaattgccttcactgagatacttgtgggtacaaggttacagagcatcgatgacgggacaagatctaatgcagatggctagaccgtactggaacatcgagctgattccatcaagaagagtcccggaagtgaatcaacaaggagagataagagagatggagcatccggctcatatattggcttactactctctggctggccagagaacagattgtccaacaactgttagagtcctgaaggagccaatatga

SEQ ID NO: 25 - DNA, 1779 bp, Arabidopsis thaliana; identical to SEQ ID NO: 24
  misc_feature 1..1779: note = ORF of protein Coi1
  source 1..1779: mol_type = unassigned DNA

SEQ ID NO: 26 - DNA, 1590 bp, Arabidopsis thaliana
  misc_feature 1..1590: note = ORF of protein Cpk3
  source 1..1590: mol_type = unassigned DNA
atgggccacagacacagcaagtccaaatcctccgatccgccaccgtcctcctcctcctcttcctccggcaacgtagtccaccatgttaaacctgccggagaacgccgcggatcatccggttccggaaccgtcggatcgtcaggctccggaacaggcggatctcgttccaccacatctactcagcagaatggtcggatcttaggtcggccgatggaggaagtacggagaacctatgagtttggtcgtgagcttggtcgtggccagtttggtgtcacgtacctcgtaactcacaaagagacgaaacaacaggtcgcttgcaagtcaatccctacgcgccgccttgttcacaaagacgacattgaagatgttcgccgtgaagtccagattatgcaccatctcagcggtcaccggaacatagtggacttgaagggagcctacgaggacagacactctgtgaatctgataatggagttgtgtgaaggaggggaattgttcgataggatcatttctaaaggtctttactcagagagagctgctgcggatttgtgtaggcagatggtgatggttgtgcatagttgtcattctatgggtgtaatgcaccgagacttgaagcccgaaaactttctctttcttagtaaagatgagaactcaccattgaaagctacagactttggtctctctgtcttcttcaagccaggtgataagtttaaggatcttgttggaagtgcatactatgttgccccagaagttctaaaacggaactatggaccagaggctgatatctggagtgctggtgtgattctatacatccttctcagtggtgttccgcctttctggggagaaaatgaaacggggatctttgatgccattcttcaagggcaacttgatttttcagctgatccatggccagcactatcagatggtgccaaagatcttgtgaggaaaatgttaaaatatgaccctaaagatcggcttacagctgcggaagtgctaaatcatccgtggattagagaagatggggaggcatcagacaaaccgcttgacaatgcggtgttatccaggatgaaacaattccgggcgatgaacaaactaaaaaaaatggccctgaaggttatcgctgagaacctttctgaagaagaaatcattggtcttaaagagatgttcaaatctctagacactgataacaatggaatagtcaccttggaggaattgagaaccggtctcccaaaacttggttcaaagatctctgaagctgaaataaggcagttaatggaagcagctgatatggatggagatggatcaattgattacttggagtttatatcagccacaatgcatatgaacagaatcgaacgtgaggatcacttgtacaccgcattccagttctttgacaatgacaacagcggctacataacaatggaagagctagagctagccatgaagaaatacaacatgggtgatgataaatccatcaaagagatcattgctgaagtagacaccgatcgtgacggaaaaatcaactacgaagaatttgtagcgatgatgaagaagggaaatccagaattggtgccaaaccgacgcagaatgtga

SEQ ID NO: 27 - DNA, 963 bp, Amaranthus hypochondriacus
  misc_feature 1..963: note = ORF of protein Cpk3
  source 1..963: mol_type = unassigned DNA
atttcacaaaatataagctggttctggccaaccccacaacacccttacctgaccttattcggatgccaaagagaagtcttcagagatcttgtcggaagcgcatattatgtagctcctgaggttttacgtcgtagttatggggctgaagctgatgtttggagtgctggagtgatattatacatcctacttagtggagttccaccattttggggagagaatgagcagggaatttttgattctattctgcggggcaatcttgatttcaattcagatccatggctctcaatatctagcagtgccaaagacctggtgacaaagatgttgcggcaggatcctaaggaaagactttctgctcgtgatgctttaaatcatccttggatgagagaggatggagaagctcctgataagcctattgatattgctgttttgactagaatgaagcagtttagggcaatgaacaaactaaaaaaagtagcactaagggtaattgccgagaacctttctgaagaaaaaataattggtttaaaagaaatgtttaaatccatagatactgataatagtggaactatcacctatgaagaattaaagagcggtcttccaaagctgggaactagactttctgagaaagaagtcaagcagttaatggaggcggctgatgttgatggtaatggaaccatagattacatggaattcatcacagctacaatgcatatgaataggatggaggaaggggaccatatatacaaagcctttgaatattttgatgtggataaaagcgggtacattaccatggaggaattggcgcaagccttgaagaagtataatatgggtgatgcaaaaacaatacgggagataattgcagaagttgatacagacaatgatggtaaaatcaattatgatgaatttgtagcaatgatgaagaaaggcaaccctgatatggttaccaacagacggaagagatga

SEQ ID NO: 28 - DNA, 1647 bp, Brachypodium distachyon
  misc_feature 1..1647: note = ORF of protein Cpk3
  source 1..1647: mol_type = unassigned DNA
atgggcgcccgcgcctcccgccaccgccaccaatccccctcccaatcccaatccagcgacgactcccagtcccaccacaagccacgacccaagcccaagcccaagcatcaccaccaaccaccacaaccccctccgcggcatcagccccgccaccaccaccacccacccccgcaacgccctccgccgcaacatcaacaacaacaccagcagcagcaccatgcggcggcggcggcggaggacacgggccgggtcctgggccgcccgctgtcggacgtgcgcgcgacctacacgttcggccgggagctgggccggggccagttcggggtcacctacctagcaacccacaagtccaccggcgcgcgctacgcctgcaagtcgatctccgcccggaagctgtcccgcggggacgacgccgacgacgtccgccgcgaggtgcagatcatgcaccacctcacgggccaccgcagcatcgtcgagctcaagggcgcccacgaggaccgccactcggtcaacctcgtcatggagctctgcgagggcggggagctcttcgaccgcatcatcgcccggggacactactccgagagcgccgcggcggcggtctgcagggaggtggttaatgtcgtccactgctgccattccatgggggtcatgcatcgggacctcaagccggagaactttttgtttctgaataagcgggaggactcgccgcttaaggctactgattttggtctctccgtcttcttcaagcccggggagcagttcagggatctcgttgggagcgcctactatgtggctccggaggtgctaaagcggcgatatggagctgaggcggacatatggagtgctggagtcatcctctacattctcctctccggtgtccctcctttctgggcagagaatgaggatggtatattcgatgctgttctgcaaggtcatattgatttctcatctgatccctggccatcaatttcacacggggctaaagacttggtcaggaggatgctgcggcaggaccccaaggagcgcctcactgctgctgaaattttgaaccatccatggattagagaggacggagaggccccagataaaccacttgatattacagtgatcagtagaatgaagcagttcagagcaatgaacaaacttaagaaggttgccttgaagattgttgcagaaagcttgtcagaagaagagatcgtgggcttaaaacaaatgttcaaatccctggatactgacaacagcgggacaattactcttgaagaactacgggctggattaccaaagctcggtaccaaaattactgaatcagaaataagacagttgatggaggcagctgatgttgatggaaatgggaccattgattatgttgaattcatatcggcaacgatgcacatgaatagactagagaaggaggatcacatatttaaagcatttgaatattttgacaaggaccacagtggctacataacagttgatgagttggaagaagccttgaagaagtatgatatgggtgatgaggcaacaatcaaggaaatcattgctgaagtggatacagatcatgatgggaaaataaactaccaagagtttgttgccatgatgaaaaacaatagcccagagattgttccaaatcgacgccgcttgttttaa

SEQ ID NO: 29 - DNA, 1581 bp, Brachypodium distachyon
  misc_feature 1..1581: note = ORF of protein Cpk3
  source 1..1581: mol_type = unassigned DNA
atgggcaaccgcacctcgcggcaccgccgcgccgccgccgccgagcagccgacgccggttccgtccccgccgccggcccagcccaaacccgagccgcaacagcagacgctccactggccgcagccgaagccccagccgccccccgccccggcggtggaagcgggcgtggtggcgatggggcgcgtgctggggcggccgatggaggacgtgcgcgcgacctacaccttcggccgcgagctcggccggggccagttcggggtcacctacctcgtcacgcacaaggccacgggccagcgcttcgcctgcaagtccatcgccacgcggaagctcgtccaccgcgacgacatcgaggacgtgcagcgggaggtgcagatcatgcaccacctcacgggccaccgcaacatcgtcgagctccgcggcgcctacgaggaccgccactcggtcaacctcgtcatggagctctgcgagggcggggagctgttcgaccgcatcatcgcgcggggccactactccgagcgagccgccgccgtgctctgccgcgagatggtctccgtcgtgcacagctgccactcgatgggggttttccatcgggatctcaagcctgagaactttctgtttctgaataacaaggaagactcgccactcaaggccactgactttggcctctccgtcttcttcaagcatggggagcagtttaaggatcttgttggaagcgcatattatgttgctcctgaggtgctgaaacggcactatggagcagaagctgacatatggagtgcaggggttattctttacatccttctgtctggtgttcctcctttttgggcagataatgaggatggcatatttaaggctgttttgcttggtcacattgatttctcgtctgacccctggccttcaatttctaatcccgcgaaagatttggtcaagaagatgctgcgacaagaccccaaagagcgcttaactgctgcagagattttagaccacccatggattaaggaagatggagaggccccggataagccacttgacattacagttatcagtagaatgaagcagttcagggcgatgaacaagcttaagaaagttgcactgaagattgttgcagagaacttgtctgaggaagagattacaggcttgaaagaaatgttcagatccctggatactgataacagcgggacaattactcttgaagagctacgatctggtttaccaaaacttggcaccaaaatatctgaatcagaaattacacaactgatggaggcggctgatgttgatggaaatgggaccattgattattctgagtttgtatcggcgacaatgcacatgaatagattggagaaggaagaccacatacttaaagcatttgaatactttgataaggaccacagcggatacataacagtagatgagttggaagaagctctgaagaaatatgacatgggggacgataaaacaattaaagaaatcattgctgaagtagatacagataatgacggaagaatcaactaccaggagttcgttgctatgatgagaaacaacagccctgagattgttccaaatcggagacgcatgttttaa

SEQ ID NO: 30 - DNA, 1314 bp, Glycine max
  misc_feature 1..1314: note = ORF of protein Cpk3
  source 1..1314: mol_type = unassigned DNA
atgggcaactgcagcggcggcggcggaatcggcagcaccacctactccgacgaccatccccgccgcaacggcatcaccgtcctccctccgaactcgaacctttcagccccaccagcgacctcctcgctgggccggcgcatggaggacatgcgatccatttacatattcgggcgggagctggggcgcgggcagttcggcgtgacgtacctggttacccacaaggccacgaaggagcagttcgcgtgcaagtcaatcgccacgcggaagctggtgaaccgcgacgacatcgacgacatccggcgcgaggtgcagatcatgcaccacctcaccggccaccgcaacatcgtcgagctcaagggcgcctacgaggaccgccactccgtcaacctcgtcatggagctctgcgccggcggcgagctcttcgaccgcatcatcaccaagagccactactccgagcgcgccgccgccaactcctgccgccagatcgtcaccgtcgtccataactgccactccatgggagtcatgcatagggacttgacgagaatttcttgttgctcaaccataacgatgattcacccctcaaggccacggattttggtctctccgtcttttttaagccaggtgctgagaagaagttacgggcctgaaactgatatttggagtgcaggagttatattgtacattttgctttctggtgttccacctttttgggcagaaaatgaacagggtatctttgatgctattcttcgtggacatattgatttcgcatcagatccttggccttccatatcaagtagtgccaaagatctggtcaagaagatgctacgagctgaccctaaagaacggctttcagcagttgaagtacttaatcatccctggatgagagtggatggagatgcatctgataagcctcttgatattgctgttttaaccagaatgaaacatttccgggcaatgaacaagctaaaaaaagtagccctgaaggttattgcggaaaacctttctgaagaagaaattattggcttgaaggaaatgttcaaatccatggacacagataacagtggaacaatcacatttgaagagttgaaagctggtctcccaaaattgggtagtaaactttctgaatctgaagttaggcagttgatggaagcgggtctgattttagcctaccttataaatacctttccctgccttgtactctctctccaacaaacccgcatgcacttccccaaccccatgatcaggtgcaatgcaccttttgcctctcattacatacaccttagtgatctttggctgtggggccacatcacttaa

SEQ ID NO: 31 - DNA, 1557 bp, Glycine max
  misc_feature 1..1557: note = ORF of protein Cpk3
  source 1..1557: mol_type = unassigned DNA
atgggaaactgcagcagcggggccggagcccccaccacctactccgacgaccatccccaccacaacggcataaccgtcctccctcccaactcaaacccttcaccccaactccccaagcccccaccgacctcctcctcctcctcctcgctgggccgggttttgggccggcccatggaggatgtccggtccatttacatattcgggcgggagctggggcgcgggcagttcggcgtgacgtacctggtgacccacaaggccacgaaggagcagttcgcgtgcaagtcaatcgcgacgcggaagctggtcaaccgcgacgacatcgacgacatccgccgcgaggtccagatcatgcaccacctcaccggccaccgcaacatcgtcgagctcaagggcgcctacgaggaccgccactccgtcaacctcgtcatggagctctgcgccggcggtgagctcttcgaccgcatcatcaccaagggccactactccgagcgcgccgccgccaactcctgccgccagatcgtcaccgtcgtccataactgccactccatgggagtcatgcatagggacttgaagcccgagaatttcttgttgctcaacaagaacgatgattcccccctcaaggccacagattttggcctctccgtcttttttaagccaggggatgtgtttagagaccttgttgggagtgcttactatgttgctcctgaggtgctgagaagaagttatggacctgaagctgatatttggagtgctggagttatattgtacattctactttctggtgttccacctttttgggcagaaaatgaacagggtatctttgatgctattcttcgtggacatattgattttgcatcagatccttggccttccatatcaagtagtgccaaagatctggtcaagaagatgctacgagctgaccctaaagagcgactttcagcggttgaagtacttaatcatccctggatgagagtggatggagatgcacctgataagcctcttgatattgctgttttaaccagaatgaaacaattccgagcaatgaacaagctaaaaaaagtagctctgaaggttattgctgaaaacctttctgaagaagaaattattggcttgaaagaaatgttcaaatccatggacacagataacagtggaacaatcacattcgaagagttgaaagctggtctcccaaaattgggtactaaactttccgaatctgaagttaggcagttgatggaagcggctgatgtagatggaaatggaaccattgattacattgaatttataactgctaccatgcacatgaatagaatggaaagagaggatcacctatataaagcctttgaatattttgacaatgataagagcgggtacatcacaatggaagagttggaatctgcccttaagaaatataatatgggtgatgagaaaacaataaaggagatcattgctgaagttgatacagacaatgatggaagaattaactatgatgagtttgtagccatgatgaggaaaggcaacccagacataacgcacataacccaaagacgtcgcaaataa

SEQ ID NO: 32 - DNA, 1518 bp, Glycine max
  misc_feature 1..1518: note = ORF of protein Cpk3
  source 1..1518: mol_type = unassigned DNA
atggggaactgtaacagcgaaccctcttcccaaacccagccacattccgatcaccaccatcatcacccccaaccctccaacggcatcactgtccttcctcccaattcgaagccctccgtaggccgggttctgggccggcccatggaggacgcccgctccacctacaccttcggccgcgagctgggccgcggccaattcggcgtgacctaccaagtgacccacaaacacacgaaacagcaattcgcgtgcaaatccatcgccacgcgcaagctcgtccaccgcgatgacttggaggacgtccggcgagaggtgcagatcatgcaccacctcaccggccaccgcaacatcgtggagctcaagggcgcttacgaggaccgccactccgtcaacctcatcatggagctctgcggcggcggcgagctcttcgaccgcatcatcgccaagggccactactccgagcgcgccgccgcagatctctgccgccagatcgtgacggtggtccacgattgccacaccatgggagtgatgcacagagatctcaagccggagaacttcctgttcctcagcaaggacgagaactcgccgcttaaggccacggacttcggtctctctgtcttcttcaagcccggcgatgtgtttaaggatcttgtcgggagtgcgtactatgttgctcctgaggtgctgcgcagaagctatggacccggagctgatatttggagtgctggcgttatattgttcattttgctctctggtgtccctcccttctggtcagaaaaggaacagggtatatttgatgctattcttcgtggccatattgatttcgcatcagatccttggccttccatatcgagtagtgccaaagatctggttaagaagatgttacgagctgaccccaaacaaagactctcagcagttgaagttcttaatcatccttggatgagagaagatggagcatctgataaacctctagatgttgctgttttatccagaatgaaacaatttagggcaatgaacaagctaaagaaagttgctctgaaggttattgctgaaaacctttctgaagaagaaatcattggtttgaaggaaatgtttaaatccatggacacagataacagtggaacaatcacttttgaagagttgaaagctggtctgccaaaactgggtactaaggtttctgagtctgaagtaaggcaattgatggaagcggctgatgtggacggaaatgggaccattgattacattgaatttataacagctaccatgcacatgaatagaatggaaagagaggaccacctatataaagcttttgaatattttgacaaggatagaagcgggtacatcacagtggaagagttggagtctgcccttaaaaaatataatatgggtgatgagaaaacaataaaggaaatcattgccgaagttgatgcagacaatgatggaagaattaactatgacgagtttgtagccatgatgaggaaaggcaatccagatttagtaaacaacagacgtcgcaaataa

SEQ ID NO: 33 - DNA, 1587 bp, Glycine max
  misc_feature 1..1587: note = ORF of protein Cpk3
  source 1..1587: mol_type = unassigned DNA
atggtttcacgtttcagtcctgatcgagtaaacaccaagaagcagcagcgcagtaacgacaccgtttcgatggggaactgtaacagcgaagcctcttcccaaactcagccacattccgatcaccatccccaaccacgctccaactccaacggcatcaccgtccttcctcccaattcgaagccctccgttggccgggtcttgggccggcccatggaggacgtccgctccacctacaccttcggccgcgaactgggccgcggccaattcggcgtgacataccttgtgacccacaaacacaccaaacaacaattcgcgtgcaaatccatcgccacgcccaaactcgtccaccgcgacgacttggaggatgtccggcgagaggtgcagatcatgcaccacctcaccggccaccgcaacatcgtggagctcaagggcgcttacgaggaccgccactccgttaacctcatcatggagctctgcggcggcggcgagctcttcgaccggatcattgccaagggccactactccgagcgcgccgccgccgacctctgccgccagatcgtgacggtggtccacgattgccacaccatgggagtgatgcacagggatctcaagccagagaacttcctgttcctcagcaaggacgagaactcgccgcttaaggccacggacttcggtctctccgtgttcttcaagcccggcgacgtgtttaaggatcttgtcgggagtgcgtactatgttgctcctgaggtgctgcgtagaagctatggacccgaagctgatatttggagtgctggggttatattgttcattttgctctccggtgtccctcccttctggtcaaaaaaggaacaaggtatctttgatgctattcttcgtggccatattgatttcacatcagatccttggccttccatatcgagtagtgccaaagatctggtcaagaagatgttacgagctgaccccaaacaaaggctctcagcagttgaagtccttgatcatccttggatgagagaagatggagcatctgataagcctctagatgttgctgttttatccagaatgaaacaattcagggcaatgaacaagctaaagaaagttgctctgaaggttattgctgaaaacctttctgaagaagaaatcattggcttgaaggaaatgtttaaatccatggacacagataacagtggaacaatcacttttgaagagttgaaagctggtctgccaaaactgggtactaaggtttctgagtctgaagtaaggcagttgatggaagcggctgatgtagacggaaatgggaccattgattacattgaatttataacagctaccatgcacatgaatagaatggaaagagaggaccacctatataaagcttttgaatattttgacaaggatagaagcgggtacatcacaatggaagagttggaatctacccttaaaaaatataatatgggtgatgagaaaacaataaaggaaatcattgtggaagtcgatacagacaatgatggaagaattaattatgatgagtttgtagccatgatgaggaaaggcaaaccagatttagtaaccaacagacgtcgcaaatga

SEQ ID NO: 34 - DNA, 1698 bp, Oryza sativa
  misc_feature 1..1698: note = ORF of protein Cpk3
  source 1..1698: mol_type = unassigned DNA
atggtctcctcctcctcctccccccaatccaaacccccaaaaccaaagccaaaacccctctcgccaccgatgggcgcccgcgcctctcgccaccgccaatcccccgaccaatcccaatcccaatccccatccccacaccacaagcaccaccaccaccaccaaaccacccgggcccccaagcccaagcccaagccccagccaccgccgccgcagcagccgcgatctcagcctccgccgccgccgcggcaccagccccagcaggcgccccagcaggcggcggcggaggatggggtggggcgggtgctggggcggcccatggaggacgtccgtgcgacctacaccttcgggcgggagctggggcgggggcagttcggggtgacctacctcgccacccacaagcccaccggccgacgctacgcctgcaagtccatcgccgcccgcaagctcgcccgccccgacgacctcgacgacgtccgccgcgaggtccacatcatgcaccacctcaccggccaccgcaacatcgtcgagctgcgcggcgcctacgaggaccgccactccgtcaacctcgtcatggagctctgcgagggcggcgagctcttcgaccgcatcatcgccaggggccactactccgagcgcgccgccgccgccctctgcagggagatcgtctccgtcgtccacagctgccactccatgggggtcatgcacagggacctcaagcccgagaacttcctcttcctcaacaagcgcgaggactccccgctcaaagccaccgattttggcctctccgtcttcttcaagcccggtgagcagttcagagatcttgttggaagtgcatattatgtggctcctgaggtcctaaaacgactatatggagctgaggcagacatatggagtgctggagttatcctttacatccttctatcaggggttcctccattctgggcagaaaacgaggacggtatatttgatgctgttctgcaaggtcatatcgacttctcatctgaaccatggccttctatatctagtggtgcaaaagacttggtcaagcggatgcttcggcaggacccaaaggagcggttaactgctgctgaaattttgaaccacccatggattagagaggatggagaggccccagataaaccacttgatattacagtgatcagtagaatgaagcagttcagagcaatgaacaaacttaagaaggttgccttgaaggtcgttgcagagaacttgtcagaggaagagattgtgggtttaaaggaaatgttcaaatctttagatactgataacagtgggacgataactcttgaagaactaagagctggtctaccaaagcttggcactaaaatttcggaatcagaattaaggcagttgatggaagcggctgatgttgatggaaatgggtccattgattatgttgaatttatatcagcaacgatgcacatgaatagattagagaaggaagatcacatatataaagcatttgaatattttgacaaggaccacagcgggttcataacagtcgatgaattggaagaagctctgacaaagtacgacatgggtgatgaagcgacaattaaagaaataattgctgaagtggatacagaccatgatggaagaattaactaccaggagtttgttgccatgatgaagaacaacagccctgagattgttccaaatcgacggcggatgttttaa

SEQ ID NO: 35 - DNA, 1557 bp, Oryza sativa
  misc_feature 1..1557: note = ORF of protein Cpk3
  source 1..1557: mol_type = unassigned DNA
atgggcaaccgcacgtcgcgccaccaccgcgccgctcccgagcagccgccgccccagcccaagccgaagcctcagccgcagcagcagcagcagcagtggccgaggccgcagcagccgaccccgccgccggcggcggcgccggacgcggcgatggggagggtgctggggcggccgatggaggacgtccgcgcgacctacaccttcgggcgcgagctcgggcggggccagttcggggtcacctacctcgtcacccacaaggccacggggaagcgcttcgcctgcaagtccatcgccacgcggaagctcgcccaccgcgacgacatcgaggacgtgcgccgggaggtgcagatcatgcaccacctcacgggccaccgcaacatcgtcgagctccggggcgcctacgaggaccgccactcggtcaacctcatcatggagctctgcgagggcggggagctcttcgaccgcatcatcgccagggggcactactccgagcgcgccgccgccgcgctctgccgcgagatcgtcgccgtcgtgcacagctgccactccatgggggtcttccaccgggatctcaagccggagaactttttgttcctcagtaaaagcgaggactcgccgctcaaggccactgacttcggtctttccgttttcttcaagcctggggagcattttaaggaccttgttggaagtgcgtattatgttgcacctgaggtgctgaaacgaaactatggagcagaggctgacatatggagtgctggcgttattctttacatccttctttctggtgttccacctttttgggcagagagtgaagatggtatatttgatgccgtattacgtggtcacattgatttctcgtctgaaccttggccttcgatatccaatggtgcaaaagatttggttaagaagatgttgcggcaagacccaaaagagcgccttacttctgctgaaattttgaaccacccatggattagagaggatggagaagccccagacaagccacttgacattacagttatcagcaggatgaagcagttcagggcaatgaacaagcttaagaaagttgcattgaaggttgttgcagagaacttgtcagatgaagagattacaggcttaaaagaaatgttcagatccttggatactgataacagtgggacaataactcttgaggagctaagatctggtttaccaaaacttggcaccaaaatttctgaatcagaaattagacagctgatggaggcggctgatgttgatggaaatggaaccattgactatgctgagttcatatcagcgacaatgcacatgaacagattggagaaggaagaccacatacttaaagcatttgagtattttgataaggaccacagcggatacataacagtagatgagttggaagaagctctgaagaagtatgatatgggggacgataaaacgattaaggaaatcatcgctgaagtagatactgatcatgatggaagaattaactaccaggagtttgtcgccatgatgaggaacaacaaccctgagattgctcccaaccggcggcgcatgttctaa

SEQ ID NO: 36 - DNA, 1551 bp, Solanum lycopersicum
  misc_feature 1..1551: note = ORF of protein Cpk3
  source 1..1551: mol_type = unassigned DNA
atgggcaactgtaacagcttatcttccgaccaagccaccaccacttccggcggcggcggaggcggcggcgctccttcaacgccgtcaaccaccggtatcagaatcatcccttcctcacaaccaccgcctccacgaccactctccggcatcggccgtgttctcggccggccaatggaggatgtccggtccatctacattttcggcggcgaactcggccggggtcaattcggagtcacctacttagttactaacaaaaaaacgcgccagcagttcgcgtgcaagtcaatcgctacacggaaactcatcaacaaggacgatgtcgacgacgtccgccgtgaagttcagatcatgcatcaccttaccggtcaccggaatatcgttgagcttaaaggaactttcgaggataaacaccatgttcatctcgtcatggaactttgtgccggcggcgagctttttgaccggataattgctaaaggacactattccgagcgagctgctgctggagtttgccggcagatggtaactgttgttcacaattgtcattctatgggagttatgcatagggatttaaaacctgaaaatttcctttttttgagctccgatgaaacttcaccattaaaggctactgattttggactatcagtctttttcaaaccaggtgatgtgttcaaagacctggttggtagtgcatattatgttgctcctgaagttctgcgccgagaatatggacctgaagctgatatttggagtgctggagttattctgtacattctactcagtggtgttccaccattttatggagagaatgatcagagtatctttgatgccgttcttcgaggtcatcttgatttttcatctgatccttggccttcagtatcgagtagcgctaaagatcttgttaagaagatgctgcgatctgatcctagagagaggatttctgcagctgaagtcctaaatcatccatggatgagagaagatggtgatgcatctgacaaacctcttgacattgctgtcttatctagaatgaaacaattccgtgctatgaacaagctgaagaaagtagcactgaaggtaattgctgaaaatctttctgaagaagaaatcattggcttgaaggaaatgttcaaatctatagacacagatgacagtgggacaattacatttgaagaattgaaagccggtcttacaaaaatgggtacaaagctttcagagtcggaagtcaggcagttgatggaagcggctgatgttgatggaaatggaacaattgattaccttgagttcataacagctacgatgcacatgaatcgcatggaaagagaagatcacttgtacaaagcctttgagtattttgataaggataagagcgggtatattacaatggaagaattggagcatgcgctgaaggaatacaacattactgatgacaaaacaataaaagagatcattgctgaagttgacacagacaatgatggaagaatcaattacgatgagttcgctgccatgatgaggaaaggtaatccagattttgtcaacaataggcgccgcagatga

SEQ ID NO: 37 - DNA, 1620 bp, Zea mays
  misc_feature 1..1620: note = ORF of protein Cpk3
  source 1..1620: mol_type = unassigned DNA
atgggcggccgcgcctcccgccaccgcgcgcagccgcacgaccagtcttctcaccctcctcctcggccgccgcccaagcagcagcagggccagcagcaccccaacaaccgccccaacaagcccaagcagcagcagcagcccccacctccacgtcctccgccgccacaccaggctccccccgccgcctcccccgccgccccgggcgcggccaccgtcggccgcgtgctgggccggccgatggaggacgtgcgcgcgtcctacacgttcggccgggagctgggccggggccagttcggcgtcacctacctggccacgcacaagcccaccggccgccggtacgcgtgcaagtccatcgcgacgcggaagctcgcgcacagcgacgacgtcgacgacgtccgccgggaggtgcagatcatgcaccacctcacgggccaccgcagcatcgtcgagctccggggcgcctacgaggaccgccactccgtcaacctcgtcatggagctgtgcgagggtggggagctcttcgaccgcatcatcgcgagggggcactactccgagcgtgccgccgccgccctctgcagggagatcgtctccgtcgtccacagctgccactccatgggggtcatgcatcgggacctcaagcccgaaaacttcctcttccttaacaagagggaggactcgccgctcaaggccacggatttcggactctccgtcttcttcaagcccggcgagcagttcagggatcttgtcggcagcgcttactacgtcgccccagaggtgctcaagcggcgatacggagctgaggcagacatatggagtgccggagttatcctctacatcctgctatccggcgtccctcctttctgggccgagaacgaggatggcatatttgatgctgttttgcgaggtcatattgacttcgcatctgatccctggccttccatatcgaatagcgcaaaagatttggtcaagaagatgctgcgccaagaccccaaggagcggcttactgctgctgaaattttgaaccatccatggattagagaggatggagaggccccagacaagccactagacataacagtaataagcagaatgaagcagttccgggccatgaacaagcttaagaaggttgccttgaaggttgttgctgagaacttgtcagaggaagagatcgtgggcttgaaggaaatgttcaaatccctggatactgacaacagtgggacaataacgctggaagaattgagagctggtttaccaaagctcggcaccaaaatctctgaatcagaaataaggcagttgatggaagcagctgatgttgatgggaatggcaccattgattatgttgaatttatatcagcaacaatgcacatgaatagactagagaaggaggatcatatatttaaagcattcgagtattttgacaaggatcatagcgggcacattacagttgatgagttggaagaagctttgaagaagtatgatatgggcgatgaggcaacagttaaagaaattattgctgaagtagacacagatcacgatgggagaatcaactaccaggagtttgttgccatgatgaagaataatagccccgagattgttccaaatcggaggcgcatgttttaa

SEQ ID NO: 38 - DNA, 1593 bp, Zea mays
  misc_feature 1..1593: note = ORF of protein Cpk3
  source 1..1593: mol_type = unassigned DNA
atgggcaaccgtgcgtcgcgccaccaccgccaagccgccgagcagcaaccggcgacgccgaccctgccgaagccccagccccagatccagccgccgcctccgcctccgccgacgcagacgcagcagtaccagcaggcgccgccgcagtcgcagcagaagccgaagccggcggcggacgtgggggcggtggggcgcgtgctggggcggcccatggaggacgtgcgcgcgacctacactttcggccgcgagctcgggcggggccagttcggggtcacctacctcgtcacgcactcggagaccgggcagcgcttcgcctgcaagtccatcgccacgcggaagctcgtccaccgcgacgacattgaggacgtgcgccgggaggtgcagatcatgcaccacctcacgggccaccgcaacatcgtcgagctccggggagcatacgaggacaggcactcggtcaaccttgttatggagctctgcgagggcggggagctcttcgaccgcatcatagccaagggccactacaccgagcgagccgccgcctcgctctgccgcgagatcgtcgccgtcgtccacagctgccactccatgggggtcttccatcgggatctcaagcccgagaacttcctgttcctcaataacaaggaggactccccgctcaaggccacggacttcggtctctccgtcttcttcaagcacggggagacgtttaaggatcttgtgggaagtgcatattatgttgctcctgaggtactgaaacggaaatatggggcagaggctgacatatggagtgctggggtcattctttacatccttctgtctggtgttcctcctttttgggcagagaatgaggatggcatatttgatgctgtcttgcgcggtcacatcgatttctcttctgacccttggccttcaatatccaatggtgcaaaggatttggttaagaagatgctgcgacaagacccaaaagaacgcctgactgctgctgaaattttgaaccacccatggattagagaggatggagaggccccagataagccacttgacattactgtcattggtagaatgaaacagttcagggcaatgaacaagctcaagaaagttgcattgaaggttgtggctgagaacttatcagatgaagagattatgggcctaaaagaaatgttcagatccctggatacagataacagtgggacaattactcttgaagagctaagatctggtttaccaaaacttggtacaaaaatttctgaatcagaaattaaacaattgatggaggcggctgatgttgatggaaatggtaccattgattatgcggagtttatatcagccacaatgcatttgaatagattggagaaggaagaccacatactcaaagcttttgaatatttcgataaggatcacagcggatacataaccgtagatgagttggaagaagctttgaagaagtatgatatgggagatgacaaaactataaaagaaatcattgctgaagtagatacagatcatgatggaagaattaattaccaggagtttgttgccatgatgaggaacaacagccctgagattgttccaaatcggaggcgcatgttctaa

SEQ ID NO: 39 - DNA, 1575 bp, Zea mays
  misc_feature 1..1575: note = ORF of protein Cpk3
  source 1..1575: mol_type = unassigned DNA
atgggcaaccgtgcgtcgcgccaccaccgccacgccaccgagcagcagccagagacgccgacccttcccaagccccagatccagccgccgccgccgccgacgcagacgcagcagtaccagcaggccccgccgccgccgaagccgaagccggcggcggacgcgagggcggtggggcgcgtgctggggcggcccatggaggacgtcaaggcgacctacaccttcggccgcgagctcgggcggggccagttcggggtcacctacctcgtcacgcaccgggagactgggcagcgcttcgcctgcaagtccatagccacgcggaagctcgtccaccgcgacgacatcgaggacgtgcgccgggaggtgcagatcatgcaccacctcaccggccaccggaacatcgtcgagctccggggagcctacgaggacaggcgctcggtcaacctcgctatggagctctgcgagggcggggagctcttcgaccgcatcattgccaggggccactacaccgagcgcgctgccgccgcgctttgccgggagatcgtcgccgtcgtccacagctgccactccatgggggtcttccaccgggacctcaagcccgagaacttcctgttcctcaataacaaggaggactccccgctcaaggccacggacttcggcctctccgtcttcttcaagcccggggacacgtttaaggatcttgtgggaagtgcatattatgttgctcccgaggtattgaagcggcactatggggcagaggctgacatatggagtgctggggttattctttacatccttttatctggtgttcctcctttttgggcagagaatgaggatggcatatttgatgctgtcttgcacagtcacattgatttctcttctgatccttggccttcaatatctaatggtgcaaaagatttggttaagaagatgctgcgacaagacccaaaagaacgcctgactgctgctgaaattttgaaccacccatggattagagaggatggagaggccccagataagccacttgacattacggtcatcagtagaatgaaacagttcagggcaatgaacaagctcaagaaagttgcattgaaggttgtggcagagaacttatcagatgaagagattatgggcctaaaagaaatgttcagatccctggatactgataacagtgggacaattactcttgaagagctaagatctggtttaccaaaacttggtactaaaatttctgaatcagaaatcaaacagttgatggaggcggctgatgttgatggaaatggtaccattgattacgcggagttcatatcagccacaatgcatttgaatagattggagaaggaagaccgtatactcaaagcttttgaatattttgataaggaccatagcgggtacataacagtagatgagttggaagaagctctgaagaagtatgatatgggagatgataaaacaataaaagagatcattgctgaagtagatacagatcatgatggaagaattaattaccaggagtttgttgccatgatgaggaacaatacccctgagattgttccaaaccggagacgcatgttctaa

SEQ ID NO: 40 - DNA, 1599 bp, Brassica rapa
  misc_feature 1..1599: note = ORF of protein Cpk3
  source 1..1599: mol_type = unassigned DNA
atgggacacagacacagcaagtccaaatcctccggtccgccaccgtcctcctcctcctcctcttcctccaccaacgtcgtccaccatgtccaaccttccggagaacgccgcggatcttccggttctggccccacacccgtcggatcttcctcaaccggcggatcccgctccgccgcatctgctcagcagaacggtcggatcttaggccgtccgatggagaatgtcaggggaacgtacgacttcggcaaggagcttggtcgtggccagttcggagtcacgtatctcgtcactcacaaagagaccaaaaaactcttcgcctgcaagtcgatccccacgcgccgcctcgtccaccgtgacgacattgaagacgtccgccgtgaagtccagattatgcaccatctcagtggtcacaggaacatagtggacttgaagggagcttacgaggacagacactcggtgaatctgataatggagctgtgcgaaggaggggagcttttcgacaggatcatagcgaaaggccattacacggagagagccgctgcggatttgtgcaggcagatggtgatggttgtgcatagttgccactctatgggtgtgatgcaccgtgatttgaagcctgagaactttctctttctcagcaaagatgagaactctccgttgaaagctacagactttggtctatccgtcttctttaaaccaggagataagtttaaggatcttgttgggagtgcatactatgtggccccggaggttttgaaacggaactatggaccagaggctgatatctggagtgccggtgtgattctcttcatccttatcagcggtgttccgccgttttggggagaaaatgagacagggatctttgatgctattctaaaaggagagcttgacttctcagctgatccgtggccaacggtgtcggctggtgccaaagatcttgtgaagaaaatgttgaagtatgatcctaaagaccggcttacagctagcgaagtgctgaaccatccgtggattaaagaagatggagaggcctcagacaaaccgcttgacaatgctgtgttgtccaggatgaaacagttcagagccatgaacaaactaaagaaaatggcactgaatgtaattgcagagaacctgtctgaagaagaaatcattggtctgaaagagatgttcaaagctctagacaccgataaaaacggaatagtcaccttggaggaattgagaaccggtctcccaaagcttggcaataagatctctgaggctgaaataaaacagttaatggaagcagctgatatggatggagatggatcgatagattacttggagtttatatcagcgacaatgcatatgaacagaatcgaacgtgaggatcatttgtacactgctttccagtacttcgacaaggacaacagcgggtacataacaatggaagagctagagcaggccatgaagaaatacaacatgggtgatgacaaatccattaaagagatcattgctgaagttgacaccgatcgtgacgggaaaataaactacgaagagttcgtagcgatgatgaagaagggaaatccagaactggtgacgaaccgacgcagagtcaacatgtaa

SEQ ID NO: 41 - DNA, 1578 bp, Brassica rapa
  misc_feature 1..1578: note = ORF of protein Cpk3
  source 1..1578: mol_type = unassigned DNA
atgggacacagacacagcaagtccaagacctcctcctcctcctcttcctccgacaacgtcgtccaccatgtcaaaccttccggcgaacgccgcggatcctccggctccggttccggacccgtcaaatcctcctcaggctccggaaccggcggatcccgttctcaacagaacggtcggatcctaggcaagccgatggaggatgtcaagggaacctacgacttgggtcgcgagcttggtcgtgggcagttcggagtcacgcacctcgtcactcacaaagagaccaagaaagtcttcgcttgcaagtcaatccccacgcgccgactcgtccaaagtgacgacattgaagatgttcgccgtgaagtccagattatgcaccatctcagcggtcacaggaacatagtggacttgaagggagcttacgaggacagacagtcggtgaatctgatcatggagctgtgtgaaggaggggagcttttcgataggatcatagctaaaggtcattacacagagagagccgctgcggatttgtgcaggcagatggtgatggttgtgcacagctgccactctatgggcgtgatgcaccgagacttgaagcccgaaaacttcctctttctgagcaaagatgagagctccccgttgaaagctacagactttggcctctccgtcttcttcaaaccaggagataagtttaaggatcttgttggaagtgcatactatgttgccccggaagttttaaaacggaactatggaccagaggctgatatctggagtgccggtgtgattctgtacatccttctcagtggtgtcccgccattttggggagaaaatgagacagggatcttcgatgccattctagaaggaaagcttgatttttcagctgatccatggcctgatgtatccaacggtgccaaagatcttgtgaagaagatgttgacgtatgaccctaaagaccggcttacagcgagtgaagtgctaaaccatccgtggattaaagaagacggagaggcgtcagacaaaccgcttgacaatgctgtgttatccaggatgaaacagttccgagcgatgaacaaactcaagaaaatggcactcaaggtcattgcagagaacttatctgaagaagaaatcattggtctgaaagagatgttcaaagctctagacactgataaaaacggaatagtcaccttggaggagttgagaaccggtctccccaagcttgggtctaagatctcagaggctgagataaaacagttaatggaagcagctgatatggatggagatggatcaatagattacttggagtttatatcagcgacaatgcatatgaacagaatcgaacgtgaggatcacttgtacactgctttccaatacttcgacaaggacaacagcggctacataacaatggaagagctagaacaagccatgaagaaatacaacatgggtgatgacaaatccatcaaagagatcattgctgaagtagacaccgatcgtgacggaaaaatcaattacgaagaattcgtggcgatgatgaagaaaggacatccagagttggtgacaaaccgacgcagagtcaatatgtaa

SEQ ID NO: 42 - DNA, 1443 bp, Hordeum vulgare
  misc_feature 1..1443: note = ORF of protein Cpk3
  source 1..1443: mol_type = unassigned DNA
caacagcccccaccccagcccgccctccccccagacgtcggccgggtgctgggccggccgatggaggacgtgcgcgccacctacaccttcggccgcgagctgggcaggggccagttcggggtcacctacctcgccacccacaagtcaaccggcgcgcgctacgcctgcaagtccatcgcggcccggaagctggcccgcgccgacgacgtggaggacgcgcggcgggaggtgcagatcatgcaccacctcacgggccaccgcagcatcgtcgagctgcgcggcgcctacgaggaccgccactccgtcaacctcgtcatggagctctgcgagggcggggagctcttcgaccgcatcatcgcccgcggccactactccgagcgcgccgccgccacgctctgccgcgaggtcgtcagcgttgtccacagctgccactccatgggggtcatgcaccgcgacctcaagcccgagaacttcttgttcttgaataagcgggaggactccccgctcaaggccaccgatttcgggctctccgtcttcttcaagcccggcgagcagttcagggatcttgttgggagtgcctattacgtggctccggaggtgctgaagcgccgatatggagccgaggccgacatttggagtgctggggtgatcctctacatcctcctatccggcgttcctccattctgggcagagaacgaggaaggtatatttgatgctgttctgaaaggtcacattgatttctcgtccgatccatggccatcgatttcaaatggggctaaagacttggtcaagaggatgctacggcaggaccccaaggagcggctcacggctgctgaaattttgaaccatccatggattagagaggatggcgaggccccagacaaaccacttgatattacagtgatcagtagaatgaagcagttcagggcgatgaacaagcttaagaaggttgccttgaagattgttgcagagagcttgtcagaggaagagatcgtgggcttaagagaaatgttcaagtccctggatactgataacagcgggacaattaccctggatgaactaagggctggcttaccaaagcttgggaccaaaattactgaatcagaaataagacagttgatggaggcggctgatgttgatggaaatgggaccattgattatgttgaattcatatcggcaacaatgcacatgaatagactagagaaggaagaccacatatttaaagcattcgaatactttgacaaggaccatagcgggtacataacagttgatgagctggaggaagcgctgaagaagtatgatatgggtgatgaggcaacaatcaaagatatcattgctgaagtggatacagatcatgatgggaaaattaactaccaggagtttgttgcgatgatgaagaataacagcccagagattgttccaaatcgacggcgcctattttaa

SEQ ID NO: 43 - DNA, 1785 bp, Hordeum vulgare
  misc_feature 1..1785: note = ORF of protein Cpk3
  source 1..1785: mol_type = unassigned DNA
cccaccccaacaccgcagcgcagatcacgcggtctccacggtcgtctctcccccaaaactcccacgctagcaccgctgctgcttcaaaccccgagcgagccccctatcgcctcgatctcccttcgtcaaacccattcgcacacaatgggcaaccgcacctcgcgccaccgccgcgccgccgccgaccagccggccgcggcccctccgccgacggcccagcccaaaccccagccgcagccgaagccgaagccacagacggcgccggccccggccccgacgccggaagcgggacaggtagccatgggccgcgttctgggacgtccgatggaggacgtgcgcgcgacctacaccttcggccgcgagctcggccgggggcagttcggggtcacctacctcgtcacgcacaaggctacgggccagcgcttcgcctgcaagtccatcgccacgcggaagctcgtccaccgcgatgacatcgaggatgtgcagcgcgaggtgcagatcatgcaccacctcacgggtcaccgcaacatcgtcgagctccgcggcgcctacgaggaccgccactcggtcaacctcatcatggagctgtgtgagggaggggagctattcgaccgcatcatcgcgcgggggcactactccgagcgagccgccgccttgctctgccgcgagatggtctccgttgtgcatagctgccactccatgggggttttccaccgggatctcaagcccgagaactttttgtttctgaataacaaggaggactcgccgctcaaggccactgacttcggcctctccgtcttcttcaaacacggggagcagtttaaggatcttgttggaagtgcgtattatgttgctcctgaggttctgaagcggcactatggagcagaagctgacatatggagtgctgggattattctgtacatccttctgtctggtgttcctcctttttgggcaggtgatgtgtgccaattctcatccgagttagattttctgctttttatttatccattaacacacatgttctattttccagacaatgaggatggcatatttgaggctgtcttgcttggtcacatagatttctcatctgacccttggccttcaatatctaatggtgcaaaagatttggtgaagaagatgttgcggcaagaccccaaagagcgcttgactgctgcggagattttgaatcacccatggatcagggaagatggagaggctccagataagccacttgacattactgttatcagtagaatgaagcagttcagggcgatgaacaagcttaagaaagttgcattgaagatcgttgcagagaacttgtctgaggaagagattacaggcttgaaagaaatgttcagatccctggacactgataacagtgggacaattactcttgaagagctaagatctggtttaccaaaacttggcaccaaaatttctgaatcagaaattacacagttaatggaggcggctgatgttgatggaaatgggaccattgattattctgagttcgtatcagcgacaatgcacatgaatagattggagaaggaagaccacatacttaaagcatttgaatattttgataaggaccacagcggatacataacagtagatgagctggaagaagctctgaagaagtatgacatgggggatgataaaacaattaaagatatcattgctgaagtagatacagatcatgatggaagaatcaactaccaggagtttgttgccatgatgagaaacaacagccctgagattgttccaaatcggcggcgcatgttctaa

SEQ ID NO: 44 - DNA, 1554 bp, Solanum tuberosum
  misc_feature 1..1554: note = ORF of protein Cpk3
  source 1..1554: mol_type = unassigned DNA
atgggcaactgtaacagcttatcttccgaccaagccgccaccacttcctccgccgccggcggtggcggcggcgccacttcagcgccgtcgaccaccggtatcagaatcatcccttcctcacaaacaccacctccacgaccattctccggcatcggccgtgttctcggccggccaatggaggatgtccggtccatctacattttcggcggcgaactcggccgcggtcaattcggagtcacctacttagttactaacaaaaaaacgcgccagcagttcgcgtgcaagtcaatcgctacacgaaaactcatcaacaaggacgatgtcgacgacgtccgccgtgaagttcagatcatgcatcaccttaccggtcaccggaacatcgttgagcttaaaggaacttttgaggataaacatcacgttcatctcgtcatggaactttgtgccggcggcgaactttttgaccggataattgctaaaggacactattccgagcgagctgctgctggagtttgccggcagatggtgactgttgttcacaattgtcattctatgggagttatgcatagggatttaaaacctgaaaatttcctgtttttgagctccgatgaaacttcaccattgaaggctactgattttgggctatcagtctttttcaaaccaggtgatgtgttcaaagacctggttggtagtgcatattatgtcgctcctgaagttctgcgccgagaatatggacctgaagctgatatttggagtgctggagttattctatacattctactcagcggtgttccaccattttatggagagaatgatcagagtatctttgatgctgttcttcgaggtcatcttgatttttcatctgatccttggccttcagtatcgagtagcgctaaagatcttgttaagaagatgctacgatctgatcctagagaaaggatttctgcagctgaagtcctaaatcatccatggatgagagaagatggtgatgcatctgacaaacctcttgacattgctgtcttatctagaatgaaacaattccgtgctatgaacaagttgaagaaagtagcactgaaggtaattgctgaaaatctttctgaagaagaaatcattggcttgaaggaaatgttcaaatctatagacacagatgacagtgggacaattacatttgaagaattgaaagctggtcttacaaaaatgggtacaaagctttctgagtcagaagtcaggcagttgatggaagcggctgatgttgatggaaatggaacaattgattaccttgagttcataacagctacgatgcacctgaatcgcatggaaagagaagatcacttgtacaaagcctttgagtattttgacaaggataagagcgggtatattacgatggaagaattggagcatgcgctgaaggaatacaacattactgatgaaaaaacaataaaagagatcattgctgaagttgacacagacaatgatggaagaatcaattacgatgagttcgctaccatgatgaggaaaggtaatccagattttgtcaacaataggcgccgcagatga

SEQ ID NO: 45 - DNA, 1578 bp, Amaranthus palmeri
  misc_feature 1..1578: note = ORF of protein Cpk3
  source 1..1578: mol_type = unassigned DNA
atggggaactgtaatggcttaccttccactctgtctcactcccgtctccgctccattgacgctaccaacaacgatactctctcctccacgttatcgccgcctccgccattacagcaacctcaacctcaacctccacaacctgcatcaccacaacctccaccgtcaaactccaatctcggccgtgttctaggtcgccctatcgaggatgttcgcgcaacatacactttcggccgcgaactaggtcgcggacaattcggagtaacttatttagttactcataaacaatctaagctacaatttgcttgtaaaaagattgctactcgtaatctgcttaatcaagatgatattgatgatgttcgtcgtgaagttcaaattatgcatcatcttactggtcatagaaacattgttgaacttaaaggtgcgtttgaagatcggcattatgttaatttggttatggaattgtgtgctggtggagagctttttgataggattattgctaaaggacactattctgaacgtgaagcggctaaattgtttaggcagattgttaccgttgtgtataattgtcattctatgggagttatgcatagagatcttaagccggagaatttcctgtttttgagctcagatgaagagtcgccattgaaagctactgattttggtctatctgtcttctttaagcctggagaagtcttcagagaccttgtcggaagcgcatattatgtagctcctgaggttttacgtcgtagttatggggctgaagctgatgtttggagtgctggagtgatattatacatactacttagtggagttccaccattttggggagagaatgagcagggaatttttgattctattctgcggggcaatcttgatttcaattcagatccatggctctcaatatctagcagtgccaaagacctggtgacaaagatgttgcggcaggatcctaaggaaagactttctgctcgtgatgctttaaatcatccttggatgagagaggatggggaagctcctgataagcctattgatattgctgttttgactagaatgaagcagtttagggcaatgaacaaactgaaaaaagtagcactaagggtaattgccgagaacctttctgaagaaaaaataattggtttaaaagaaatgtttaaatctatagatactgataatagtggaactatcacctatgaagaattaaagagcggtcttccaaagctgggaactaaactttctgagaaagaagtcaagcagttaatggaggcggctgacgtcgatggtaatggaaccatagattacatggaattcatcacagctacaatgcatatgaataggatggaggaagaggaccatatatacaaagcctttgaatattttgatgtggataaaagcgggtacattactatggaggaattggcgcaagccttgaagaggtataatatgggtgatgcaaaaacaatacgggagataattgcagaagttgatacagacaatgatggtaaaatcaattatgatgaatttgtagcgatgatgaagaaaggcaaccctgatatggttaccaacagacggaagagatga

SEQ ID NO: 46 - DNA, 1551 bp, Medicago truncatula
  misc_feature 1..1551: note = ORF of protein Cpk3
  source 1..1551: mol_type = unassigned DNA
atggggggcaactgcagcaccggcgccggcgccggaatcaacaccacaaccaccaccccccattccgatcctccacccaaaaacgccccaacaatcctcccacaaaacttcactccctcaccccctcaaccacccttacccaccaccaccctcggccgcatcctgaaccgtccaatggaagacgttcgctccatcttcatcttcggccgtgaactcggccgaggccaattcggcgttacatatttagtaacccacaaagttaccaaagaacaattcgcttgtaaatcaatcgctacacgtaaactcgtcaaccgtgacgatatcgatgatgtccgccgcgaggttcaaatcatgcatcatctgaccggtcatcgtaacattgttgaactcaaaggtgcttatgaggataaacactctgttaaccttgttatggagctttgtgctggtggtgaactttttgatcggattattctcaaaggacattactctgaacgtgctgctgctaatctttgtcgtcagattgttacggttgttcataattgtcatactatgggagttatgcatagggacttgaaacctgagaatttcttgtttcttggtaaagaagaaaattcgcctctcaaggccacggattttggtctctctgtgtttttcaagccaggagacgtgtttaaagatcttgttgggagtgcatattatgttgctcctgaagtgctgcgcaaaagttatgggcctgaaactgatatttggagcgccggaattatattatatattctgctttctggtgtcccccccttctgggcagaaaatgaacagggtatctttgatgctattcttcgtggacaaattgattttgcgtcggatccttggccttctatatcaagcagtgccaaagatctggtcaagaagatgctacgaaatgacgctaaagaacggatatcagcagttgaagtactcaatcatccttggatgagagtagatggagatgcatctgacaagcctcttgatattgctgttttgagcagaatgaaacagttccgagcaatgaacaagctaaagaaagtagctttaaaggttatcgccgaaaatctttctgaagaagaaattattggcttgaaggaaatgttcaaatccatggatacagacaatagtggaacaatcacttttgaagagttgaaagctggcctccctaaactcggtagtaaaatgtcagagtctgaagtaaggcagttgatggaagcggctgacgtggacggtaatggaaccatcgattatattgagtttataaccgctaccatgcatatgaatagaatggaaagagaggatcacctatataaagcctttgagtattttgaccaagataagagcgggtacatcacaaaggaagagttggaatctgcccttaagaagtataatatgggcgacgagaacacaataaaggagatcattgccgaagttgatacagacaacgatgcaagaattaactatgatgagtttgtagctatgatgaggaaaggcaacccagatataacccacaggcgtcgcaaataa

SEQ ID NO: 47 - DNA, 1554 bp, Medicago truncatula
  misc_feature 1..1554: note = ORF of protein Cpk3
  source 1..1554: mol_type = unassigned DNA
atgggcaaccgcatcagctccaaacacaaaccctcttccgattcccaaacccgttccaaatcagaaccctcacattccactcccacttccttccatcctcctaatcgaacccccaaacaatccactcacacccccaccccttcctcctcccccctcggccgcgtcttaggcaaacccatggaagacgtccgatcaacctacaccttcggccgtgaactcggtcgcggccaattcggtgtaacctacttagttacacacaaactcaccaaagaacaattcgcctgcaaatctatagccactcggaaactaatccgccgcgatgatcttgacgatattcgccgtgaagttcagatcatgcatcatctcaccggtcaccggaacattgttgagcttaaaggtgcttatgaggatcgtcattcggttaatcttatcatggagctttgtgctggtggtgagctttttgataggattatatccaaaggtcattactctgaacgtgctgcggctgagctgtgtcgccagattgtcactgttgttcataattgtcatactatgggtgttatgcatagggatttgaagcctgagaatttcttgtttcttagtaagcatgagaattcgccgcttaaagctacagattttggtctctccgtgtttttcaagcccgaagatgtattcaaagatcttgttgggagtgcatattatgttgctcctgaggtgttgcgtagaagctatggacctgaagctgatatttggagtgctgggattattttgtacattctcctctctggtgttcctcccttctgggcagaaaatgaacaaggtatcttcgatgctattcttcgtggacatcttgatttcgcatctgatccttggcctaaaatatcgagtattgctaaagatctgatcaaaaagatgttgcgagctgaccctaaagaacgaatttcagctgttgaagtccttgaccattcttggatgaaagaagatggagcatccgataagcctcttgatattgctgtcttaaccagaatgaaacaattcagggcaatgaacaagctaaagaaagtagctctaaaggttattgctgaaaatctttctgaagaagaaattataggcttgaaggaaatgtttaaatccatggatacagataacagtggaacaatcacttttgaagagttgaaagctggtctccccaaactaggtactaagatttcggaatctgaagtaaggcagttgatggaagcggctgatgttgacggaaatggaaccattgattatattgaatttataacagctaccatgcacttgaatagaatggaaagagaggaccacctatacaaagcttttgaatattttgacaacgacaaaagcgggtatattacaaaagaagagttggaatctgcccttacaaagtacaacatgggtgatgagaaaacaataaaggagataattgacgaagttgattcagacaatgatggaagaattaactatgaggagtttgtagccatgatgaggaaaggcaatccagacttgataaccaacaaacgtcgcaaataa

SEQ ID NO: 48 - DNA, 1008 bp, Triticum aestivum
  misc_feature 1..1008: note = ORF of protein Cpk3
  source 1..1008: mol_type = unassigned DNA
atgggggttttccaccgggatctcaagcccgagaactttttgtttctcaacaacaaggaggactcgccgctcaaggccactgactttggcctctccgtcttcttcaaacacggggagcagtttaaggatctcgttggaagtgcgtattatgttgctcctgaggttctgaagcggcactatggagcagaagctgacatatggagtgcggggattattctgtacatccttctgtctggtgttcctcctttttgggcagacaatgaggatggcatatttgaggctgtcttgcttggtcacatagatttctcatctgacccttggccttcaatatctaatggtgcaaaagatttggtgaagaagatgttgcggcaagaccccaaagagcgcttgactgctgcagaaattttgaatcacccatggattagggaagatggagaggctccagataagccacttgacattactgttatcagtagaatgaagcaattccgggcgatgaacaagcttaagaaagttgcattgaagatcgttgcagagaacttgtctgaggaagagataacaggcttgaaagaaatgttcagatccctggacactgataacagtgggacaattactcttgaagagctaagatctggtttaccaaaacttggcaccaaaatttctgaatcagaaattacacagttaatggaggcggctgatgttgatggaaatgggaccattgattattctgagttcgtatcagcgacaatgcacatgaatagattggagaaggaagaccacatacttaaagcatttgaatattttgataaggaccacagcggatacataacagtagatgagctggaagaagctctgaagaagtatgacatgggggatgataaaacaattaaagatattatcgctgaagtagatacagatcatgatggaagaatcaactaccaggagttcgttgccatgatgagaaacaacagccctgagattgttccaaatcggcggcgcatgttctaa

SEQ ID NO: 49 - DNA, 903 bp, Triticum aestivum
  misc_feature 1..903: note = ORF of protein Cpk3
  source 1..903: mol_type = unassigned DNA
aaatggcgggagcagtttaaggatctcgttggaagtgcgtattatgttgctcctgaggttctgaagcggcactatggagcagaagctgacatatggagtgcggggattattctgtacatccttctgtctggtgttcctcctttttgggcagacaatgaggatggcatatttgaggctgtcttgcttggtcacatagatttctcatctgacccctggccttcaatatctaatggtgcaaaagatttggtgaagaagatgttgcggcaagaccccaaagagcgcttgactgctgcagaaattttgaatcacccatggattagggaagatggagaggctccagataagccacttgacattactgttatcagtagaatgaagcagttccgggcgatgaacaagcttaagaaagttgcattgaagatcgttgcagagaacttgtctgaggaagagattacaggcttgaaagaaatgttcagatccctggacactgataacagtgggacaattactcttgaagagctaagatctggtttaccaaaacttggcaccaaaatttctgaatcagaaattacacagttaatggaggcggctgatgttgatggaaatgggaccattgattattctgagttcgtatcagcgacaatgcacatgaatagattggagaaggaagaccacatacttaaagcatttgaatattttgataaggaccacagcggatacataacagtagatgagctggaagaagctctgaagaagtatgacatgggggatgataaaacaattaaagatattatcgctgaagttgatacagatcatgatggaagaatcaactaccaggagttcgttgccatgatgagaaacaacagcccggagattgttccaaatcggcggcgcatgttctaa

SEQ ID NO: 50 - DNA, 897 bp, Triticum aestivum
  misc_feature 1..897: note = ORF of protein Cpk3
  source 1..897: mol_type = unassigned DNA
ggcgagcagttcagggatcttgttgggagtgcatactatgtggctccagaggtgctgaagcggcgatatggagccgaggccgacatttggagtgctggagtgatcctctacattctgctctccggcgttcctcctttctgggcagagaatgaagaaggtatatttgatgctgttctgaaaggtcacattgatttctcgtctgatccctggccatcgatttcaaatggggctaaagacttggtcaagaggatgctacggcaggaccccaaggagcggctcacggctgcggaaatattgaatcatccatggatcagagaggatggagaggccccagacaaaccacttgatattacagtgatcagtagaatgaagcagttcagggcgatgaacaagcttaagaaggttgccttgaagattgttgcagagagcttgtcagaggaagagatcgtgggcttaagagaaatgttcaagtccctggatactgataacagtgggacaattactcttgatgaactaagggctggcttaccaaagcttgggaccaaaattactgaatcagaaataagacagttaatggaggcggctgatgttgatggaaatgggaccattgattatgttgaattcatatcggcaacaatgcacatgaatagactagagaaggaagaccacatatttaaagcatttgaatactttgacaaggaccacagcgggtacataacagttgatgagttggaggaagctctgaagaagtatgatatgggtgatgaggcaacaattaaagatatcattgctgaagtggatacagatcatgatgggaaaattaactaccaggagtttgttgcgatgatgaagaacaacagtccagagattgttccaaatcgacgccgcctattttaa

SEQ ID NO: 51 - DNA, 1407 bp, Triticum aestivum
  misc_feature 1..1407: note = ORF of protein Cpk3
  source 1..1407: mol_type = unassigned DNA
atgggccgcgttctgggacgcccgatggaggacgtgcgcgcgacctacaccttcggccgcgagctcggccgggggcagttcggggtcacctacctcgtcacgcacaaggctacgggccagcgcttcgcctgcaagtccatcgccacgcggaagctcgtccaccgcgatgacatcgaggatgtgcagcgggaggtgcagatcatgcaccacctcacgggtcaccgcaacatcgtcgagctccgcggcgcctacgaggaccgccactcggtcaacctcatcatggagctatgtgagggcggggagctattcgaccgcatcatcgcgcgggggcactactccgagcgagccgccgccctgctctgccgcgagatggtctccgtcgtgcacagctgccactccatgggggttttccaccgggatctcaagcccgaaaactttttgtttctcaacaacaaggaggactcgccgctcaaggccactgacttcggtctctccgtcttcttcaaacacggggagcagtttaaggatctcgttggaagtgcgtattatgttgctcctgaggtactgaagcggcactatggagcagaagctgacatatggagtgcggggattattctgtacatccttctgtctggtgttcctcctttttgggcagacaatgaggatggcatatttgaggctgtcttgcttggtcacatagatttctcatctgacccctggccttcaatatctaatggtgcaaaagatttggtgaagaagatgttgcggcaagaccccaaagagcgcttgactgctgcagaaattttgaatcacccatggattagggaagatggagaggctccagataagccacttgacattactgttatcagtagaatgaagcagttccgggcgatgaacaagcttaagaaagttgcattgaagatcgttgcagagaacttgtctgaggaagagattacaggcttgaaagaaatgttcagatccctggacactgataacagtgggacaattactcttgaagagctaagatctggtttaccaaaacttggcaccaaaatttctgaatcagaaattacacagttaatggaggcggctgatgttgatggaaatgggaccattgattattctgagttcgtatcagcgacaatgcacatgaatagattggagaaggaagaccacatacttaaagcatttgaatattttgataaggaccacagcggatacataacagtagatgagctggaagaagctctgaagaagtatgacatgggggatgataaaacaattaaagatattatcgctgaagttgatacagatcatgatggaagaatcaactaccaggagttcgttgccatgatgagaaacaacagcccggagattgttccaaatcggcggcgcatgttctaa

SEQ ID NO: 52 - DNA, 1524 bp, Lolium perenne
  misc_feature 1..1524: note = ORF of protein Cpk3
  source 1..1524: mol_type = unassigned DNA
atgggcggccgcgcctcccgcccgcgcgccccaccgcccgacgagcaccaccagtcctcccaccccaagcagcccaactggccgcgccccaagaaccggcgccaaccccctcccctccctgcccctcccccacagccgcagccccccgccccccagccccagcccgcgctccccccagacgtcggccgcgtcctcggccgcccaatggaggacgtgcgcgcgacctacaccttcgggcgcgagctgggccgcggccagttcggcgtcacctacctggccacccacaaggcctccggcgccaggcgcgcctgcaagtccatcgccgcccgcaagctcgcgcacgcctccgacgccgaggacgtgcgccgcgaggtgcagatcatgcaccacctcaccggccaccgcagcatcgtcgagctgcacggcgccttcgaggaccgccactccgtcaacctcgtcatggagctatgcgagggcggcgagctcttcgaccgcatcatcgcccgcggacactactccgagcgcgccgccgccgcgctctgcagggaggtcgtctccgtcgtccacagctgccactccatgggggtcatgcatagggatctaaagcccgagaatttcttgtttctcaataagagggaggactcgccgctcaaggccaccgatttcggactatcagtcttcttcaagccaggtgagcagttcaaggatcttgttgggagcgcgtattatgtcgctccggaggtgctcaagcggcgatatggtgccgaggcagacatatggagtgccggagttatcctctacattctcctctccggcgtccctcctttctgggcagagaacgaggatggtatattcgacgccgttcttaaaggccatatagatttctcgtcggaaccctggccatcgatttcaaatggtgcaaaagacttggtcaagaggatgctaaagcaagaccctagggagcggcttactgctgcggaaattttgaaccatccatggattagagaggatggagaggccccagacaaaccacttgatattacagtgatcagtagaatgaagcagttcagggcgatgaacaagcttaagaaggttgccttgaagatcgttgcagagagtttgtcagaggaagagatcatgggcttaagagaaatgttcaaatcgctggatactgataacagtggtacaattactcttgaagaactaagggctggcttaccaaagcttggtaccaaaatcaccgaaccagaattgagacagttgatggaagcggctgatgttgatggaaatgggaccattgattatggcgaattcatatcagcaaccatgcacatgaatagactagagaaggaagatcacatatttaaagcatttgagtattttgacaaggaccatagcgggtacataacagttgatgagttggaagaagctctcaagaagtatgatatgggtgacgaggcaacaatcaaagatatcattgctgaagtggatacagatcatgtgagtaactccagtaaactgtaa

SEQ ID NO: 53 - DNA, 1587 bp, Lolium perenne
  misc_feature 1..1587: note = ORF of protein Cpk3
  source 1..1587: mol_type = unassigned DNA
atgggcggccgcgcctcccgcccgcgcgccccaccgcccgacgagcaccaccagtcctcccaccccaagcagcccaactggccgcgccccaagaaccggcgccaaccccctcccctccctgcccctcccccacagccgcagccccccgccccccagccccagcccgcgctccccccagacgtcggccgcgtcctcggccgcccaatggaggacgtgcgcgcgacctacaccttcgggcgcgagctgggccgcggccagttcggcgtcacctacctggccacccacaaggcctccggcgccaggcgcgcctgcaagtccatcgccgcccgcaagctcgcgcacgcctccgacgccgaggacgtgcgccgcgaggtgcagatcatgcaccacctcaccggccaccgcagcatcgtcgagctgcacggcgccttcgaggaccgccactccgtcaacctcgtcatggagctatgcgagggcggcgagctcttcgaccgcatcatcgcccgcggacactactccgagcgcgccgccgccgcgctctgcagggaggtcgtctccgtcgtccacagctgccactccatgggggtcatgcatagggatctaaagcccgagaatttcttgtttctcaataagagggaggactcgccgctcaaggccaccgatttcggactatcagtcttcttcaagccaggtgagcagttcaaggatcttgttgggagcgcgtattatgtcgctccggaggtgctcaagcggcgatatggtgccgaggcagacatatggagtgccggagttatcctctacattctcctctccggcgtccctcctttctgggcagagaacgaggatggtatattcgacgccgttcttaaaggccatatagatttctcgtcggaaccctggccatcgatttcaaatggtgcaaaagacttggtcaagaggatgctaaagcaagaccctagggagcggcttactgctgcggaaattttgaaccatccatggattagagaggatggagaggccccagacaaaccacttgatattacagtgatcagtagaatgaagcagttcagggcgatgaacaagcttaagaaggttgccttgaagatcgttgcagagagtttgtcagaggaagagatcatgggcttaagagaaatgttcaaatcgctggatactgataacagtggtacaattactcttgaagaactaagggctggcttaccaaagcttggtaccaaaatcaccgaaccagaattgagacagttgatggaagcggctgatgttgatggaaatgggaccattgattatggcgaattcatatcagcaaccatgcacatgaatagactagagaaggaagatcacatatttaaagcatttgagtattttgacaaggaccatagcgggtacataacagttgatgagttggaagaagctctcaagaagtatgatatgggtgacgaggcaacaatcaaagatatcattgctgaagtggatacagatcatgatgggaagattaactatgaggagtttgttgcaatgatgaagaataacggcccagagattgttccaaacagacggcgactattttaa

SEQ ID NO: 54 - DNA, 1581 bp, Lolium perenne
  misc_feature 1..1581: note = ORF of protein Cpk3
  source 1..1581: mol_type = unassigned DNA
atgggcaaccgcacctcgcgccaccgccgcgccgccgccgagcagccggcgacgcccccgccgcccgcgcagcccaagccccagcccaagcaggagacgccgccgcaccgcccgcagccgacgacggcgccgcccccctgcccagccccggcgccggaggcggcggggcaggcgatgggccgcgtcctggggcgcccgatggaggacgtgcgcgcgacctacaccttcgggcgcgagctgggccggggccagttcggggtcacctacctcgtcacgcacaaggccacggggcagcgcttcgcctgcaagtccatcgccacccggaagctcgtccaccgcgacgacatcgaggacgtgcagcgggaggtgcagatcatgcaccacctcacgggccaccgcaacatcgtcgagctccgcggcgccttcgaggaccgccactcggtcaacctcatcatggagctctgcgagggcggggagctcttcgaccgcatcatcgccaggggacactactccgagcgggccgccgcggggctatgccgcgagatggtctccgtcgtgcacagctgccactccatgggggtattccaccgggatctcaagcccgagaactttctattcctcaataacaaggaggactcgccgctcaaggcaactgacttcggcctatccgtattcttcaagcatggggagcagtttaaggatctcgttggaagtgcatattatgttgctcccgaggttttgaaacgccactatggagcagaagctgacatatggagtgctgggattattctctacatccttctgtccggtgttcctcctttttgggcagacactgaggatggcatatttgaggctgtcttgcttggcaacatagatttctcatctgacccttggccttcagtatccaatggtgcaaaagatttggtgaagaagatgctgcggcaagaccccaaagagcgcttgactgctgcggagattttgaatcacccatggattagagaagatggagaggccccagacaagccacttgacatcacagttatcagtagaatgaagcagttcagggcaatgaacaaactcaaaaaagttgcattgaagatcgttgcagagaacttgtctgcagaagaaattactggcttgaaagaaatgttcagatccttggatactgataacagtggtacaattacactggaagagctaagatctggtttaccaaaacttggcaccaaaatttctgagtcagaaattgcacagttaatggaggcggctgacgttgatggaaatgggaccattgattattctgagttcgtgtcagccacaatgcacatgaatagattggagaaggaagaccacatactgaaagcatttgaatattttgataaggaccacagcggatacataacagtagatgagttggaagaagctctgaagaagtatgacatgggagatgataaaacaatcaaagaaatcatcgctgaagtagatacagataatgatggaagaatcaactaccaggagtttgttgccatgatgagaaacaacagccctgagatcgttccgaatcggcggcgcatgttctaa

SEQ ID NO: 55 - DNA, 1581 bp, Lolium perenne; identical to SEQ ID NO: 54
  misc_feature 1..1581: note = ORF of protein Cpk3
  source 1..1581: mol_type = unassigned DNA

SEQ ID NO: 56 - DNA, 2031 bp, Arabidopsis thaliana
  misc_feature 1..2031: note = ORF of protein Crk34
  source 1..2031: mol_type = unassigned DNA
atgaagctaaagatttcgtttttaccaaccttcttgatttttctcataagcttagattctgttactgctcaagaaatctgtttcagtggattctttaaacccaatagtacatacgatttaaaccgtcgtcaaatcctctctactctttcttctaatgtcacaagtcacaacggctttttcaactccaagtttggtcaagctcccaatagagtcttcatcaatggcatgtgcatccctgggactaaaccagagacttgttcagattgtattaaaggtgcatcagacaagatatcagagagttgtcctaacaaaacagatgcttatacatggcctgattgttgtatggtacgttattccaacgtttctttctctggttcactcgttatggaaccttctgaaactctctatcatacgggagatatcgaagatacaggcactaatttaacagtgtttgatagaatttgggaagagttaatgctccgtacaataactgcagcttcattgagtagtagtaatggatcatcatttggacaaaagtactttgcagctgaggttgcttcattgactactttccaaaccatgtatgcgatgatgcaatgtacgccggatgtttcttcgaaagattgtgagttttgtttgaagacaagtgttggtgattatgagtcatgttgccgtggtaaacaaggtggtgcggtgataagaccaagttgctttgtccggtgggatttgtatccttatgctggagcttttgaaaatgttacatttccaccgcctccaccgcagtctttacctcagccaccggtttctttgattcctcctccagttagcgatagggcaaatacaaccatcaaagatggaaaatcattttctacagggattattgtggctattgttgttcctatcattgttatcttggtttcacttgttgtactactcgtggtttgcagaagaaagaagtcatataaaacaactgaggttcaagctactgatgagatcacaacaacacactcactgcagtttagttttaagacaattgaagctgcaactgacaagttttcggacagtaacatgatcgggcgaggtggatttggtgaagtttacaggggaaagctttcaagtggacctgaagtagcagtgaagagactgtcgaaaacatcaggacaaggcgcggaagagttcaagaatgaggctgttcttgtgtcgaagcttcaacataagaatctggtcaggcttcttggattttgtttggaaggagaagaaaagatactcgtctatgagtttgtacccaacaaaagccttgactattttctctttgaccctgcaaaacaaggtgaactagattggacaagacggtacaatatcattggggggattgctcgaggaattctatatcttcatcaagattcacggcttacaatcatacaccgtgatctcaaagcgagtaacattctcttagatgctgatatgaaccccaaaattgcagattttggcatggcgaggatttttggagttgatcaaagtcaagccaacacaagaagaatcgccggaacatttggttatatgtctccagaatatgcaatgcgtggccatttctccatgaagtctgatgtctatagctttggagtattggtactagagattataagtggcaaaaagaatagcagtttctacaacattgatgattctggtagcaatttggtaacacacgcttggagactttggagaaatgggtcaccgttagaactggtggatccaaccataggagaaagttatcagagtagcgaggctactagatgcatccatatcgcactattatgtgttcaagaagatcctgcagatcgtccattgttaccagcgatcatcatgatgcttactagtagcacaaccactttacatgtgcctcgagcgcccggattttgcctaagcgggcgtgatctggaacaggatggtgtagagtataccgaatccacaagcaggtctattcctggttctatcaatgatgcatcgattacagagttctatcctcgttga

SEQ ID NO: 57 - DNA, 1575 bp, Arabidopsis thaliana
  misc_feature 1..1575: note = ORF of protein Cyp705a18
  source 1..1575: mol_type = unassigned DNA
atggcaacacttatcagcgttgactttcagcactgtttcatgtacataatcctcctatgcctcttctcacttctctgttactctctcttcttcaggaaaccaaagggcacacgagttggctgtgatctgcctccgagccctccgtctcttcctatcattggtcatcttcaccatctcctctcttctctaactcacaagtctttacaaaaactctcctccaagtatggacctttcctccatctccggatctttagtgttcccatcatccttgtctcatctgcttcagtggcatatgagatctttaaggcccacgatgtgaacgtctcatctcgtggtgctgctgcgatcgatgagtccctcttgtttggatcttctagcttcatttacgctccctatggagattactggaagttcatgaagaagatcttcgccactaagctgctccgaccacaggtgctggagaattcacgtggtgttcgtgctgaagagttaaagcggttttacaggagaattctcgataaggcgaggaagaatgagaacatcgagatcagtaaggaatcggcgatgctcatgaacaacatcttatgcaggatgagcatgggaaggagtttttcagaggaaaatggtgaggcagagagagtcaggggcctggtgggtgaatcatatgccttggtgaagaagattttcttcgcagctacattgcgcagactgcttgagaagcttcggatcccactattcaggaaggagataatgggtgtgtccgacagatttgatgagctgctagagaggattattgtggaacacaaagacaaacttgaaaaggagcatcaagttatggatatgatggatgtgttgttggcagcttatcgagacaaaaacgcagagtataagatcactaggaatcatatcaagtcgttttttgtggaccttttcgttgggggcactgatacctcggtccaaacaacacaatggactatggctgagatcattaataaccctaacattcttcagacactgagaaaagaaatcgattcagttgtagggaaatcaaggttgattcacgaaacggatataccaaaccttccttatttgcaagcagtggttaaggaaggactaagattgcatcctccagggcccctcttgataaggacttttcaagaaagatgtgagatgaaagggttctacataccggagaagacaacactcgttatcaacgcttatgctgtgatgagagatcccgactcttgggaagatcctgacgagtttaagccagagaggtttctaagttattcaagatcagggcaagaagacgagaaagagcaaacacttaagtacctttcttttggtggcggaaggagaggatgtcctggagtaaatctaggttatatatttgttggaactgcaataggaatgatggtgcagtgctttgactggaaaatcgaaggagataaggtcaacatggaagagacttatggaggaatgaacctaaccatggttaacccgcttaagtgcactccagttcctcgaacccaacctttttcttttacttctaatctcgagattcttagttcctga

SEQ ID NO: 58 - DNA, 1503 bp, Arabidopsis thaliana
  misc_feature 1..1503: note = ORF of protein Cyp71b26
  source 1..1503: mol_type = unassigned DNA
atggatagcatttggattctatcacttctcttcttcatcatctttcttcttctggccgccttcaaacgcaaaaatcatgggaaacatcgacgaataccatctcctcctggttttccaatcatcggaaacttacatcagctcggagaattacaacatcagtctctatggaagctctcaaagaagtatggccctgtgatgcttttgaagcttggaaaagtccccacactcatactttcttcatcagaaacagcaaaacaagctctaagagactatgatctccattgttgtagccgtccttccttagcaggaggaagagagctctcttacaacaatctagatatgtcttcatctccttataatgaatattggaaggaactaaggaagctctgcagtcaagaactctttagtgctaataaaattcaatcgattcaacctattaaggacgaggaggtcaagaaagttatcgattcaatcgcggaatcatcttctctaaagaatccggttaacttgagcaagacgtttcttgctttaactacaagtgtagtatgcaaggcagcatttggtgtgagttttgagggaagtgtgctcaacagtgatagattcaataagttagtccgagatactttcgagatgttgggaagcttctctgcctcagattttattccgtatgtcggatggatcatcgataagttcaatggtttacaagggtggagaaagaaaagctttcgagatcttgatgcgttctatgaacaaatttttgatctgcataaggaagagaaggaagtaggaagtgaagatttagtggatgtgctcttgaggttggagaaagaagaaattgttgttggaaatggcaagctcacaagaaatcatatcaaagcaattttgatgaacattcttttaggaggaatcgatacttctgcaataacaatgacatgggcaatggcagaacttgctaaaaaccctagagtgatgaagaaagttcaagcagaaatcagaaaccaaatcaagaacaaagaaagaatcagctttgatgacactgataagctcgagtacttgaaaatggtgatcaaagaaacatggaggttacatcctccaacacctcttctcctcccaagagatgtaatcactgaatttgagatcaatggctacacgattcctgccaaaacacggcttcatgtgaatgtttgggctatcgggcgtgatcctgatacatggaaagatccagaaatgtttctccccgaaaggtttaatgatagtaacattgatgcaaaaggacagaactttgagttgttgtcgtttgggagtggtaggagaatttgtcctggactgtacatgggaacaacaatggtggagtttggcctagctaatatgttgtatcattttgattggaagttaccagaaggcatggtagtcgaagatatcgatatggaagaagctcctggacttactgtgagcaaaaaaagtgagcttgtacttgttccagtgaagtatttagaccattga

SEQ ID NO: 59 - DNA, 1608 bp, Arabidopsis thaliana
  misc_feature 1..1608: note = ORF of protein Cyp78a8
  source 1..1608: mol_type = unassigned DNA
atgagaaccgaaatagaaagtttgtgggtatttgctcttgcatcaaaattcaatatttacatgcaacaacattttgcttcccttctcgtcgccattgctatcacttggtttaccataaccatcgtattttggtctactccgggtggaccggcttggggaaaatacttcttcactcgccggtttatttctctcgattacaaccgaaaatacaagaatctcattcccggtcctagagggtttccacttgtgggaagcatgagccttaggtcaagccacgtggctcatcagcgcatagcgtctgtggctgagatgagtaacgccaagcggctcatggcgtttagcctcggtgatactaaggtggtggtgacgtgtcatcctgccgtggcaaaggagatactaaacagttcggtttttgctgaccgaccggttgacgaaaccgcttacggtttgatgtttaaccgagccatgggatttgctcccaatggtacttattggcgtacgctgcgtcggttaggctcgaaccatctttttaacccgaagcaaatcaaacaatcggaggatcagagacgggtgatagcgactcagatggtgaatgcgtttgcacgtaaccctaaatccgcgtgtgcagtgcgtgatttgctcaaaacagcgtcgttgtgtaacatgatgggtttggttttcgggagagagtatgaattggagtcaaataacaacttggaatctgaatgcttaaagggtttggttgaagaagggtacgatcttctaggtacgttaaattggaccgaccatcttccttggttagccggtttagatttccaacaaatccggtttaggtgctcgcagctcgtaccgaaagtaaatctgttattgagccgtatcatacatgaacaacgtgctgccacgggtaactttcttgacatgttactttctcttcaaggttcagaaaaattatcagaatccgacatggttgctgttctttgggaaatgatatttaggggaacggacactgttgcggttttggtcgagtgggtgctagcgaggattgtgatgcatcccaaagttcaattaacggtccacgatgagcttgaccgagtcgttggcagatcaagaaccgtggatgagtcagaccttccatcactcacgtatctaacggctatgatcaaagaagtgttgaggctgcatccaccaggtccactgctttcttgggcacgactgtctataacagacacttccgtagatggatatcacgtgccggctgggaccaccgcgatggtcaacatgtgggctatagcacgtgacccacacgtgtgggaggatcctttagagtttaagcctgagaggttcgtggctaaagagggtgaagctgagttctctgttttcgggtcggatctgaggttggcaccgttcgggtcgggtaagagggtttgccctggaaagaatttgggacttacaacggtgtcgttttgggttgcaacgctcttgcatgagtttgagtggcttcctagcgtcgaagctaaccctccagatctctcggaggttttgaggctctcgtgtgagatggcttgtccactcatcgttaacgtaagctcaaggcgtaagataatgtaa

SEQ ID NO: 60 - DNA, 1743 bp, Arabidopsis thaliana
  misc_feature 1..1743: note = ORF of protein Cyp97b3
  source 1..1743: mol_type = unassigned DNA
atggtagcagccatggcttttcctgccgctgctacttatcccacccatttccaaggcggcgctcttcatctgggtaggaccgatcattgcctcttcggtttctaccctcaaaccatttcctctgtgaattctcggagagcttctgtttccatcaagtgccaatctacggagccaaagacgaatggtaacatattggacaatgcgagcaaccttttgacaaattttttaagtggtggaagtttggggtcaatgcctactgctgaaggctctgtctctgatttgtttggaaagcctctctttttatctctttacgactggttcttggagcatggaggaatttataaacttgcgtttggtccaaaagcctttgttgtcatctcagatcccattattgcaaggcatgtcctccgggaaaatgctttttcttatgacaagggagttcttgctgagatcttagagccgattatgggaaaagggttaataccggctgatctagatacgtggaagttaagaagaagagctatcactcccgcattccataaattgtatctagaggccatggtcaaagtatttagtgactgttcggagaaaatgatattgaaatctgagaaactcataagggagaaagaaacttcaagcggggaggacaccattgagttggatctggaagcagaattctcgagtctggctcttgatattataggtcttagcgtgttcaactacgattttggctctgtcacaaaagagtcccctgtgatcaaggcagtttatggaactcttttcgaggcagagcatcggtctactttctacttcccttattggaactttcctccagctagatggatagttccgaggcaacgaaagttccaaagcgatctgaagattataaacgattgccttgatggcctcattcaaaatgctaaagagacaagacaggaaacagatgttgagaagctccaggaaagggactacactaatctcaaggatgcaagtcttttgcggttcttagtcgatatgcgcggtgttgacattgatgaccggcagctgagggatgacttgatgactatgctaattgctggtcatgagacaacagcagcagtacttacttgggctgttttccttctgtcacaaaatcctgaaaaaattaggaaagctcaagctgagattgatgctgtgcttggtcaaggtccacccacttatgaatcaatgaaaaagctcgagtacatacgactgatcgttgtagaagtccttcgtctctttcctcagccacctttgctcatcagacgcactctcaaaccagaaacattacccggaggacacaaaggggaaaaagaaggtcataaagttccaaaagggactgatatcttcatttctgtgtataatctccatagatctccatacttttgggataatccccacgattttgagcctgagaggtttttaagaacaaaggagagcaatggaattgaaggatgggctggctttgatccatctcgtagccccggggcactatatccgaatgagataatagcagactttgcattcttaccatttggtggaggaccaagaaaatgcattggagaccagtttgcactaatggaatcgaccgtcgcactagctatgttgtttcagaaattcgatgtggagctgcgtggaacgccagaatctgttgaactcgtgagcggcgcaacgattcatgccaaaaatgggatgtggtgcaaactaaagagaagatcaaagtga

SEQ ID NO: 61 - DNA, 5730 bp, Arabidopsis thaliana
  misc_feature 1..5730: note = ORF of protein Dcl1
  source 1..5730: mol_type = unassigned DNA
atggtaatggaggatgagcctagagaagccacaataaagccttcttattggctagatgcttgcgaggacatctcttgtgatcttatcgatgatctcgtgtctgaatttgatccttcctctgttgctgtcaatgaatccactgatgaaaacggcgtcatcaatgattttttcggtgggattgatcacattttagatagtatcaagaacggtggaggcttaccaaacaatggcgtttctgataccaattctcaaatcaacgaggttactgtaactcctcaggttattgctaaggagacagtgaaggagaatgggttgcaaaagaatggcggtaagagagacgaattctcgaaagaggaaggagacaaggataggaagagagctagggtttgtagttatcagagtgaaaggagtaacctttcaggtagagggcatgttaataattctagggagggagataggtttatgaataggaaacgtactcgtaattgggacgaggcgggtaacaataagaagaaaagggaatgtaacaattacagaagagatggtagagatagagaagttaggggttattgggagagggataaagttggttccaatgagttggtttataggtcagggacttgggaagctgatcatgaaagagatgttaagaaagtgagtggtggaaaccgcgaatgcgatgtcaaggcagaggagaacaagagtaagcctgaagaacgtaaagagaaggttgtggaagagcaagcaaggcgataccagttggatgttcttgaacaagctaaagcgaaaaacacgattgctttccttgagaccggtgctggaaagacacttatcgcgattcttcttattaaaagtgttcataaggatctgatgagccagaacagaaaaatgctctcggtgttcttggttcccaaagtgcctttggtttatcagcaagcagaagtgatccgtaatcaaacttgttttcaagttggacattattgtggtgagatgggacaggacttttgggattctcgaaggtggcaacgagagtttgagtctaagcaggttctagttatgacagcacaaattctgttgaatatactgagacacagtatcattagaatggaaacaattgatcttcttattctcgacgagtgtcaccacgctgtcaagaaacatccatactctttagtgatgtcagagttttaccatacaactcctaaagataaaagacctgccatctttggaatgactgcttcgcctgttaatttaaagggtgtttcaagccaagtagattgtgcgataaagatacgtaacctcgagaccaagttggattctacggtttgtactataaaagatcgaaaagaattagagaaacatgtgcctatgccttcagagatagtcgtcgagtatgacaaagctgctactatgtggtctcttcatgagacaataaagcaaatgattgcagctgttgaagaagcggcacaagcaagttcaaggaaaagcaagtggcaatttatgggggctagggatgctggagcaaaggatgaattgagacaggtttatggcgtctctgaaagaacggagagcgatggtgctgccaatttgattcataaacttagagctatcaattatactcttgctgaattgggtcaatggtgtgcttacaaggtgggacaatcattcttgtctgctttgcaaagtgatgagagggtgaatttccaagtcgacgtgaagtttcaagaatcatacctcagtgaggtggtgtcactcttgcaatgtgagcttctggaaggcgctgctgctgaaaaagtcgcggcggaagttggcaaaccagaaaatggtaatgcacatgacgagatggaggagggagagctccctgatgatcctgtggtctcgggaggggagcacgttgatgaagtaataggcgccgcagtggctgatgggaaagttactccaaaagtacaatcattgatcaaactactcctcaaatatcagcacacagctgattttcgagctattgttttcgttgagagggtggttgctgctttggttcttcctaaggtttttgcggagctgccttcgcttagttttatacggtgtgccagcatgattggacacaataacagccaggagatgaaatcatctcaaatgcaggatacaatttccaaattccgagatgggcatgtgacactgttagttgccacaagcgttgctgaggaaggacttgatattaggcaatgtaacgttgttatgcgtttcgaccttgcaaagacggtgctggcatacattcagtctcgtggccgggcaagaaagcctggatcagactacatactcatggttgagagaggaaatgtatctcacgcagcgttcctaaggaatgctaggaacagtgaggagacacttcgaaaagaagcaatagaaaggactgatcttagtcatctcaaagatacatcgagattaatctcaattgatgctgtgcctggtacagtttataaggtggaggcaactggtgccatggttagcttgaattccgcggttggtcttgtacatttctactgctctcagcttcctggtgacaggtatgcaatccttcgtcctgagtttagcatggagaagcatgaaaagcctgggggccacacggaatattcatgtaggcttcagcttccttgcaatgcaccgtttgaaatacttgagggtcctgtttgcagttcaatgcgtcttgcacaacaggctgtatgtttagctgcttgcaagaaactgcatgagatgggtgcatttaccgatatgctattaccggacaaaggaagtggtcaagacgctgagaaggctgaccaagatgatgaaggtgagcctgttcctggaactgctagacatagagagttctatcctgaaggtgtggcggatgtacttaagggagaatgggtttcatctggaaaggaagtttgtgagagctcaaagctattccatttatacatgtataatgtcagatgtgtagattttggctcttcaaaagatccattcctaagcgaagtttcagagttcgcgattctttttggcaatgagctggatgcagaggtattatcgatgtctatggatctttatgttgctcgggccatgatcactaaagcatctcttgctttcaagggatcacttgatattacagaaaaccagctatcatctctaaaaaagtttcatgtgaggttaatgagtatcgtgttggatgttgatgttgaaccctccacgacaccatgggatcctgcaaaggcctacctgtttgtccctgttactgacaatacgtctatggaacccataaaagggatcaactgggaattggttgaaaagattacgaaaaccacagcgtgggacaaccctcttcagagagctcgtcccgatgtatatctcgggactaatgagagaactcttggtggggacagaagggaatatgggtttggtaaacttcgtcacaacattgtatttgggcagaaatctcacccaacttatggtattagaggagctgttgcatccttcgatgttgtgagagcttctggattgttacctgtgagagatgcttttgagaaggaagtagaagaggatttatcaaaaggaaaattgatgatggctgatgggtgcatggttgcagaagatcttattgggaaaatagtgacagccgcacattccgggaagcggttttacgtagattcaatttgttatgacatgagtgcagaaacatctttccctaggaaagagggatatcttggtcccctagagtacaacacgtacgctgactattacaagcaaaagtatggagttgatttgaactgtaagcaacaacctttgattaaaggacgtggtgtttcgtattgcaagaaccttctttctcctcggtttgaacagtcaggtgaatctgagacagtccttgataagacatattacgtgtttcttccacctgaactatgcgttgtgcatccgctttcgggttcacttatccgaggtgctcagaggttaccctctataatgagaagagttgagagcatgttactcgctgttcaactcaaaaatttgattagttatcctattcccacatcaaagattcttgaagccttgactgccgcctcgtgccaggaaacgttctgctacgagagagctgagcttttaggagatgcgtatctaaaatgggttgttagtcgttttctgtttctcaagtatcctcaaaagcacgagggtcagcttacaaggatgaggcaacaaatggttagtaatatggttctttatcagtttgctctggttaaagggcttcagtcatatatccaggcggatcgattcgccccgtctaggtggtctgctcctggtgtgcctccggttttcgacgaggacacaaaagatggaggatcttcgtttttcgatgaagagcaaaaacctgtttccgaggaaaacagcgatgtgtttgaagatggggagatggaggatggtgaactagagggtgatttgagttcgtaccgagttttatctagcaaaacgttagctgatgttgttgaggctttgattggtgtttattacgtcgaagggggtaagattgcagctaatcatttgatgaaatggattgggattcacgtggaggatgatcctgatgaagtcgatggaacattgaaaaatgttaatgttccagagagtgtgctcaagagcatcgactttgttggtcttgagagagctcttaaatatgagtttaaagagaaaggtcttcttgttgaagctataacacatgcttcaagaccatcttcaggtgtttcgtgttaccagagattggaatttgttggtgacgcggtcttggatcatctcatcacaagacatctatttttcacatacacaagccttcctcctggtcggttaacagatcttcgagctgcagcggttaacaacgagaattttgctcgcgttgcggttaaacataaactccacttgtaccttcgtcacggttcaagcgccctcgaaaaacagattcgggaatttgtgaaggaggttcaaaccgagtcatcgaaaccggggtttaactcttttggtttgggagactgcaaagcaccaaaagttcttggagacattgttgaatctattgcaggtgctatttttcttgatagtggaaaagatacaactgctgcttggaaggtttttcaacctttgcttcagcccatggtgacaccagagacacttccaatgcatccggtgcgagagctacaagagcggtgccagcaacaagcagaagggttagaatacaaagcgagtaggagtggtaacacagcgactgtggaagttttcatcgacggtgttcaagttggagtagcgcaaaacccgcagaagaaaatggctcaaaagctagctgcgaggaacgcacttgcagctttgaaagagaaagaaatagcagaatcaaaggagaagcatatcaacaacggtaatgcgggagaggatcaaggcgagaatgagaatgggaacaagaagaatgggcatcagccgtttacgagacaaacgttgaatgatatttgtttgaggaagaattggccaatgccttcttacagatgtgtgaaagaaggaggaccggctcatgcaaagagatttacgtttggggtaagagttaatacgagcgatagaggatggaccgatgagtgtattggcgagccaatgccgagtgttaagaaagctaaggattcagctgcggttcttctacttgagcttttaaataaaactttttcttga

SEQ ID NO: 62 - DNA, 5733 bp, Arabidopsis thaliana
  misc_feature 1..5733: note = ORF of protein Dcl1
  source 1..5733: mol_type = unassigned DNA
atggtaatggaggatgagcctagagaagccacaataaagccttcttattggctagatgcttgcgaggacatctcttgtgatcttatcgatgatctcgtgtctgaatttgatccttcctctgttgctgtcaatgaatccactgatgaaaacggcgtcatcaatgattttttcggtgggattgatcacattttagatagtatcaagaacggtggaggcttaccaaacaatggcgtttctgataccaattctcaaatcaacgaggttactgtaactcctcaggttattgctaaggagacagtgaaggagaatgggttgcaaaagaatggcggtaagagagacgaattctcgaaagaggaaggagacaaggataggaagagagctagggtttgtagttatcagagtgaaaggagtaacctttcaggtagagggcatgttaataattctagggagggagataggtttatgaataggaaacgtactcgtaattgggacgaggcgggtaacaataagaagaaaagggaatgtaacaattacagaagagatggtagagatagagaagttaggggttattgggagagggataaagttggttccaatgagttggtttataggtcagggacttgggaagctgatcatgaaagagatgttaagaaagtgagtggtggaaaccgcgaatgcgatgtcaaggcagaggagaacaagagtaagcctgaagaacgtaaagagaaggttgtggaagagcaagcaaggcgataccagttggatgttcttgaacaagctaaagcgaaaaacacgattgctttccttgagaccggtgctggaaagacacttatcgcgattcttcttattaaaagtgttcataaggatctgatgagccagaacagaaaaatgctctcggtgttcttggttcccaaagtgcctttggtttatcagcaagcagaagtgatccgtaatcaaacttgttttcaagttggacattattgtggtgagatgggacaggacttttgggattctcgaaggtggcaacgagagtttgagtctaagcaggttctagttatgacagcacaaattctgttgaatatactgagacacagtatcattagaatggaaacaattgatcttcttattctcgacgagtgtcaccacgctgtcaagaaacatccatactctttagtgatgtcagagttttaccatacaactcctaaagataaaagacctgccatctttggaatgactgcttcgcctgttaatttaaagggtgtttcaagccaagtagattgtgcgataaagatacgtaacctcgagaccaagttggattctacggtttgtactataaaagatcgaaaagaattagagaaacatgtgcctatgccttcagagatagtcgtcgagtatgacaaagctgctactatgtggtctcttcatgagacaataaagcaaatgattgcagctgttgaagaagcggcacaagcaagttcaaggaaaagcaagtggcaatttatgggggctagggatgctggagcaaaggatgaattgagacaggtttatggcgtctctgaaagaacggagagcgatggtgctgccaatttgattcataaacttagagctatcaattatactcttgctgaattgggtcaatggtgtgcttacaaggtgggacaatcattcttgtctgctttgcaaagtgatgagagggtgaatttccaagtcgacgtgaagtttcaagaatcatacctcagtgaggtggtgtcactcttgcaatgtgagcttctggaaggcgctgctgctgaaaaagtcgcggcggaagttggcaaaccagaaaatggtaatgcacatgacgagatggaggagggagagctccctgatgatcctgtggtctcgggaggggagcacgttgatgaagtaataggcgccgcagtggctgatgggaaagttactccaaaagtacaatcattgatcaaactactcctcaaatatcagcacacagctgattttcgagctattgttttcgttgagagggtggttgctgctttggttcttcctaaggtttttgcggagctgccttcgcttagttttatacggtgtgccagcatgattggacacaataacagccaggagatgaaatcatctcaaatgcaggatacaatttccaaattccgagatgggcatgtgacactgttagttgccacaagcgttgctgaggaaggacttgatattaggcaatgtaacgttgttatgcgtttcgaccttgcaaagacggtgctggcatacattcagtctcgtggccgggcaagaaagcctggatcagactacatactcatggttgagagaggaaatgtatctcacgcagcgttcctaaggaatgctaggaacagtgaggagacacttcgaaaagaagcaatagaaaggactgatcttagtcatctcaaagatacatcgagattaatctcaattgatgctgtgcctggtacagtttataaggtggaggcaactggtgccatggttagcttgaattccgcggttggtcttgtacatttctactgctctcagcttcctggtgacaggtatgcaatccttcgtcctgagtttagcatggagaagcatgaaaagcctgggggccacacggaatattcatgtaggcttcagcttccttgcaatgcaccgtttgaaatacttgagggtcctgtttgcagttcaatgcgtcttgcacaacaggctgtatgtttagctgcttgcaagaaactgcatgagatgggtgcatttaccgatatgctattaccggacaaaggaagtggtcaagacgctgagaaggctgaccaagatgatgaaggtgagcctgttcctggaactgctagacatagagagttctatcctgaaggtgtggcggatgtacttaagggagaatgggtttcatctggaaaggaagtttgtgagagctcaaagctattccatttatacatgtataatgtcagatgtgtagattttggctcttcaaaagatccattcctaagcgaagtttcagagttcgcgattctttttggcaatgagctggatgcagaggttgtattatcgatgtctatggatctttatgttgctcgggccatgatcactaaagcatctcttgctttcaagggatcacttgatattacagaaaaccagctatcatctctaaaaaagtttcatgtgaggttaatgagtatcgtgttggatgttgatgttgaaccctccacgacaccatgggatcctgcaaaggcctacctgtttgtccctgttactgacaatacgtctatggaacccataaaagggatcaactgggaattggttgaaaagattacgaaaaccacagcgtgggacaaccctcttcagagagctcgtcccgatgtatatctcgggactaatgagagaactcttggtggggacagaagggaatatgggtttggtaaacttcgtcacaacattgtatttgggcagaaatctcacccaacttatggtattagaggagctgttgcatccttcgatgttgtgagagcttctggattgttacctgtgagagatgcttttgagaaggaagtagaagaggatttatcaaaaggaaaattgatgatggctgatgggtgcatggttgcagaagatcttattgggaaaatagtgacagccgcacattccgggaagcggttttacgtagattcaatttgttatgacatgagtgcagaaacatctttccctaggaaagagggatatcttggtcccctagagtacaacacgtacgctgactattacaagcaaaagtatggagttgatttgaactgtaagcaacaacctttgattaaaggacgtggtgtttcgtattgcaagaaccttctttctcctcggtttgaacagtcaggtgaatctgagacagtccttgataagacatattacgtgtttcttccacctgaactatgcgttgtgcatccgctttcgggttcacttatccgaggtgctcagaggttaccctctataatgagaagagttgagagcatgttactcgctgttcaactcaaaaatttgattagttatcctattcccacatcaaagattcttgaagccttgactgccgcctcgtgccaggaaacgttctgctacgagagagctgagcttttaggagatgcgtatctaaaatgggttgttagtcgttttctgtttctcaagtatcctcaaaagcacgagggtcagcttacaaggatgaggcaacaaatggttagtaatatggttctttatcagtttgctctggttaaagggcttcagtcatatatccaggcggatcgattcgccccgtctaggtggtctgctcctggtgtgcctccggttttcgacgaggacacaaaagatggaggatcttcgtttttcgatgaagagcaaaaacctgtttccgaggaaaacagcgatgtgtttgaagatggggagatggaggatggtgaactagagggtgatttgagttcgtaccgagttttatctagcaaaacgttagctgatgttgttgaggctttgattggtgtttattacgtcgaagggggtaagattgcagctaatcatttgatgaaatggattgggattcacgtggaggatgatcctgatgaagtcgatggaacattgaaaaatgttaatgttccagagagtgtgctcaagagcatcgactttgttggtcttgagagagctcttaaatatgagtttaaagagaaaggtcttcttgttgaagctataacacatgcttcaagaccatcttcaggtgtttcgtgttaccagagattggaatttgttggtgacgcggtcttggatcatctcatcacaagacatctatttttcacatacacaagccttcctcctggtcggttaacagatcttcgagctgcagcggttaacaacgagaattttgctcgcgttgcggttaaacataaactccacttgtaccttcgtcacggttcaagcgccctcgaaaaacagattcgggaatttgtgaaggaggttcaaaccgagtcatcgaaaccggggtttaactcttttggtttgggagactgcaaagcaccaaaagttcttggagacattgttgaatctattgcaggtgctatttttcttgatagtggaaaagatacaactgctgcttggaaggtttttcaacctttgcttcagcccatggtgacaccagagacacttccaatgcatccggtgcgagagctacaagagcggtgccagcaacaagcagaagggttagaatacaaagcgagtaggagtggtaacacagcgactgtggaagttttcatcgacggtgttcaagttggagtagcgcaaaacccgcagaagaaaatggctcaaaagctagctgcgaggaacgcacttgcagctttgaaagagaaagaaatagcagaatcaaaggagaagcatatcaacaacggtaatgcgggagaggatcaaggcgagaatgagaatgggaacaagaagaatgggcatcagccgtttacgagacaaacgttgaatgatatttgtttgaggaagaattggccaatgccttcttacagatgtgtgaaagaaggaggaccggctcatgcaaagagatttacgtttggggtaagagttaatacgagcgatagaggatggaccgatgagtgtattggcgagccaatgccgagtgttaagaaagctaaggattcagctgcggttcttctacttgagcttttaaataaaactttttcttga

SEQ ID NO: 63 - DNA, 1713 bp, Amaranthus hypocondriacus
  misc_feature 1..1713: note = ORF of protein Dcl1
  source 1..1713: mol_type = unassigned DNA
atgagacgggtggaaagcatgcttcttgctattgaacttaagaatatcatcaaatatcctgtccctgcttctaagattctggaagctttgactgcagcctcttgtcaagaaacattttgttatgaaagagccgaactcctaggagatgcttatttgaaatgggtcgttagtagatatctttttcttaaatatccccagaaacacgagggtcagctcacacggatgagacaacaaatggtgagcaatttggtcttataccatcatgcactggagaaagggcttcagtcgtatatccaagcagatcgctttgctccgtctaggtgggctgctcctggtgttcctcctgtttttgatgaggacaccaaagaagaggaatcatctttatttgatcaggaaaaaactaatttaaagattgatcatgaaagagtcaataattatgatgttgacgatgatgaagaaatggaagatggtgaacttgaaggtgattcaagctcttacagggtgctttctagcaaaacattggctgatgttgttgaagcactaattggagttcattacgtcgaaggcggaaagaacgccgctaaccacttcatgaaatggattggaattgatatagagttcaatcctgaagatattgagtgtccaaagaggccaattatggttgcagaaggtgttcttagaagtgtggattttgatacattagaagctgctttaaatattaagttcaaagataggactttgttgattgaagcaattacgcatgcttcacgcccgtcttctggagttgcatgctaccagcggcttgagtttgtgggtgatgcagttttggatcatctaataactaggcatctgttcttcaggtatacagatctgcctccaggccgtctgacagatctgcgtgctgcagctgttaacaatgaaaattttgcccgggttgccgtcaaacataagcttcaccagcatttgcggcatgggtctactgccctggagaagcagatacgggattttgtgaaggaagttcaagaggagttgttaaaacctggttttaattccttcggattaggagattgtaaagccccgaaagtacttggtgatattgttgaatcgattgctggagctatttttcttgatagtggacatgacgcaacagttgtttggagagtttttgagcctttgcttcaaccaatggttactcccgatacacttcccatgcatcctgtccgagagttgcaagaacgatgtcagcagcaggctgaaggcctggaatataaggccactagagaagggaatttagccactgttgaagtttacgtcaacggaatccaagttggagtcgctcataatccacagaaaaagatggctcagaaattggctgctcggaatgctctgcttgttttgaaggagaaggaaactgctgaagctaaggtcaaggctgaggagagcgagaaaaaaaacaagaatggggtagtaacattcacaaggcagacactgaatgatatatgcttacgcagaaattggcccatgccactctataggtgtgtgaatgaaggaggccctgctcatgccaagcgattcacatttgcagttcgggttaataccagtagtaaaggttggaccgaggaatgcattggagagcctatgcctagtgttaagaaggccaaagactcggcagcggctcttcttttggaactgttgaacaaacgatattcctaa

SEQ ID NO: 64 - DNA, 5793 bp, Brachypodium distachyon
  misc_feature 1..5793: note = ORF of protein Dcl1
  source 1..5793: mol_type = unassigned DNA
atggctggaggaggcggcggcgccggggccggcgagcacgcggcggcggcctactggtacgacgcgtgcgaggacggggcgtcgctgctgtgcggcatcgacttcgccgcgtccgccgacttcgacccgggcctcatgcccgccatggacagcggcgtcgacgacggcttcgtcgccgagatcgaccggatactcgagagcatcaacgcggagagcacccccgcgccgccgccgccgcagcagcagcaagcgcttgtggacccctcgccgcagccgccggtggagcaagggcaggaggctgctgtggccgtggccgacaatgccgtggaggtggtgggctcagggcagaggatccagggcgccgagacgaggaaggagatgaagaaggagtcgcacagcagttccacgaatggcagttccgcgaatggcggggaccggagagaccagaagcgtccgcccctcgcctctggggtcgccagcgggccgcgccctgattggcggcggcggccgatgccaccaccgccctcgcgtggattggaggatcggcgtgggaggcgcgacttcgacaggcctcgcaagcgcgaccgcgacggccactctggctccgagcatcaccggcgcgaggctcggggattttgggagcgtgaccgcggcggtaagatggtgttccggccgggcacttgggagaaggagtctgaccgcgaggcaaagcgtgccaggacacaggacgggggttctgtggagaagaaagcagaggcggatagggcgggggcagcgcagagggcgaagcctgtcgctgaggagcaggcgaggcattaccagttggaggtgcttgagcaagcaaagagcaggaacaccatagcctttctcgagaccggcgccgggaagactctcattgctgtgctgctcatcaaaagtatatgtgacaaaatgctcaaggagaacaagaagatgcttgctgtcttcttggttcccaaggtgcctcttgtgtaccagcaagcggaagtgatacgcgagaggactggctatcggattgggcattactgcggagagatgggtcaggatttctgggacgctagaaagtggcagcgtgagtttgattcaaaacaggtactagttatgacagctcaaattttgttaaacatcctcaggcacagtattattaaaatggatgcgatacatcttttaattttagacgagtgccatcatgcagtgaaaaaacatccttattctctagtgatgtcagaattttatcacacgactccaaaggataagagaccagttgtttttggtatgacagcttctcctgtcaacctaaagggagtcactagccaagaggattgtgccatcaagataagaaatcttgagagtaaacttgattctgtagtttgtactataaaagatcggaaggagcttgagaaacatgtccccatgccttttgaagttgttatccattacgacaaagcagccactctttgttccttgcacgagcggatcaaacaaatggaagctgcagttgaagaagcggcactctctagctctaagagaaccaggtggcagtttatgggagcaagggatgctgggtctagagaggagctgcgccttgtctatggtgtttctgagcgtacagaaagtgatggtgcagctaatttgatccagaagttgagagctataaattatggtttaggtgaactaggacaatggtgtgcttacaaggttgcacagtccttcttgacagcgttacaaaatgatgaaagggcaaactatcaggttgatgtaaagtttcaggagtcctatttaaagaaggttgttgatttgttacactgccagttgactgaaggagctgcaatgaaaagcgagactaatgatgttgagatgcatagtactgaaaaccataactcaagtgaacttgaggatggggagctacctgacagccatgctgtttcggtaggtgaacatgtagatgaagtcatcggagctgccgtggcagatgggaaagttacccctagggtgcaggctttgatcaaaatccttctcaagtaccagcataccgaggatttccgtgctatcatctttgtggagcgggttgtcacggcattagttcttcctaaggtgtttgcggagcttccatctttaagctttattcgatgcgctagcttgataggccataacaacaatcaagagatgcgcacaagccaaatgcaggatacaattgccaaatttcgcgatggccgtattacattgttagttgcgactagcgtcgcagaggaagggcttgatattcgacagtgcaatgttgtcattcgctttgaccttgcgaagactgtcttggcctacatacagtctcggggtcgtgcaaggaagcctggatctgactacatattgatggtcgaaagggcaaatttatcacatgaaactttcttgaggaatgctcgaaatagtgaggagacgttaagaaaagaagctatagaaagaactgatctcagtcatcttgatggcattcctatgttaaatcctatccatacatctcctgattcgatgtatcaggtggaatcaacaggtgctgttgtcagcttgaattctgcagttggactcatacatttttactgttcacagcttccaagtgacaggtactcaattcttcgtccagaatttattatgcaaaaacatgagaaaccagggggttccgtggaatattcttgcaaacttcaactcccctgcaatgcaccatttgagaaacttgagggtcctatatgcagctcgattcgtctggcacagcaagctgtttgtttggctgcctgtaagaaactacatgagatgagtgccttcacagatatgcttttacctgatagaggaattggggaaggagaaaagactgaacagaatgatgaaggtgatccagtccctggaacagcacggcatagagagttttatcctgaaggtgttgcggaagttctgagaggagaatggattttatctggaagagattgctgccaaagctctcaatttattaagttgtatatgtattacgtgaactgtgtggatgtggggacatctaaggaccccttccttgcacaactttcaaatttttctatagtttttggcaatgagctggatgcagaggttttatcgatgacaatggatctctttgttgccaagacaatgataacaaaggcatctcttgtattccatggaccaattgaaattacagagagacagttggtattgctgaagagctttcatgttaggctcatgagtatagtccttgatgttgatgtcgatccctcaactacaccttgggatccagcaaaagcgtacctctttgtccctgtaggagctgagaagtgtacagatgctttcagagagatcgattggactttagtcaacagcattgtgaacactgatgcctggaataatccacttcagaaggcacgcccagatgtctacctaggcaccaatgagcggacacttggtggagataggagagagtatgggtttgggaaattacgtaatggaaccacttttggacagaaggcacatccgacttatggtattagaggagccattgctgagtttgatgttgtcaaagcatctggattagttcctgctcgtgacagagggtttcttaatgactctcagaaacacgggaagttgttcatggcagattcatgctgggatgctaaagatcttgctggaatggttgtcactgctgcccactcaggaaagaggttctatgtggactctatttgttataacatgaatgcagaaaattcatttcctaggaaggaaggctacctggggcctttggaatatagctcgtatgctgattactacaagcagaaatacggcgtggagctagtgtatacgaaacaacctcttatacgtgcacgtggtgtttcgtactgcaagaatctcctgtctcctagatttgagcattctgaagctaaagaaggtgtcaacttttctgagaaccatgacaaaacatactatgtgtatctaccacctgaactgtgcgttgtgcaccctcttcctggatcacttgttcgtggagctcagaggttgccttcaataatgagaagggttgagagcatgcttcttgcagttcagttgaaggaaataattgattatcctgttcccgcaacgaagatattagaagccttgactgctgcatcatgccaggaaacgttttgctatgaaagagcagagctactgggtgatgcatacctgaaatgggttgtgagtagattcctttttctaaaatatcctcagaagcatgagggacagcttacgaggatgcggcagcagatggttagtaacatggttctgtaccaattcgctctgaataaaactcttcagtcgtacattcaagcagatcgttttgccccatcaagatgggcagctccaggagtgctacctgtatttgacgaggagacaagagaatctgaaccatccatctttggtgaggaaccagttcttggtaatgaagtacagaaggattttgatgacgattacgcagatagcatccaagaagacggtgaaattgatggagattctggctgctatcgtgttctctcaagcaaaactttagcagatgttgttgaagcacttattggggtctattatgtagcaggagggaaaattgctgcaaaccacctgatgaagtggattgggattcatgcagagctggatcctcaagagatccctcctccgaagatatataatatacctgatagcataatgagaagcatcaactttgacaccttagaaagcatcatgggtatcaaattccagaataaaggttttctggttgaggctataacgcatgcatcgaggccgtcttctggtgtttcctgttaccagcggttggaatttgttggtgatgctgtgttggatcatctcatcacaaagcatttgttttttacatatactgatctacctcctggtcgcttaaccgacttgagagctgcggctgttaataatgagaactttgcaagggttgcagttaagcataagctgcatgtacatcttcgccatgggtcatctgcgctggaaacacagattcgggagtttgtgaaggatgtccgagaagagatttcaaaatcaggtttcaattcttttgggcttggggactgcaaggctcctaaagttcttggagacattattgagtctattgctggtgcaatatttctcgatagtgggtatgacacctcggcagtctggaaggtttttcaaccgcttcttgatcctatggtaacgccagaaacccttcccatgcatccaattagggagctccaagaacgctgccagcagcaagctgaaggcttagaatacaaagcatcccgagcaggcaatgtagctactgttgaagtcttcgtggatggtatccagattggtgtagctcagaacccgcagaagaagatggcacagaagttggcggcgaggaatgcacttgtggtcctgaaggagaaggaaagcgcagaagccaaggagaccgaaaaggatgacgataagaagaacggtgctcatgtgttcaccaggcagaccctgaacgacatctgcttgagaaggcagtggccgatgccacaatacaggtgcataaacgagggcggccctgctcatgctaaaagatttgtctacgcggtaagggtgaacacatctgatcgtggatggaccgacgaatgcattggagagccaatgcctagtgtgaagaaggccaaggactctgctgccgttcttctccttgagcttctgaatggaagtttccctgacaagtctgacggcaaaaagccctag

SEQ ID NO: 65 - DNA, 5844 bp, Glycine max
  misc_feature 1..5844: note = ORF of protein Dcl1
  source 1..5844: mol_type = unassigned DNA
atggaggatgggagcagggttccggcgggggatgacccctcttactggctggatgcgtgtgaggacatatcgtgcgatgatttcattgattttgatgtctcttccatcgtctctgaccagcccgacaatccttccaatcaggacttcttcggtggcattgataagattttggacagcatcaagaacggtgctggtctccctctcaatcatgctgttgaaccacccaataataatggcactgcagcaggagaagtgtgcttgccttccaatgccactcttgaggatggtgctcctgctgctgatgcttttgaccattctggtggcgttgctcggagtaacggctccagtaagctgtctaatgggaacgaaacaggagttttggttaattattctcaggaaagaggggcaccccctctgaatggaggccacgattttgatggtgaagaaaggtgcagcaaaagggcttggcttggcggctacaataatgagaggccttattattgtagagggaattatcaaggcaaggagagggagaggtgttttaataataataacaggaagaggccacggggtgatagggatgaaattgacaggaaagataaggatggtggtggaaggaaaagagaacattatggtgctgttgccaggagggatgttagagatagggattgtagggatagagaaactaggggttactgggagagggataagtcaggttccactgatatgatctttcgcactggcgcttgggaacctgatcataatcgagatgacaaaatggtgattgacaccaaactggagaactatggaaagcttgataagaagtccgaggatgctattgagagggttcctgaggaaaaagctagacagtatcagttagatgttcttgagcaatcaaaaaggaaaaataccatagcttttcttgaaactggagcagggaaaaccttaattgctgttcttctcattaaaagtatacaagatagtttgcagaagcaaaataagaaaatgcttgcagtatttttagttccaaaagttccacttgtttaccagcaagctgaagtaattcgtgagcggactggttatcaagtaggccactattgtggagaaatggggcaggatttctgggatgctcgaaggtggcagcgtgaatttgacaccaaacatgttttagtcatgactgcccaaattcttttaaacattttgaggcatagcataataaaaatggaagcaatcaatctcttgattctggatgagtgccaccatgcagtgaagaaacacccatattcactggtgatgtccgagttctaccatacaacacccaaagagaacaggccatctgtatttggaatgactgcttctcctgttaacttgaagggtgtctccagccaagtagattgtgcaataaaaattcgtaatcttgaaagtaagctagattctatagtttgcaccattaaagatcggaaggagctggagaaacatgtgccgatgccctctgaagttgtggtggagtatgataaagctgccagtttatgttatctacacgagcagataaagcagatggaggttgaagttgaagaagctgcaaaatatagttcaaggagaagcaaatggcagtttatgggagctagagatgctggagctaaagaagagctgcgccaagtatatggcgtttctgaaagaacagaaagtgatggagctgcaaacttaattcagaagttgagagctgttaattatgcacttggtgaactgggacaatggtgcgcttataaggttgcactatcctttttagcagctttgcaaaatgatgaaagggccaactaccagcttgatgtcaagtttcaggaaacttatttgagtaaagttgtctcgcttctgaaatgccaactgtcagagggtgcagcttctgacaaaaatgctggtattgatgactcagagaatggtgcagctcaaagtgggtctgaacatgaagagatggaagaaggagagcttccagacagtcatgttgtctctggcggggagcacgtggatgttattataggagctgctgtggctgatggcaaggtgacccccaaggtgcaggcactaatcaaaatacttctcaagtatcagcatacagaagatttccgtgcaatcatctttgttgagcgtgttgtgtctgcattagttctgcccaaggtctttgcagagcttccatcacttagttttgtgaagtgtgcaagtttgattggtcataataacagtcaggaaatgcgaacttaccaaatgcaggatacaattgccaaattccgtgatggacgggtcacattgttggttgccactagtgttgctgaagaaggacttgatatccgtcaatgcaatgttgttattcgctttgaccttgcaaagactgttctggcatatattcagtccaggggacgtgctagaaagcctggatctgattacatcctgatggttgagagggacaatttgtcacatgaagcattcctaaggaatgccaagaacagtgaggaaactttgcgcaaagaagcaatagagagaacagaccttagtcatctgaaggacacttcaagattgatttctgttgacacccggcccggaacagtttaccaggtgaagtcaaccggtgcagttgtgagcctaaattctgctgtaggtcttatccacttttactgctctcagttacctagtgacagatattcaattcttcgccctgagtttattatggagaggcatgaaaaaccaggaggtcccacggaatattcttgcaagcttcaactaccgtgtaatgcaccgtttgaaaatcttgagggtccaatatgcagttctatgcgtctggcacaacaggctgtttgtctggctgcttgcaagaaactgcatgagatgggtgcattcactgacatgctattgcctgataaaggaagtgggggagaaagagaaaaggatgaacaaactgatgaaggagatccacttccagggactgctagacatagggagttctaccctgaaggtgtggctgacatactgaagggagagtggatcttatctgaaaaagatgcttgcaacaactacaaattacttcatctttatatgtatgccgtgaagtgtgaaaatcttggccattcaaaggatccgttcttgactcaagtttcaaattttgcagtactttttggcaatgagctggatgcagaggtgttatcgatgtcaatggatctatttatcgctcgaactgtgactacaaaggcatctcttgtctttagtgggttgataaatatcactgagagtcagctggcatccctgaaaagctttcatgtaagattaatgagcattgtcttggatgtggatgttgaaccatctaccactccttgggatcctgcaaaggcatatttgtttgtcccaatggttggcgataagtccgtagatcctatgaaccaaattgactggcatctggttgagacaataattggagctgatgcatggaagaatcccctccagaaagctcgaccagatgtttaccttggtactaatgagagaacattaggtggagacagaagggaatatggatttgggaaattgcgtcatggcatggcttttgggcaaaaatcacatcctacctatggaatcagaggagctgtggcccagtttgatgtggtgaaagcttcaggattggttcctaacagagacgccatgcaaacacaaaagcacatcaatatgactaccaatggaaaattaatgatggcggatatctgtactaatgcagaggatctggttgggagaattgtaactgctgctcattcagggaagaggttttatgttgattcaatatgctatgacatgtcagcagagaactctttccctaggaaagaaggttatcttggtcctctggagtacagttcatatgctgattactacaagcaaaagtatggggttaatttgatttacaagcagcaacctcttataagagggcgtggtgtatcatactgcaagaatcttctgtctcctcgatttgagcacagtgaagctcatgaaggtgaatctgaagagatacatgacaaaacttattatgttttccttcctcctgagctatgcctcgtacacccactgcctggatcacttgtccgtggtgcccagaggttgccttcgataatgaggagggttgaaagtatgctacttgcagttcagcttaagaatatgataaattatcctgtcctggcttcaaagatcttgggagccttgactgccgcctcttgccaggagacattctgctatgaaagggcagagcttcttggagatgcttacctgaaatgggttgttagtcgatttcttttcctaaaatatccacagaaacatgaaggtcaacttactaggatgagacaacaaatggtcagtaacatggtattatatcagtatgcattaagtaaagggcttcaatcatatattcaagcagatcgttttgctccatccagatgggcagctcctggggtgctgcctgtctttgatgaggataccaaggatggggagtcatctttgtttgaccaggagcgatccatttccaagatcgagaggatggattgtcacacgaatggatatgaggatgagatggaagatggtgagcttgagagtgattcaagttcttacagagttctatccagtaagacacttgcagatgttgtggaagcactgattggggtatattatgtggaaggtggtaagaatgctgccaaccaccttatgaaatggattggcattcaaatagagtttgatcctgatacaatggagtgtacaaaaaagccatttaatgttcctgacagcatacttaggagtgttgattttgatgctttagaaggtgctttaaatatgaagtttaacgacagggggctgctggtagaatccataactcatgcctcccggccatcttcaggagtatcctgttatcagcggttggagtttgttggcgatgctgtcttggatcatcttattaccaggcacttgtttttcacttacacaaatctgccaccgggacgtctgactgaccttcgggctgctgctgtaaacaatgaaaattttgcgcgtgttgcagttaagcacaaccttcatgtgcatctcagacatggatccagtgccctagaaaaacagattaaagaatttgtgaaggaagttcaagtcgaattgtcaaagccaggttttaattcctttggtctgggagactgtaaagcccccaaggtccttggtgacattgttgaatctattgcaggtgccatttttcttgacagtgggagggacactactgttgtttggaaggtttttcagcctcttttgcatcctatggttaccccagagactctgccgatgcatcctgtgcgtgagctgcaagagcgatgccagcagcaagctgaaggcttagagtataaagcaagtcgtattggcaatttggccactgttgaagtattcattgatggggtccaagtaggggctgctcaaaatccacagaaaaaaatggcgcagaaattagctgcaagaaatgcccttgctgctttgaaagagaaagaggtgggaaaaactcaggagaagaatgatgaaaacgggaaaaagaatggaaaccagacatttactaggcaaacattgaatgatatatgtttacgaagaaattggcctatgccattctacaggtgtgtgaatgagggtggtccagcacatgcaaagaggtttacatttgcggtgcgtgtcaatactactgatagaggatggacggatgaatgtgttggagagccaatgccaagtgtcaagaaggccaaagactcagctgctgttcttctcttagaactactgaacaaattatactcgtga

SEQ ID NO: 66 - DNA, 5838 bp, Glycine max
  misc_feature 1..5838: note = ORF of protein Dcl1
  source 1..5838: mol_type = unassigned DNA
atggaggacgggagtagggttccggcgggggatgacccgtcttactggctggatgcgtgtgaggacatatcgtgtgatgatttcattgattttgatgtctcttccatcgtcgtctctgaccagcctgacaacccttccaatcaggacttcttcggtggcattgataagattttggacagcatcaagaacggtgctggtctccctctcaatcatgctgctgctgaaccacccagtaatgtcactgcagcagcatcaggaggagcagaagtgtgcttgccttccaatgccactccggaggattcttttgaccattctggtggcgctgctctgagtaacggctccagtaagcagtctaatgggaacgaaacgggagttttggttgattattctcaggaaagggggacacccactctgaatggaggccttgattttgatggtgaagaaaggtgcagcaaaagggctcggcttggtggctacaataatgatcggccttatcatggtagagggaattatcaaggcaaggagagggagaggtgttttagtaataacaggaagaggccgcgggggggtagggatgaaattgataggagggataaggatggtggtggaaggaaaagagaacattgtggtgctgttggcaggagggatgttagagatagggattggagggatagagaaactaggggttactgggagagggataagtctggttccactgatatggtctttcgcacgggtgcttgggaacctgattgtaatcgagaagacaaaatggcgattgacatgaaactggagaaaaatggaaaccttgataagaagtccgaggaggctaaggagagggttcctgaggaaaaagctagacagtatcagttggatgttcttgagcaggcaaagaggaaaaatactatagcttttcttgaaactggagcagggaaaaccttaattgctgttcttctcattaaaagtatacaggagagtttgcataagcaaaataagaaaatgcttgcagtatttttagttccaaaagttccacttgtttaccagcaagccgaagtaattcgtgagcggactggttatcaagtaggccactattgtggagaaatggggcaggatttttgggatgctcgaaggtggcagcgtgaatttgacaccaaacatgttttagtcatgactgctcaaattcttttgaacattttgaggcatagcataataaaaatggaagcaatcaatctcttgattctggatgagtgccaccatgctgtgaaaaaacacccatattcactggtgatgtctgagttctaccatacaacacccaaagagaacaggccatctgtatttggaatgactgcttctcctgttaacttgaagggtgtctccagccaagtagattgtgcaataaaaattcgtaatcttgaaagtaagctagattctatagtttgcaccattaaagatcgtaaggagctggagaaacatgtaccgatgccctctgaagttgtggtggagtatgataaagctgccagtttatgttatctacacgagcagataaagcagatggaggttgaagttgaagaagctgcaaaatgtagttcaagaagaagcaaatggcagtttatgggagctagagatgctggagctaaggaagagctgcgccaagtatatggtgtttctgaaagaacagaaagcgatggagctgcaaacctaattcagaagttgagagctgttaattatgcacttggtgaactgggacaatggtgtgcatataaggttgcacaatcctttttagcagctttgcaaaatgatgaaagggccaactaccagcttgatgtcaagtttcaggaaacttatctgagtaaagttgtttcacttctgaaatgccaactgtcggagggtgcagtttctgacaaaaatgctggtattgatgactcagagaatggtgcagttcaaagtggctctgaacatgaagagatggaagaaggagagcttccagacagtcatgttgtctctggcggggagcatgtggatgttattataggagctgctgtggctgatggcaaggtgacccccaaggtgcaggcactaatcaaaatacttctcaagtatcagcatacagaagatttccgtgcaatcatctttgttgaacgtgttgtgtctgcattagttctgcccaaggtctttgcagagcttccatcacttagttttgtgaagtgtgcaagtttgattggtcacaataacagtcaggaaatgcggacctaccaaatgcaggatacaattgccaaattccgtgatgggcgggtcacattgttggttgccactagtgttgctgaagaaggacttgatatccgtcaatgcaatgtcgttattcgctttgaccttgcaaagactgttctggcatacatacaatccaggggacgtgctagaaagcctggatctgattacatcctgatggttgagagggacaatttatcacatgaagcattcctaaggaatgccaggaacagtgaggaaactttgcgcaaagaagcaatagagagaacagaccttagtcatctgaaggacacttcaaggttgatctctgttgacacccggcctggaacagtttaccaggtgaagtcaactggggcagttgtgagcctaaattctgctgtaggtcttatccacttctactgctctcagctacctagtgacagatattcaattcttcgccctgaatttattatggagaggcacgaaaaacctggaggtcccacagaatattcttgcaagcttcaactaccatgtaatgcaccctttgaaaatcttgagggtccaatatgcagttctatgcgtctggcacaacaggctgtttgtctggctgcttgcaagaaactgcatgagatgggtgcattcactgacatgctattgcctgataaaggaagtgggggagaaaaagaaaaggatgaacaaactgatgaaggagatccacttccagggactgctagacatagggagttctaccctgaaggtgtggctgacatactgaagggagaatggatcttatctggaaaagatgcttgcaacaactccaaattgcttcatctttatatgtatgctgtgaagtgtgaaaatctaggccattcaaaggatccgttcttgactcaagtttcaaattttgcagtactttttggcaatgagctggatgcagaggtgttatcgatgtcaatggatctatttatcgctcgaactgtgactacaaagtcatctcttgtctttagggggttgataagtatcactgagagtcagctggcatccctgaaaagctttcatgtaagattaatgagcattgtcttggatgtggatgttgaaccatctaccactccttgggatcctgcaaaggcatatttgtttgtcccaatggttggcgataagtctgtagatcctacgaaccaaattgactggcatctggttgagacaataattggagctgatgcatggaagaatcccctccagaaagctcgaccagatgtttaccttggtactaatgagagaacattaggtggagacagaagggaatatggatttgggaaattgcgtcatggcatggcttttgggcaaaaatcacatcctacctatggaatcagaggagctgtggcccaatttgatgtggtgaaagcttcaggattggttcctaacagagatgccatgcaaacacaaaagcacatcaatatgactaccaatggaaaattaatgatggcggatacctgtactaatgcagaggatctgattgggaaaattgtaactgctgctcattcagggaagaggttttatgttgattcaatacgctatgacatgtcagcagagaactctttccctaggaaagaaggttatcttggtcctctggagtacagttcatatgctgattactacaagcaaaagtatggggttgatttgatttacaggcagcaacctcttataagagggcgtggtgtatcatactgcaagaatcttctgtcccctcgatttgagcacagtgaagctcatgaaggtgaatccgaagagacacatgacaaaacttattatgtttttcttcctcctgagctatgccttgtacacccattgcctggatcacttgtccgtggtgcccagaggttgccttcaataatgaggagggttgaaagtatgctacttgcagttcaacttaagaatatgataaattatcctgtccaggcttcaaagatcttggaagccttgactgccgcctcttgccaggagacattctgctatgaaagggcagagcttcttggagatgcttacctgaaatgggttgttagtcgatttcttttcctaaaatatccacagaaacatgaaggtcaacttactaggatgagacaacaaatggtcagtaacatggtattatatcagtatgcattaagtaaagggcttcaatcatatattcaagcagatcgttttgctccatccagatgggcagctcctggggtgctgcctgtctttgatgaggataccaaggatggggagtcatctttgtttgaccaggagcgatccatttccaagatcgagaggatggattgtcacacggatggatatgaggatgagatggaagatggtgagcttgagagtgattcaagttcttacagagttctatccagtaagacacttgcagatgttgtggaagcactaattggggtatattatgtagaaggtggtaagaatgctgccaaccaccttatgaaatggatgggcattcaaatagagtttgatcctgatacaatggattgtacaagaaagccatttaatgttccagatagcatacttaggagtgttgattttgatgctttagaaggtgctttaaatatgaagtttaaagacaggggactgctggtagaatccataactcatgcctccaggccatcttcaggagtatcctgttatcagcggttggagtttgttggtgatgctgtcttggatcatcttattaccaggcacttgtttttcacttacacaaatctgccaccgggacgtctgactgaccttcgggctgctgctgtaaacaatgaaaattttgcgcgtgttgcagttaaacacaaccttcatgtgcatctccggcatggatccagtgccctagaaaaacagattaaagaatttgttaaggaagttcaagatgaattgtcaaagccaggttttaattcctttggtctgggagactgcaaagcccccaaggtccttggtgacattcttgaatctattgcaggtgccatttttcttgacagtgggagggatactactgttgtttggaaggtttttcagcctcttttgcatcctatggttacccctgagactctgccaatgcatcctgtgcgtgagctgcaagagcggtgccagcagcaagctgaaggcttagagtataaagcaagtcgtattggcaatttggccactgttgaagtattcattgatggggtgcaagtaggggctgctcaaaatccacagaaaaaaatggcgcagaaattagctgcaagaaatgcccttgctgctttgaaagagaaggaggtgggaaaaactcaggaaaagaatgatgacaatgggaagaagaatggaaaccaaacatttactaggcaaacattaaatgatatctgtttacgaagaaattggcctatgccattttacaggtgtgtgaatgagggtggtccagcacatgcaaagaggtttacatttgcagtgcgtgtcaatactactgataaaggatggacagatgaatgtgttggagagccaatgccaagtgtcaagaaggccaaagactcagctgctgttcttctcttagaactactgaacaaattatactcgtga

SEQ ID NO: 67 - DNA, 5652 bp, Oryza sativa
  misc_feature 1..5652: note = ORF of protein Dcl1
  source 1..5652: mol_type = unassigned DNA
atggcgggaggaggaggagttggaggaggcgctggcgagcacgcggcggcggcgtactggtacgacgcgtgcgaggacggcgcgtcgctgctgtgcggcatcgacttcgccgcgtccgcggacttcgaccccggcctcatacccgcgatggacaccggcgccgacgacggcttcgtcgccgagatcgacaggatactcgagagcatcaacgcggagagctcccctgcgcccccacccccgccgccgccgccgctacctgagcccgtgcctgtggctccgccggagctaccgattcaggaaaagcaattacaggtggcctctgcgcccgtggccaacaatgccgtggcggtggtgggcgttgtgcagagaagtaaaggcgtggtggcgaggaaggagccgaggagggagtcgcatggctgtgctgcaaatggcggcggcggtggggagtggagggacgggaagcgcccgcgcctcgcctccggtggtgtcgggggaccgcgtcaggagtggcggcggaggccgatgctgccaccaccgccctcgcgtgggtgggatgacaggcgtggaaggcgcgatttcgacagggtccgcaagcacgagcaccatcggcgcgaggcccggggtttctgggagcgtgaccgcggcggcaagatggtgttccgctcaggcacctgggagcaggagtctgaccgcgaggcaaagcgtgccaggacgcaagatggaggctccatggagaagaaggcagaggcagataggatgggggctgcgcagcgggagaagcctgtcgcggaggagcgtgcaaggcaataccagctagaggtgcttgagcaagctaagagcaggaacacaatagccttcctggagactggcgcagggaagactctcatcgccgtgcttctcatcaaaagtgtgtgcgataaaatgctcaaggagaacaagaagatgcttgctgtcttcttggttcccaaggtgcctcttgtgtatcaggtactagttatgacagcccaaattttgttaaacatcctcaggcacagtattattaaaatggatgcgatacatcttttgattttagatgagtgccatcatgcagtgaaaaaacatccatattctctagtgatgtcagaattttatcacacgactccgaaggagaagaggccagcagttttcggtatgacggcttctcctgtcaatctaaaaggagtgacgagtcaagaggactgtgccatcaagataagaaatcttgagagcaaactagattcagtagtttgtaccatcaaagatcgtaaagagcttgagaagcatgtccccatgcctttggaagttgttgtccaatatgacaaagcagccactctttggtccttgcatgagcaaatcaaacaaatggaaagcacggttgaagaagctgcactctctagctctaagagaactaaatggcagttcatgggggctagggatgctgggtctagagatgagctgcgtcttgtctatggtgtttcagagcgtacagagagtgatggtgcggctaatttaatccagaagttgagagctataaattatgctttaggtgaacttggacaatggtgtgcatataaggttgcacagtcctttttgactgcattacaaaatgatgaaagggcaaactatcaggttgatgtcaagtttcaagagtcttatttaaaaaaggttgttgatctgttacactgccagttgactgaaggggctgcaatgaaaagtgagaccagtgatgttgaaatgcaaaatactgaaaaacacaacacaaatgaccttgaggagggggagctgcctgacagtcatggtgagcatgtagatgaagttatcggtgctgccgtagcagatggtaaagttactcctagggtgcaggctttgatcaaaatacttctcaaatatcagcatactgaggattttcgtgccataatctttgtggagagagttgtgacagcattagttcttcctaaggtattggcagaacttccatctctaagttttattcgatgtgcaagcttgataggccataacaataatcaagagatgcgcgcgtgccagatgcaggatacaatttcaaaatttcgtgatggccgtgttacacttttagtcgcaacaagcgttgcagaggaagggcttgatattcgacagtgcaatgttgtcattcgatttgaccttgcaaagaccgtcttagcctacatacagtcacgtggtcgtgcaagaaagcctggatctgattatatattaatgcttgaaaggggcaatatatcacatgaaacttttttgaggaatgctcggaatagtgaggagacgttgagaaaggaagctatggaaagaaccgatcttagtcatttggatggtacttctgtgttgagtcctgttgatacatcacctggttcaatgtatcaggttgaatcaacaggtgctgttgtcagcttgaactcagcagttggactcatacatttttactgttcacagttgccaagtgacagatactcaattcttcatccggaattcattatgcaaaagtatgagaagccaggtggttcagtggaatattcttgcaagcttcagctcccttgtaatgccccgttcgaaaaacttgagggtcctatatgcagttcaatacgactggcacagcaggctgtttgtttggctgcttgtaagaagctgcatgagatgggtgccttcacagatacgctcttacctgacagaggaagcggggaaggggaaaaaactgagcagaatgatgaaggtgaaccacttcctggaacagctcgccatagagagttttatcctgaaggtgttgctgatattctacggggagaatggattttatctggcagagatggctaccaaaactctcaatttattaagttatatatgtattctgtgaactgtgtaaacgttgggacttctaaggaccctttcgttacacaactttcaaattttgcaattatttttggcaatgagctggatgcagaggttttatcgacgacgatggatctctttgttgcaaggacaatgataacaaaggcatctctcgtattccgtggacgaattgaaattacagaaagtcagctggtgttgctgaagagctttcatgttaggctcatgagcatagtacttgatgttgatgttgatccctcaactacaccttgggatcccgcaaaagcatacctctttgtccctgtaggagctgagaagtgtacagatcctttaagagagattgattggactttggttaacaacattgttaacactgatgcctggaacaatccacttcagagggcacgcccagatgtctatctaggcaccaatgaacgaacacttggtggggataggagggaatacgggtttgggaaattgcgccatgggactgcttttggacaaaaggcccatcctacttatggtattaggggagctattgctgaatttgacattgtgaaagcatctggattagttcctgctcgtgatagaggacattttagtgattatcagaaccaaggcaagttgttcatggcagattcatgttggaatgctaaagatcttgctggaatggttgtcactgctgcccactcagggaagaggttttatgtggactgcatttgctataacatgaatgcagaaaattcattccctaggaaagaaggctacctggggccattggagtatagttcatatgctgattactacaagcagaagtacggtgtggagttaatctataggaaacagcctcttatacgggcccgtggtgtttcatattgcaagaatcttctttctcctagatttgagcattctgacgctagagaaggtgacttctctgagaaccttgacaaaacatactacgtgtatttgccacctgagttgtgccttgtgcacccacttcctggatcactagttcgtggagctcagaggttgccctcaataatgagaagggtggagagcatgcttcttgcagttcaattgaaggacataattgattaccctgttcctgcaactaagatactagaagccttgactgctgcatcatgccaggagacattatgctatgaaagagcagagctgctgggtgatgcatacctgaaatgggtcgtgagtagattcctcttcctaaaatatcctcaaaagcatgagggacagcttacaaggatgcggcagcagatggttagtaacatggtcctgtaccaatatgctctgaataaaaccctccaatcatatattcaagcagaccggtttgctccatcaagatgggcagctccaggagtgctacctgtattcgatgaagagtctagagagtatgaaccttccatctttgacgaggaatcaactggttgcgagttacagaaggaatcctatgatgactatgcagataacatgcaagaagatggtgaaattgaaggagactctagctgctatcgggttctgtcgagcaaaactttagcggatgttgttgaggcacttattggggtgtattatgtagcaggagggaaaatcgctgcaaaccacctgatgaaatggattggaattcatgcagagttggatcctgaagagatcccacctccaaagccatatgacattcctgagagcataatgagaagcatcaactttgacacgttgaaaggtgtcttgggtatcgaattccagaataaaggtttacttgttgaggccataacacatgcatcgagaccatcttctggtgtttcctgttaccagcgtttggaatttgttggtgatgctgtgttggatcatctcataaccaggcatttgttttttacatatactgacctacctcctggccgcttgactgatttgagagctgcagctgttaataatgagaattttgcaagggttgcagttaagcataagctgcatgtacatcttcgccatggatcatctgcattagaaacacagattcgagagtttgtgaaagatgtccaagaggagcttttgaaaccaggtttcaattcttttgggcttggggattgtaaggctcctaaagttcttggtgacattgttgagtcgattgctggcgcgatattcctggatagtggatatgatacctcagttgtctggaaggtttttcagccactccttcatccaatggtgacgccagagacccttccgatgcatccagttagggagctccaagaacgctgccaacagcaggctgaaggcctggaatacaaagcatcccgagcaggcaacatagctactgttgaagtatttgttgatggtgttcagatcggtgtagctcagaaccctcagaagaagatggcacagaagttggcagcgaggaatgcacttgtggtcctgaaggagaaggaaaccgcaacaaagaaagaagatgaaagggatggcgagaagaaaaatggtgctcagatgttcaccaggcagaccctgaacgacatctgcttgagaaggcagtggccaatgcctcagtacaggtgcgtaaacgagggtggccctgcccatgctaaaagattcgtctattcggtgagggtgaacacttctgatcgtggatggacggatgaatgcattggagagccaatgcctagtgtgaagaaggctaaagactctgctgcagttcttctcctcgagcttctgaatagagatttccctgacaagcctgacggtaagcagccgtag

SEQ ID NO: 68 - DNA, 5667 bp, Solanum lycopersicum
  misc_feature 1..5667: note = ORF of protein Dcl1
  source 1..5667: mol_type = unassigned DNA
atggatggtgatggtagtggagtttctgagaaaccttcttattggattgatgcttatgaggacatctcatgtgatgatctcatccaagattttgttgggtccaacactgtacccgttgcaccattgaatcaattgggtccggatgagactcttgatccttgtttctttggtggtattgatgggattctcgagaaattaaagaatggtgatggttctgtacattctgtggatggaaacaacagtaatgtacacactgaagctttagctgcacctggaatttgcaaggacaacgggccacaagttaaaaaggatgtggaccacaacaaaggtgatttggggaagaagtctttacaagaaggaaatggattttccaggcataaggaacggggttgtagtgataaagaagagaaaaatggaaaaagggcgcggcttggtgatgatagctaccagaggagaggctgtgatagacctttggctagaaagagacttcgcgagaatgatgagatcaatagagtagggagggatcaaagaaaaagaagagaatatcatggtggtcgtggtggaagagataggaactggagggaagggagtggcttttgggaatgggacaaagaaaagaaagagatgatctatcgtgtgggttcatgggaagctgatagaaatcgagaggggaagttaccaactgagagaagccgtgaaccttctggagctattgagaagaaagatgacaaattaaaggaacaagctcccaaagaggaagcacgcaagtatcagttggatgtacttgagcatgccagaaagagcaatacaatagcttttcttgaaacaggagcaggaaaaacgcttattgccatcctcctcatgaagagtctatgtagtgacttgcaaaagaagaacaagaaaatgctggcagtttttctggttcctaaggttccacttgtttatcagcaagcagaagttattcgtgagcaaactggctatcaagttggccattattgtggtgaaatgggccaagatttttgggatgcacgaaggtggcagcgcgagttcgaaactaaacaggtcctagttatgactgctcagattctcttgaacatattgaggcacagtataataaaaatggaagcaattaatctccttattatggatgaatgccaccatgctgtgaagaagcatccatattctttagtaatgtctgagttctatcatacaacacaaaaggagaaaaggccatctgtttttggcatgactgcttcacctgttaacttgaagggtgtttccagccaggttgactgtgcaataaagattcgtaatttggagactaaattagattctgtcgtctttaccataaaagaccgcaaggagctggaaaaacacgtgccaatgccgtcagaagtagtagtggagtatgataaagctgctagtctgtggtcttttcatgaacaaataaaaaaaatggaatcagaggttgaagaagctgctctgttaagctccagaagaagtaaatggcagtttatgggggcttgtgatgctggagctagaggagagctgcgccaagtctatggtgtttctgaaagaactgaaagtgatggagctgctaatctaattcaaaagttgagggctatcaactatgctcttggtgaacttggacagtggtgtgcctataaggtagcacattcttttttgacagctctacaaaatgatgaaagggcaagttaccagcttgatgtcaagttccaagaatcctacctggataaggttgtctctctcttgcaatgtcagttatcagagggagctgtagcacaaagtaacttgaatgcagagacgcataaaggtgacaatccaaatagtgataggcctgatgagatggaggagggcgagcttcttgaaagtcatgttgtatctgttggagagcatgtagatgtgattctaggagctgcagtggctgatgggaaagtgacacctaaagttcaatcattaattaagattcttctcaagtatcagcatactgaagattttcgtgctataatatttgttgaacgcgttgtgactgcattggttcttccaaaggtttttgaggagctcccttccctaagttttatcaattcatcaagtttaatcgggcataacaatagtcaagaaatgcgaacaggtcaaatgcaggacacaatagccaaatttcgagatggtcgtattaatttgctagtagctactagtgtagctgaagaaggacttgacatccggcaatgcaatgttgtcattcggtttgatctggccaaaactattttggcgtatatacagtctaggggccgtgcccgtaagcccggatcagattatattttgatggttgagagagataattcatcacatgaagcatttctgaggaatgccagaaacagtgaagaaacgctgcggaaagaagcaattgagcggactgatattagtcatctcaaagatgcctccaagttgatctctgcggaggcaccaacagattcagtttatcaggtggagtcaactggagctgttgtgagcttaaattctgctgttgggctgatccacttttattgctcccaattaccaagtgacaggtactcgattctccgtccggagtttataatggagagtcacgagaagtcaggttgccctactgaatattcatgcaggcttcaactgccctgcaatgcacctttcgagacacttgatggccctgtatgcagctcaatgcgcctcgcccagcaggctgcttgcttggatgcttgcaagaagctccaccagatgggggcatttacggacatgctcttgcctgacaagggaagtggggtggagtcggagaaagttgaacaggatgaagaaggagatccaattcctggaacttccaggcacagggaattttatcctgaaggtgtagctgatattctgaggggtgattggattttgtcgggaaaggatcctcttgtcagctcaaagttcattcatctttatatgtatgctatcaaatgtgtaaatattggcccctcgaaggacccattcttaactgatgtgtcagagtttgcaatactatttggcaacgagctggatgcagaggtattatcgatgtcaatggatttatttattgctcgaactgtagtaacaaaggcgactcttgtcttcagagggccaatagatgttacagagtttcagttggcgtccctcaagagttttcatgtaagaatgatgagcattgttttggatgttgatgttgagccgtccaccactccatgggaccctgcaaaggcatatctatttgcccctgttagtggtgatgaatctggagatcctataaaagatatcaactgggatcatattaaaaaaattaccgaaactggtgtatggagcaatcctcttcagagagctcgcccagatgtatatcttggtacaaatgagcgttctcttggtggagaccgaagagaatatggctttgcgaaactgcgacatggcatggccatcggactgaagtctcatcctacatatggtgttagaggtgctatagcacactatgatctggtccaagcatctggtttggtccctaaccgaagtagccttgatgatgttgaagttgatttgaataaagacaagataatgatggctgattgttctcttagagccgaagatattgtaggaagaattgtcactgcagctcactctggaaagagattttatgttgattgcattcgtagtgatatgactgcagagaactcatttccaaggaaagaaggttacctaggtcctcttgagtacagcagttatgctgcatattataagcaaaagtatggagttgatttggtctataaaaagcaacctctaataagagggcgtggtgtttcatattgcaaaaaccttctgtcaccacgatttgaacattcagaagaacatgagggtgaactcgaagaggcaactgacaaaacgtattatgttttcctccctcctgagctctgtgttttgcatccacttcctggatcccttgttcggggtgctcaaagattgccctcgatcatgaggaggattgagagcatgctgcttgctgttcagcttaaggaaatgattggttaccctgtaccagcactgaagatcttggaagcattgaccgctgcttcttgtcaggagactttctgctatgaaagagcagaacttcttggagatgcatatctgaaatgggttgttagtcggtatctttttcttaagtatccccagaaacatgaaggccaactcaccaggatgagacagcaaatggtaagcaacatggtactgtatcaaaatgccttgagtaagggaatccagtcgtatatccaagcagatcgattttctccatcaagatgggccgctcctggggtgttgcctgtctatgacgaggacctcaatgaggatgaaacatcgatatttgatcatgaaacagcagaaaatggtactgtagcagcaaaggctctagctggtgatgagtttgaagatgaggaaactgaagaaggagtatattacgttgagggtggaaagtatgctgcaaaccacttcatgaaatggattggagttgaggttgatttcgatttcaatttcaaagagacagaatactctattaggtcttgtagcattcctgagaatgtactcaagagtgttgagtttgatgcattacaaggtgctttgaacatcagcttcaatgataaaggcctactgctagaagcaattacccatgcttcacgcccatcttctggggtctcctgttatcaaagactggagtttgtgggtgatgcagtattagatcatctcatcacgaggcacttgttctttacatacacagatcttcccccaggccgattgactgacttgcgagctgcggctgtcaacaatgaaaattttgcacgtgttgctgttaagcacagtcttcatttacaccttcgtcatggatccagtgctctagaaaaacagattcgtgactttgtgaatgaggttaaaaatgaactatcaaaaccaggtttcaactcctttggtttgggagattgcaaagctcctaaagttcttggtgatattgttgaatcaattgctggggctatttttctagacagtggatgtaatactaaatctgtatgggaggtttttcaaccgctattgcatccaatggttacaccagaaacactgccgatgcatccagttcgagaattgcaagagcgttgtcaacaacaagctcaaggcttagagtacaaagcaactcgaagtgggaatatggctacagttgaagtatatgtggatggtgttcaggttggaatggcgcagaatcctcagaagaaaatggcacagaagttggctgccaggaatgcccttgttgtgttgaaagagagggaggaagctgaagctaagaaggctgatgatacaaagaaaaagaagaatggcaacccgagttttactaggcagacactaaatgatatatgcttgcgcaaaaattggcctatgccactgtatcgatgtgtgcacgagggtggtccagcacatgcaaagaggtttacatatggagtacgcgtaaatatttcagacaaaggatttacagatgaatgtattggggaacctatgccgagtgtaaagaaagccaaggattccgcagcagctcttctattggaactcttaaatagacggtactcgtaa

SEQ ID NO: 69 - DNA, 3924 bp, Zea mays
  misc_feature 1..3924: note = ORF of protein Dcl1
  source 1..3924: mol_type = unassigned DNA
atgaaaagtgacagtaatgatgttgaaatgcataattctgaaaaccccaagccaaatgaacttgaggagggagagctacctgacagtcatggtgaacatgtagatgaagtcattggtgccgcagtggccgatggaaaagttaccccaagggtgcaagctttgataaaaatacttctcaagtatcagcacactgaggattttcgtgctatcatatttgtagagcgagtggttacagcattagtcctccctaaggtctttgcagaacttccatcgttgggctttatacggtgtgcaagcttgataggtcataacaataaccaggagatgcggtcgtgccaaatgcaggatacaatcacaaaatttcgtgatggccgtgttacactgttagttgcaactagtgttgcagaggaaggacttgatattaggcagtgcaatgttgtcattcgctttgaccttgcaaagactgttttggcctacattcagtctcggggtcgtgctagaaagcctggatctgactacatattgatgcttgaaagaggaaatatgtctcatgaagctttcttgagaaatgctcggaatagtgaagagacattgagaaaagaagctattgagagaactgatctcagtcatctcgatggcacttctatgctaagccctgttgatatatcacctgattctatgtatcaggttgaatcaacaggtgccgttgtcagtctgaactctgcagttggactcgtacatttttactgctcacagctgccgagtgacagatactctattcttcgaccggaatttattatgcaaaagcatgagaaaccagggggttcaacagaatattcttgcaaacttcaactcccttgtaatgccccattcgagaaacttgaaggtcctatatgcagttcaatacgcctggcccaacaagctgtttgtttagctgcttgtaagaagctacatgagatgggtgccttcacagatatgcttttacctgacagaggaagtggggaaggagaaaagactgaacaaaatgatgaaggtgatccactgcctggaacagcacgccatagagagttctttcctgaaggggttgctgaaattctacatggagaatggattttatctggaagagatggttaccaaagttcacagtttattaagttatatttgtattctgtgaactgtgtaaacattgggacttcaaaggaccctttcgttacacaactttcaaattttgctcttatttttggcaatgagctggatgcagaggttttatcgacgacgatggatctctttgttgctaggacaataataacaaaggcatctctcgtattccgtggaccaattgaaattacagaaagtcagctgatcctgcttaagagctttcatgttaggctcatgagcattgtacttgatgttgatgttgatccctcaacaactccttgggatccagcaaaagcatacctctttgttcctgtgggagctgagaaatgtatggatcttttaagagagatagattggactctagttaatagcattgtgaattctgatgcctggaacaaccctcttcagagggcacgcccagatgtctatctaggcacgaacgaaaggacacttggaggggataggagggaatatgggttcgggaagttgcgccatggaactgcatttggacagaaagcccaccctacgtatggtatcagaggagccatcgctgaattcgatgttgtcaaagcgtctggattagttcctggtcgtggatggggacattttaatgattatcaaaataaaggcaagttgttcatggcagattcatgttgggatgccaaagatcttgctggaatggttgtaactgccgcccactcaggaaaaaggttctatgtggacagtatttgctataatatgaatgcagaaaattctttccctaggaaagagggctatctgggtcccttggaatacagctcgtttgctgattactacaagcagaagtatggtgtggagttagtttataagaaacaacctcttatacgggcacgcggtgtttcatactgcaaaaatctactttctcctagatttgagcattctgaagctagcaatggagagttctcagagaatcttgacaagacatactatgtatatttaccacccgaattgtgccttgtgcatcctctccccggatcacttgttcgtggagctcagaggttaccctcaataatgagaagggtggagagcatgcttcttgcaattcaactgaaggacataattgggtatccagttcctgcaaataagatattagaagccttgactgctgcgtcatgccaggagacattctgctatgaaagagcagagctgctgggtgatgcatacttgaaatgggttgtgagtagattccttttcctaaaatatcctcaaaagcatgagggacagctcacaaggatgcggcagcagatggttagcaacatgatcctgtatcaatacgccctgaataaaaacctccaatcttatatccaagcagatcgatttgctccatcaagatgggcagctccaggagtgttacctgtatttgatgaagaaacgagagattctgaaccatccatctttgatgaggaatttactcctagcagtgagttacaaaagaacttgtatgatgactatgatggcgatagcatgcaagaagacggtgaaattgagggagattctagctgctatcgtgttctatcaagcaaaacattagcagatgttgtggaagcacttattggtgtctattatgtagctggagggaaaattgctacaaaccatctgatgaaatggatcggtattaatgcagagttggatcctcaagagatcccatcttcgaagccatataacatacctgagagcataattaaaagcatcaattttgacacattagagggtgcattgggtatcaagtttcagaataaaggcttacttgttgaagctataacacatgcatcaagaccatcatcaggtgtttcctgttaccagcgcctggaatttgttggtgatgctgtattggaccatctcattacgaagcacttattttttacttatacggacctacctccaggccgcttgactgacttgagagctgcagctgttaataatgagaactttgcaagggttgcagttaaacataagctacatgtacatcttcgccatggatcatctgcattggagacacagattcgagagtttgtgaaggatgtccaggaggagctttcgaaaccaggtttcaattcttttggccttggggattgtaaggctccaaaggttctcggagacatttttgagtctattgctggcgcaatattccttgatagtgggtgtgctacctccgtagtctggaaggtttttcagcctctgcttgatccaatggtgacgccggacacccttccgatgcaccctgtaagggagctccaagagcgctgccagcagcaagccgaaggattagaatacaaagcatcccgtacaggcaatgtagctacagttgaagtcttggttgatggcatacagattggtgtagctcagaacccacagaagaagatggcacaaaagttggcggctagaaatgcacttgttgtcttgaaagagaaggaaaccgcagcaaagaaagacactgaaaaggatggcgagaagaaaaacggttctcagttcactaggcagactctgaacgacatctgcttgagaaggcaatggccgatgccacagtacaggtgtataaacgagggtggccctgcccatgccaaaagatttgtttatgcggtaagggtaaatacatctgacagtggatggactgacgagtgcattggagagccaatgccgagtgtgaaaaaggccaaggactctgcagctgttcttctgctcgagctgctgaatcgaaattaccgtggcaagcctgatggaaaataa

SEQ ID NO: 70 - DNA, 5601 bp, Brassica rapa
  misc_feature 1..5601: note = ORF of protein Dcl1
  source 1..5601: mol_type = unassigned DNA
atggtaatggaggatgagcgtagtaataaggatgtcaacaacaagccatcttactggctagatgcttgcgaggacatctcctgcgatctcatcgatgacctcgtctccgatttcgatccttcctctgtcgccgtcgctgagtccatcgtcaacaacgacttcttcggtgggatcgatcacattctagacaccatcaagaacggtggaggcttgccaaacaacgaggctcctcagttgaatcctggagatgttgttactaaggagaatggtttgcaaaagactggtcttaaaagagacgatgcatcgaaagaggaaggagacaagaacaggaagagagctagagtttgtagttaccagagcgagaagaataggaaacggactcgaagctacgaggaggctggtggtcataacaagagaagagatggttacaattacagaagagacggtagagataatagagaaggtagaggctattgggagagggataaagtcggttcgaacgagcttgtttatcggtctggtacttgggaagctgatcacgaaagagatgttaagaaagagagccgtagaagccaccgagaagaggcagaggaggataataagaagagtaagcctgaagaacagcgtaaagagaaagttgtggaagagcaagcgaggcggtaccagttggacgtgcttgaacaagctaaagcgaagaacacgatcgctttccttgagaccggtgcggggaagacacttatagccattctcttgatcaaaagtgtgcataaggatctaatgagccagaacagaaaaatgctctctgtcttcttggttcctaaagtgcctttggtttatcagcaagcggaggtgatacgtaaccaaacttgttttcaagttgggcactactgtggtgagatggggcaagacttctgggatgcgagaaggtggcagcgagagttcgagtctaagcaggttcttgtaatgacggcacagattctgttgaacatactgaggcacagcattatcagaatggaatctattaatctactgatcctcgacgagtgtcaccacgctgtcaagaaacatccttactcactggtgatgtcagagttctaccacacaacgtctaaggataaaagacctgccatctttggcatgactgcttctcctgttaatctaaaaggtgtttcaagccaggtagattgtgctataaaaatacgcaatctcgagacaaagttggattctacggtttgtactataaaagatcggaaggaactagagaaacatgttcccatgccttcagagattgtggtggagtatgacaaagctgctactatgtggtcttttcatgagaagataaagcaaatgattgcagctgttgaggaagcggcgaaggcgagttcgaggaaaagcaagtggcagtttatgggggctagggatgctggagctaaggaggagctgaaacaggtttatggcgtctctgagagaacagagagcgatggagctgctaatttgattcataagcttagagctattaattatactcttgctgagttgggtcaatggtgtgcttacaaggtggcacaatcgttcttgactgcgttgcaaagtgacgagagggtgaatttccaagtggatgtgaagtttcaagaatcttatctcagcgaggtggtgtcgctcttgcaatgtgagcttgtggaaggagctgctgctgagaaagcagccacggaatgtagcaaaccagagaatggtaatgcaaatgatgagattgaggaaggagagctccctgatgatcatgtggtctctggaggggagcacgtcgatgaagtaataggcgccgcagttgctgatgggaaagttactccaaaggttcagtcgctgatcaaactactcctcaagtatcagcacacatctgattttcgtgctattgtgtttgttgagagagtggttgctgctttggttcttccaaaggtcttcgcggagctgccttctcttggttttataaggtgtgccagcatgattggtcacaataacagccaggaaatgaaatcatctcaaatgcaggatactatttccaaattccgagatggtcaagtgacactgttagtggccacaagcgttgctgaggaaggtcttgatatcaggcaatgtaacgttgttatgcgttttgaccttgcaaagacggtgctggcatacattcagtctcgtggtagggcaagaaagcctggttcagactacatactcatggttgagagggaaaatgtatctcacgcagcctttttaaggaatgctaggaacagtgaggagacactgcggaaagaagcaatagaaaggactgatctgagtcacctcaaagatacatcgaggctaatctccattgacgctgtccctggtacagtttataaggtggagacaactggtgccatggtcagcctgaactccgcggttggtctcatacatttctactgctctcagcttcccggtgacaggtatgcgattcttcgacctgagttcagcatggagaaacatgaaaagcctgggggacacaccgagtattcatgcaggcttcagctcccttgcaacgctccatttgaaatactcgagggtcctctttgtagttcaatgcgccttgcacaacaggctgtatgtctagctggttgcaagaaactgcacgagatgggcgcatttacggatatgctgttaccggacaagggaagtggtcaagacgctgagaaggctgaccaggatgaagaaggtgaacccgttcctggaactgctagacacagagagttctatccagaaggtgtagcagatgtacttaagggagattggattttatcaggaaaggaagattgtgagagctcaaagctattccatctatacatgtacagtgttagatgtgaagattctgggtcttcaaaagacccattcttaaccgaagtttcagagttcgcggttctttttggcaatgagctggatgcagaggtattatcgatgtctatggatctttatgttgctcgagccatgatcaccaaagcatctcttgttttcaagggatcactggatattacagaaagccagctatcatctattaaaaagtttcatgtgaggttgatgagtatagtcttagatgttgatgtcgagccctccacaacaccatgggatcccgcaaaggcctacttgttcgtccccgtgactgacaacacgaaagggatcaactgggaactaattgaaaagataactgagaccaccgtgtgggacaacccgcttcagagagctcgtcctgatgtgtaccttgggactaacgagagaactcttggtggggacaggagggagtatgggtttggtaaacttcgtaacaacattggattggggcagcagaaatctcacccgacttatggtattagaggagcggttgcatcttttgatgtcgtgagagcttctggattgttacctgtgagagaggaagtaagaagtgatgtatcacaagggaaactgatgatggcagatgggtgcatggttgcagataatctaatagggaaaatagtgacggccgcacattcagggaagcggttttatgtggactctatatgctacgatatgagtgcagaaacatcattcccgaggaaagaggggtatcttggtcctttggaatacaacacttacgctgactactataagcaaaagtatggagttgatttaaactgtaaacaacagcctttgatcaaaggacgaggtgtttcctattgcaagaaccttctttctcctcggtttgaacagtcaggtgaatctgagacgatacttgataaaacatattacgtgtttcttccacctgagctgtgcgttgtgcatccgctttcgggttcacttgtccgaggggctcagaggttgccctctataatgagaagagttgagagcatgttgctcgctgttcagctgaaaaacttgattagttatcccattccaacttcaaagattctggaagcgttgacagcagcctcgtgccaggagacgttctgctacgagagagcagaactcttaggagatgcgtatctgaaatggatcgttagtcgttttctgtttctcaagtacccgcagaagcacgagggtcagcttacgaggatgagacagcagatggtcagcaacatggttctttaccagtacgctcttctcaaagggctccagtcatacattcaagcggatcgtttcgcaccttctaggtggtctgctcccggcgtgcctcccgtttacgacgaggacaccaaagacggaggaggaggatcttccttcttcgacgaagagcagaaacaagcctcctccgaggacgtgtttgaagatggggagatggaggatggcgagctagagggtgacttgagctcttaccgagtcctatctagcaagacgttggctgatgttgttgaagctttgatcggtgtttactacgtggaaggaggtaaagctgctgcgaaccatttgatgaaatggattgggatccacgtggaggatgatcccgaagaaaccgaaggggccgtgaaaccggttagtgttccggagagcgtgctcaagagcatagactttgttggtttagagagagctttgaagtacgagtttacggagaaaggtcttcttgtggaagctatcacgcatgcttcgagaccgtcttcgggtgtttcgtgttaccagaggttggagtttgttggggacgcggtgttggatcatcttattacaagacacttgtttttcacgtacacgagccttcctcctggtcggttaactgatcttcgagctgctgcggttaataatgagaatttcgctcgtgttgcggttaaacataagctccacttgtaccttcgtcacggctctagcgctctcgagaaacagattagagagtttgtgaaggaagtcctaaccgagtcttctaaaccggggtttaactcgttcggtttgggagattgtaaagcgccaaaggttcttggagatatagttgaatccattgcaggtgctatttttcttgatagtggaagagacacaagtgctgcttggaaggtttttcaacctttgctacaaccaatggtgacaccagagacacttccgatgcatccggtgcgagagctgcaagagcgttgccagcaacaagcagaagggttagagtacaaagcgagccggagtggtaacacagcgactgtggaagttttcatcgacggtgttcaagtgggagtggcgcagaatcctcagaagaaaatggctcagaagctagcggcaaggaacgcgctggcagctttgaaagagaaagagatagctgagtcaaaagagaaggcggcaaacgggaatgcaggagacgagagtgagaatgggaacaagaagaatgggaatcagacgtttacgagacagacgttgaatgatatttgcttgaggaagaattggccaatgccttcttacagatgtgtgaaggaaggaggaccggcgcatgcaaagaggtttacatttggggtaagagtgaatacaagcgatagaggatggactgatgaatgtattggagagcccatgcctagtgttaagaaggctaaagattctgctgcttctcttcttcttgagcttcttaataaaacttattcttga

SEQ ID NO: 71 - DNA, 5994 bp, Hordeum vulgare
  misc_feature 1..5994: note = ORF of protein Dcl1
  source 1..5994: mol_type = unassigned DNA
atggcgggaggaggaggaggaggtggcgcgggcgagcacccgggggcggtgtcctactggtacgacgcctgcgaggacgacgaggacgcgtcgctgctgtgcggcatcgacttcgcctcgtccgccgacttcgacccggggctcatgcccttgcccgccatggactgcggcatcgacttcgccgcctccgcggatttcaaccccggcttcatgcccgccatggactgcggcggcgtagacgacggattcgtcgccgagatcgaccggattctcgagagcatcgacgccgagacagcccccgcgccacagcccccgcctgtggctcccccgcaaccgccggcggagcgggtgcaggaggctgctgctattgcggctgtggccaacaatgccgtggcggtggtgtgctcggagcagaggagccaggccggcgggcccaggaacgaggcgagaagcgtcgaggcgaggaaggagacgagaagcgtcgggccgaggaaggagacgacaagcgccgagccgaggaaggagacgagaagggagacgcaaggcggttcggcgaacggcggtcgcggcggggagtggagagacgggaagcgtcctcggctcgcccctggggctgacgggggcccacgccctgattcccggcggcgcccgatgctgccgccgccctcgcgtggctgggatgaacggcgcgggaggcgcgacttcgtcgacaggcctcgcaagcgcgaccgcgaccgcgacagtaaccctgcccaccggcgcgaggctcgaggattctgggagcgggaccgcggcggcaagatggtgttccgccctggcacctgggagcaggagtctgagcgcgagggaaagcgtgccaggacacagaacggaggctccatggagaagaatgcagacgcagataaggcggcttccgcccacaaggagaagcctctcgccgaggagcaggccagacattaccagttggaggtgctcgagcaagcaaagagcaggaacacaatagccttcctcgagaccggcgcagggaagaccctcattgctgtgctgctcatcaaaagcatatgtgacaaaatgatgaaggagaacaggaaaatacttgccgtgttcttggttcccaaggtgcctcttgtgtatcagcaagctgaggtgatacgcgagaggactggctatcgggttgggcactactgcggagagatgggtcaggatttctgggatgctaggaagtggcagcgagagtttgattccaaacaggtactagttatgacagctcaaattttgttaaacatcctcaggcacagtattattaaaatggacgccatacatcttttaattttagacgagtgccatcatgcagtgaaaaagcatccatattctctagtgatgtcagaattttatcacacaactccgaaggataagagaccagttgtttttggtatgacagcttctcctgtcaacctaaagggagtcactagccaagaagattgtgccatcaagataagaaatcttgagagtaaactggattccgttgtttgtactatcaaggatcggaaggagcttgagaaacatgttcccatgccttctgaagttgttgtccaatatgacaaagcagccaccctttgttccctgcacgagcagatcaaacaaatggaagctgctgttgaagaagctgcactctgtagctctaagagaacaaagtggcagtttatgggagccagggatgctgggtctagagatgagctgcgccttgtctatggtgtttctgagcgtacagaaagtgatggtgcagctaatttaatccagaagttgagagctataaattatgctttaggtgaactaggacaatggtgtgcttataaggttgcacagtccttcttgacagtattgcagaatgatgaaagggcaaactatcaggttgatgtaaagtttcaagagtcgcatttaaagaaggttgttgatttattgcactgccagttgactgagggagctgcagtgaaaagcgagacgaatgatgttgagatgcataacactgaaaatcacaacccaagtgaacttgaagatggggagctgcctgatagccatgctgtttctgtaggtgaacatgtagatgaggttatcggagctgccgtggcagatgggaaagttacccctagggtgcaggctttaatcaaaatccttctcaagtatcagcatactgaggatttccgtgctatcatctttgtggagcgagttgtcacggcattagttcttcctaaggtgcttgcagagcttccatctttaggctttatccgatgtgcaagcttgataggtcataacaacaatcaagagatgcgcacaagccaaatgcaggatacaattgcaaaatttcgtgatggtcgtgttacactgttagttgcgactagtgtcgctgaggaaggacttgatattcgacagtgcaatgttgtcattcgctttgaccttgcaaagacggttttggcctacgtacagtctcgtggtcgtgcaagaaagcctggatctgactacatattgatggttgaaaggggaaatttggcacatgaaactttcttgaggaatgctcgaaatagtgaggagacgttgagaaaagaggctatagaaagaactgatctcagtcatcttgatggtactctgttaaattccattcatataccacctgattcgatgtatcaggtcgaatcaacaggtgctgttgtcagcttgaattctgcagttgggctcgtacatttgtactgttcacagcttccaagtgacaggtactcaattcttcgtccagaatttattatgcaaaaacatgagaagccagggggttccatggaatattcttgcaaacttcaacttccctgtaatgctccatttgagaaactcgagggtcctatatgcggatcaattcgcctggcacagcaagctgtttgtttggctgcctgtaagaagctgcacgagatgagtgccttcacagatatgcttttacctgatagaggaagtggggaaggagagaagaccgaaaagaatgaaggtgatccactccctggaacggcacgccatagagagttttatcctgaaggtgttgctgaaattctgagaggagaatggattttatgtggaagagatggctgccaaagcactcaatttattaagttgtatatgtattctgtcaattgtgtggatgtagggacatctaaggacccttcccttgcacaaatatcgaatttttctattatttttggcagtgagctggatgcagaggtatgttccatcatctggaatatcctgaaacttgaatataatgttgtgggttcaccattttttctttctgattttcaggttctgtcgacaccaatggatctctttgttgccaggacgatgataacaaaagcatctcttgtattccgtggaccaattgaagttacagaaagtcagctggttctgctgaagagctttcatgttaggctcatgagcatagtccttgatgttgatgttgatccctcaactacaccttgggatccagcaaaagcatacctctttgtccctgtaggagctgagaagtgtgcaggtgctttaagagagatcgattggactttaatcaacaacattgtgaacactgatgcctggaacaacccacttcagaaggcacgaccagatgtctatctaggcaccaatgagaggacactcggtggagataagagagagtatgggtttggaaaattgcgtaatggaaccgcttttggacagaaggcacatccaacttatggtattagaggagccattgctgactttgatattgttaaagcatctggcttagttccttctcgtgatagagggtattcaagtgactatcagaaccaaggcaagttgttcatggcagattcatgctgggatgctaaagatctttctggaatggttgtcactgctgcccactcaggaaagcggttctatgtggactgtatttgttataacatgaatgcagaaaattcatttcctaggaaggaaggctatctggggcctttggaatatagctcatatgctgattactacaagcaaaaatatggtgtggagctagtgtataagaaacaacctcttatacgtgcacgtggtgtttcctattgcaagaatcttctgtctcctagatttgagcattctgaagctagagaaggtgatttttcggagaacgttgacaaaacatattatgtctatttaccacccgaactttgccttgtgcacccacttcctggatcactcattcgtggagctcagaggttgccatcaataatgagaagggttgagagtatgcttcttgcagttcaattgaaggaaacaattgattatcctgttcccgcagttaagatattagaagccttgactgctgcatcatgccaagaaacattctgctatgaaagagcagagttactgggtgatgcatacctgaaatgggtcgtgagtagattccttttcctaaaatatcctcagaagcatgagggtcagcttacaaggatgaggcagcagatggtcagtaacatggtcctctatcaatttgccctgaataaaactcttcaatcatttattcaagcagatcggtttgccccatcaagatgggcagctcccggagtactacctgtatttgatgaagagacgagagaatctgaaccgtccatctttggtggggagtcaatacctggtaatgaattacagaaggattatgacgatgactacaccgatagcattcaagaggagggcgaaattgatggagattctagctgctatcgtgttctctcaagcaagacattagcagatgttgttgaagcacttattggggtctattatgtagcaggagggaaaattgctgcgaaccacctgatgaagtggattgggatccacgccgaattggatcctcaagagatccctcccccaaagccatataatatacctgagagcatattgaaaggcattgactttgagacattggaaggcatcttgggtaccaaattccagaataaaggttttcttattgaggctataacgcatgcatccaggccatcttctggtgtttcctgttaccagcggttggaatttgttggtgatgctgtgttggatcatctcatcacgaagcatctattttttacatatactgatctacctcctggtcgcttgactgacctgagagctgcggctgttaataatgagaactttgcaagggttgcagttaggcgtaagctgcacggacatcttcgccatggctcatctgcactggaaacacagattcgggagttcgtgaaggatgtccgaaaagagatttcaaaatcaggtttcaattcttttggacttggggattgcaaggctcctaaagttcttggagacattattgagtctatagctggggcagtattcctggatagtgggtatgacacctcggcagtctggaaggtgttccaaccgctcctcgaacctctggtaacgccagagacccttccgatgcatccaattagggagctccaagaacggtgccagcaacaagcagaagggttagaatacaaagcatcccgggcaggcaatgtagctactgtggaagtctttgttgatggtgttcagattggtgtagctcagaatccccagaagaagatggcacagaaattggcggcgaggaacgcactcgtggtcctgaaggagaaggaaactgcagcaaagaaagagacagaaaaggatggtgataagagcaatgctgggttcaccaggcagaccctgaacgacacttgcttgagaaggcagtggccaatgccgcagtacaggtgcataaacgagggcggccctgctcatgctaaaagatttgtgtacgcggttagggtgaatacgtcggatcgtggatggaccgacgagtgcgtcggagagccgatgccgagcgtgaagaaggccaaggactctgccgccatgcttctccttgagcttctgaatcgaagtttccctgataagcctggcggtaagaagtga

SEQ ID NO: 72 - DNA, 5745 bp, Solanum tuberosum
  misc_feature 1..5745: note = ORF of protein Dcl1
  source 1..5745: mol_type = unassigned DNA
atggatggtgatggtggtggagtctctgacaaaccttcttattggattgatgcttatgaggacatctcatgtgatgatctcatccaagattttgttgggtcgaacacagtacccgttgtgccatcgaatcaattgggtccggatgggagtcttgatccttgtttctttggtggtattgatgggattcttgagaaattaaagaatggtgatggttctgtacattctgtggatggaaacaacagtaatgtacatagtgaagctttagctgcacctggaatttgtaaggacaatgggccacaagctaaaaaggatgtggagcacaacaaaggtgatttggggaagaagtctttacaagaaggaaatggattttccaggcataaggaacgaggttgtaatgataaagaagagagaaatggaaaaagggcgcggcttggtgatgatagctaccagaggagaggctgtgatagacctttggctaggaagagacttcgtgagaatgatgagatcaatagagtagggagggatcaaagaaaaagaagagaatatcatggtggtcgtggtggaagagataggaactggagggaagggagtggcttttgggaatgggacaaagaaaggaaagagatggtctatcgtgtgggttcatgggaagctgataggaatcgagaggggaagttaccaactgagagaagccgtgaaccttctggagctattgagaagaaagatgacaaattaaaggaacaagctcccaaggaggaagcacgcaagtatcagttggatgtacttgagcatgccagaaagaacaatacaatcgcttttcttgaaacaggagcaggaaaaacgcttattgccatcctcctcatgaagagtctatgtagtgacttgcaaaagaagaacaagaaaatgctggcagtttttctggttcctaaagttccacttgtttatcagcaagcagaagttattcgtgagcaaactggctatcaagttggccattattgtggtgaaatgggccaagatttttgggatgcacgaaggtggctgcgtgagttcgaaactaaacaggtcctggttatgactgctcagattctcttgaacatattgaggcacagtataataaaaatggaagcaattaatctccttattatggatgaatgccaccatgctgtgaagaagcatccatattctttagtaatgtctgagttctatcatacaacacaaaaggagaaaaggccatctgtttttggcatgactgcttcacctgttaacttgaagggtgtttccagccaagttgattgtgcaataaagattcgtaatttggagactaaattagattctgtcgtctttaccataaaagaccgcaaggagctggagaaacacgtgccaatgccgtcagaagtagtagtggagtatgataaagctgctagtctgtggtcttttcatgaacaaataaaacaaatggaatcagcggttgaagaagctgctctgttaagctccagaagaagtaaatggcaatttatgggggcttgtgatgctggagctagaggagagctgcgccaagtctatggtgtttctgaaagaactgaaagtgatggggctgctaatctaattcaaaagttgagggctatcaactatgctcttggggaactgggacagtggtgtgcctataaggtagcacattcttttttgacagctctacaaaatgatgaaagggcaagctaccagcttgatgtcaagtttcaagaatcctacctggataaggttgtctctctcttgcaatgtcagctatcagagggagctgtagcacaaagtaacttgaatggagagatggataaaggtggcaatccaaatagtgataggcctgacgagatggaggagggcgagcttcttgaaagtcatgttgtatctgttggagagcatgtagatgtgattctaggagctgcggtggctgatgggaaagtgacacctaaagttcaatcattaattaagattcttctcaagtatcagcatactgaggattttcgtgctataatatttgttgaacgcgttgtgactgcattggttcttccaaaggtttttgaggagctcccttccctaagttttatcaattcatcaagtttaatcgggcataacaatagtcaagaaatgcgaacaggtcaaatgcaggacacaatagccaaatttcgagatggtcgtattaatttgttagtcgctactagtgtagctgaggaaggacttgacatccggcagtgcaatgttgtcattcggtttgatctggccaaaactattttggcgtatatccagtctaggggccgtgcccgtaagcctggatcagattatattttgatggttgagagagataattcatcacatgaagcatttctgaggaatgccagaaacagtgaagaaacgttgcggaaagaagcaattgagcggactgatattagtcatctcaaagatgcctccaagttgatctctgcggaggcaccaacagattcagtttatcaggtggagtccactggagctgttgtgagcttaaattctgctgttgggctgatccacttttattgctcccaattaccaagtgacaggtactcgattctccgtcctgagtttataatgaagagtcacgagaagtcaggttgcccaactgaatattcatgcaggcttcaactgccctgcaatgcacctttcgagacacttgatggccctgtatgcagctcaatgcgccttgcccagcaggctgcttgcttggatgcttgcaagaagctccaccagatgggggcatttacggacatgctcttgcctgacaagggaagtggggtggagtcagagaaagttgaacaggatgaagaaggtgttccaattcctggaacttccaggcacagggaattttatcctgaaggtgtagctgatattctgaggggtgattggattttgtcgggaaaggattctcttgtcagctcaaaggtcattcatctttatatgtatgctatcaaatgtgtgaatattggcccctcgaaggacccattcttaactgatgtgtcagagtttgcaatactgtttggcaatgagctggatgcagaggtattatcgatgtcaatggatttatttattgctcgaaccgtagtaacaaaggcgactcttgtcttcagagggccaatagatgttacagagtttcagttggcgtcccttaagagctttcatgtaagaatgatgagcattgttttggatgttgatgttgagccgtccaccactccatgggaccctgcaaaggcatatctatttgcccctgttactggtgatgaatctggagatcctataaaaggtatcaactgggatcatattaaaaaaattactgaaactggtgtatggggcaatcctcttcagagagctcgcccagatgtatatcttggtaccaatgagcgtgctcttggtggagacagaagagaatatggctttgcgaaattgcgacatggcatggccgtcggactgaagtctcatcctacatatggtgttagaggtgctatagcacactatgatctggtccaagcatctggtttggtccctaaccggagtagccttgatgatgttgaagttgatttgaataaagacaagataatgatggctgattgttctcttagagccgaagatattgtaggaagaattgtcactgcagctcactctggaaagagattttatgttgattgcattcgtagtgatatgactgcagagaactcatttccaaggaaagaaggttacctaggtcctcttgagtacagcagttatgctgcttattataagcaaaagtatggagttgatttggtctataaaaagcagcctctaataagagggcgcggtgtttcatattgcaaaaaccttctgtcaccacgatttgaacattcagaagaacacgagggtgaacttgaagaggcaactgacaaaacgtattatgttttcctccctcctgagctctgtgttttgcatccacttcctggatctcttgttcggggtgctcagagattgccctcgatcatgaggaggattgagagcatgctgcttgctgttcagcttaaggaaatgatcggttaccctgtcccagcactgaagatcttggaagcattgaccgccgcttcttgccaggagactttctgctatgaaagagccgaacttcttggagatgcatatctgaaatgggttgttagtcggtatcttttccttaagtatccccagaaacatgaaggccaactcaccaggatgagacagcaaatggtaagcaacatggtgctgtatcaaaatgccttgagtaagggaatccagtcatacatccaagcagatcgattttctccatcgagatgggcagctcctggggtgttgcctgtctatgacgaggacctcaatgaggatgaaacatcgatatttgatcatgaaacagcagaaaatggtactgtagcagcaaaggctctagctggtgatgagtttgaagatgaggaaactgaagaaggtgagcttgataatgattcagggtcatatcgtgtactgtctagcaagactatggctgatgttgtggaagcactgattggagtatattacgttgagggtggaaagtatgccgcaaaccacttcatgaaatggattggggttgaggttgatttcgatttcaatttcaaagagacagaatactctattaggtcttgtagcattcctgagaatgtactcaagagtgttgactttgatgcattacaaggtgctttgaacatcagcttcaatgataaaggcctactgctagaagcaattacccatgcttcacgcccatcttctggggtctcctgttatcaaagactggagtttgtgggtgatgcagtattagatcatctcatcacgaggcacttgttctttacatacacagatcttcccccaggccgattgaccgacttgcgagctgcggctgtcaacaatgaaaattttgcacgtgttgctgttaagcacagtcttcatttacaccttcgtcatgcatccagtgctctagaaaaacagattcgtgactttgtgaatgaggttaaaaatgaactatcaaaaccaggtttcaactcctttggtttgggagattgcaaagctcctaaagttcttggtgatattgttgaatcaattgctggggctatttttctagacagtgggtgtaatactaaatctgtatgggaggtttttcaaccactgttgcatccaatggttacaccagaaacactgcccatgcatccagttcgagaattgcaagaacgttgtcaacaagaagctcaaggcttagagtacaaagcaactcgaagtgggaatatggctacagttgaagtatatgtggatggtcttcaggttggaatggcgcagaatcctcagaagaaaatggcacagaagttggcggccaggaatgcccttgttgtgttgaaagagagggaggaagctgaagctaagaaggctgatgatgcaaagaaaaagaagaatggcaacccgagttttactaggcagacactaaatgatatatgcttacgcaaaaattggcctatgccactgtatcgatgtgtgcacgagggtggtccagcacatgcaaagaggtttacatatggagtacgcgtaaatatttcagacaaaggatttacagatgaatgtatcggggaacctatgccgagtgtaaagaaagccaaggattccgcagcagctcttctgttggaactcttaaatagacggtactcataa

SEQ ID NO: 73 - DNA, 5643 bp, Medicago truncatula
  misc_feature 1..5643: note = ORF of protein Dcl1
  source 1..5643: mol_type = unassigned DNA
atggaggatgatgccgatgccgctgctgccgccgctgccgtgaataacaacaatagggtttcttcgtcaaatccttactggctggatgcctgtgaagatatttctatatcttgtgatgatatcattgattttgatgtttctaatgattctgaccaacaacccaacaacaacaaccaagacttttttggtggtattgatcgaatctttgacagcatcaaaaacggcgctggtctccctgatcatcctcctgcttctgctgctgatgatgggaatcaactacctcctactcatcttcaactagaagaacaacaccaacaggtagaagaagaagaacaagaacaagaaaggttcaacaaaagggctcgcctcaccgaaggagaaaggagaatcaatcatcaacccaacaacaacaacaaggacaggtatgagaggtgtttcaacaataataggaagagaccaaggaataataataatcataatcattcctttaaaagagatgtcaagggttattgggagagggataagtcttctaatgacttggtctttcgctccggaacttgggaacccaatcttcatacttcccaacacaaaatggacatagacaaccccatcaaacaggaaaaacccaaggagactgttcctgttcctgaggaaaaagccagacagtatcagttggatgttcttgagcaggcaaagaccagaaatacaatagcttttctagaaactggagctggcaaaaccctcattgccgttcttctcatcaagtctatacatgagacattacatctccaaaataagaaaatgcttgctgtgtttttggttccaaaagttccacttgtttaccagcaagctgaggtaatccgtgagcgcactggatatcaagtggggcactattgtggagaaatggggcaggatttctgggatgctcgaaggtggcagcgtgaatttgacaccaagcatgttttagtcatgaccgctcaaatacttttgaacattttgaggcatagcatcataaaaatggaagcaatcaatctcctgattcttgatgagtgccatcatgctgtgaagaaacacccatattcgctggtgatgtctgagttctaccatacaacgcccaaagaaaagaggccatctgtatttggaatgactgcttctcctgttaacttaaagggtgtttccagccaagtagactgtgcaataaaaattcgtaatcttgaaagtaagctagattcaatagtttgtaccattaaagatcgtaaagagctagagaaacacgtgcctatgccctctgagattgtggtgaagtatgataaagcggccagtctatgttatttacatgagcaaataaagcagatggagactgaagttgaggaagctgcaaaatctagttcaagaagaagcaaatggcagtttatgggagctagagatgctggagctaaggaagagctgcgccaagtatatggtgtttctgaaagaacagaaagtgatggagctgcaaacctaattcagaagttgagagctgttaattatgcacttggtgaattgggacagtggtgtgcatataaggttgcacaatcttttcttgcagctctgcaaaatgatgaaagggcaaactatcaacttgatgtcaagtttcaggaatcttatctgagtaaagttgtgtctcttctgaaatgtcagctgtcagagggtgccatttctgacaaaaatgatggagtagatgactcacaaaatggtgcagctcacagtgggtctgatgaacatgaagagattgaagaaggagagcttccagacagtcacgttgtctctggtggggagcatgttgatgtcataataggtgctgctgtggctgatggcaaggtgacccccaaggtgcaggctttaatcaaaatactcctcaagtatcagaacacagacgacttccgtgcaatcatctttgttgaacgtgttgtgtctgcattagttctacccaaggtttttgccgagcttccatctcttagttttgtcaagtgtgcaagcttgattggccacaacaatagtcaggaaatgcggacccaccaaatgcatgatacaattgccaagttccgtgatgggcgggtcacgttgttagttgccactagtgttgctgaagaaggtcttgatatccggcaatgtaacgttgttattcgctttgatcttgcgaagactgttctggcatacattcagtctaggggacgtgctagaaagcctgggtctgattacatcctaatggttgagaggggaaatttgtcacatgaagctttcctaaagaatgccaggaacagtgaggaaactttgcggagagaagcaatagagagaacagaccttagtcatctgaaagacagttcaaggttgatttctgtagatacccatccagaaacaatgtaccaggtgaaatctaccggtgcagtagttagcttaaattccgctgtaggtcttgtccacttctattgctcccagctacctagtgacagatattcaattcttcgtcccgagtttattatggagaagcatgaaaactccggaggtcccactgaatattcttgcaggcttcaactgccatgtaatgcaccatttgaaaatctggagggtcctatctgcagttcaatccgcttggcacaacaggctgtttgtctagctgcttgcagaaaattgcacgaaatgggagcattcactgacatgctattgccggataaaggaagtggggaagaaaaagaaaaggctgagcaaaatgatgaaggggatgcacttccagggactgctagacatagggagttctatcctgaaggtgtggctgacatactgaagggagaatggatcttatctggaaaagatgcttgtaacaactccaaattgcttcatctttatatgtataccataaagtgcgagaatgtaggccattctaaggatccgttcttgactcaggtttcagattttgcagtactttttggcactgaactggatgcagaggtcttatcgatgtcgatggatctatttatcgctcgaaccgtgactacgaaggcgtctcttgtctttagagagtcaatagatatcactgagagtcagttgacgtccctgaaaagctttcatgtaaggttaatgagcattgtcttggatgtggatgttgaaccatctaccactccttgggatcctgcaaaagcatatctgtttgtcccaatggttggcgataagtcttcagatcctatgaaccaaattgactggcatctggttgagacaataattggagccgatgcatggaaaaatcccctccagaaggctcgaccagatgtttaccttggcactaatgagagaactttaggtggtgacagaagggaatacggatttgggaaattgcgtcatggcttggcttttgggcaaaaatcacatccaacctatggaatcagaggagctgtggcccagtttgatgtagtgaaagcttcaggattggttcccaacagagattccatggaaacacaaaatcatatcaatatgactaccaaaggaaaattgatgatggcagatacatgtaccagtccagatgatctagtagggagaattgtaactgctgctcattcagggaagaggttttatgttgattctatacgctatgaaatgacagcagagaactctttccctaggaaagaaggttatcttggtcctctggaatacagttcatatgctgattactacaagcaaaaatatggtgttgatttggcttacaagcaacaacctcttataagagggcgtggtgtatcatactgcaagaatcttttgtcccctcgatttgaacacagtgaaggacatgaagatgaaactgaagagacccacgacaaaacttactatgttttccttcctccagagctatgcctagtacacccactgcctggatcactcgttcgtggtgctcagaggctgccttcaataatgaggagggttgaaagcatgctgcttgcagttcagcttaagaatatgataaattatcctgtccaggcttcaaagatcttggaagccttgaccgctgcctcttgccaggagactttctgctatgaaagggcagagcttctaggagatgcttacctgaaatgggttgttagtcgttttcttttcctaaaacatccacagaaacatgaaggccagcttactaggatgagacaacaaatggtcagtaacatggtcttatatcggtacgcattaagtaaaggtcttcaatcatatatcctagcagatcgttttgccccatccagatgggcagctcctggggtgctacctgtctttgatgaggatacaaaggatgaggagtcgtctttatttgaccaggagcgttccatttttaaggccgagaggatggacaacactgatgaatttgaggatgaaatggaagatggtgagcttgagagtgattcaagttcttaccgagttctgtccagcaagacacttgcagatgttgtggaagcactgattggggtatattatgttgaaggtggtaagaatgctgctaaccaccttatgaaatggattggtattcacatagagatcgatcctgatgagatggagtgtataacgaagccatccaatgttccagacagcatacttaggagtgttgattttgatgctttagaaggtgctctaaatattaagtttaaagacaaggggctgctgatagaatccattactcatgcctccagaccatcttcaggagtatcctgttatcagcggttggagtttgttggtgatgctgtcttggaccatcttataaccaggcacttatttttctcgtacacagatctgccaccaggacgtctgactgaccttcgagctgctgctgtaaacaatgaaaattttgcacgtgttacagttaaacacaatcttcacctgcatctgcggcatgggtccagtgccctagaaaaacagattaaagattttgtgaaggaagttcaagatgaattgtcaaaaccagggtttaactcttttggtcttggagactgcaaagctcccaaggtccttggtgacatccttgaatctattgctggtgccatttttcttgacagtgggaggaatactgctgttgtttggaaggtttttcagcctcttttgcatcctatggttacaccagagactttgccaatgcatcctgtgcgtgagcttcaagagcgatgccagcagcaggctgaaggcttggagtatagagcaagtcgtgctggaaatttagccactgttgaagtattcattgatggggtacaagtaggggctgctcaaaatccacagaagaaaatggcacagaaattagctgcccgaaatgcccttgctgctttgaaagagaaggaagagagtaaaattcaggagaaaaatgatgaaaaggagacaaagaatgggaaccagacatttactagacaaacattaaatgatatatgtttgcgaagaaactggcctatgccattctaccggtgtgtgagtgagggtggtcctgcacatgcaaagaggtttacatttgcagtgcgagtgaatactactgataaaggttggacggatgaatgtgttggagaggcaatgccaagcgtcaagaaggccaaagactcagctgctgttcttctcttggaactaataaacaaattatactcgtcatag

SEQ ID NO: 74 - DNA, 969 bp, Triticum aestivum
  misc_feature 1..969: note = ORF of protein Dcl1
  source 1..969: mol_type = unassigned DNA
atgagaagggttgagagtatgcttcttgcagttcaattgaaggaaacaattgattatcctgttcctgcagttaagatattagaagccttgactgctgcatcatgccaggaaacattctgctatgaaagagcagagttactgggtgatgcatacctgaaatgggttgtgagtagattccttttcctaaaatatcctcagaagcatgagggacagcttacaaggatgcgacagcagatggtcagtaacatggtcctctaccaatttgccctgaataaaactcttcaatcgtatattcaagcagatcggtttgccccatcaagatgggcagctccaggagtactacctgtatttgatgaggagacgagagaatctgaaccgtccatctttggtgaggggtcaatacctggtaatgaagtacagaaggattatgatgatgactacacacatagcattcaagaggagggtgaaattgacggagattctagctgctatcgtgttctctcaagcaagactctagcagatgtcgttgaagcacttattggggtctattatgtagcaggagggaaaattgctgcaaaccacctgatgaggtggattgggattcatgctgagttggatcctcaagagatccctcctccgaagccatataatatacctgagagcatattgaaaggcattgactttgagacattggaaggcgtcttgggtatgaaattccagaataaaggttttcttattgaggctataacgcatgcatccaggccatcttctggtgtttcctgttaccagcggctggaatttgttggtgatgctgtgttggatcatctcatcacaaagcatctattttttacatatactgatctacctccaggtcgcttgactgacctgagagctgcagctgttaataatgaaaactttgcaagggttgcagttagacgtaagctgcacggacatcttcgccatggatca

SEQ ID NO: 75 - DNA, 5340 bp, Triticum aestivum
  misc_feature 1..5340: note = ORF of protein Dcl1
  misc_feature 213..280: note = n is a, c, g, or t
  source 1..5340: mol_type = unassigned DNA
acggcggtcgcggcggggagtggagagacgggaagcgtcctcgcctcgcccctggggctcacggggagccgcgccctgattcgcggcggcggccgatgcttccgccgccctcgcgtggctgggaggaacggcgcgggaggcgcgacttcgttgacaggcctcgcaagcgcgaccgcgacagtaaccctgcccaccgccgcgaggctcgagggnnnnnnnnnnnnnnnnnnnnnnnnnnnnnnnnnnnnnnnnnnnnnnnnnnnnnnnnnnnnnnnnnnnnccaggacacacaacggaggctccgtggagaagactgcagaggcggataaggcggcttccgcccagaaggagaagcctctcgccgaggagcaggcgagacactaccagttggaggtgcttgagcaagcaaagagcaggaacaccatagccttcctcgagaccggcgcggggaagaccctcattgctgtgctgctcatcaaaagcatatgtgacaaaatgatgaaggagaacaggaaaatacttgctgtgttcttggttcccaaggtgcctcttgtgtatcagcaagctgaggtgatacgcgagaggactggctatcgggtcgggcactactgtggagagatgggtcaggatttctgggatgctagaaagtggcagcgcgagtttgattcgaaacaggtactagttatgacagctcaaattttgttaaacatcctcaggcacagtatcattaaaatggatgccatacatcttttaattttagacgagtgccatcatgcagtgaaaaaacatccatattctctagtgatgtcagaattttatcacacaactccgaaggataagagaccagttgtttttggtatgacagcttctcctgtcaacctaaagggagtcactagccaagaagattgtgccatcaagataagaaatcttgagagtaaactggattccattgtttgtactatcaaagatcggaaagagcttgagaaacatgtccccatgccttttgaagtcgttgtccaatatgacaaagcagccaccctttgttccctgcatgagcagatcaaacaaatggaagctgctgttgaagaagctgcactctgtagctctaagagaacaaagtggcagtttatgggagccagggatgctgggtctagagatgagctgcgccttgtctatggtgtttctgagcgtacagaaagtgatggtgcagctaatttaatccagaagttgagagctataaattatgctttaggtgaactaggacaatggtgtgcttataaggttgcacagtcctttttgacagtattgcagaatgatgaaagggcaaactatcaggttgatgtaaagtttcaagagtcgcatttaaagaaggttgttgatttattgcactgccagttgactgagggagctgcagtgaaaagcgagacgaatgatgttgagatgcataacactgaaaatcacaacccaagtgaacttgaagacggggagctgcctgatagccatgctgtttctgtaggtgaacatgtagatgaggtcatcggagctgccgtggcagatgggaaagttacccctagggtgcaggctttaatcaaaatccttctcaagtatcagcatactgacgatttccgtgctatcatctttgtggagcgagttgtcacggcattagttcttcctaaggtgcttgcagagcttccatctttaagctttatccgatgtgcaagcttgataggtcataacaacaatcaagagatgcgcacaagccaaatgcaggataccattgcaaaatttcgtgatggccgagttacactgttagtcgcgactagtgttgctgaggaaggacttgatattcgacagtgcaatgttgtcattcgctttgaccttgcaaagacggtcttggcctacgtacagtctcgtggtcgtgcaagaaagcctgggtctgactacatattgatggttgaaagggaaaatctggcacatgaaactttcttgaggaatgctcgaaatagtgaggagacgttgagaaaagaggctatagaaagaactgatctcagtcatcttgatggtactctgctaaattccattgatacatcacctgattcgatgtatcaggtcgaatcaacaggtgctgttgtcagcttgaattctgcagttgggctcgtacatttttactgttcacagctaccaagtgacaggtactcaattcttcgtccagaatttattatgcaaaaacatgagaagccagggggttccatggagtattcctgcaaacttcaacttccctgtaatgctccatttgagaaacttgagggtcctatatgcggctcaattcgtctggcacaacaagctgtttgtttggctgcctgtaagaagctgcatgagatgaatgccttcacagatatgcttttacctgatagaggaagtggagaaggagagaagaccgaacagaatgaaggtgatccactccctggaacggcacgccatagagagttttatcctgaaggtgttgctgaaattttgagaggagaatggattttatgtggaagagatggctgccaaagcactcaatttattaagttgtatatgtattctgtcaattgtgtggacgtaggggcatctaaggaccctttccttgcacaactttcgaatttttctattatttttggcagtgagttggatgcagaggttctatcgacgccaatggatctctttgttgccaggacaatgataacaaaggcatctcttgtattccgtggaccaattgaagttacagaaagtcagctggttctgctgaagagctttcatgttaggctcatgagcatagtccttgatgttgatgttgatccctcaactacaccttgggatccagcaaaagcatacctgtttgtccctgtaggaactgagaagtgcacagatgctttaagagagatcgattggactttggtcaacagcattgtgaatactgatgcctggaataatccacttcaaaaggcacgaccagatgtctacctaggcaccaatgagaggacacttggtggagataagagagagtatgggtttggaaaattgcgtaatggaaccgcttttggacagaaggcacatccgacttatggtattagaggagccattgctgactttgatattgtcaaagcatctggattagttccttctcgtgatagaggttattcaagtgactatcagaatcaaggcaagttgttcatggcagattcatgctgggatgctaaagatctttctggaatggttgtcactgctgcccactcaggaaagcggttctatgtggactgtatttgctataacatgaatgcagaaaattcatttcctaggaaggaaggctacttggggcctctggaatatagctcatatgccgattactacaagcagaaatatggtgtggagctagtgtataagaaacaacctcttatacgtgcacgtggtgtttcctattgcaagaatcttctgtctcctagatttgagcattctgaagctagagaaggtgacttttcggagaacgttgacaaaacatactatgtgtatttaccacctgaactttgccttgtgcacccccttcctggatcacttattcgtggagctcaaaggttgccatcgataatgagaagggttgagagtatgcttcttgcggttcaattgaaggaaacaattgattatcctgttcctgcagttaagatattagaagccttgactgctgcatcatgccaggaaacattctgctatgaaagagcagagttactgggtgatgcatacctgaaatgggttgtgagtagattccttttcctaaaatatcctcagaagcatgagggacagcttacaaggatgcgacagcagatggtcagtaacatggtcctctaccaatttgccctgaataaaactcttcaatcgtatattcaagcagatcggtttgccccatcaagatgggcagctccaggagtactacctgtatttgatgaggagactagagaatctgaaccgtccatctttggtgaggggtcaatacctggtaatgaagtacagaaggattatgacgatgactacacggatagcattcaagaggagggtgaaattgacggagattctagctgctatcgtgttctctcaagcaagactctagcagatgtcgttgaagcacttattggggtctattatgtagcaggagggaaaattgctgcaaaccacctaatgaagtggattgggattcatgccgagttggatcctcaagagatccctcctccgaagccatataatatacctgagagcatattgaaaggcattgactttgagacattggaaggcatcttgggtatgaaattccagaataaaggttttattattgaggctataacgcatgcatccaggccatcttctggtgtttcctgttaccagcggttggaatttgttggtgatgctgtgttggatcatctcatcacaaagcatctattttttacatatactgatctacctcctggtcgcttgactgacctgagagctgcagctgttaataatgaaaactttgcaagggttgcagttagacgtaagctgcacggacatcttcgtcatggatcatctgcactggaaaaacagattcgggagttcgtgaaggatgtccgagaagagatttcaaaatcaggtttcaattcttttggactaggggactgcaaggctcctaaagttcttggagacattattgagtctattgctggggcagtattcctggatagtgggtatgacacctcggcagtctggaaggtgttccaaccgctccttgaacctctggtaacgccagagacccttccgatgcatccgattagggagctccaagaacggtgccagcaacaagcagaaggcttagaatacaaagcatcccgggcaggcaatgtagctactgttgaagtctttgttgatggtgttcagattggtgtagctcagaatccccagaagaaaatggcacagaagttggcggcgaggaacgcacttgtggtcctgaaggagaaggaaaccgcagcaaagaaagagaccgaaaaggacggtgataagaacaatgccgggttcaccaggcagaccctgaacgacacttgcttgagaaggcagtggccgatgccacagtacaggtgcataaacgagggcggccctgctcatgctaaaagatttgtctacgcggtcagggtcaatacatccgatcgcggatggaccgacgagtgcgtcggagagccgatgccgagcgtgaagaaggccaaggactctgccgcgatgcttctcctcgagcttctgaaccgaagtttccctgaaaagcctgacggtaagaagtga

SEQ ID NO: 76 - DNA, 5025 bp, Triticum aestivum
  misc_feature 1..5025: note = ORF of protein Dcl1
  source 1..5025: mol_type = unassigned DNA
atggtgttccgccctggcacctgggagcaggagtctgagcgcgagggaaagcgtgccaggacacagaacggaggctccgtggagaagaatgcagaggcggataaggcggcttctgcccagaaggagaagcctctcgcagaggagcaggcgagacattaccagttggaggtgcttgagcaagcaaagagcagaaacaccatagccttcctcgagaccggcgcggggaagactctcattgctgtgctgctcatcaaaagcatatgtgacaaaatgatgaaggagaacaggaaaatacttgctgtgttcttggttcccaaggtgcctcttgtgtatcaggtactagttatgacagctcaaattttgttaaacatcctcaggcacagtatcattaaaatggatgccatacatcttttaattttagatgagtgccatcatgcagtgaaaaaacatccatattctttagtgatgtcagaattttatcacacaactccgaaggataagaggccagttgtttttggcatgacagcttctcctgtcaacctaaagggagtcactagccaagaagattgtgccatcaagataagaaatcttgagagtaaactggattccattgtttgtactattaaagatcggaaagagcttgagaaacatgtccccatgccttttgaagtcgttgtccagtatgacaaagcagccaccctttgttccctgcatgagcagatcaaacaaatggaagttgctgttgaagaagctgcactctgtagctctaagagaacaaagtggcagtttatgggagccagggatgctgggtctagagatgagctgcgccttgtctatggtgtttctgagcgtacagaaagtgatggcgcagctaatttaatccagaagttgagagctataaattatgctttaggtgaactaggtcaatggtgtgcttataaggttgcagtgtcctttttgacagtattgcagaatgatgaaagggcaaactatcaggttgatgtaaagtttcaagagtcacatttaaagaaggttgttgctttattgcactgccagttgactgagggagctgcagtgaaaagtgagacgaatgatgttgagatgcataacagtgaaaatcacaacccaagtgaacttgaagatggggagctgcctgatagccatgctgtttctgtaggtgaacatgtagatgaggtcatcggagctgccgtggcagatgggaaagttacccctagggtgcaggctttaatcaaaatccttctcaagtatcagcataccgaggatttccgtgccatcatctttgtggagcgagttgtcacggcattagttcttcctaaggtgcttgcagagcttccatctttaagctttatccgatgtgcaagcttgataggtcataacaacaatcaagagatgcgcacaagccaaatgcaggataccattgcaaaatttcgtgatggccgtgttacactgttagttgcgactagtgttgctgaggaaggacttgatattcgacagtgcaatgttgtcattcgctttgaccttgcaaagacagtcttggcctatgtacagtctcgtggtcgtgccagaaagcctggatctgactacatattgatggttgaaaggggaaatttggcacatgaaactttcttgaggaatgctcgaaatagtgaggagacgttgagaaaagaggctatagaaagaactgatctcagtcatcttgatggtactctgttaaattccattgatacatcacctgattcgatgtaccaggtcgaatccacaggtgctgttgtcagcttgaattctgcagttgggctcgtacatttttactgttcacagctaccaagtgacaggtactcaattcttcatccagaatttattatgcagaaacatgagaagccagggggttccatggaatattcttgcaaacttcaacttccctgtaatgctccatttgagaaacttgagggtcctatatgcggctcaattcgtctggcacagcaagctgtttgtttggctgcctgtaagaagctgcatgagatgaatgccttcacagatatgcttttacctgatagaggaagtggggaaggagagaagaccgaacagaatgaaggtgatccactccctggaacggcacgccatagagagttttatcctgaaggtgttgctgaaattttgagaggagaatggattttatgtggaagagatggctgccaaagcactcaatttgttaagttgtatatgtattctgtgaattgtgtggatgtagggacatctaaggacccttcccttgcacaactttcgaatttttctattatttttggcagtgagctggatgcagaggttttatcgacaccaatggatctctttgttgccaggacaatgataacaaaggcatctcttgtattccgtggaccaattgaagttacagaaagtcaggttgatctgcttgagaagttccaactggttctgctgaagagctttcatgttaggctcatgagcatagtccttgatgttgatgttgatccctcaactacaccttgggatccagcaaaagcatacctctttgtccctgtaggagctgagaagtgtacagatgctttaagagagatcgattggactttagtcaacagcattgtgaatactgatgcctggaataatccacttcagaaggcacgaccagatgtctacctaggcaccaatgagaggacactcggtggagaaaagagagagtatgggtttggaaaattgcgtaatggaacagcttttggacagaaggcacatccgacttatggtatcagaggagccattgctgactttgatatcgtcaaagcatctggattagttccttctcgtgatagagggtattcaagcgagtatcagaaccaaggcaagttgttcatggcagattcatgctgggatgctaaagacctttctgggatggttgtcactgctgcccactcaggaaagcggttctatgtggactgtatttgctataacatgaatgcagaaaattcatttcctaggaaggaaggctacctggggcctttggaatatagctcatatgccgattactacaagcagaaatatggtgtggagctagtgtataagaaacagcctcttatacgtgcacgtggtgtttcctattgcaagaatcttctgtctcctagatttgagcattctgaagctagagaaggtgacttttcggagaacgttgacaaaacatactatgtctatttacctcccgaactctgtcttgtgcacccacttcctggatcacttattcgtggagctcagaggttgccatcaataatgagaagggttgagagtatgcttcttgcagttcaattgaaggaaacaattgattatcctgttcctgcagttaagatattagaagccttgactgctgcatcatgccaggaaacattctgctatgaaagagcagagttactgggtgatgcatacctgaaatgggttgtgagtagattccttttcctaaaatatcctcagaagcatgagggacagcttacaaggatgcggcagcagatggtcagtaacatggtcctctaccaatttgccctgaataaaactcttcagtcgtatattcaagcagatcggtttgccccatcaagatgggcagctccaggagtactgcctgtatttgatgaggagacaagagaatctgaaccgtccatctttggtgaggagtcaatacctggtaatgaattacagaaggattatgatgatgactacacagataacattcaagaggagggtgaaattgatggagattccagctgctatcgtgttctctcaagcaagactctagcagatgtcgttgaagcacttattggggtctattatgtagcaggagggaaaattgctgcaaaccacttgatgaagtggattgggattcatgccgagttggatcctcaagagatccctcccccgaagccatataatatacctgagagcatattgaaaggcatcgagtttgagacattggaaggcatcttgggtatgaaattccagaataaaggttttcttattgaggctataacgcatgcatccaggccatcttctggcgtttcctgttaccagcggttggaatttgttggtgatgctgtgttggatcatctcatcacaaagcatttattttttacatatactgatctacctcctggtcgcttgactgacctgagagctgcagctgttaataatgaaaactttgcaagggttgcagttaggcgtaagctgcacacacatcttcgccatggatcatctgcactggaaaaacagattcgggagttcgtgaaggatgtccgagaagagatttcaaaatcaggtttcaattcttttggactaggggactgcaaggctcctaaagttcttggagacattattgagtctattgctggtgcagtattcctggatagtgggtatgacacctcggcagtctggaaggtattccaaccgctcctcgaacctctggtaacgccagagacccttccaatgcatccaattagagagctccaagaacggtgccagcaacaagcagaaggcttagaatacaaagcatcccgggcaggcaatgtagctaccgttgaagtctttgttgatggtgttcagattggtgtagctcaaaacccacagaagaagatggcacagaagttggcggcgagaaacgcactagtggtcctaaaggagaaggaaaccgcagcaaagaaagagaccgaaaaggccggtgataagagcaatgctgggttcaccaggcagaacctgaacgacacttgcttgaaaaggcagtggccaatgccacagtacaggtgcataaatgagggcggccctgctcatgctaaaagatttgtctacgcggttagggttaatacatccgaccgcggatggaccgacgagtgcatcggggagccgatgccgagcgtgaagaaggccaaggactctgccgccatgcttctcctcgagcttctgaatagaagtttccctgataagcccgacggtaagaagtga

SEQ ID NO: 77 - DNA, 1122 bp, Triticum aestivum
  misc_feature 1..1122: note = ORF of protein Dcl1
  source 1..1122: mol_type = unassigned DNA
atgtattctgtgaattgtgtggacgtagggacatctaaggacccttcccttgcacaactttcgaatttttctattatttttggcagtgagctggatgcagaggttctatcgacgccaatggatctctttgttgccaggacaatgataacaaaggcatctcttgtattccgtggaccaattgaagttacagaaagtcagctggttctgctgaagggctttcatgttaggctcatgagcatagtccttgatgttgatgttgatccctcaactacaccgtgggatccagcaaaagcatacctgtttgtccctgtaggagctgagaagtgtacagatgctttaagagagatcgattggactttagtcaacaacattgtgaatactgatgcctggaataatccacttcagaaggcacgaccagatgtctacctaggcaccaatgagaggacacttggtggagataagagagagtatgggtttggaaaattgcgtaatggaactgcttttggacagaaggcacatccgacttatggtattagaggagccattgctgactttgatattgtcaaagcatctggattagttccttctcgtgatagaggttattcaagtgactatcagaatcaaggcaagttgttcatggcagattcatgctgggatgctaaagatctttctggaatggttgtcactgctgcccactcaggaaagcggttctatgtggactgtatttgctataacatgaatgcagagaattcatttcctaggaaggaaggctacctggggcctttggaatatagctcatatgccgattactacaagcagaaatatggtgtggagctagtgtataagaaacaacctcttatacgtgcacgtggtgtttcctattgcaagaatcttctgtctcctagatttgagcattctgaagctagagaaggtgacttttcggagaacgttgacaaaacatactatgtgtatttaccacctgaactttgccttgtgcacccacttcctggatcacttattcgtggagctcaaaggttgccatcgataatgagaagggttgagagtatgcttcttgcagttcaattgaaggaaacaattgattatcctgttcctgcagttaag

SEQ ID NO: 78 - DNA, 5757 bp, Lolium perenne
  misc_feature 1..5757: note = ORF of protein Dcl1
  source 1..5757: mol_type = unassigned DNA
atggcgggcggcggcggcgtcacgtactggtacgacgcgtgcgaggacgacgacgacgacgaggacagctcctcgctgctctgcggcatcgacttcgccgcgtccgctgacttcgacccggggttcttgcccaacttgggcctcgacagcggcggcgtcgacgacggcttcgtcgccgaaatcgaccggattctcgagagcatcaacgccgacaccgcccctgcgcccgcgcccgcgcccgcgccgccaccgcccccgcccccgtctcagcccgtggctacgccaccgccgcagccacagccgcagcaaaaggaggctcctttggccgtggcccacaatgccctagcggtggtggcccctgcctcggggcagggggtcgagccgaggaaggagacgcggagggagtcccacggcagctccgccaatggcggcggggattacaggggcgggaagcgccctcgcctcgtctccgggcctgacagggcgccgcgccccgacccgcggcggcgccccccgcccccgccctcgcgcgggtgggatgaccggcgcgggaggcgcgacttcgacaggcctccgcacaagcgcgaccgcgaccaccaccaccgccgcgaggcccgggggttctgggagcgggaccgcggcgggaagatggtgttccgccccggcgcctgggaccaggaggagtccggccgcgacgcgaagcgcgccagggcagagaacggaggcaccgtggggaagaaagcagaggcggctgctgcacagaaggagaagcccctcaccgaggagcatgcgaggcagtaccagctggaggtgctcgagcaggcaaagagcaggaacaccattgccttcctcgagaccggcgccgggaagacgctcattgccgtgctgctcgtcaagagtttatgcgacaagatgctcaaggagaacaagaagatacttgctgtgttcttggttcccaaggtgcctcttgtgtaccagcaagctgaggtgatccgtgacaggactggctatcgggttgggcattactgcggagagatgggtcaggatttttgggattctaggaagtggcagcgcgagtttgattctaaacaggtacttgttatgacagctcaaattttgttaaacatcctccggcacagtattattaaaatggatgcgatacatcttctaattttagacgagtgccatcatgcagtgaaaaaacatccatattctctagtgatgtcagaattttatcatacaactcccaaggataagagaccagttgtttttggtatgacagcttctcctgtcaacctgaagggagtcaccagccaagaggactgtgccatcaagataaggaatcttgaaactaaattagattccatagtttgtactatcaaagatcggaaggagcttgagaagcatgttcccatgcctttggaagtcgtcgtgcaatatgacaaagcagccactctttgttccttgcacgagaagatcaaacaaatggaaactgcagttgaggaagctgcactctctagctctaagagaagcaagtggcagttcatgggagccagggatgctgggtctagagatgagctgcgccttgtctatggtgtttctgagcgtacagaaagtgatggtgcagctaatttattccagaaattgagagctataaactatgctttaggtgaactaggacaatggtgtgcttacaaggttgcacagtccttcttgacagcgttacagaatgatgaaagggcaaactatcaggttgatgtaaagtttcaagagtcgtatttaaaaaaggttgttgatttattacactgccagttgactgaaggagctgctatgagaagcgagacaaatgatgttgagatgcataatgctgaaaacgacaatccaagtgagctagaggacggggagctgcctgatagccatgctgtttctgtaggtgaacatgtagatgaagtcatcggagccgccgtggcagatgggaaggttacccctcgggtgcaaagtttgatccaaatacttctcaagtatcagcataccaaggatttccgtgctatcatctttgtggagcgagttgtcaccgcattagttcttcctaaggtgttggcagagcttccatctctaagctttatacgatgtgcaagcttgataggccataacaacaatcaagagatgcgcacaagccaaatgcaggatacaattgcaaagtttcgggatggccgtgttacgctgcttgttgcgactagtgttgcagaggaaggtcttgatattcgacagtgcaatgttgtcatccgctttgaccttgcgaagactgtcttggcctacatacagtctcggggtcgtgcaagaaagcctggatctgattacatattgatggttgaaagggaaaatatgtcacatgaaactttcttgaggaattgtcgtaatagtgaggagacattgagaaaagaagctatagaaagaactgatctcactcatcttgatggcactcctgcgttaaatccaattgatacctcccctgattcgatgtaccaggtcgaatcaacaggtgctgttgttagcttgaattctgcagttggcctcgtacatttttactgctcacagcttccgagtgacaggtactcaattcttcgtccggaattttttatgcaaaggcacgagaagccagggggttcagtggaatattcttgcaaaattcaactgccctgtaatgctccatttgagaaacttgagggtccaatatgcagttccattcgtctggcacagcaagctgtttgtttggctgcgtgcaagaagctacatgagatgagtgccttcacagatatgcttttacctgatagaggaagtgaggaacgagaaaagactgaacagaatgatgaaggtgatccactccctggtacagcaaggcatagagagttttatcctgaaggagttgccgaaattctaagaggagaatggattttgtctggaaaaaatagctgccagagctcgcagtttattaagctgtacatgtattctgtgagctgcatagatgttgggacatctaaggaccctttccttgcacagctttcgaatttttctattattttcggcaatgagctggatgcagaggttttatcgacgacaattgatctctttgttgccaggacaatgataacaaaggcatctcttgtattccgtggaccaattgagattacagaaagtcagctggtcttgctgaagagctttcatgttaggctcatgagcatagtccttgatgtggatgttgatcccttaacgacaccttgggatccggcaaaagcatacctctttgtgcctgtaggagctgagaagtgtagtacagacgctttaagagagatcgattggaccttaattaacagcattgtgaacactgaagcttggaataacccacttcagaaggcacggccagatgtctacctaggaaccaaagagcggacacttggtggagataagaaagagtatgggttcggcaaattgcgtaatggcaccacttttggacagaaggctcatccgacttatggcataagaggagccattgcacagtttgatattgtcaaagcatctggattagttcctggtcgtgatagagggtattttaatgactatcagaatcaagggaagttgttcatggcagattcatgctgggatgctaaggatcttgctggaatggttgtgactgctgcccactcaggaaagaggttctatgtggactgtatctgttataacatgaatgcagaaaattcgtttcctaggaaggagggctacctggggcctttggagtatggttcgtatgctgactactacaagcagaagtatggcgtagagctagtgtataagaagcaacctcttatacgtgcacgaggtgtttcctattgcaagaatcttttgtctcccagatttgagcattcggaagctaaagaaggtgaccttccagagaaccttgacaagacgtactatgtgtatttaccacccgaactgtgctttgtacacccacttcctggatcacttgttcgtggagcccagaggttgccatcaataatgagaagggtggagagcatgcttcttgcagttcaattgaaggaaataattgactaccctgttcctgcaactaagatattagaagccttgactgctgcatcatgccaggaaacattctgctatgaaagagcagagttactgggtgatgcataccttaaatgggttgtgagtagattccttttcctaaaatatccccagaagcacgagggacagcttacaaggatgcggcagcagatggttagtaacatggtcctgtacgaatttgccctgaataagactcttcaatcgtatattcaagcagatcggtttgccccatcaagatgggcagctccgggagtgctacctgtatttgatgaggaaacaagagaatccgagccgtccatctttggcgaagaatcaattcctggtaatgaattgccgaaggattacgatgatgactacgcagatagtatccaagaagacggtgaaattgatggagattctggctgctatcgtgttctctcaaccaagactttagcagatgtggttgaggcacttattggggtctattatgtagcaggagggaaaattgctgcaaatcacctgatgaagtggattgggattcatgctgagttggatcctcacgagatccctcctccgaacccatataatatacctgagagcatattgagaagcatcaactttgacacattagaaggcatcttgggcatgaaattccagaatagaggttttcttattgaggctataacacatgcttcacggccatcttctggagtgtcctgttaccagcggttggaatttgttggtgatgcagtgttggatcatctcattacaaagcatttattttttacatatacagacctacctcctggccgcttgaccgacttgagagctgcagctgttaataatgagaacttcgcaagggtggcagtgaaacataaactgcatgtacaccttcgccatggatcatctgcactggaaacacagattcgggagtttgtgaaggatgtccaagaagagattgcaaaatcagatttcaattcttttgggcttggggactgcaaggctcctaaagttcttggagacattattgaatctattgctggcgctatattcctggatagtgggtataatacctcggcggtctggaaggtttttcaaccgctccttagtcctatggtgacgccagaaacccttcctatgcatccaattagggagctccaagaacggtgccagcagcaagccgaaggcttagaatacaaagcatcccgagcaggaaatgtagcaactgttgaagtgtttgttgatggtgttcagattggtctagctcagaacccgcagaagaagatggctcagaagttggcggcgaggaacgcacttgtggtcctgaaggaaaaggaaactgcaacgaaaaaggagaccgaacaggatggagataagaaaaacggtgctagtgtgttcaccaggcagacgctgaatgacatgtgcctgagaaggcagtggccgatgccacagtacaggtgcataaacgagggcggccctgctcatgctaagaagtttgtctacgcggtgagggtgaacacatccgatcgtggatggactgacgaatgcattgggcagccaatgccgagcgtgaagaaggccaaggactctgctgccgctcttctacttgagcttctgaatgaaagttgtaagaagtcatag

SEQ ID NO: 79 - DNA, 5211 bp, Lolium perenne
  misc_feature 1..5211: note = ORF of protein Dcl1
  source 1..5211: mol_type = unassigned DNA
atggcgggcggcggcggcgtcacgtactggtacgacgcgtgcgaggacgacgacgacgacgaggacagctcctcgctgctctgcggcatcgacttcgccgcgtccgctgacttcgacccggggttcttgcccaacttgggcctcgacagcggcggcgtcgacgacggcttcgtcgccgaaatcgaccggattctcgagagcatcaacgccgacaccgcccctgcgcccgcgcccgcgcccgcgccgccaccgcccccgcccccgtctcagcccgtggctacgccaccgccgcagccacagccgcagcaaaaggaggctcctttggccgtggcccacaatgccctagcggtggtggcccctgcctcggggcagggggtcgagccgaggaaggagacgcggagggagtcccacggcagctccgccaatggcggcggggattacaggggcgggaagcgccctcgcctcgtctccgggcctgacagggcgccgcgccccgacccgcggcggcgccccccgcccccgccctcgcgcgggtgggatgaccggcgcgggaggcgcgacttcgacaggcctccgcacaagcgcgaccgcgaccaccaccaccgccgcgaggcccgggggttctgggagcgggaccgcggcgggaagatggtgttccgccccggcgcctgggaccaggaggagtccggccgcgacgcgaagcgcgccagggcagagaacggaggcaccgtggggaagaaagcagaggcggctgctgcacagaaggagaagcccctcaccgaggagcatgcgaggcagtaccagctggaggtgctcgagcaggcaaagagcaggaacaccattgccttcctcgagaccggcgccgggaagacgctcattgccgtgctgctcgtcaagagtttatgcgacaagatgctcaaggagaacaagaagatacttgctgtgttcttggttcccaaggtgcctcttgtgtaccagcaagctgaggtgatccgtgacaggactggctatcgggttgggcattactgcggagagatgggtcaggatttttgggattctaggaagtggcagcgcgagtttgattctaaacaggtacttgttatgacagctcaaattttgttaaacatcctccggcacagtattattaaaatggatgcgatacatcttctaattttagacgagtgccatcatgcagtgaaaaaacatccatattctctagtgatgtcagaattttatcatacaactcccaaggataagagaccagttgtttttggtatgacagcttctcctgtcaacctgaagggagtcaccagccaagaggactgtgccatcaagataaggaatcttgaaactaaattagattccatagtttgtactatcaaagatcggaaggagcttgagaagcatgttcccatgcctttggaagtcgtcgtgcaatatgacaaagcagccactctttgttccttgcacgagaagatcaaacaaatggaaactgcagttgaggaagctgcactctctagctctaagagaagcaagtggcagttcatgggagccagggatgctgggtctagagatgagctgcgccttgtctatggtgtttctgagcgtacagaaagtgatggtgcagctaatttattccagaaattgagagctataaactatgctttaggtgaactaggacaatggtgtgcttacaaggttgcacagtccttcttgacagcgttacagaatgatgaaagggcaaactatcaggttgatgtaaagtttcaagagtcgtatttaaaaaaggttgttgatttattacactgccagttgactgaaggagctgctatgagaagcgagacaaatgatgttgagatgcataatgctgaaaacgacaatccaagtgagctagaggacggggagctgcctgatagccatgctgtttctgtaggtgaacatgtagatgaagtcatcggagccgccgtggcagatgggaaggttacccctcgggtgcaaagtttgatccaaatacttctcaagtatcagcataccaaggatttccgtgctatcatctttgtggagcgagttgtcaccgcattagttcttcctaaggtgttggcagagcttccatctctaagctttatacgatgtgcaagcttgataggccataacaacaatcaagagatgcgcacaagccaaatgcaggatacaattgcaaagtttcgggatggccgtgttacgctgcttgttgcgactagtgttgcagaggaaggtcttgatattcgacagtgcaatgttgtcatccgctttgaccttgcgaagactgtcttggcctacatacagtctcggggtcgtgcaagaaagcctggatctgattacatattgatggttgaaagggaaaatatgtcacatgaaactttcttgaggaattgtcgtaatagtgaggagacattgagaaaagaagctatagaaagaactgatctcactcatcttgatggcactcctgcgttaaatccaattgatacctcccctgattcgatgtaccaggtcgaatcaacaggtgctgttgttagcttgaattctgcagttggcctcgtacatttttactgctcacagcttccgagtgacaggtactcaattcttcgtccggaattttttatgcaaaggcacgagaagccagggggttcagtggaatattcttgcaaaattcaactgccctgtaatgctccatttgagaaacttgagggtccaatatgcagttccattcgtctggcacagcaagctgtttgtttggctgcgtgcaagaagctacatgagatgagtgccttcacagatatgcttttacctgatagaggaagtgaggaacgagaaaagactgaacagaatgatgaaggtgatccactccctggtacagcaaggcatagagagttttatcctgaaggagttgccgaaattctaagaggagaatggattttgtctggaaaaaatagctgccagagctcgcagtttattaagctgtacatgtattctgtgagctgcatagatgttgggacatctaaggaccctttccttgcacagctttcgaatttttctattattttcggcaatgagctggatgcagaggttttatcgacgacaattgatctctttgttgccaggacaatgataacaaaggcatctcttgtattccgtggaccaattgagattacagaaagtcagctggtcttgctgaagagctttcatgttaggctcatgagcatagtccttgatgtggatgttgatcccttaacgacaccttgggatccggcaaaagcatacctctttgtgcctgtaggagctgagaagtgtagtacagacgctttaagagagatcgattggaccttaattaacagcattgtgaacactgaagcttggaataacccacttcagaaggcacggccagatgtctacctaggaaccaaagagcggacacttggtggagataagaaagagtatgggttcggcaaattgcgtaatggcaccacttttggacagaaggctcatccgacttatggcataagaggagccattgcacagtttgatattgtcaaagcatctggattagttcctggtcgtgatagagggtattttaatgactatcagaatcaagggaagttgttcatggcagattcatgctgggatgctaaggatcttgctggaatggttgtgactgctgcccactcaggaaagaggttctatgtggactgtatctgttataacatgaatgcagaaaattcgtttcctaggaaggagggctacctggggcctttggagtatggttcgtatgctgactactacaagcagaagtatggcgtagagctagtgtataagaagcaacctcttatacgtgcacgaggtgtttcctattgcaagaatcttttgtctcccagatttgagcattcggaagctaaagaaggtgaccttccagagaaccttgacaagacgtactatgtgtatttaccacccgaactgtgctttgtacacccacttcctggatcacttgttcgtggagcccagaggttgccatcaataatgagaagggtggagagcatgcttcttgcagttcaattgaaggaaataattgactaccctgttcctgcaactaagatattagaagccttgactgctgcatcatgccaggaaacattctgctatgaaagagcagagttactgggtgatgcataccttaaatgggttgtgagtagattccttttcctaaaatatccccagaagcacgagggacagcttacaaggatgcggcagcagatgatcggtttgccccatcaagatgggcagctccgggagtgctacctgtatttgatgaggaaacaagagaatccgagccacctacctcctggccgcttgaccgacttgagagctgcagctgttaataatgagaacttcgcaagggtggcagtgaaacataaactgcatgtacaccttcgccatggatcatctgcactggaaacacagattcgggagtttgtgaaggatgtccaagaagagattgcaaaatcagatttcaattcttttgggcttggggactgcaaggctcctaaagttcttggagacattattgaatctattgctggcgctatattcctggatagtgggtataatacctcggcggtctggaaggtttttcaaccgctccttagtcctatggtgacgccagaaacccttcctatgcatccaattagggagctccaagaacggtgccagcagcaagccgaaggcttagaatacaaagcatcccgagcaggaaatgtagcaactgttgaagtgtttgttgatggtgttcagattggtctagctcagaacccgcagaagaagatggctcagaagttggcggcgaggaacgcacttgtggtcctgaaggaaaaggaaactgcaacgaaaaaggagaccgaacaggatggagataagaaaaacggtgctagtgtgttcaccaggcagacgctgaatgacatgtgcctgagaaggcagtggccgatgccacagtacaggtgcataaacgagggcggccctgctcatgctaagaagtttgtctacgcggtgagggtgaacacatccgatcgtggatggactgacgaatgcattgggcagccaatgccgagcgtgaagaaggccaaggactctgctgccgctcttctacttgagcttctgaatgaaagttgtaagaagtcatag

SEQ ID NO: 80 - DNA, 2115 bp, Arabidopsis thaliana
  misc_feature 1..2115: note = ORF of protein Dur3
  source 1..2115: mol_type = unassigned DNA
atggctacatgtcctcctttcgatttctcgaccaaatattacgatggtgacggcggatgccagaggcagagcagcttcttcggaggcacgacggtgctcgatcaaggcgttggctacgccgtgattcttggttttggtgctttctttgccgtcttcacatcttttttggtatggttagaaaaacgatatgtgggagctcgtcatacatcggaatggttcaatacagcgggaagaaacgttaagacaggacttattgcaagtgtcattgtatcacagtggacatgggctgcgaccatactgcaaagttcaaacgtagcatggcagtatggggttagtggaccgttttggtacgctagtggagccaccatacaggtgctgttgtttggtgtgatggcgattgagataaaaagaaaggctcctaatgctcacacggtctgcgaaatcgtgaaggctcgttggggaaccgcaacacatatagtgtttttggtgttttgtttggcgacaaacgtcgttgtgacggcgatgctcttgctcggcggctctgctgtggtgaatgctctaaccggagtgaatctatacgccgcaagctttctcattcctctcggcgtcgttgtctatactttggctggaggacttaaagctactttcctcgcaagctacgttcactccgttatagttcacgtggctctagtcgtctttgtgtttctggtctacacatcgagtaaagagcttggcagtccgtccgtggtctatgaccgactaaaagatatggtcgctaagtcaaggtcatgcactgagccactctcgcaccacggtcaagcttgtggtccggttgatggcaactttagaggctcctatctcaccatgttgagctctggcggcgctgtcttcggtctcatcaacattgtcggaaacttcggcactgtcttcgtagataatggttattgggtgagtgcaatagctgctcggccttcgtcgacccacaaagggtatttgcttggtggactcgtgtggttcgctgtaccgttctctctagctacctcgttaggtctcggcgcactagcccttgacttgcctatatctaaggacgaggctgaccgaggtttggttccacctgccacagccattgctctcatgggtaaatcaggatccttgcttctactcactatgctatttatggctgtaacatcggctggttcttccgagctaattgcagtctcatcgctctttacatatgacatctaccgaacatacataaaccctagagcaaccggtagacaaatcttgaaaatctcaagatgcgctgttcttggatttggttgtttcatggggattcttgcagtggtcttgaacaaagctggtgtttcacttggatggatgtatctcgcaatgggagtactcattggttctgcggtcattcccattgctttcatgctcttgtggagtaaagccaatgcctttggtgcaattcttggagcgacctcgggttgtgtttttgggatcattacttggctaactaccgcaaagacacagtacggacgcgttgatcttgatagcaccgggaaaaatggccctatgttagctggtaacctcgttgcgattctaacaggggggttgatccatgcggtgtgtagtctagtccggccacagaattacgattggtcgacgacgagggagatcaaagtagtggaagcgtatgcttcaggggacgaagatgttgatgttcctgccgaagagctaagagaagagaagctgagaagagccaaagcttggattgtgaaatggggtcttgtcttcactatcctcattgttgtcatttggccagttctctctctcccagctcgcgtatttagcagaggatatttctggttttgggcaattgtggcaatagcttggggaactataggatccatcgtgatcattggtttgccattggtggaaagttgggacacgatcaagagtgtttgtatgggaatgttcacaaacgatagggtgatgaaaaagcttgacgacttgaaccataggcttcgagctttaaccatggctgtgccagaagctgagaagatatatttgcttgaacttgagaagaccaagaaaaatgatgaagaaggttga

SEQ ID NO: 81 - DNA, 3885 bp, Arabidopsis thaliana
  misc_feature 1..3885: note = ORF of protein Ein2
  source 1..3885: mol_type = unassigned DNA
atggaagctgaaattgtgaatgtgagacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcgcataagcgttgtgactggtaaacacttggctcagatctgcaatgaagaatatgacaagtggacgtgcatgttcttgggcattcaggcggagttctcagcaattctgctcgaccttaccatggttgtgggagttgcgcatgcacttaaccttttgtttggggtggagttatccactggagtgtttttggccgccatggatgcgtttttatttcctgttttcgcctctttccttgaaaatggtatggcaaatacagtatccatttactctgcaggcctggtattacttctctatgtatctggcgtcttgctgagtcagtctgagatcccactctctatgaatggagtgttaactcggttaaatggagagagcgcattcgcactgatgggtcttcttggcgcaagcatcgtccctcacaatttttatatccattcttattttgctggggaaagtacatcttcgtctgatgtcgacaagagcagcttgtgtcaagaccatttgttcgccatctttggtgtcttcagcggactgtcacttgtaaattatgtattgatgaatgcagcagctaatgtgtttcacagtactggccttgtggtactgacttttcacgatgccttgtcactaatggagcaggtatttatgagtccgctcattccagtggtctttttgatgctcttgttcttctctagtcaaattaccgcactagcttgggctttcggtggagaggtcgtcctgcatgacttcctgaagatagaaatacccgcttggcttcatcgtgctacaatcagaattcttgcagttgctcctgcgctttattgtgtatggacatctggtgcagacggaatataccagttacttatattcacccaggtcttggtggcaatgatgcttccttgctcggtaataccgcttttccgcattgcttcgtcgagacaaatcatgggtgtccataaaatccctcaggttggcgagttcctcgcacttacaacgtttttgggatttctggggttgaatgttgtttttgttgttgagatggtatttgggagcagtgactgggctggtggtttgagatggaataccgtgatgggcacctcgattcagtacaccactctgcttgtatcgtcatgtgcatccttatgcctgatactctggctggcagccacgccgctgaaatctgcgagtaacagagcggaagctcaaatatggaacatggatgctcaaaatgctttatcttatccatctgttcaagaagaggaaattgaaagaacagaaacaaggaggaacgaagacgaatcaatagtgcggttggaaagcagggtaaaggatcagttggatactacgtctgttactagctcggtctatgatttgccagagaacattctaatgacggatcaagaaatccgttcgagccctccagaggaaagagagttggatgtaaagtactctacctctcaagttagtagtcttaaggaagactctgatgtaaaggaacagtctgtattgcagtcaacagtggttaatgaggtcagtgataaggatctgattgttgaaacaaagatggcgaaaattgaaccaatgagtcctgtggagaagattgttagcatggagaataacagcaagtttattgaaaaggatgttgaaggggtttcatgggaaacagaagaagctaccaaagctgctcctacaagcaactttactgtcggatctgatggtcctccttcattccgcagcttaagtggggaagggggaagtgggactggaagcctttcacggttgcaaggtttgggacgtgctgcccggagacacttatctgcgatccttgatgaattttggggacatttatatgattttcatgggcaattggttgctgaagccagggcaaagaaactagatcagctgtttggcactgatcaaaagtcagcctcttctatgaaagcagattcgtttggaaaagacattagcagtggatattgcatgtcaccaactgcgaagggaatggattcacagatgacttcaagtttatatgattcactgaagcagcagaggacaccgggaagtatcgattcgttgtatggattacaaagaggttcgtcaccgtcaccgttggtcaaccgtatgcagatgttgggtgcatatggtaacaccactaataataataatgcttacgaattgagtgagagaagatactctagcctgcgtgctccatcatcttcagagggttgggaacaccaacaaccagctacagttcacggataccagatgaagtcatatgtagacaatttggcaaaagaaaggcttgaagccttacaatcccgtggagagatcccgacatcgagatctatggcgcttggtacattgagctatacacagcaacttgctttagccttgaaacagaagtcccagaatggtctaacccctggaccagctcctgggtttgagaattttgctgggtctagaagcatatcgcgacaatctgaaagatcttattacggtgttccatcttctggcaatactgatactgttggcgcagcagtagccaatgagaaaaaatatagtagcatgccagatatctcaggattgtctatgtccgcaaggaacatgcatttaccaaacaacaagagtggatactgggatccgtcaagtggaggaggagggtatggtgcgtcttatggtcggttaagcaatgaatcatcgttatattctaatttggggtcacgggtgggagtaccctcgacttatgatgacatttctcaatcaagaggaggctacagagatgcctacagtttgccacagagtgcaacaacagggaccggatcgctttggtccagacagccctttgagcagtttggtgtagcggagaggaatggtgctgttggtgaggagctcaggaatagatcgaatccgatcaatatagacaacaacgcttcttctaatgttgatgcagaggctaagcttcttcagtcgttcaggcactgtattctaaagcttattaaacttgaaggatccgagtggttgtttggacaaagcgatggagttgatgaagaactgattgaccgggtagctgcacgagagaagtttatctatgaagctgaagctcgagaaataaaccaggtgggtcacatgggggagccactaatttcatcggttcctaactgtggagatggttgcgtttggagagctgatttgattgtgagctttggagtttggtgcattcaccgtgtccttgacttgtctctcatggagagtcggcctgagctttggggaaagtacacttacgttctcaaccgcctacagggagtgattgatccggcgttctcaaagctgcggacaccaatgacaccgtgcttttgccttcagattccagcgagccaccagagagcgagtccgacttcagctaacggaatgttacctccggctgcaaaaccggctaaaggcaaatgcacaaccgcagtcacacttcttgatctaatcaaagacgttgaaatggcaatctcttgtagaaaaggccgaaccggtacagctgcaggtgatgtggctttcccaaaggggaaagagaatttggcttcggttttgaagcggtataaacgtcggttatcgaataaaccagtaggtatgaatcaggatggacccggttcaagaaaaaacgtgactgcgtacggatcattgggttga

SEQ ID NO: 82 - DNA, 2691 bp, Arabidopsis thaliana
  misc_feature 1..2691: note = ORF of protein Emb175
  source 1..2691: mol_type = unassigned DNA
atgtccaccgttaatcatcactgcctcctcaattttcctcatatcccaccttcaattcctccgaatcatcgtcccaaacttctttcttctctctccctctatcgaaaaccagaacgcctctttgctctctctgcgtctctgtctctttcaccagcaacaatccatgaatgttcttcttcctcatcttcttcttcttcatcgttcgataaagaagaaacggaagacattgaatctgttattgatgggtttttctatctgcttcgattgtctgctcaataccacgacgtcgaggtaacaaaagctgtgcacgcttcgtttcttaagctcagagaagaaaaaacccgtcttggaaatgcccttatctctacttatctcaaacttggtttccctcgtgaagctattctcgtcttcgtgtctctgtcttcgccaacggtggtttcttacacagctcttatctcgggtttctcaaggttaaaccttgaaattgaggctcttaaggttttcttcaggatgagaaaagcaggtcttgttcagccgaatgagtatacttttgtagcgattttgactgcttgcgtgagagtttcccgattctcgcttgggattcagattcatggattgatagttaaatcagggtttttgaattctgtgtttgttagtaattcgctgatgagtttgtatgataaggattctgggtcttcgtgtgatgatgtgcttaagttgttcgatgaaattcctcagagagatgtggcgtcttggaacactgtggtttcgagtttagtgaaagagggcaagtctcataaagctttcgatttgttttatgagatgaatagagtcgaaggatttggagttgatagttttacgctttcaacgcttttgagctcttgtactgattctagtgtcttattgagaggtagagaactccatggtcgtgcaattaggatagggttgatgcaggagttgagtgtgaacaacgctctgattgggttttattctaaattttgggatatgaaaaaggtagagagtttatacgagatgatgatggcgcaagatgctgttactttcacggagatgatcacagcttacatgtcatttggaatggtggattccgctgttgagatattcgcgaatgtaactgaaaaaaacactattacgtataatgcgctcatggctgggttctgtagaaatggccatggcttgaaggcactaaaattattcactgatatgctgcagagaggtgttgagttgacggattttagtttaacaagcgctgttgatgcttgtggtttagtttcagagaagaaagtaagcgaacagattcacgggttttgtattaagtttggaactgctttcaacccttgtatccagactgcattgcttgatatgtgcacaaggtgtgaaaggatggcagatgctgaggagatgtttgaccaatggccttctaatctcgatagctcgaaagcaacgacgtctataatcggtggttacgctcggaatggcttacctgataaggctgtatcgctattccatcgcactctctgtgaacagaagttgtttttagatgaagtttcgttgactttgattcttgcggtctgcgggacattaggttttcgggaaatgggttatcagatccattgctatgctcttaaagctgggtacttttctgatataagcttgggaaattctttgattagcatgtacgcgaaatgttgtgactcagatgatgccataaagattttcaacactatgcgagaacatgatgttatttcttggaacagcttgatttcttgctacatccttcaaagaaatggtgatgaggcactggctctatggtcgaggatgaatgagaaagagatcaagcctgacataatcaccctcactcttgttatttctgcatttcggtatacagagtcaaataagcttagtagctgccgtgatctgtttctctccatgaagactatctatgacattgaaccaacgactgaacattacactgcattcgtccgtgttcttggtcattggggtcttcttgaagaagcggaagacacaataaactcaatgccggttcagcccgaggtctctgttttgagagctctgcttgatagctgcagaatacattcaaacacaagcgttgcgaaacgagtagcaaagcttatactatctacaaagccagaaaccccgtctgagtacatcctcaaatccaacatctactcagcttccggattctggcatcgctctgaaatgataagagaggaaatgcgggaaagaggttaccggaaacatcctgctaaaagctggataatccatgagaacaaaatacattcctttcacgcgcgtgacacatcccacccgcaagagaaagacatatacaggggattggagatactgataatggaatgtctgaaagttggatacgaaccaaacacagagtatgttcttcaggaggttgatgagtttatgaagaagagtttcttgtttcaccacagcgcgaaactcgcagtgacttatggaattctctcgagcaatactcggggcaagccggttcgggttatgaagaacgtaatgctctgtggtgattgccacgagttcttcaagtatatttcagttgtggtaaaaagagagatagttttgagagattcctcagggtttcatcattttgtgaatggcaagtgttcttgtagagatctgtggtga

SEQ ID NO: 83 - DNA, 2130 bp, Arabidopsis thaliana
  misc_feature 1..2130: note = ORF of protein Emb2726
  source 1..2130: mol_type = unassigned DNA
atggctacgattacaccttcttctattagcaatgcctggctcattcctggagcttcttttactgtaaagaagagtgattgttccatcaaatgtagcttttcgcgtaaagctgggaaacagattctatcatcaacgcagagacttgtgttacctttgtcgacttcacttagattgtttcctacacatggaaggcagtttgtgttgcatcctcatcgtagagctactgggactgatgttgtggcggctgtagaggaacaagattctactcctgttgttgcggaggataaagaaaccgtggctagtgagaagtcagatgctcctgctccaacatctcagtcaaggggaacagcaagacctggaaggaagagtgagatgcctgcggttaagaacgaggagctggttcctggagctactttcactgggaaagtacgagctattcagccttttggagcttttgttgattttggtgcttttactgatggattagtccatgtttctcaattgagtgacaactttgttaaggatgtttcgagtgttgtcactattggacaagaggtgaaggtaaggctagtggaagctgacattgagtcaaaacgtatttctttaactatgcgtgagaatgatgatcctcctaagcgtcagtctggtggtagtgataagccgagatcaggagggaaaagggatggttctaaaggaggaggacagaggaagggagaagggtttaactcaaagtttgccaaagggcagatgttggatggagtggtcaagaatttaacgaggtcaggagcgtttataactataggagaaggcgaagaagggtttttaccgacggctgaggaagcagatgatggtattggaagcatgatgatgggtggctcttcgttgcaagctggacaagaggtgaaggttcgtgtgttgaggatagcaaggggacgtgttactttaacaatgaaagaggaagatgatggtaagtttgatgaaacaaccactcagggagttgtgcatacagccacaaatccgtttatgcttgctttccgtaagaacgaagagattgctgcgtttcttgacaagagggaggaagaggcagagaagccacctgttgagacaccagtagagcctgaagctgaagcctctgtcacctcagctgaagtcgaggaatcggtatgtgtcccagctgaagtaacaagtgaagaggttccctcgtcagaaactcccaaggtagtagaagaagaagtgatagctaccaaagctgaagatgattcgccggaaaaagaagagcaaacggaaacacttgctgcagcagctgaagctgaagaggtggttcctccaattccagaaaccaaaagtgaagaggaaattgttgagaactctattcctccaaactcagccactgatgaagtttcatctccagaagctttagcaagtgaagaggttgagaaggagcaagtggttgccgaaacacctgttgatgaagtcaagactcctgcacctgtagtcacagaagcttcttctgaggaaagtggcaacaccgctactgctgaaagcatcaaaggcatttcaccagctctagtgaagcagctgagagaagaaacaggggcaggaatgatggactgcaagaatgctctttcagagagtgagggcgatatggtcaaagctcaagagtacctccgcaagaagggactagcgagcgcagataagaaagcaagcagagccacatccgagggaagaattggtgcttatatccacgacagcagaatcggtgtcctcttggaggttaattgtgagactgattttgtctcacgtggcgacattttcaaggaacttgttgatgatctggcgatgcaggtgcaatatgtttcaatagaggacattccagaggagataaagcagaaagaaaaagagattgagatgcaaagagaggatcttttgtcgaaaccagagaacataagggagaagattgtggaaggaagaatctcaaagaggcttggagaatgggcattgcttgagcagccttacatcaaagacgacagtgttctggtgaaggatctggtgaagcaaacagtggctactcttggggaaaacatcaaagtcagaaggtttgtgaagtttactcttggagaagataactga

SEQ ID NO: 84 - DNA, 1593 bp, Arabidopsis thaliana
  misc_feature 1..1593: note = ORF of protein Emb9
  source 1..1593: mol_type = unassigned DNA
atgaggacactctggaatcactttagtaccatttcctatatcaaaatttctccgagaatgagaagaatttctgcagcaaatttgatctcaaatcgaaatctttcaaccatttcttctactgaagatcccgagctcagagattttgtgggatttttagaatctctcaaaaactatgagaaatcaggtgtaccaaaaggagctggaactgattctgatgatggattcgatctgggtcgaatgaaacgtctcatgcttcgcctccgtaatcctcattacaaatacaaggttgttcatgttgctggaactaagggaaaaggatcaacttctgcttttctctctaatatcttacgagctggaggatattctgttggttgttattctagcccacatattctgagtatcaaagaacggatttcttgtaatggagaacctgtctctgcttccactcttaatgatcttttctattcagtcaaaccgattcttgaacagtctattcaagaggaaaatggttctttgagtcattttgagattctcactgggatagccttctctttatttgaaaaggaaaatgtcgacattgcggttatagaggctgggctaggaggagctcgagacgctacaaatgtcattgaaagttcaaatcttgctgcatcggtcataacaacgataggtgaggaacacatggcagcacttgggggttccttggaaagtatagcagaggctaaatctggaattattaaacacggtcgcccagtggttttaggtggaccgtttcttcctcatatcgagggcattctccgttctaaagcagcttcagtgtcgtcatcggttattttggcatctaatattggatctagttcttcaatcaaaggcatcattaacaaaaatgggatcgggctttgccagtcctgtgacatcgtgatacagaatgagaaagatgatcaaccaattgttgagcttagtgatgtaaacctacgtatgcttggacatcaccagctccaaaatgctgttactgccacatgtgtgtctctttgtcttcgcgatcaaggatgtgggagagtgacagatgaagcaattaggattggtttagagaacactcgtttacttgggagaagtcagtttctgacaccaaaagaagcagagactttactattacccggagcaacagtgcttcttgatggagctcacaccaaagagtcagcgcgagccctcaaggagatgataaagaaggattttccagagaaaagattggtatttgtggttgccatggctagtgacaaagatcatgtgtcctttgcaaaagaacttctctcaggtctaaaaccagaagcagtgattctaacagaagctgacatcggtggaggtaagattagatcaacggagtcttcggtgttgaaagaatcatggataaaagctgctgatgaattggggtcaaggtctatggaagcttcagaaaacaaaactgtgttgggttccttaaagcttgcctacaagatactcagcgatgacacaacaagtagtgactcaggaatggttatagtcacgggttcgcttcacattgtatcttcagtcttagcttctcttcaacattaa

SEQ ID NO: 85 - DNA, 1563 bp, Arabidopsis thaliana
  misc_feature 1..1563: note = ORF of protein Epsps
  source 1..1563: mol_type = unassigned DNA
atggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcggagaaagcgtcggagattgtacttcaacccattagagaaatctccggtcttattaagcttcctggctccaagtctctatcaaatcggatcctgcttctcgctgctctgtctgagggaacaactgtagtggacaacttgttgaatagcgatgacatcaattacatgcttgatgcgttgaagagattgggacttaatgtggaaactgacagtgaaaataatcgtgctgtagttgaaggatgtggcgggatattcccagcttccatagattcaaagagtgatatcgaactttacctcggtaatgcaggaacagcaatgcgtccacttaccgctgcggtcactgctgcaggtggaaacgcaagttatgtgcttgatggggtgcctcgtatgagagaaagacctataggggatttggttgttggtcttaagcagcttggtgctgatgttgaatgtactcttggaactaactgccctcctgttcgtgtcaacgctaatggtggccttcccggtggaaaggtgaagctttctggatcaattagtagtcagtacttgactgctctgctcatgtctgctcccttagctcttggagacgtcgagattgagattgtcgataaattaatttctgttccatatgttgaaatgacattgaagttgatggaacgtttcggggttagtgtcgagcatagtgatagctgggatcgtttctttgtcaagggcgggcaaaaatacaagtctccgggtaatgcgtatgtagaaggtgatgcttctagtgctagttatttcttggctggtgctgccattaccggtgaaactgtcacagtcgaaggttgtggaactaccagcttgcagggagatgtaaaattcgccgaggtccttgagaaaatgggatgtaaagtgtcctggacagagaacagtgtgactgtgacaggaccacctagagatgcttttggaatgagacacttgcgggctattgatgtcaacatgaacaaaatgcctgatgtagccatgacccttgccgtcgttgctctctttgctgacggtccaaccaccattagagatgtggctagctggagagtaaaggagacagaaaggatgattgccatttgcacagagcttagaaaactgggagctacagtggaagaaggttcagattattgtgtgataactccgcccaaaaaggtgaaaacggcagagattgatacatatgatgatcatagaatggcaatggcattctctcttgcagcttgtgctgatgttccaatcaccatcaacgatcctggttgcaccaggaaaaccttccccgactacttccaagtacttgaaagaatcacaaagcactaa

SEQ ID NO: 86 - DNA, 1083 bp, Arabidopsis thaliana
  misc_feature 1..1083: note = ORF of protein Fnr1
  source 1..1083: mol_type = unassigned DNA
atggctgctgctataagtgctgcagtctctttaccttcctccaagtcatcctctctcctcaccaaaatctcctctgtatcccctcaaaggattttcctcaagaagagcacagtgtgttacagaagagttgtgtcagtgaaggctcaggtgacaacagatactaccgaggcaccaccagttaaagtagtcaaggagtctaagaaacaggaagaagggattgttgtcaacaaattcaaacctaagaacccttacactggtcgctgccttttgaacaccaagatcaccggtgatgacgctcccggtgagacttggcacattgtcttcaccaccgaaggtgaggttccgtatagagaaggacaatcgataggagtgattccagagggaatagacaagaacgggaagccgcacaagctcaggctttactctatcgcgagtagtgccattggtgactttggagactccaagaccgtttctctctgtgtcaagagactagtttacacaaatgatggcggagagattgttaagggggtctgctccaacttcttgtgtgacttgaagccgggtgatgaagctaagatcactggacctgttggcaaggaaatgcttatgccaaaagaccccaatgccaccatcatcatgcttggaacaggaactggaatagctccattcagatcatttttgtggaaaatgttctttgaggagcacgaggactacaagttcaatggtttggcgtggcttttcttgggtgtacccacaagcagctcactgctatacaaggaggagtttgagaagatgaaggagaagaacccagacaacttcaggctggactttgcggtgagcagagagcagacgaacgagaagggagagaaaatgtacattcagacaagaatggcagagtatgcagaagagctgtgggagttgctgaagaaagacaacacctttgtttacatgtgtggtcttaagggtatggagaagggtatcgatgacattatggtctcgcttgctgctaaagatgggatcgattggttggagtacaagaagcaattgaagaggagtgaacagtggaatgttgaagtctactaa

SEQ ID NO: 87 - DNA, 1524 bp, Arabidopsis thaliana
  misc_feature 1..1524: note = ORF of protein Fve
  source 1..1524: mol_type = unassigned DNA
atggagagcgacgaagcagcagcagtgtctcctcaagcaacgacaccgagcggaggaaccggagcttctgggccgaagaagagaggtcggaaacctaaaaccaaggaagattctcagacgccgtcgtctcagcaacagagcgatgttaaaatgaaagaaagtgggaagaaaacgcagcagtcgccgagtgttgacgagaagtactctcagtggaaaggtctcgtccccattctctacgactggctcgctaaccataacctcgtctggccttcactctcttgcagatggggtccgcagcttgagcaagcaacctacaagaatcgccagcgtctgtacctctcagagcaaactgatggaagtgtgcccaatactttggtcatagcaaattgtgaagttgttaagccaagggttgctgcagcagagcacatttctcagttcaatgaagaagcacgttctccatttgtgaagaagtacaagaccatcattcaccctggagaggttaacagaatcagggaactcccacagaatagtaagattgttgctactcacaccgacagtcctgatgttctcatttgggatgttgaaacccaaccaaaccgtcatgctgtgcttggagctgcaaattcccgtccagatttgatactaactgggcaccaagataatgctgaatttgctcttgccatgtgcccaacggaaccctttgtgctctccggaggcaaggacaagtcagttgttttgtggagtatccaagatcacatcacaacgattgggacagattccaaatcatctggatctatcatcaaacagactggtgaaggtactgataagaatgagagtcctactgttggcccacgaggtgtatatcatggccatgaagatacagttgaagatgtggcattcagcccgacgagtgcacaagaattctgcagtgttggtgatgattcttgccttatactatgggatgcgagaactggcacaaaccctgtcacgaaggttgaaaaagcgcatgatgctgatcttcattgtgttgattggaatcctcatgacgacaatctgatcctgacagggtcagcagacaacactgtccggttgtttgatcgtaggaagcttaccgctaatggagttggttcgcctatttacaaatttgagggacacaaagctgctgttctttgtgttcagtggtctcctgataagtcatccgtctttgggagctctgcagaagatggtctcttgaacatctgggattatgacagggtcagtaagaagtctgatcgtgcagctaaaagccccgctgggctcttcttccagcatgctggtcacagggacaaagttgttgatttccactggaatgcttcagacccttggactattgtcagtgtttctgatgactgtgagactactggtggaggtggaacattgcagatatggcggatgagtgacttgatttacagaccagaagaagaagtcgtggcagaattggagaagttcaagtcgcatgttatgacttgtgcctccaagccttaa

SEQ ID NO: 88 - DNA, 1011 bp, Arabidopsis thaliana
  misc_feature 1..1011: note = ORF of protein Ga2ox7
  source 1..1011: mol_type = unassigned DNA
atggcttctcaacctccctttaagacaaatttctgctcgattttcggaagctcatttccaaattcaactagtgagagcaatacaaacacatcaactatccaaacctcaggcataaagcttcctgtgatcgatctcagccatctaactagtggtgaggaggtcaaacgcaaaagatgtgtgaaacaaatggttgcagctgcgaaagagtggggattttttcaaattgtgaaccatggaattcccaaagacgtctttgagatgatgctcctcgaagagaagaaactctttgaccaacctttttctgtgaaagtcagagaacgtttttcggacttatcgaagaatagttaccgttggggaaaccctagcgccacttctcccgctcagtactccgtttcggaagcgtttcacatcattctttcagaggtttcaaggatttctgatgatcgcaacaacctcagaacaatcgttgaaacgtatgtgcaagagatagctcgagtggcacaaatgatatgtgaaatactggggaaacaagtgaacgtgagttcggagtatttcgaaaacatttttgagcttgaaaacagttttctaaggctcaataagtaccatcctagtgtttttggttctgaagtgtttggtttggttcctcataccgatacaagttttctcactatactctctcaagatcaaatcggagggttagaattggaaaataatggacaatggatcagcgtaaaaccttgcttggaagcccttacagtcaacattggggatatgtttcaggcactgagtaatggagtgtaccaaagcgtgagacatagagtgatttctccagcaaatatcgagaggatgtcaatagctttcttcgtatgtccttatctcgaaactgagatcgattgctttgggtatccaaagaagtatagaagattcagtttcagagagtacaaagagcagagtgaacatgatgttaaagaaactggtgataaggtaggcttgtccaggtttctcatttga

SEQ ID NO: 89 - DNA, 1023 bp, Nicotiana benthamiana
  misc_feature 1..1023: note = ORF of protein Gapc
  source 1..1023: mol_type = unassigned DNA
atggcatctgacaagaagatcaagatcggaatcaatggatttggaaggattggtcgtttggtggctagagttgctctgcagagagatgatgttgaactagttgcagtgaacgacccatttatctctactggttacatgacatacatgtttaagtatgattcagttcatggacaatggaaacaccatgagcttaaagtcaaggatgaaaagacccttctttttggtgagaagtccgtcagagtctttggaatcaggaaccctgaagaaattccatgggctgaagctggtgctgatttcgttgtggaatccactggtgtcttcactgacaaggacaaggctgctgctcacttgaagggtggtgccaagaaggttgtgatctctgctcctagcaaggatgcccccatgtttgttgtgggtgtcaatgagaaggaatacaagccagaatatgacattgtctccaatgctagttgcactaccaactgccttgcacctttggctaaggtcatcaatgataggtttggcattgtggagggtctcatgacaactgtccactccctcactgccacccagaagactgttgatggtccatccatgaaggactggagaggtgggagagctgcttcattcaatatcattcctagcagcactggtgctgccaaggctgttggaaaagtgctcccagctcttaatggaaaattgacgggaatggccttcagagttccaactgttgatgtttctgttgtggaccttactgtcagactagagaaagaggcctcttatgatgacatcaaagctgcaatcaaggaggaatcagagggtaagttgaagggtatcttgggattcaccgaagatgatgtggtttccacagactttgttggtgacagcaggtcaagcatttttgatgccaaggctggaattgccttgagcaagaactttgtgaaacttgtgtcgtggtatgacaatgaatggggttacagctctcgtgtgattgatttgatctgccatatggcttctgttgcttga

SEQ ID NO: 90 - DNA, 2037 bp, Arabidopsis thaliana
  misc_feature 1..2037: note = ORF of protein Gcn2
  source 1..2037: mol_type = unassigned DNA
atggtattaacgacgaatctctatagcttaaaccttcgttcgaccttcttcttcactaatactattacttgccctacacttttcaccttcaaactctcctctgtttctaaccctagacgagtattccccaatattagagctcacgtttccgcagccagctccaattcagagctcgaatctctcttatctaccgatcggaagctgatcagtaagcaatcaaacaatggtgcgtcttcgatttcgtcgggagtgagacttgagaacattagtaagagctatgaaggcattacggtgctaaaggatgtgacttgggaagtgaagaaaggagaaaaagtagggttaatcggcgtcaacggtgcagggaagacgacacagctcaggatcataacgggtcaagaagaacctgattcaggaaacgtgatttgggccaaacccaatttgaaagttgctttcttgagtcaagagttcgaggtttcaatggggaagacagtgaaggaagagttcatgtgtacgtttaaggaagagatggaaatagcgaggaagcttgagaacttgcagaaggcgattgaagaagctgttgatgatttagaactaatgggaaagttactagacgaattcgatttgttgcagagacgagctcaagaggttgatttggattctatccatgctaaaatcagtaagctgatgtcggaattagggtttgtgtcggaggatgctgataggcttgtggcttcttttagcagtggatggcaaatgagaatgtcacttgggaagattttgcttcagaatccagatttactgcttttggatgagccaacgaatcatttggatcttgatactattgagtggcttgaaggttacttgattaagcaagatgtgcctatggtcataatatcacatgacagggcgtttcttgatcagttgtgtactaaaattgtggaaaccgaaatgggagtttcgaggactttcgatggtaattactctcagtatgtaatttcaaaggcagagttggttgaagctcagtatgctgcttgggagaagcaacagaaagagattgaagcaacaaaggatttgattagtaggctaagtgctggagcaaattctggtcgagcttcttcagcagaaaagaaactggagaagcttcaggaagaggagctaatagagaagccatttcagcggaaacaaatgaaaatccggtttccggaatgtggattaagtggaagatctgtagtcactgttaagaatcttgtatttggttttgatgataagatgttatttaacaaggcgaatctagctatagaaagaggagagaaagtcgctataattggtccaaacgggtgcggaaagagtacattgctgaaacttattatgggtttggagaagcctatgagaggtgaggtgatacttggggagcacaatgtattgccaaactacttcgagcagaatcaggcagaggctcaagatttggataaaacggtgattgagacagttgttgaagctgctgtagattggagaattgatgatataaaagctcttcttggtcggtgcaacttcaaagctgacatgttggatagaaaggtctctcttttgagtggtggtgagaaggcacgccttgctttctgcaaattcatggtgaaaccatccactctacttgtgttggacgaacccaccaatcacttagacataccttcaaaagaaatgcttgaggaagctataaatgagtacaaaggcacggtcatcacagtctcacacgatagatacttcattaaacagattgttaacagagttattgaagtcagagatggtggtttaatggactatgcaggagactacaattatttcctggagaagaatgttgaagctagagcaagggagctggagagggaagcagaattggaagaaaaagctccaaaagtgaaagcaaagtcaaagatgtcaaaggctgagagagaagcacggaagaagcaaaagatgaaagcgtttcaagcttccaaaaagaagtcaaagtccagcaaaaatgccaagagatggaattaa

SEQ ID NO: 91 - DNA, 1335 bp, Arabidopsis thaliana
  misc_feature 1..1335: note = ORF of protein Gdi2
  source 1..1335: mol_type = unassigned DNA
atggatgaagagtacgaggttattgttctcggcaccggtctcaaggagtgtatcctcagcggtctcctttccgtcgatggtgtcaaggtgcttcacatggacaggaatgactactatggtggagaatcaacttcccttaatctcaatcagctttggaagaagttcaggggagaagagaaggctcctgagcatttaggcgctagccgggattacaatgttgacatgatgcctaagtttatgatgggaaatggcaagcttgttcgtacccttattcatacagatgttacaaagtatttgtccttcaaagctgttgatggaagctatgttttcgtcaaaggaaaggttcaaaaggtgccagctactcctatggaggccctgaaatcttctctcatgggcatatttgagaaacgtcgagccggcaagtttttcagttttgttcaagaatacgatgagaaggacccaaaaacacacgatggaatggatttgaccagagttacaacaaaggaactgattgcgaaatatggtcttgatggcaacactattgactttattggtcacgcagtggcacttcacacgaatgaccaacatctcgaccaacccgcctttgatactgtaatgagaatgaagctctatgcggaatctcttgcacgtttccaaggaacatctccatatatttatcctctctatgggttgggagaactaccccaggcatttgcacgacttagtgctgtctatggtggcacatatatgttgaacaaacctgagtgcaaggtagagtttgacgagggaggtaaggttattggtgtaacatctgagggagagactgctaaatgcaaaaagattgtgtgtgacccttcatacctgccgaacaaggttaggaagattggcagggttgctcgggccatcgccattatgagccacccgattccgaacaccaatgattctcactcagtgcaggtcatcataccccagaaacagttggcccgcaaatcggatatgtatgtcttctgttgttcgtactcccacaacgttgctcccaagggaaaattcattgcatttgtgtctacagatgcagagactgataaccctcaaaccgaactaaagcctggaactgatcttttgggtcctgttgatgagatattcttcgacatgtatgatagatacgagcctgtcaacgagccagagttggacaactgctttatttcaacgagctatgatgctacaacacactttgagacaactgttgcggatgtgttgaacatgtataccctaatcaccggaaagcaactggacctaagtgttgacctgagtgccgcaagtgctgcagaggaatga

SEQ ID NO: 92 - DNA, 1293 bp, Arabidopsis thaliana
  misc_feature 1..1293: note = ORF of protein Gln2
  source 1..1293: mol_type = unassigned DNA
atggctcagatcttagcagcttctccaacatgtcagatgagagtgcctaaacactcatcagtcattgcatcatcatccaagttatggagctctgttgtgttgaaacagaagaagcagagcaacaacaaagtcagaggctttagagttcttgctctccaatctgataacagtactgtcaatagagttgagactcttctcaatttagacaccaaaccttactctgacaggatcattgctgaatacatttggatcggaggatctggaattgaccttagaagcaagtcaaggactatcgaaaagccggtggaggatccttctgagctacctaagtggaactatgatggttcgagtaccggtcaagcacctggtgaagatagtgaagtgattctatacccgcaagctatcttcagagatcctttccgtggaggcaataacatcttggttatctgtgatacttggacaccagctggtgagccaattccaacaaacaaacgtgctaaagctgctgagatcttcagtaacaagaaggtctctggcgaggttccatggttcggcattgaacaagagtacactttacttcagcaaaacgtcaaatggcctttaggttggcctgttggagcgttccctggtcctcagggtccttactactgtggagttggagctgacaagatttgggggcgtgacatttcagatgctcattacaaagcttgtttatatgctggaattaacattagtggtactaatggtgaagttatgcctggacagtgggagttccaagttggcccgagcgtaggaattgatgcaggtgatcatgtttggtgtgctagataccttcttgagagaatcacagaacaagctggtgttgtcctaacacttgatcccaaaccgatagagggtgactggaacggtgctggttgccacaccaattacagtaccaagagcatgagagaggaaggaggatttgaagtgatcaagaaggctatcttgaacctctcgcttcgccacaaggagcacatcagtgcctacggtgaaggaaacgagagaaggttgaccggaaagcacgagacagctagtattgaccagttctcatggggcgtggctaaccgtggatgctctattcgtgtgggacgtgacaccgaggcgaaaggaaaaggttacttagaagatcgccgtccagcatctaacatggacccatacattgtgacctcacttttggcagagaccacactcctgtgggagccaactcttgaggctgaagcccttgcagctcaaaagctttctttgaatgtttaa

SEQ ID NO: 93 - DNA, 5853 bp, Arabidopsis thaliana
  misc_feature 1..5853: note = ORF of protein Gsl3
  source 1..5853: mol_type = unassigned DNA
atggctcaaaggaaaggacctgatccgcctccgcctcaaaggcggattctgcggactcaaactgctggtaatctcggagaggctatgttggacagtgaagtggtgccatcgtctcttgtggagattgctccgatccttcgtgtagctaatgaagttgaagctagtaacccccgtgttgcttacttatgtcggttttatgcgtttgagaaggcacacaggcttgatcccacatcaagtggacgtggtgttcgccagtttaagactgcgcttcttcaacggttagaacgggagaatgaaacaactcttgcaggaaggcagaagagtgatgcacgcgaaatgcaaagtttctatcaacactactacaagaaatacatccaagctttgcaaaacgccgctgacaaagctgaccgtgctcaacttacaaaggcatatcaaactgctgctgttctctttgaagtcttgaaagctgttaatcagacagaagatgtggaagttgctgatgagattttggaggctcacacaaaggtggaagaaaaaagccaaatctacgtgccttacaacattcttccccttgatcctgatagtcaaaatcaggctataatgagatttcctgagatacaagctacagtaagcgcacttcgtaacactaggggtttaccatggccagctggtcacaagaagaaactcgatgaagacatgctagactggcttcaaactatgtttggttttcagaaagataatgtttcaaatcaaagagagcacttgattttgttacttgctaatgtccacattcgacagtttccaagacctgaacaacaaccaaggctggatgatcgtgcattgactatagtgatgaagaaacttttcaaaaactacaaaaagtggtgcaagtatttgggtcggaagagcagcctttggttgccgacgattcaacaagaggttcagcagcggaaactgctatacatgggactctatcttttaatatggggagaagctgcaaacctgagatttttgccagaatgcctttgctatatatatcaccatatggcttttgagctgtatgggatgttggctgggagtgttagtccaatgacaggggagcatgtaaaaccggcatatggtggtgaagacgaagctttcctgcaaaaagtagtgactcctatatacaagacaatagcaaaggaagcaaagagaagtagaggaggaaaatcaaagcattctgagtggagaaactatgacgacttgaatgaatatttttggtcaattcggtgctttcggttagggtggcctatgcgagctgatgctgatttcttttgtcagacagcggaagaacttcgacttgacagaagtgagaataaaccaaaaactggagatcgatggatgggcaaagtcaattttgttgagatacgctccttctggcatatatttagaagtttcgatagaatgtggagtttctatatcctgtctttgcaggcaatgatcataattgcttggaatggatctgggaaattaagcggcatcttccaaggtgatgtcttcctaaaggttttgagcatcttcattacggcagctatattaaagcttgcacaagctgtccttgatatagctctaagctggaaatcaagacatagtatgtcattccatgtgaagctcagatttatcttcaaggcagttgcagctgcaatctgggtggttctcatgccactaacttacgcatacagctggaagaccccttctggttttgcagagaccataaagaactggtttgggggacaccaaaattcctcaccttcctttttcattattgtcattcttatctacttatccccaaatatgctctctacattgctcttcgcgttcccattcatccgacgttatcttgaaaggtctgactataaaatagtgatgcttatgatgtggtggtctcagccgagactctatatagggaggggtatgcatgagagtgcattgtcacttttcaagtacacgatgttctgggttgtacttctaatatcaaagcttgcattcagtttttatgcagagattaagcctcttgttaaaccaaccaaagacattatgcgagttcatatatcagtctaccgctggcatgagtttttccctcacgccaagagtaatatgggtgtggtgatcgctctctggtcacctgttattcttgtttatttcatggacactcaaatatggtatgctatagtttcaactctggtgggaggtttaaatggagcttttcgacgtcttggagagatccgaactctaggaatgttgaggtctagatttcagtccttaccagaggcctttaatgcttgcttggttccaaatgagaagagcgaaactccaaagaaaaaggggattatggcgaccttcactcgcaagttcgatcaggttccatctagtaaagacaaggaggccgcacgatttgctcagatgtggaacaaaataatcagtagtttccgagaggaggatttgattagcgatagggaaatggaactcctgcttgtgccatattgggctgaccgtgatctggatcttatccgctggccaccttttctgctggctagtaagatcccaatagctttggatatggcaaaagatagcaatggaaaagatcgtgaactgaccaagagattgagtgtcgacagttacatgacttgtgcggttagagagtgctacgcctcttttaaaaatctcataaactttttggttgttggagaacgagaaggacaggtcataaacgaaatcttctccaggattgatgaacatatagagaaagaaacactaataaaggacttgaacttgagtgctcttcccgatctgtatggtcaatttgttcgactaattgaatatctgatggaaaacagagaggaggacaaggaccaaattgttattgtcttgctgaacatgttagaagtggtgacaagagacataatggacgaagaggtcccaagcatgctagagtcaactcacaatggaacttatgtcaagtacgacgtaatgactcctcttcatcaacagcgcaaatatttcagccaacttcgattcccagtgtactctcagacagaagcctggaaagaaaaaatcaagaggcttcatcttttacttactgtcaaagagtcggctatggatgtgccatccaacttggaagctcgaaggcgacttacattcttttcaaattcattattcatggaaatgcctgatgcccccaaaatccgcaacatgctttccttttcggtcctaacaccatactattcagaggatgttctattctccatttttggtttggagaagcaaaatgaagatggagtctctatactcttttacttgcagaagatctttccagatgagtggacaaactttttggaacgcgtgaaatgtggcagcgaagaagaactcagagcaagagaagagttggaagaggaacttcgtctgtgggcatcttacagaggccaaacactgaccaaaactgtgcgaggcatgatgtattaccgaaaggctttggagctccaggccttccttgatatggcaaaggatgaagaattaatgaaagggtacaaggctctcgagttaactagtgaagacgcatcgaagagtggaacgtcgctctgggctcagtgtcaagcacttgctgacatgaaatttacctttgtggtttcatgccaacaatacagtgttcagaagagatccggtgaccaacgtgcgaaggacatattgaggcttatgactacgtacccgtctctacgtgttgcttatattgacgaggttgagcaaacacataaagaaagctataaaggggcagacgaaaaaatttactactcagcccttgtcaaagctgctccgcagaccaaatctatggattcttctgaatctgtgcaaacactagaccaggtcatataccggattaagctaccaggacctgcaatactaggagaggggaagccagagaatcagaaccattctatcatttttactcgcggagaagggttacagacaatagacatgaaccaggacaattacatggaggaagctttcaaaatgaggaacttgcttcaagaatttcttgttaagcatggaggcgtaagaacccctacaatacttggacttagagagcatattttcacgggaagtgtttcttctcttgcatggtttatgtcgaatcaggaaaacagctttgttactattgggcagagggtgctagctagccccttgaaggtacgattccattacggtcatccagatgtttttgatcgactgtttcaccttaccagaggtggtgtctgcaaagcttctaaagttatcaatctaagtgaagacatatttgctggtttcaactctactcttcgtgaaggcaatgtgactcatcatgaatacatacaagttggtaaagggagagatgtaggactcaaccagatttcaatgtttgaggcaaagatcgctaacggaaatggggagcaaactctaagccgtgacctatataggctggggcaccgatttgatttcttccgcatgctttcttgttatttcactacaattgggttctacttcagtactatgttaaccgtgcttacggtctatgtatttctatatggacggctgtatctcgttcttagtggtcttgaagaaggattgagcaaccagaaagcttttcgatccaacatgcctcttcaagctgcacttgcttctcaatcatttgtgcagattggtttcttgatggccttacctatgatgatggagataggtcttgagaggggattccataacgccctaattgattttgtactgatgcagttacaactcgcttccgtgtttttcacgttccaactggggacaaaaactcattactatggtcgaacattgtttcatggcggcgcagaatacagaggcactgggcgtggttttgtggtgttccatgccaaatttgctgaaaactacagattctactcgcgcagtcattttgtgaagggtattgagctgatgatactcctgctagtgtatcagatatttggccatgcctatagaggagttgttacatatattctgatcactgtttcgatatggttcatggtggtgacttggctctttgctcctttcctgttcaatccctctggtttcgagtggcaaaagatcgtagatgattggacagattggaataaatggatatataaccgagggggaattggtgttcctccagaaaagagttgggagtcttggtgggagaaggaaataggacatcttcgacactcgggaaaacggggtatcatcctcgagattgttttggctttacgcttcttcatctttcagtacggactggtttatcaactcagcacctttaaacaggaaaatcaaagtttgtggatttatggagcttcatggttcgtgatcttattcattttacttattgtaaagggtctgggtgtgggaagacaacggttcagcacaaacttccagcttctcttcaggatcatcaagggttttgtgttcctcacatttcttggacttctcattactttcttagctcttcgttttttaacacccaaggacatcttcctttgtatgctcgctttcatgccaaccggttggggaatgttactcattgcacaagcgtgtaagcctctgattcaacggctaggattctggtcatcggttaggacgctggctcgaggctatgagattctcatgggtttacttttgttcactccggtagcgttcctggcttggttcccgttcgtgtctgagtttcagacaagaatgctctttaaccaagcatttagcagaggattgcagatttcacgtattcttggtggtcagagaaaagaccgctcttccaagaacaaggagtga

SEQ ID NO: 94 - DNA, 1338 bp, Arabidopsis thaliana
  misc_feature 1..1338: note = ORF of protein Hag5
  source 1..1338: mol_type = unassigned DNA
atgggatcgtcagcgaatacagaaaccaacggcaacgcaccgccaccgtcgtcgaatcaaaagcctccggctacgaacggcgttgatgggtctcatcctcctcctcctcctttaactcctgatcaagctattatagagtcggatccgtcgaagaagaggaaaatggggatgcttcctctagaagtgggtactcgtgtgatgtgtcggtggagagacgggaaacaccatccggtgaaagtaattgagcgccggcggatacataacggcggtcaaaatgattacgagtattacgttcattacactgagtttaataggaggctggatgaatggactcagctggaccaactggaccttgattcagtagagtgcgctgtagatgaaaaagtggaagacaaggtaacaagcttgaagatgacacgtcaccagaagaggaagatcgatgagacacatatagagggtcatgaagagctggatgcagcaagtttgcgtgaacatgaagagttcacgaaagtgaagaacatatcaacaattgagcttggaaaatatgagattgagacttggtacttctccccttttccgccagaatacaatgactgtgtgaagctctttttttgtgagttttgcctgaacttcatgaaacgcaaagagcagcttcaaaggcatatgaggaagtgtgacctgaagcacccacctggtgatgaaatttaccgaagtggtaccttgtcaatgtttgaggtagatggcaaaaagaacaaggtttatgcacagaatctctgctacctggcaaagttatttcttgaccacaaaactctttactacgatgttgatttgtttctattctacgttctttgcgaatgtgatgaccgaggatgccacatggttgggtacttttcaaaggagaagcattcggaagaagcatacaacttagcttgcattctaaccctgccttcatatcaaagaaaaggctatggaaagttcttaatagccttttcctatgaactgtcaaagaaagagggaaaagttgggacaccggaaagacccttgtcggatctaggcttactaagctacagaggttattggactcgtgttctattagaaatcttgaaaaaacataagggaaacatttctatcaaggagctgagcgacgtgacagcaatcaaagcggaagatatattaagcacacttcagagcctagaactgatacagtacaggaaaggacagcatgtgatctgtgcggatccaaaggttctggaccgacatctgaaagctgcaggccgaggtggtcttgatgtagatgctagcaaactgatttggacaccttacaaggaccagagttaa

SEQ ID NO: 95 - DNA, 2049 bp, Arabidopsis thaliana
  misc_feature 1..2049: note = ORF of protein Hda18
  source 1..2049: mol_type = unassigned DNA
atgcttttgaagtttgaagcttcttcagagttgagactagttgacccttcagtttctctcactgttcttcgaaagataagactgtcacatctcccggatatgacaatgaccagcgaatcttccggcaagaaatgcggagagggtgacggcaaagtcgccggtaaaagccagcgaaaggtgggtcttgtttatgacgagaccatgtgcaaacacgacacacctaacggtaaggttgacgtagagtgtcccgatcgtatcagagttatctgggagaagcttcagctcgccggagtcactcaaagatgtgtggttcttggtggtagtaaagctgaagataagcatctcaaattggttcacacaaagaaacatgttaatttagtgaagagtattagcacaaaaaagaaagattctcggagaaacaagattgcttcacagttggattcgatttatctgaatggaggttcatctgaagctgcatatcttgcagctggatctgttgtaaaggtggcagagaaagttgcagaaggagagttagattgtggctttgctattgtcaggcccccaggacaccatgccgagtcagatgaagccatgggtttttgtttattcaacaatgttgccgttgctgctagttttttactaaacgaaagacctgatctagatgtcaagaaaattctgattgtcgattgggatatccatcacggaaatggtacacagaagatgttctggaaagattctcgcgtactaatattctctgttcataggcatgatcacggaagtttctatccatttggcgatgatggagactttaacatggttggcgaaggtccaggtgaagggtttaacattaacgttccatgggagcaaggaggatgtggagatgcagactatcttgccgtatggaaccacatcttgatccctgtgaccaaggaattcaaaccagatattattttgctttcagctggatttgatgcagctattggtgatccgcttgggggttgttgtgttaccccatatggatattcagttatgttgaagaagctgatggagtttgcacatggaaagatagtgctggcattagagggaggatacaatcttgagtcattgggaaagtcttcactcgcatgtgtccaagttttgcttgaagacaaacaaattcatggttcatctgaaacatacccattagaatctacacggcgtgtcatacaagcggtacgcgagagactatgcacatattggccttcacttgacgcgtcaatggcgtctaatgagaatttaaagaacccaagtgcagaaagaaactccgctgacgccctccttcgtgaagtggaagagttaaagtctctaatggctgcccgagatggagaactggaggcaaggagaaaagagttaaaggccaaaaataaggagctagaagcgaacgagaaggagctagaagcgggactaatgttgattcgcgctcgggaggatgtaatctgtggtcttcatgcaaaaatagagagtttgcagcaggagcgagatgaggcggtagcgaaagctgaacgcatagataaggaactccaagaggatagggcaagaagccaggagttcaaagaagacacagagttttgcttgagtacgttaaggagggagaaggagcttgccatcatggccaaaaataaggacctagaagcgaaagagaaggagctagaagcgcggctaatgttggttcacgctcgggaggataaaattcatgccaaaatagagaggttgcagcaggagcgagatgaggccgtagcaaaagctgaacgcatagataaggaactccaagaggataggtcaagaagccgagttgggaatggatcttttgccttttctcaggagttctatgaagacatggacttagacgagctcgaacctctgtctccggagttcaatgaagacatggactcggaagagcttgaaccgtttcaggtcatcaagaaaaacatggagcgttcccacaagaagttcataaaagatatggagtgtatcaagtttatcgccagtgagagagccagggtactttag

SEQ ID NO: 96 - DNA, 1626 bp, Arabidopsis thaliana
  misc_feature 1..1626: note = ORF of protein Hexo1
  source 1..1626: mol_type = unassigned DNA
atgtcaacgaatcttcttcgtcttattctcctattcatcactttatctatcacttcatctctgtcaactccgtcgccggctgattctcctccttatctatggccgttaccggcggagttttcgttcggaaacgaaacgctctcggtggatcccaccgtgactctaatcgtagctggaaatggcggtggatctctgattattagagctgcgtttgataggtatatgggtataatctttaaacacgcttctggtagaggttctctattgtcgaggattagatttctgaaaatggttgagtatgatatcacttctttgaagatcgttgttcattctgattctgaggagcttcaattgggtgtggatgagagttatacattgatggtgtcgaagaagaacgagcaatcgattgttggagcagccacgatcgaggctaatacagtttatggtgcacttagaggattggagacatttagccagttgtgtgcctttgattatataaccaaatctgtgcaaatatataaagcaccatggtatattcaagataaacctagatttggataccgcggtctgctgatagatacatcaagacattatttaccaattgatgttattaagcaaataatcgaatccatgtcctttgccaaacttaatgtcctgcattggcacattgtagacgagcaatcatttcctcttgaaactcctacatatcctaatttgtggaaaggagcttattcgagatgggaacgttatacagttgaggatgcctctgagattgtcaggtttgccaaaatgagaggtattaatgtcatggctgaagtagatgtccctggccatgcggaatcgtggggaactggataccctgatctttggccgtctctgtcttgccgagagccccttgatgttactaaaaactttacattcgatgtgatttctggcattttagcagatatgagaaagatattcccatttgagcttttccacttgggtggtgatgaagttaatacagattgctggaaaaacacaactcatgttaaagaatggcttcagggtcgcaacttcacgactaaggacgcatataaatactttgtactccgagcgcaacagatagctatatcgaaaaattggacacctgtcaactgggaagaaaccttcagttcctttggaaaagatcttgatccgcggactgtgattcaaaactggctagtgtctgatatatgtcaaaaagcagtagcaaaaggattcaggtgcattttcagtaaccaaggatattggtacctcgaccatttagatgttccgtgggaagaggtttataacaccgagcctctaaatggaatcgaggatccttctctccaaaagcttgtcatcggaggagaagtttgcatgtggggtgaaacagctgatacatccgttgtccttcagaccatttggcctcgtgctgcagcagctgcagaacggatgtggagcaccagagaggccgtgtcaaaggggaacataacgttaaccgcattgccacgtctacactacttcagatgtctactgaataaccgtggcgttccagcggctccagtggataacttttacgcacgaagaccccccttgggacctggctcttgctatgctcagtga

SEQ ID NO: 97 - DNA, 1338 bp, Arabidopsis thaliana
  misc_feature 1..1338: note = (ORF of protein Hppd
  source 1..1338: mol_type = unassigned DNA
atgggccaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcttcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 98 - DNA, 2331 bp, Arabidopsis thaliana
  misc_feature 1..2331: note = ORF of protein Hsl1
  source 1..2331: mol_type = unassigned DNA
atggagtcaataaaggtttgcatgaacgcactgtgcggagcggcctctacgtcgggcgagtggaaaaaaggctggcctatgcgatccggtgatttagcttctctctgcgataagtgtgggtgtgcatacgagcaatccatattctgtgaagtgttccatgccaaggaatctggttggagagagtgtaattcatgtgacaagcgtcttcactgtggatgcattgcttctagatttatgatggagcttctagagaatggtggtgttacctgtataagttgcgccaagaaatccggactaatttctatgaatgtgagccatgaatctaacggtaaggacttcccctcatttgcttcagcagagcatgtaggcagtgttcttgagaggacaaatctcaagcacttgcttcactttcaaagaatcgaccccactcattcttctcttcaaatgaaacaagaagaatcgctgcttccttccagcctagatgctcttagacacaaaactgaaaggaaagaattgtctgcacagccaaacttgagcatttcacttggacctacgcttatgacaagcccatttcatgatgctgctgttgatgacagaagtaagactaattcgattttccaactggcccctcggtccaggcagctgcttccaaaacctgcaaattcagctcccattgctgctggcatggagcctagtgggagcctggtgtcacagattcatgtcgctcggcctcctccagaaggtcgcgggaagacccaattgcttccccgttactggcctaggattactgaccaagagctgctgcaattatctggacactcaaattccaaaattataccactctttgaaaaagttctgagtgcgagcgatgcgggtcgtattggtcgactggttcttccgaaagcatgtgcagaggcatatttcccccctatatctctacccgagggtctcccgttaaagatacaagacataaaagggaaagaatgggtgttccagttcaggttttggcctaataataacagcaggatgtacgttttggagggtgtgactccttgcatacagtccatgcagttgcaagctggtgacactgtaacattcagccgtacagaacctgaaggaaaactcgtaatgggataccgtaaagcgacgaactctacagcgacacagatgttcaagggaagcagtgaacccaatctgaacatgttttccaacagcttgaatccgggatgtggtgacatcaattggtctaaactagagaagtctgaggacatggcaaaggataacttatttcttcagtcgtccttaacttctgctaggaaacgggttcggaacattgggactaagagcaagcgtctgctcattgatagcgtagatgttctggaactgaaaataacttgggaggaggcacaggagctgttgcggcctccccaatccaccaaacccagcatctttacgctggaaaatcaagattttgaagaatatgacgaaccaccagttttcgggaagaggaccctttttgtctcacgtcaaacaggggaacaagagcaatgggtgcagtgtgatgcttgtgggaaatggcgacagctgccggtggatattcttcttccaccaaagtggtcgtgctctgataatctcttggatcctggcaggtcttcatgttccgcacctgatgaactctctccaagagaacaggatacacttgtccggcagagcaaagagttcaaaaggaggagactggcatcatcaaacgaaaagctaaaccagtcgcaggatgcatctgctctgaatagtttaggaaatgcaggcatcaccacaaccggtgaacagggggaaatcacggttgcagccacgaccaagcatccaagacaccgggcagggtgttcgtgcatcgtctgcagccaaccaccgagcggaaaaggcaaacacaagccgtcatgcacttgcactgtgtgcgaggcagtgaagagacgattcaggacgctcatgctgcggaagcggaacaaaggagaggcaggacaggcaagccagcaggcgcagtcacagtcagagtgcagggacgagacagaagtggagagcattccagcggttgaactagccgcaggggaaaacatcgacttgaactcagacccgggggcttcccgagtaagcatgatgaggcttctccaagctgcagcgtttcctctggaagcatatctgaaacaaaaggctatttccaatacagcaggagaacagcaaagcagtgatatggtcagcacagaacacggttcgtcctcagccgcacaagaaactgagaaagacacaacaaatggagctcatgatcctgtgaactaa

SEQ ID NO: 99 - DNA, 477 bp, Arabidopsis thaliana
  misc_feature 1..477: note = ORF of protein Iaa31
  source 1..477: mol_type = unassigned DNA
atggaggtctctaactcttgttcttcattttcttcatcctctgtcgacagtactaaaccttctccttctgaatcttctgttaatctctcccttagtctcacatttccttctacttctccacaaagagaagcaagacaagattggccaccgataaagtctagattaagagatacactaaagggtcgtcgtcttcttcgtcgtggtgatgacacttctctctttgttaaggtttatatggaaggtgttcccattggaagaaaactcgacctttgcgtattctcaggctacgagagtctattagaaaatctctctcacatgttcgatacttcaatcatctgcggtaatcgagatcgaaaacatcatgttttgacatatgaagacaaggatggagattggatgatggtcggagatattccatgggatatgtttcttgaaaccgtgagaagactaaagatcacgagaccggagaggtattaa

SEQ ID NO: 100 - DNA, 1995 bp, Arabidopsis thaliana
  misc_feature 1..1995: note = ORF of protein Iqd28
  source 1..1995: mol_type = unassigned DNA
atgggaaagactcctggtaaatggatcaagactttgcttctcgggaagaagtcgcctaagtcgaattcggataaccgaagccagaaactgaaatctgctaagaaagaagagctggtggaatcggtgacagaagatttgtcaaacttaacagttgatcctccggtggtttcatcacaaccagttccagcttctactgctcaaaatgtggtttctccaatcaatggtgatgagtccaaggataatctcgagtcaaggaatgacttgggagaagtcgagcttgaacaagctgcaattaaggttcaagctaccttcagagctcatcaggcacgtagagcatttcgaactcttaaaggtatcatcaggctgcaggcagttatccgtggtcacttggtgagaagacaagccattgctacatattcatgtatatggggaattgtgaagtttcaggctcttgttcgtggccagaaagctagatcttcagatattgcgattcagtttcagaaaaaacatatggaagcaagtgattcagaagttttgcaatcgagcacgtgtagttggatggataatcccacaaagttcgtctttgttgataagcttctagcttcttcaccaactgcattgcctctgaagatccagtatggtcctgaggaacctaactcagccaaggtttggcttgaacgctggacacaattacaagtttggtcttctggttcacgagtacctaggattgagattccaaaatctcagtcaaagaagcgaaactaccaagcagtggttgaagcagaaaagaaaaggccaaagcgaagtatcaagaaaccatctggcacaactagtggaactggccctagccgttttacagctgaaagaaataaacctaaacgaaatgtgaggaaggcttccacgcttagtaaagatcccctgagaaatgagagtgacaaagcaaatcacaattcgagaaaaagtagaagcggctcaaaggaggggtcgcctttggagattaaagatgagaagcccagtcctagtctcaaaaggtcttctctttcgaacgggtcaaagaaagccacccttagatctgctgagaagaagaagaaagatattccagattcttctgtgcaaatacagcctgaagggaaagtttcagagaatgttcttgaaggaggagacaatattgagtttgctgaaaaggagaaagatactacagattctgtgcagatagagtctgaagggaaggtttcagggaatgttcttgaaggaggagaaggagaaaatattgtgtttactgaaaaggagaaagatactgcagatcctgtgcagatagagcctgaaaggaaggttcttgaagaaggagacaatatcgagtcttctggaaaagaaaaagatactggagattctgtgcagatagagtctgaaggaaaggttcttgaaggaggagacaatattgaatttggtgaaaaagagaaagataaagctgatgcagttcccatcgaatttgatattgtaaaagatgagaagtcaccagttttggataggacggaggaagatgaactgaaaactgcagagaccagtgacaaagccgaagcccttaaatgtgcagatgtgaaggtaagctctgagaatggtaatgttggttctgataacacaaagcaaagtgaaaaacgagcattgctgcctgcaaatattgacaaacaagatgatgggcttacccttagcggacggaaaatccccagctacatggccccgactgcatctgcaaaggccagagtcaaaggagaagcctctccgaggtttgcccaagcgaagactgagataaatggggcattacggcgccattcactgccttctccagccaatggtaagctgagtactactactatgtctccaagggctcaaaaacttctcttagcctctgccaaaggatctatgaacggcgacaaatcttttacttcttccaaggacataactcacaagtcaaccagaaccgactggaaacggtga

SEQ ID NO: 101 - DNA, 1956 bp, Arabidopsis thaliana
  misc_feature 1..1956: note = ORF of protein Jac1
  source 1..1956: mol_type = unassigned DNA
atgcagacattaccaagctcagaaaccgttcttttaggctccaactcagcaccacctgtgttgcgtagtcccggtggcgatgacgtggacatcgattttggtgacgtgtttggtggtcctcctaagagacgctctaaagttactagtaacgaagtcacacgacatagcttcagtgaatccgctctccggcgtcgtgacgtcatcgtcgacgtcggcgatctgcttccgcaagacgagaagcctgtattcggcgaagatacatcatctgtccggcgtcgtttcaccactgatgatttcttcgacgatattttcagagttaatgaatcatcatcacttccgggttctcggatccttagcccggctcacaaacccgaatcttcttcagggacttcgtctccatcccaattcagtcttcctgcaaaagcaacagagattccaacatttaatttagcagctactcgtagtttgaacaagaacaaagaaactgtttcgagctctcctttgtcaagaacttcaagcaaagcagatgtggtctctactgctaaatcatattcagatgattgtgatgatcctccacaagtttttgttactgggaaaggaaggcaatttcatttctctatctacaagtggcctaataaaggagttcctgttgttatttggggaagctctagattgagttctatgtctaaagctgaggagacaacacctgtaccattgagtgattatcgaaaaacttcagttgttgagaaattgggtaaaaatgaagaaggagatggaaaaagtggtttatctggtttgaaagatgtgaagaagacctcgctaaagcgtcctggtgtccagacaaaagaagagaaaacagaaacagatttgaagtctgaacaagccttctttggtgtctcaaaagcacgtgaggctaatgtgaagcctcttgattcagttgagtctgaacaggccttctctggtgtgtcaaaagcacatgaagctactactgtgaagcctcttcattcaatttttcatgaagaagatgagagacaagatgagaaaatagtatcagagagagaagtgagaaaaggtaaaagcaaagcgaagaacactcgaagttttaccgaagattcaagaaccaagaagaaatcacaaggcactaaaagttctttggatagtagcccgatacccgataaatccagcttcgcaagttcatcagcagcaccagaagttggcaaagatggagtcaaaggaaaagtgagtgacttcgttaagattttcagtaaaggagcttcagttggagcaggtggggaatctcttggacaaagctctagatggagagctaaagaaactcccaagactgatatcattcatgatggttctaatgccaaggaaactgtaaacattcctgatcaacaaaagaaatcgactccagatatccccgctatgaatcgggatcagaaaccttcacagtcaactcagaaaaaagattcagaccgggagagcatgaactataaggctcctggtgatactgtacaagaagagagacaagaacctagcacaacacatactacctcagaggatatagatgagcccttccatgtaaatttcgatgtggaggatataacacaagatgaaaacaaaatggaagaggccaataaggatgctgaagaaatcaagaacatcgatgctaaaatccgcaagtggtcaagcggaaagagtggaaacattcggtctcttctatccacgctgcaatatattctttggtctgggagcgggtggaagccggttcctctaatggatatgatcgaaggaaacgcggttcgaaaatcgtaccagagggcattactaatacttcatccagacaaactacaacaaaagggtgcttctgcaaaccagaaatacatggccgagaaagttttcgaattattacaggaagcatgggaccacttcaacactctcggaccggtttaa

SEQ ID NO: 102 - DNA, 1728 bp, Arabidopsis thaliana
  misc_feature 1..1728: note = ORF of protein Jar1
  source 1..1728: mol_type = unassigned DNA
atgttggagaaggttgaaactttcgacatgaacagagtcattgatgaattcgatgaaatgactagaaacgctcatcaagtccagaaacaaactttgaaagagattcttctcaagaaccaatccgccatttacttacaaaactgtggtctcaatggaaacgctactgaccctgaagaagctttcaaatctatggttcctttagtcactgatgttgaattagagccttacatcaaaagaatggttgatggtgacacttcacctattctcactggtcaccctgttcctgccatttccttaagctctggaactagtcaaggccgtccaaagtttattcctttcactgatgaattaatggagaacacattgcaactgtttcgcactgcttttgccttcagaaacagagattttcccattgatgacaatggcaaagctttgcaatttatcttcagcagcaagcagtacatatcaacgggaggtgtcccggttggaaccgcaaccacaaacgtgtaccgcaaccctaacttcaaagctgggatgaaatctataacgtctccttcctgtagtcccgacgaagttatctttagtcctgatgtccatcaagccttgtattgccatctcttatccggaattctcttcagagatcaagttcaatacgtctttgccgtctttgctcacggtctagttcacgcttttagaacctttgaacaggtttgggaagagattgttaccgatatcaaagatggtgtcttaagcaaccgtattactgtcccatcggtccgtactgcaatgtcgaagctgctcacacctaaccctgagctagctgagacgatccgcaccaaatgtatgagtttgagcaactggtacggattgattcccgcgctcttccccaacgcaaagtatgtttacgggatcatgactggctccatggagccgtatgtgccaaagctgagacattacgcgggtgatctacctcttgtgagccatgactacggtagctcggaaggatggattgctgctaatgttaccccaaggttatctccagaggaagctacatttgctgtgattccgaatctcggttatttcgagtttctccctgtatctgaaacaggggaaggagaggagaaaccggttggtttaactcaagtcaagattggagaagagtatgaagttgtcatcacaaattacgcagggttgtatcgataccggcttggagatgtggtaaaggtcattggtttctacaacaacactccccaactaaaattcatatgcaggagaaacctgattctctcgatcaacatcgacaagaacaccgaaagagaccttcagctttcagtagaatcggctgcaaagagactctcagaagaaaagatcgaggtcatagacttctctagctacatcgatgtctcgacagatccaggacattacgctatcttctgggagatttcaggagaaacaaacgaagacgttcttcaagattgctgcaactgtttagaccgagcattcatcgatgcgggttacgttagttcccggaaatgcaagactattggagccttggagctgagagttgtcgcaaaaggaactttccggaagattcaagaacattttctcgggcttggttcatcggctggacagtttaagatgccaagatgtgtgaagccaagtaatgctaaggttctccaaattctgtgtgagaatgtggtgagtagctacttcagcacagcgttttga

SEQ ID NO: 103 - DNA, 3399 bp, Arabidopsis thaliana
  misc_feature 1..3399: note = ORF of protein Kp1
  source 1..3399: mol_type = unassigned DNA
atgccacaagaattaatgagaggattaaagggtttggtggtgtcgtccacaagcaatggagattatgagcttgctcaacgaaaagctgaagaaacagctctgagaagataccaagcggtggcatggctgagacagatggaccaaggcgcgatggagacactcccggaaaaaccgtcggaagacgagttctccctcgccctccgcaacggcctcattctctgcaacgtcctcaacaaagtcaatcccggttctgttctcaaggtggtggagaatccgattacgccagctattcagtacgcggatggagcggcgcaatcggcaattcaatattttgagaacatgaggaactttcttaaagctgtagaggatatgcagcttttaacatttggagcttctgatctcgagaagggaggatcgtcgaacaaagtagtagattgtattttgtgtttgaaaggtttttacgagtggaaacaagcgggtggagttggagtatggaggtatggagggactgtgaggatagtatcattcaatcgaaaaggcagttctccgccgcagtatgggattggtagtgaaagtactaccgatgagtctgtttctttggatgaatctgagtcttctcagtatgaccagcttttggattttcttcatctttctaatgagatctcagctgaggaatctgaaactgcaatctccttggcttttctttttgatcatttcgcgcttcagcttctacatggttaccttaaggagagtgatgggatcaatgatatgcctttaaatgaaatggtgattgatacattgctgaacagggtagttaaggatttctctgcaatactagtttctcagggagctcagctgggttcgtttctaaggaaaatactgaaatgtgataacggggatctgtcaagatcagagtttctagcagcagtcttcagataccttcagcacagaaaagatttggtgtcaaaggagttttcaaagttttgtaaatgtggcggaaagcttgagtttagtagactaaacgcccgcgaattttctccgggtcatgtagaagctattggtcttcaacagaaagagctggaggaagttaaatcaaattttgtggaaacaagaagccaagttaaacaaatgcagtcagaatggcagaaagagcttcagaggatagtccatcacgttaaggccatggaagttacatcttcttcttaccacaaggttctggaggaaaatcgcttactttacaatgaggttcaagatctcaaaggcaccattagagtttactgcagagtcagacctttcttccaagaacaaaaagatatgcaatcaactgtggattatattggagaaaatggtaatattatcattaacaatcctttcaagcaggagaaagatgcacggaaaatattttccttcaacaaagtatttggacagactgtctcacaagagcaaatctacattgacactcaaccggtaataaggtccgtcctcgacggattcaatgtttgtatcttcgcgtatgggcaaaccggttcagggaaaacgtatacgatgagtgggccagatctgatgacggagacaacttggggcgtgaactaccgagctctacgcgatctcttccagctttcaaatgcaagaacacatgtggtgacttatgaaattggagtccaaatgatcgaaatatataatgaacaagttagagatctactagtcagtgatggttcctccagaagattagacatacgcaataattctcaactcaatggccttaatgtacccgatgcaaacttgattccagtttctaatactcgagatgtccttgatttgatgaggattggccaaaagaatcgtgctgtgggtgccactgctttgaatgagaggagcagccgttctcatagtgtattaactgttcacgtccaaggaaaagaattggcctcaggatccattttaagaggctgccttcatttagtggatttggctggaagtgaaagagtagaaaaatccgaagctgtaggcgagagactcaaagaagctcaacacataaacaaatcgttgtctgcattaggagatgtcatttatgcacttgcacaaaagagttcccacgttccctacagaaatagcaagcttacacaagttctgcaggactccttaggtggacaagcaaaaactttaatgtttgtacatattaatcctgaagttaatgcggtaggggagactatcagcacccttaagtttgcccaaagggttgcatccattgagcttggagcagctcgatcaaacaaggaaaccggtgaaattcgagatcttaaggatgagatatccagccttaaatcggccatggagaagaaggaagcagagctggaacaattgcgatcaggtagcattcggaacacaactgaatgtcagagagcaagagcagtttctcctttccatctcccaagaactggtaacggtgctggaacaaaggctgaggcaagtccacagccaaacgacggtaccagaagctacgagaccagaagttgctcaactggcaagcagagaaagtcagggtttccgtctgcactgagaaatagagaagcaagcccgaggatgccgaatctagcagaagagaggttaaatccgagtccaagtaggagatcactatccacagacagagcatcaaccatcaaaagtaggaataagcccgacgttacccaaaaccttccggtttcaagaacaccttttccagctagagtaccagtagtcaaatccttttcaactgttccattgaacccatcagcagaaaataaccacaggcttcacactgataactcttcagaggcatttcaaaatcatcagaagttaagcgctcgaaagctcttccctgagatcgaggaggaacacataaggcatgcactacacatacgtcaaggtggtgttaagaagaccagagccgagagcagtaaagccaaggcaaagcaaccatcaccagctaggtttcagaaactcgatgttgggatcagtttgcgttctgatgcagattctgaagcaaaggtaggaaattatcagacccaaaaggggaacaacaaccacaatgtgatacactcaagattccaaaacttcgacgtgggtatcagcctattctctgacctttgtgccggtgacaaatcagattcaacactcaagagtgattcctcagagacagacaacgaaccaccctcgaaatctaagaatgcccaaagaaactcgtccaaaaactccctcaaccacaaactaaggacgatctatgctcatgaggacacttcactcgtggacgacaagccttctaatggtacagcccatatcaaagaaggtaataataatatatctatgcctgagttcaggagaagtcgttcaacacatcatgcaaggttcatggtaccatag

SEQ ID NO: 104 - DNA, 2361 bp, Arabidopsis thaliana
  misc_feature 1..2361: note = ORF of protein Lrx2
  source 1..2361: mol_type = unassigned DNA
atgttgttgtttccttctacttctttacgtctcttcttctttctcttcctcttgttctcctcttgtttcttacagattagaggagatgatgatgatgatgatataagcgatgataatatcaaagtcgacccgagtctcaagttcgaaaacccaagtcttcgacaagcctacattgctcttcaatcatggaaacaagctattttctctgatccttttaacttcactgctaattggaatggctctgatgtttgttcctacaacggtatcttttgtgctccttctccttctagtccgaaaactcgagttgttgcgggtattgaccttaaccatgcggacatggctggttatttacctcgtgagcttggtcttctcactgatcttgctctctttcatctcaactcaaaccgcttttgtggagaagtacctctcacgtttaaacacatgaagcttctcttcgagcttgacttaagtaacaaccgttttgttgggaagttccctaatgttgtcttgtctttaccttccctcaagttcttggatcttcggtacaacgagtttgaaggtagtatcccttcgaagctattcgataaagagctcgacgcgatattcttaaaccataaccggtttatgtttgggatacccgaaaacatgggaaactctccggtgtctgctctagttcttgcggataatgatcttggaggttgcataccgggaagtatcggtttaatgggaaagactctcaacgagatcatcctctctaacgataacttaaccggttgcttaccaccacagattgggaatctcaagaatgtgacggttttcgatatcagtttcaaccgtttaagcggtccattgccatcaagcattggaaacatgaagagtttggaacagctcaatgttgctaacaatagattcacaggagttataccaagcagcatttgtcagctctctaaccttgagaacttcacttattcttccaacttcttcaccggtgatgccccgagatgtgtggctcttttgggagacaatgtggtggttaatggctcgatgaattgtattgatggtaaagaagatcaaagatcgtctaaagagtgttcttctccagcctcacgctctgttgattgtagtaagtttgggtgtaacaactttttctctcctccaccaccgtcttttaagatgtcacccaccgttcgagtacttcctccaccgcctccgtcttccaagatgtcgcctacatttagagcaactccgccaccgccgtcttctaagatgtcaccatcttttagagcaactccgccaccgccttcttctaaaatgtcgccttcttttagagcaactccgccaccaccttcttctaagatgtcgccttccgtcaaggcgtatcctcctcctccaccaccgccagagtatgagccatctccgcctcctccttcttccgaaatgtcaccttccgttagggcataccctccaccaccaccactatcaccaccaccaccatctcctcctccaccatacatctactcatctcctccaccaccgtcaccatctcctcctccgccatacatttactcatctcctccgcccgtagttaactgtccacccacaacacaaagtcccccacctccgaaatatgagcaaactccttcaccaagggaatactatccatcaccttcaccaccttactaccaatacacgtcttctccgccaccgcctacttactacgccactcaatctccaccgccaccaccaccgcctacttactacgctgttcaatctcccccgccaccaccaccggtatactacccacctgtaacagcaagtcctcctccaccaccggtctactatacaccggttatacaatctcctcctcctccaccggtttactattcacctgttacccaaagtcctccaccaccaccaccggtttactatcccccggtaacacaaagtccaccaccatcaccggtctactatccgccagttacccaaagtccacctcccccaccagtttattatcttccagtaactcaaagccctccaccgccatcaccagtctactatccaccggtagcaaaaagccctccaccaccatcaccagtctactatccaccggtaacacaaagcccacctccaccgtcaacaccagttgagtaccatccaccggcaagcccaaaccagtctccaccaccggagtatcaatcaccaccaccaaagggatgcaacgacagtccaagtaacgaccatcactaccaaacaccaacaccaccttctctgccgcctccatattacgaagacacacctcttccgcctatccgcggtgtctcttatgcgtctcctcctccaccatcaatcccatactactaa

SEQ ID NO: 105 - DNA, 1830 bp, Arabidopsis thaliana
  misc_feature 1..1830: note = ORF of protein Mapkkk3
  source 1..1830: mol_type = unassigned DNA
atgcctacttggtggggaagaaagtcctgcaagaacaaggacgataatcacagaggaatcatctccacagatagagatatcaagagctctgctgttgttgttgatcctcctctcactcctactcgtggtggtacacctcgttgcagtcgtgaattcgctggagcttcttctgctttctctggttttgactctgattctaccgagaagaaaggtcaccctcttcctcgtcctttgctctctcctgtttcgatccatcatcaagatcatgtaagcggatccacttctggatctacatctgtttctagcgttagctcatctggatcagctgatgatcagagtcaacttgttgcttctaggggtcgtggtgatgtgaaattcaatgtagcagcagcaccacgtagtcctgagagagtttctccaaaggcagctactattactactaggcccacgtctccacggcatcagcgtttgtctggagttgtgagcttagagtcttctacagggaggaatgatgatggaaggtcctcctctgagtgtcatcctttgcctcgaccacctacttctcctacaagcccttctgctgtgcatggttccaggattggaggaggctacgagacctctccttcggggttttccacgtggaaaaaagggaaatttcttgggagtggcacctttggtcaagtctatcttggtttcaacagtgagaaagggaaaatgtgtgctattaaagaggtcaaggtcatttctgacgatcaaacatcaaaagaatgtctgaagcaactaaatcaggagataaatttgcttaaccagctttgtcatccgaatattgtccagtattacggaagtgaactgagtgaagaaaccttgtccgtctacttggagtacgtgtcaggtggttcaatccataaactacttaaggattatggttctttcactgaacccgttatccaaaactacacgcggcagattcttgctgggcttgcctatttacatggacgaaatacagtacatagggacatcaaaggagcaaatatattagtggatccgaatggtgaaatcaagttggcagactttgggatggccaaacatgtaacagccttttctactatgctttcttttaaagggagtccttactggatggcacccgaggttgtgatgagccaaaatggctacactcatgcagtcgatatctggagtttgggttgtactattctggaaatggcaacatcaaagccaccttggagccagtttgaaggggttgctgcgattttcaaaatcggaaacagtaaagacaccccggaaatacctgatcacctttcaaatgatgcaaagaattttataaggctttgtctgcaacgaaatccgacagtacgtcctacagcttctcagcttctagaacacccttttctacgtaacacaacaagagtggctagtactagtttgcccaaagacttccccccacgttcctatgatggaaacttctcactgcagcctacaagggaaccctatccagggagactgagccatgataattatgcaaaacagccattgtctagaactataaagagcccgagcagagaaaacgtaagagctatcacatccttaccagtatctccatgttcaagccctttacgccaacttgggccagcatacaaaagttgtttcctgtcacctcctcacccgtcttacgcatttcctgggcaagacagtgggtacaacctagcagagtttgctgcaagccctttcaggatgaagaaagacgcaatgatggaaccatctagtttcaggactcaaacaccgaattcaccattgagatcaagactggtgtag

SEQ ID NO: 106 - DNA, 2151 bp, Arabidopsis thaliana
  misc_feature 1..2151: note = ORF of protein Mapkkk5
  source 1..2151: mol_type = unassigned DNA
atgcgttggcttccgcaaatctcgttctcgtctccttcttcttctccatcttcttctctaaaacccgtggcttcttactctgaatctccggatccagatcgtaatcaggatcgggatcggtttcatcgccgcttgtttcgttttaaccgtggcaggctcacccgtcagcggaagcttcgtcacttgacggatgacgatgttttgttgggagaacgtcgtgcttctacctcttcctccaccttcgattccggtttaactcgctctcccagcgctttcaccgccgttcctcgctctccttctgccgttcccttacccttacctctccccttaccggaggtcgccgggattcgaaatgccgcaaacgctagaggattggatgacagagatcgagatcccgagagacttatttctgatcgcacctcttctggtcctcctctcaccagcgtcaatggcggctttgcccgtgactcgaggaaagctacggagaattcgtcatatcaagattttagtccgagaaacagaaatggttattgggtgaatattccaaccatgagtgcaccaacgagtccatacatgagtcctgtgcctagtccacaaaggaagagtactggccacgatttgccttttttttatttgcctcctaaaagcaatcaagcttggtctgctccagatatgccacttgatacctctggtcttcctcctcctgcattttatgatattactgcctttagtaccgataattctcccatccatagtccacaacctcggagtccacggaaacagatcagaagcccacaacctagcagaccatcttcaccgttgcattcggttgatagctcagctccaccgcgagatagtgtttcctcacctttgcatccgaggttgtctactgatgttacgaatgggcgacgtgattgctgcaatgttcatcctttgcctctccctcctggagctacttgttcctcttcatcagctgcttctgttccgtccccgcaggctcctctcaaactggattcattcccaatgaattcccagtggaagaaagggaagctaataggtcgtggtacttttggaagtgtttacgttgcaagcaacagcgaaactggagcattgtgtgcgatgaaagaagttgagctatttcctgatgaccccaaatccgcagagtgtataaagcaattagagcaggagatcaaacttctaagtaaccttcaacatccaaacattgtgcagtattttggtagcgagacagtagaagatcgtttctttatatacctggaatatgttcacccgggttcaataaacaaatatatccgtgatcattgcggcaccatgacagaatctgttgttcgcaattttactcgtcacattttgtctgggctggcttatttgcacaataaaaagactgtacatagggatatcaaaggtgctaatctccttgttgatgcttctggggttgtcaagcttgctgatttcggcatggctaaacaccttactggacaaagagctgatctctcgttaaagggaagcccgtactggatggcaccagagctcatgcaagctgtgatgcaaaaagatagcaacccagatctggcttttgctgttgatatatggagtttaggatgtacaatcattgagatgttcactgggaagcctccttggagtgagtttgaaggggctgcagctatgttcaaggtcatgagagatagcccaccgatacctgaatcaatgtcacctgagggtaaagacttcctgagattatgcttccagagaaacccagctgagcgaccaaccgcatctatgttgctagaacaccggttcctaaagaactctttgcaaccaacctcaccaagcaacagtgatgtctctcaattatttaatgggatgaacataacggaaccaagcagtagaagggagaagccaaatttcaaactagaccaggtcccgcgagctagaaacatgacatcctcagagagtgaaagtgggcaacagcagcagcaacaacagtaccggtctcccgatctaacaggaaccgtgaaccgtctgtctcctcgttccactctggaggctatcccaagcccgtgtccttcccaacgacctaagcccagcagcagtgacaggagaagaacgggcgtcacttcagatcacctttga

SEQ ID NO: 107 - DNA, 2178 bp, Arabidopsis thaliana
  misc_feature 1..2178: note = ORF of protein Mfp2
  source 1..2178: mol_type = unassigned DNA
atggattcacgaaccaaggggaagacggtgatggaagttggtggcgatggcgttgccgtcataacactcatcaatcctcccgtcaattctctatccttcgacgtgctatacaatctcaaaagtaattacgaggaggccttgagcaggaatgatgttaaagctattgttattactggtgcaaaggggaggttctctggtggctttgatatatctggttttggtgaaatgcagaaggggaatgtcaaagagccaaaggctggttacatttcgattgacatcataaccgacttgcttgaagctgctaggaaaccgtctgtcgctgccattgatggacttgccttgggaggaggattagaacttgctatggcttgtcatgctaggatatcagctcctgctgcacagttaggcttgcctgagctgcaacttggtgttattcctggttttggaggaacgcagcgtctcccgcgtcttgttggtctcaccaaagcccttgaaatgattttgacatctaagccagttaaagctgaggaaggtcattctttgggtcttattgatgctgtggtgccacctgcggagttagtaaccactgctcgtcgctgggcccttgacatagtcgggaggagaaaaccatgggtatctagtgtttccaagactgataagttacctcctcttggagaggcaagggagatactgacgtttgccaaggcacagacactcaagcgtgcccccaatatgaaacaccctttaatgtgccttgacgctattgaagtaggtattgtttctggtccaagggctggtttagaaaaggaagctgaagtcgcctcacaagtggtaaaactggataccaccaaaggcttgatccacgtcttcttttctcagcgaggaactgctaaggttcctggagttactgatcgtgggttggtgccaaggaagattaagaaggtagccataattggaggcgggttgatgggatctggaatagctactgcgttgatcctaagtaactatccagtgattctcaaggaggtaaatgagaaattcctggaggctggaattggcagagttaaagctaatctccagagccgtgtaaggaagggaagcatgtctcaggaaaagtttgagaaaaccatgtctctccttaagggttctcttgattatgaaagctttagagatgtggacatggtcattgaggctgttattgagaatatatctttgaagcaacaaatttttgctgatctggagaaatactgtcctcagcattgtatccttgctagcaacacatcgaccattgacttgaacaaaattggggagcggaccaagtctcaggatcgaattgttggagcacattttttcagtccagcacatatcatgccgctacttgaaatagttcggaccaatcatacctctgcccaagtaattgttgacctgttagatgttgggaagaagattaagaaaacaccagttgtggtgggaaactgcacagggtttgcagtgaataggatgttcttcccttacacacaggcagctatgttccttgttgagtgtggagcagatccatatctaatcgacagggcaatcagcaagtttggaatgccaatgggtcccttcagactgtgtgacctggttggatttggtgtggcgattgcaaccgcaacgcagtttatcgagaacttctcagaacggacttacaaatcaatgattattccacttatgcaagaggacaagagagctggtgaagccactcgcaaaggtttctatttgtatgatgataagcgcaaggcgaaacctgatcctgagctaaagaaatatatcgaaaaggcaagaagcatatctggagtaaagcttgaccctaagttggcgaatttgtcggagaaggacattattgaaatgacattcttcccagtagtaaacgaggcgtgtagggtttttgctgaaggtatcgctgtcaaagcagcagaccttgacattgctggcataatgggaatgggttttccaccttacagaggaggaatcatgttctgggctgattccatcggatcgaaatacatttactcgaggctggatgagtggtcgaaggcttatggtgaattcttcaagccttgtgcttttttggctgagaggggatctaaaggagttcttttgagcgctcccgtgaaacaagccagctcacggttgtaa

SEQ ID NO: 108 - DNA, 1314 bp, Arabidopsis thaliana
  misc_feature 1..1314: note = ORF of protein Mrb1
  source 1..1314: mol_type = unassigned DNA
atggcgacgaacggatcgtcggagcatcttctcccggagacgacaagaccaagcgcgatcatcaacaaacaacaaccggagtttcctgcatcacgattcacgttcatgtccctcgtgctctggttcgaccagtcaaatttcggtacggctctgctttcgtggtccgttttcttccttctcgtcgtcatcgttcctttgatctctcacttcttgctcgtttgctccgactgcgatttccatcaccgacgtccgtacgacgttattgtgcagctttcgttatcgattttcgcaggaatctcgttcgttagcttatcgatttggtcgagaaaatttgggatgcgacggtttttgttccttgataagctttgggatgttagcgacaaagttagaatcgaatacgaagctgaaattcagagatcactaaaacggctaatgatcttcgtcctcccatcactaacactcgaagcaacctacagaatctggtggtacatctctggcttcaaccagattccttacatcatcaatcctattcttagccacgtcgtcgcttgcactctccagctctcttcttggctttaccgtaactccttattcatcattgtctgcattctctacaaaatcacatgtcatctacaaactcttcgtcttgatgacttcgctcgttgcttcgcttctgagatcactgacgttagatcagctctcggcgagcatcaaaagattcgtcgtaatttgaggattgttagtcatcgattcaggagatttattcttttgtctttgattcttgttactgctactcagttcatggctttgcttactactacgagagctagtgttgctgttaatatctatgaagttggcgaactcgcgttatgttcgttgagtttggttacaggagtattcatatgtttgagaagtgcaacgaagataactcacaaagctcaatctgtaaccagccttgcagctaaatggaacgtttgcgcaacagttgactcgtttgatcatcttgatggcgaaactcctactggttccattattgaatctcaagtttccctacgcggcaatgcgatagagacgtctgatgatgaagaaggagaaggagacgatgatctcgataataccaagatacatcccatctatgccaatacaatttcttaccaaaaacgtcaagctctagtgacgtatctagagaacaacaaagctgggatcactgtgtatggattccttgtagatagatcatggttgaatacgattttcgggattgagctcgctcttctactatggttgctcaataaaacaatcgtgaatataccatga

SEQ ID NO: 109 - DNA, 1665 bp, Medicago truncatula
  misc_feature 1..1665: note = ORF of protein Nsp1
  source 1..1665: mol_type = unassigned DNA
atgactatggaaccaaatccaacatcagatcacattcttgattggcttgaaggttctgtctctttctttccatcatttttggatgatccatataacaatggttacatccatgagtatgagatatggaatcagaatcaagatataagtaaccagtaccaaattgatgccaatactaattcttctaatgccacaaatagcacaaccaacattgttgctgctagtactaccacaagtacaacatcccttgagcctaatagctttaataacatacccttttcagatttgccgaagaaacgaaatgccgaagatgaactaagtctcaaaaaacaacctcaaaaccaaaagaacaagagacttaagagtcgtccgatgaatgaaagtgataatggagatgcagctcttgaagggacagtggttagaaaatctggtgggaacaagaaaggtgcagctaaggccaatggaagtaacagtaacaatggaaacaataaggatggtaggtgggctgagcagttgctcaacccttgtgctgtagccataaccggtggaaatctgaatcgtgtgcaacatctcttatatgttctccatgagctagcctcaactaccggtgatgccaaccaccggcttgcagcacatggtcttcgagcattgacacaccatttatcttcatcttcgtcatctaccccatcagggactattacttttgcatctacagaaccgcgattcttccaaaagtcattactgaagttctatgagtttagcccttggttttcctttcctaataacatagcaaatgcttccattcttcaagttctagctgaagagccgaacaatttgcgcacccttcacatccttgacattggagtctctcatggtgtgcaatggccaacttttctagaggccttgagtcgtcgacctggtggacctcctcctcttgttcgccttactgtcgttaatgcctcttcatccactgaaaatgaccaaaacatggagaccccattttcaataggtccatgtggtgatactttctcttctggactccttggttatgctcagtccttaaatgtcaatctgcagataaaaaagcttgataatcatccattgcagacattgaatgctaaaagtgttgacacatcctctgatgaaacccttattgtctgtgctcagttcaggttgcatcacttgaatcataacaatcctgatgaaagaagtgagtttctgaaggtgttgagaggcatggagcctaaaggggtgatattaagtgagaacaacatggagtgttgctgcagtagttgtggcgatttcgccactggattctcacgaagagtggagtacttatggaggtttttagattcaaccagttcggcattcaaaaaccgtgacagtgatgaaaggaaaatgatggaaggcgaggctgcaaaagcattgacaaaccagcgtgaaatgaacgaaaggagagaaaaatggtgtgaaagaatgaaagaagcagggtttgcaggggaagtatttggagaggatgcaattgatggaggtcgagctttgttaaggaagtatgataataattgggagatgaaagtagaagaaaatagtacaagtgtggaactatggtggaagagccaacctgtttctttctgttctttgtggaaactggataaacaaccagagtag

SEQ ID NO: 110 - DNA, 1533 bp, Arabidopsis thaliana
  misc_feature 1..1533: note = ORF of protein Nsp1
  source 1..1533: mol_type = unassigned DNA
atgttgttggaagaaacagaaccaccaaaccagactctagatcatgtcctaagctggcttgaggattctgtgtccttatccccattaccaggatttgatgattcttatttgctccacgagtttgatgggtctcaaacgtgggaatgggatcagactcaagatccggagcatggttttattcaaagctatagtcaagatcttagtgcagcatatgttggttgtgaagcaactaacctggaagtggtaacagaagctccatccattgatttggatcttccacctgaaattcagcaaccaaacgatcagtccaggaaaaggagccacgacgggtttctcgaggcacaacaggtgaaaaaatcggcaaggagcaagagaaaagcaatcaagtctagtgagaagagctccaaagatggtaacaaggaagggagatgggcagagaagttgcttaacccttgtgccttggccattacggcaagtaactcatcaagggttcaacattacctttgtgttctctctgaactggcctcttcttctggtgatgctaatcgtcggcttgcagcttttggtcttcgggctttgcaacatcatctttcctcatcctctgtgtcatcatccttttggcctgtttttacttttgcttcagcggaagtgaagatgtttcaaaagactctgcttaagttctacgaggtaagcccttggtttgctttgcctaacaacatggcaaactcagctatcctgcagattttagcacaggatcccaaagataaaaaggatcttcatattattgatattggtgtttctcacggtatgcaatggcccactttgttggaggctctgagctgcagactagaaggacctcctcctcgtgttcgaataaccgttatatcagatctaaccgcagacatacctttctctgttggtccaccaggttacaattatggttctcaactcctaggctttgctcggtctctcaagatcaaccttcagattagtgtacttgacaagttacaactcattgatacctcacctcacgagaacttaatcgtgtgtgctcagtttaggctgcatcacctgaagcatagcatcaatgatgagagaggcgagactttgaaagcagtgagaagtttaaggccaaaaggagtggttctttgtgagaacaatggagaatgcagtagtagtgcggactttgcagcaggattctcgaagaaactggagtatgtatggaagtttctggattcaacaagctcgggatttaaagaagagaatagcgaagagagaaaactaatggaaggagaggcaacaaaggtgttgatgaatgcaggagatatgaatgaaggaaaagagaaatggtatgagaggatgagagaagctggtttttttgtagaagcatttgaagaagatgcagttgatggagccaaatccttactaagaaagtatgacaacaattgggaaataagaatggaagatggagatacctttgctggattaatgtggaaaggagaggcagtttccttttgttcattgtggaagtag

SEQ ID NO: 111 - DNA, 1581 bp, Brachypodium distachyon
  misc_feature 1..1581: note = ORF of protein Nsp1
  source 1..1581: mol_type = unassigned DNA
atgaaggccatggactaccatgagccggacgagcaagagctcaccaccgccgtgaccgcgatcgccgacggcggcggcctggactggctggaggactccatctccttgctgtccgcggacggggatctcagcggcagctacggttggtggtgcgatccaccgctgctgccggcgcagcaagacgacattgggatcgtcgtcgcgcagacgctgccgcctcccgcggcgctcacgacgaccggttcgccccccacaagcattgcgtcgccggccccggcgtcgtccaagaagcgcaagtcgtccccggcgtccggccatagcggcggcggcagccagcgtcggcgcgcggagcaggagagggagaggcctgcaggcggcggcggcaagaagaaggggggcggttcggactgcagggaggctcggtgggcggagcagctgctgagcccgtgcgcggtggccgtggaggcccggaacctgtcgcgcgtgcagcacctcttctacgtgctgggcgagctggcgtccttctcctcgggcgacgccaaccaccgcctggccgcctacggcctgcgcgcgctctccctccgcctccccgccgcggttggccccgccgccgcagccgccgtgcgcgtcccggccttgggcgccgagtgcccgtcgtcgttcgcggccgcggagccccgggtgttccgcgcgtcgctgatccggttccacgaggtgagcccctggttcgcgctccccaacgcgctggccaacgccgccatcgcccagcagctgctgcaggcgccaggccggccgctcagcctcgtcgacgtcggcgtctcgcacggcgtgcagtggcccaccctgctcgagtccctcacccgcctccccgccggccgcgcgccgccctccgtccgcctcacggtcgtctccgcggcagcagctacacgggcagcgccgttctcggcgtcgcctccggggtacgacttcgcgccgcacctcctccgctacgccaagtccatcaacctcgacctccacatcgcccgcgccggcgacttgtccgcccagctgggcgaggaggaggcgctcgtggtcgtgtgcctccagttccgcctcggccacgtggcccccgaggagcggacggaggtcctccggaaggtccgggatctgaaaccggatctcgtggttctctcggagctggacgttgtggtcggaggcggcagtgcaggcgcggcgggcgagttctcggcgaggctggagctgctgtggcggttcctggagtccacgtcggcagcgttcaaggggagggacggcgaggacaggcggctcatggaggccgaggcgggggcgcccgcgggcatgatcacggcgccggcgtccggggaagggagggaggcgtggcgggaccgcatgagagcggcggggttccaggaagcggccttcgggggcgaggccgtggagtcggccaggtcgctgctgaggaagtacgacagcggctgggagatggcgggagcagccagcggagccgccgcggtggccttgcgctggaagggccagccggtctccttctgctccttgtggcgcccggcgccgccgccgtag

SEQ ID NO: 112 - DNA, 1629 bp, Glycine max
  misc_feature 1..1629: note = ORF of protein Nsp1
  source 1..1629: mol_type = unassigned DNA
atgatcatggaaccaaacccaacaccagatcacagtctggactggtttgaaggttctgtgtcttactttccaccattcttagattatccatacaactctagtgacatccaagagtatcagttgtgggaacaagacatagctagcaactaccaaactgataccaataccagttcccctaatgccaccaacattgttaccatcacaacaccccttgagacttgtgactctaacaacctaccactttctgatttgcccaagaaaagaagtgccactgatgagtcaagtcccaaaccaccacaaaaccacaagcacaagaaaatcaagagcaagcccaggaacaatgaagcagataacggagatgcagaagcgacaacaacaacaacagcaacagttagaaaatctggtgggaacaagaaaggtggagctaaggccaatggaaataactgcaacaataaggatgggaggtgggcagaacagttgttgaacccttgtgctgcagccataactggtggaaatctgaaccgtgtgcaacacctcttgtatgttctccatgagttagcatcaccaactggtgatgctaaccacaggcttgcagcacatggtctcaaagcactgacacaacacttatcttcttctccaacttctacctcttcagggtctataacttttgcgtctgctgagccacggttcttccaaaagacactactcaagttctatgaagttagcccttggttttcctttcctaacaacatagcaaatgcttccattcttcaagttcttggggaagacactgataattcacgcacccttcacatccttgacattggagtctctcatggcatgcaatggccaacgtttcttgaggcgttgagtcgcagagctggtggacctcctccattggttcgcctcaccgtggtcactgcttcttcctccactgaaaatgacaccccattttgtattggtcctcctggtgataacttctcttctagactccttggttttgctcagtcaatgaatgttaatttgcagataaacaagctagataattgcccattgcattcactgaatgctcaaagtgttgacgcctccccggatgaaatctttgttgtgtgtgcacagtttaggttgcatcagttaaatcacaatgccccagatgaaaggagtaagtttctgacagtgttgaggaacatggagcccaaaggggtgatactaagtgacaacaacctggggtgttgttgcaattgttgtggggatttcgccactggattctctcggagggtggagtacttgtggaggtttttggactcaacgagctctgcattcaaaggcagggagagcgaggagaggagagtgatggaaggtgaggctgcgaaggcattgacaaaccagcgcgagacgaatgaagggaaggagaaatggtgtgagagaatgaaagaagcagggtttgtggaggaagtatttggagaagatgcaatagatggaggtagagctttgctgaggaagtatgaaagtaactgggagatgaaagtggaagatgacaatagaagtgttggactatggtggaagggacaaagtgtttctttctgttctttgtggaagctggatgggaatgatcagagctcaacatga

SEQ ID NO: 113 - DNA, 1632 bp, Glycine max
  misc_feature 1..1632: note = ORF of protein Nsp1
  source 1..1632: mol_type = unassigned DNA
atgatcatggaaccaaatccaacatcagatcacagtctagactggtttgaaggttctgtgtcttactttccaccactcttagattatccatacgactctggtgacatccaagactatcagttgtgggaacaagacataaccagccactaccaaactgataccaataccagttcttcccctaatgctaccaacatttctacaaccacaacaccccttgagacttgtgactctaataacaacctaccactttctgatttgccaaagaaaagaaatgccactgatgagtctagtcccaaaccaccacaaaaccacaagcacaagaaaatcaagagcaaaccaataaacaatgaagcctataccggacacgcagaagggacaacaacaacaacagttagaaaatctggtgggaacaagaaagggggagctaaggccaatggaaataactgcaacaacaaggatggtagatgggcagaacagttgctgaacccttgtgctgcagccataacaggtggaaacctgaaccgtgtgcaacacctctcgtatgttctccatgagttagcatcacctactggcgatgctaaccacaggcttgcagcacatggtcttaaagcactgacacaacatctatcatcttctccctcttcagggtctataacttttgcttccagcgagccgcggttcttccaaaagacactcctcaagttctatgaggttagcccttggttttccttccctaacaacatagcaaatgcttccatccttcaagttcttggcgaagacactgataataattcacgcacccttcacatccttgacattggagtctctcatggaatgcaatggccaacttttcttgaggccttgagtcgcagacctggtggacctcctccgttggttcgcctcaccgtggtcactgcttcttcctccactgaaaatgacacaccattttgtattggtcctcctggtgataacttctcttctagactccttggttttgctcagtcaatgaatgttaacttgcagataaacaaactagataactgtccattgcatactctgaatgctcaaagtgttgacacctccccggatgaaatctttgttgtgtgtgcacagtttaggttgcatcagttgaatcacaatgcccctgatgaaaggagtgagtttctgaaggtgttgagaaacatggagcccaaaggggtgatactaagtgacaacaacatgggatgttgttgcaattgttgtggggatttcaccactggattctctcggagggtggagtacttgtggaggtttttggactcaacaagctccgcgttcaagggccgggagagcgacgagaggagagtgatggaaggtgaggctgcgaaggcattgacaaaccagcgcgagacgaatgaagggaaggagaaatggtgtgagagaatgaaagaagcagggtttgtgggggaagtatttggagaggatgcaatagatggaggtagagctttgctgaggaagtatgatggtaattgggagatgaaagtggaagatgacaatacaagtgttggactatggtggaaggggcaaagtgtttctttctgttctttgtggaagctggatgggaattataagacctcaacatttacatcatga

SEQ ID NO: 114 - DNA, 1572 bp, Oryza sativa
  misc_feature 1..1572: note = ORF of protein Nsp1
  source 1..1572: mol_type = unassigned DNA
atgagctaccatgaggaggagcgccatggcggcaacggcttggactggttcgaggagtccatgtcctctttgctcgccgccgacgtcgacctcgctggtggtggtggtgatgcaggcggcggcggctacgcgtggtggtgggctgcttcgccggcggcgcagcaggatgacattgggagcgtcgtggcgcagacgctgtcgcctccgtcgacggcggcaccggccgcggcgtcgccgagcattgcgtcgcccgcggcgtcgtcgccgtcggacgtcccgtcgtcgtcgtccaagaagcgcaagtcccccgcgcacagggcgcccgggcacaccggcggcaagaaggggggcggcggcaagggcggcggcggagggtccgaccgggacatgaggtgggcggagcagctgctgaacccgtgcgcggtggccgtcgaggccggcaacctgtcgcgcgtgcagcacctgttctacgtcctcggcgagctcgagtccttctccggagacgccaaccaccgcctggccgcgcacgggctgcgcgcgctcgcgcgctggctccccgccgccgttggccccgcggctgcagcggccgtgcgggtgccgccctgctccgagcgccccacgacggcgttcgcggccgccgagccgcgcctgttccgcgcctccctcatcaggttccacgaggtgagcccctggttcgcgcttcccaacgcgctggccaacgccgccatcgcgcaggcctcgacgtgcggcgctgccggggcgacgccccggccgctccacgtcgtcgatctcggcgtctcccacggcgtgcagtggccgacgctcctcgagtcgctcacgcggcagccgggcggccgcgcgccgccgtccgtccgcctcaccgtggtcgggcccggggccacggccacctccccggtggcgccattctcggcgtcgcctcccgggtacgacttctcgccgcacctcctccggtacgccaagtccatcaacctggacctccgcatctcccgcgccgccaccctggacgacgccgtgcccggcgacgacggcgaggcgctcgtcgtgtgcctccagttcaggctcgggcacgccgccgccgaggagcgaagagaagtcctccgcaaggcgagaggcctcaatccggagctggtcgtcctctcggagctcgactccggcgtcggcgtcgtcggcggcgacggcggcagcgcggcgggcgagttcgcggcgaggctggagctgctgtggcggttcctggagtcgacgtcggcggcgttcaaggggaaggacgtggaggagcggcggctgctggaggccgaggccggcgcgatcctcgcggcggcggacgtcgccgccgctggggaagggagggaggggtggagggagcgcatggcggcggcggggttcgaggaggcgccgttcggcgcggaggcggtggagtccgccaggtcgctgctccgcaagtacgacagcgggtgggagatgtcagcgccgtcgccggcggccgccgcggtggcgctgcggtggaaggggcagccggtgtccttctgctcgctgtggcggccggcggcgtag

SEQ ID NO: 115 - DNA, 1629 bp, Solanum lycopersicum
  misc_feature 1..1629: note = ORF of protein Nsp1
  source 1..1629: mol_type = unassigned DNA
atggccattgaagaacctgagcctaattccaactccgaccccatatcagagtggctagctagtactttatccgatgttccatccttcttccatgaaccttacagttatgccgatgacttaaatttctacgcggacccatggtgggttcctgatcaagaggatcagatcgttaatcacaacatcatcatcgacaatacttgtaactcattcaacattagctccccagtcaacacagctattaacaatatccctttggagcccatcattttggatcatccacagccggtggatctgtctaagaaaaggaaaaaatctgatcaaaatccaaaggcgtcaaagaagaatcataaacttcagattaatgaggcagcacatgctcctactatagttgatcaagaaggagtgcaattaaaaaaatcaataggaccgaagagagtaacaacaggaaataacagtaacaacaaagaaggaagatgggcagaacatttgctcaacccctgtgctgctgcaatcactgtaggtaacatgaaccgcgtgcaacatctgttgtacgttctccatgaactggcgtcattcacaggcgatgccaaccacaggctagctgctcatggactccgcgccttgacacatcatctgtcttcccctggctcatcatcagcatcctctggaactattggagttactaatttctcttctgccaatcataaattcttcagggactcgttgattaatttcatcgacgttagtccctggttccgaatccccaataacattgcgaattcatctgttctccaaataattggacaacaggatcggctaaagaaccttcatatccttgatattggagtttctcatggttttcaatggccaactcttcttgaagaattgactaggcggtcaggtggaccgccgccactggttcgcctgaccgttattacaccaaccacagaaaacggagaattgacaggaactccatttgtgatcgggccacctggctatgatttttcctcgcaactactagcctatgcaaaggctatcaacatcaatctacagatcaacagactggataattttcctcttcaaaacctcaattcccaaatcataaattcttcatctgatgaaaccttagtcatctgtgctcaatttagactgcataacttgaatcatactatcccggatgatcgaacagatttattgaagatattgaagagtttggacccgaaaggattagttctcagcgagaataacacggagtgcagctgcaacagctgtggggactttgcgaccacgttctcaaggagagtggagtacttatggaggtttttggattccacgagtgttgcctacaaagggcgggagagcgaagaaaggaggatgatggaaggagaagcagcaaaggcattgacgaacatgggagaaatgaatgaaagaaaagagaaatggtgtgagagaatgaggagcgctggttttgtaaaagcggtgtttggtgaagatgccattgacggagctcgggctttgctgaggaagtatgacagcaattgggagataagggtggaggagaaagatggctgtgtggatctatggtggaaaggacagccaatttcattctgctcattatggaagatttag

SEQ ID NO: 116 - DNA, 804 bp, Solanum lycopersicum
  misc_feature 1..804: note = ORF of protein Nsp1
  source 1..804: mol_type = unassigned DNA
atgaccattgatgaaactacttctgatcccatctcagagtggctggatagtcccttatcatactttccgtcgttactcgatgatgagtcatatagcatcgatgatttactcgatgagtcatatagcctcgatgatatttacgaggactcatggtgggctcctaatgagagcataaaccaagaaatacatatcaataacaataacgataatagtttaccgtgcttcgatactagcttccctgtcacaaccgcgagcactgtctctctggagccggtcatttcaagtcatcttccgccattggattcatccaagaagaggaaagggaatgattctggttacaatcccaaggcgtcccggaagaatcagaaccgtaggatcaatgatgcagatacaagaagaacaacaggacacaagaaagcaacacacaagtctagtacaggaaataatggtaacaacagagaaggaagatgggcagagcagttacttaacccttgtgctgctgcaattacagcaggaaatctgaaccgtgtgcagcacttgttgtacgttctgcgcgagctctcctcactaaacggagatgccaaccatcggttggctgctcgtggactccaggcgctgacgtattatctttctactccatgcccaacttcactatcttctgcttctgctgctacttttgcttcaacaaatccgaagttcttcaagaactcactgatcaacttcaatgagatcagtccctggttccgcatacccaatagcatttttctcatggtgttcaatggccaacattgctcgaggagttga

SEQ ID NO: 117 - DNA, 1638 bp, Zea mays
  misc_feature 1..1638: note = ORF of protein Nsp1
  source 1..1638: mol_type = unassigned DNA
atgaacaatgagagagaaagccaagaaccggcagccgccacgacctcggattggctcgatgagtccatcgccttcctagccgccaacctcgacctcggctttaacgactatggctggccgtcggcatcggaagcgcaggagcggcacgacgacgtccgcagcatgatcgtggagacgctagcacctactgctacaaccctgcaccagccaagcctgaaccttgatattagcgttgcgtcgtcgtcgtcaccgatgacctcgcagcaggagtttgggcagcccaagaaacgcaaatcttctggaaacagcagcagccagcgaccccatgttggcggcggcggtcagcaagaaaagggcggagcgagctgcgccaggccgaagccgggcaagaagggcagtggcaagggcgccgcggcaactgtggacagggaggcgaggtgggcggagcagctgttgaacccgtgcgcggccgccatcgaagcgggcaacctgccgcgcacgcagcacctgctgtacgtgctcggcgagctggcctccttctccggcgaccccaaccaccgcctggccgcgcacgggctgcgcgccctcacgcgacgcctcccgcacgccacgggccaagcggctctggcgaccgtgaaggtgcccccttgcgagtgcccgacgtcggtgttctccggcgtcgacccgcggctgttccgagcctcgctcatcaggttcaacgaggtcagcccatggttcgccgtcccgaacgcgctcgccaactccgcgattgcgcaggcggcgtcgaggaagaagggcgcggtggccgagccccgtcaggttcacgtcgtggacatcggcgtctcgcacggcgtccagtggccgacgctgctcgaggcgctcacccgcgtgccgcggggctccacgccgccctccgtccgcctcacgctcgtcggtcccacggctacaccacccgtgcccttctccgcatcgcctccaggctacgactgctcgccgcagctgctccggtacgccaagtccatcgacctcgacctcgccatcgtccatgcgccatgtttggacaccttgcgcggcactctcacccccggcgagaccctcgtcgtctgcctccagttccggctcggcctcaccaccgccgacgatcaagcagctatattccgaaaggtcagggacctaaacccggagctaatggtcctcactgagctagacggcggtcgccgcaacgatgacagcgcaaccagcgagttcgcggcgaggctggagctgctttggagattccttgactcgacggcggcggcgttcaaggggatggacgcgtatgagaggcgggtgatggaagcggaggccgggacagcattggcgctcacgccgcggtgcggagcgaccgtggacggggggagggaggcgtggcgagcgcgtttggccgcggcggggttcgaggaagcggcgttcgggggcgaggcggtggagacagcgaaggcgctgctgaggaagtacgatggcgggtgggagctggtgccaccgtctccgtccgcggcggcagcggtggggctccggtgggagggtcagcccgtgtcattctcatccatgtggcggccggcgcaggccagcccggtgctgattgggccttgcacataa

SEQ ID NO: 118 - DNA, 885 bp, Zea mays
  misc_feature 1..885: note = ORF of protein Nsp1
  source 1..885: mol_type = unassigned DNA
atggcagccacaatccggcgctcgtgcgcatgtatcgcgacaaaacggaaatatgcaactaactacatgcgaggcttatccaacatcttcttgcaacgagcaggagcagcacgggtgtcttcttgcaagattgcatgcatgaagcccatgagctgccacgagcaggagcagcacgcggagctcgccgccgccacgaccaacagcggcttcgactggcttgaggactccgtctccttcctcacagcagacgttgacgccgccggctgccgcggctacgggtggtgtccgcccctagcgcacacatcgccaagcattgcgtcaccggccgtgtcggagcctttgtccaagaagcgcaagtccccagcgcacagagcgtccgcccacagtggcagcaaccagcgtcgtcgcgctgaccaggcggacaggccaggtgctggcggcggcaagaagggcgggggagcgggatccgaccgcgacacgaggtgggcggagcagctcctgaacccgtgcacgaccgccgtcgaggccggcaacctgacgcgcgtgcagcacctgttctacgtgctcggcgagctcacctccttctccggcgacgccaaccaccgcctggccgcgcatgggctgcgcgcgctcgcccgcagcaggctccccgccgccgtcgggccggcggccgcgacggccgtgcgggtgccgccctgcgactgccccacgccggcgttcgcgggcacggagccgcggctgttccgcgcgtcgctcatcaggttccacgaggtgagcccctggttcgcgctggccaacgccgccatggagtggaatagggagaagggtaacagatgtagagacgccgtgagtgtcactggtttaatcgtagatgttaccttgtaa

SEQ ID NO: 119 - DNA, 1581 bp, Zea mays
  misc_feature 1..1581: note = ORF of protein Nsp1
  source 1..1581: mol_type = unassigned DNA
atgaagcccatgagctgccacgagcaggagcacccggagctcgccgccaccacgaccaacagcggcttcgactggcttgaggatcccatctccttcctcaccgcagacgtcgacatcgccggcggcggctatgggtggtggtccgatcccgcggcggcgcagcaagatggtattggcagcgtcgtggcgcagaccctttcgcccccggcgccgctgctggcgcacacctcgccgagcattgcgtcaccggtcgtgtcggagccttcgtccaagaaacgcaagtccccggcgcacagggcgtccgccagtactggcagcaaccagcgtcgccgcgccgaccaagacaggccaggtgctggtagcagcagcggcggcagcaagaggagcggctccaagggcggtggagcgggatccgaccgggacacgaggtgggcggagcagctcctgaacccgtgcgcgaccgccgtcgaggccggcaacctgtcgcgcgtgcagcacctgttctacgtgctcggcgagctcgcctccttctccggcgacgccaaccaccggctggccgcgcacgggctgcacgcgctggcccgcatgctccccgccgccgtcggcccggcggccgcggcgaccgtgcgggtgcctccctgcgagtgcccgacgccggggttcgcgggcgcggagccgcggctgttccgcgcgtcgctcatcaagttccacgaggtgagcccgtggttcgcgctccccaacgcgctcgccaacgccgccatcgcgcaggccgccgcgtcgggctcgggcgggcgcgggggctccgagccccggtcgctccacgtcgtcgacctcggcgtctcgcacggcgtgcagtggccgacgctgctggagtcgctgacccggctgcccggcgggcgcgcgccgccctccgtccgcctcaccgtcgcgggcgccccgccggcgcccttctcggcgtcgcctccggggtacgacttctcgccgcacctcctccggtacgccaagtccatcgacctgcggctcgcgatcagccgcgcggcctccctagactccgtgcacgggctcgcctcctccccgtcgggcgaggcgctcgtcgtgtgtctccagttccggctcggccacgccggcgccgacgagcggacagagattctaaggaaggtccggggtctcaacccggagctggtggtggtctcggagctcgacggcgggggcgacggcaccgcggcgggcgagttcacggcgaggctggagctgctgtggcggttcctggagtcgacgtccgcggcgttcaaggggagggatggggaggagaggcgcctgctggaggccgaggctggcacgtccgtcgcgtcggaggcggcaggggcagggagggaggcgtggcgggagcgcatggtggtggccgggttcgaggcggcggcgttcggagacgaggcggtggagtcggccaagtcgctgctgaggaagtacgacggcggctgggagatgtcggcgtcggggacggccggcgcggtggcgctgcggtggaaggggcagccggtgtcgttctgctcgctgtggcggccggcgtgcagcggctaa

SEQ ID NO: 120 - DNA, 1608 bp, Zea mays
  misc_feature 1..1608: note = ORF of protein Nsp1
  source 1..1608: mol_type = unassigned DNA
atgaagcccatgagctgccacgagcaggagcagcacgcggagctcgccgccgccacgaccaacagcggcttcgactggcttgatgaccccatctccttcctcacagcagacgttgacgccgccggctgcggcggctacgggtggtggtcagacgatcccgcggcggcgcagcaagataatattggcagcgtcgtggcgcagacgctttcgcccccggcggcgccgctgctgacgaccagtccgcccctggcgcacacctcgccaagcattgcgtcaccggccgtgtcggagccttcgtccaagaagcgcaagtccccggcgcacagggcgtccgcccacagtggcagcaaccagcgtcgccgcgctgaccaggcggacaggccaggtgctggcggcggcaagaagggcgggggagcgggatccgacgaccgcgacacgaggtgggcggagcagctcctgaacccgtgcgcggccgccgtcgaggccggcaacctgacgcgcgtgcagcacctgttctacgtgctcggcgagctcgcctccttctccggcgacgccaaccaccgcctggccgcgcacgggctgcgcgcgctcgcccgcagcaggctccccgccgccgtcgggccggcggccgcggcggccgtgcgggtgccgccctgcgactgccccacgccggcgttcgcgggcgcggagccgcggctgttccgcgcgtcgctcatcaggttccacgaggtgagcccctggttcgcgctccccaacgcgctggccaacgccgccatcgcgcgggccgcgtcgcgcggcgggtccacgcccgcggagcagccccggccgctccacgtcgtcgaccttggcgtctcgcacggcgtgcagtggccgacgctgctggagtcgctgacccgcctgtccggcggccgcgcgccgccctccgtctgtctcatcaccgtcgcggccgccccgccggcgcccttctcggcgtcgcctccggggtacgacttctcgccgcacctcctccggtacgccaagtccatcagcctgcagctcgccatcagccgcgcggcctccctggactccgtgcgtgggttggcctccccgtcggcgtcgggcgaggaggccctcgtcgtgtgtatccagttccggctcggccacgccagcgccgacgagcggacagagatcctaaggaaggtccgaggtctcgacccggagttggtggtggtctcggagctcgacggcgggggcgacggcaccgcggcgggcgagttcacggcgaggctggagctgctgtggcggttcctggagtcgacgtccgcggcgttcaaggggagggacggggacgagaggcgtctgctggaggccgaggctggcgcgtctctcgtcgcgtcggaggcgccggcgctggaagggagggaggcgtggcgagaggccatggcggcggccgggttcgaggaggcgtcgttcggagacgaggcggtggagtcggccaagtcgctcctgaggaagtacgacggcggctgggagatgtcggcgtcgggggcggccggcgcggtggcgctgcggtggaaggggcagccagtgtccttctgctcgctgtggcggccggcgtgcagcggctaa

SEQ ID NO: 121 - DNA, 1530 bp, Brassica rapa
  misc_feature 1..1530: note = ORF of protein Nsp1
  source 1..1530: mol_type = unassigned DNA
atgtcgctagaagaaacagaaccaccaagccaaactctagataatgtcctaggctggtttgatgattctatctccttaacgccattacctggattcactgattctgatttgctcaacgagtttgactggtctcaaacgtgggaatgggatcatcagactcaagctcaagatccaggatgtgattttcttcatagctatagtcaagatcttgatgcatatatagggtgtgaagcaactaatcttgaagtggtaacaggagttccagtcattgattcagatcctccaccagtggttcagctaccaaacgatcagaccaagaaacggaaccgtgatgagcttaacgatccccacgttgtgaaaagatcagcaaggagcaagaaaaaagcagacaagtctactgaggtgagccgcaaagatagcaacaaggcagagagatgggcagaacaacttcttaacccttgtgccttggcaatcacggcaaggaactcatctagggttcaacattacatttgtgttctctctgaacttgcctcttcttgtggtgattctaatcataggcttgcatcttttggtctttgtgctttgaaaaaacacatttcaacttcctttgtgccatcatccactcttacttttgcttcagcagaagtgaagatgtttcagaagactttgctcaagttctatgaagtaagcccttggtttgctttgcccaacaacatggcaaactcagccatcctacagattctatcacaagagacaagagataaaacagatcttcacattcttgatattggtgtttctcatggtatgcaatggcccactttgctggaagctctctgctgcagaccagaaggacctcctcttcaagttagaatcaccgttgtatccgatctaaccgcagacatacctttctctgttggtccaccatcttacaactatgcctctcagctcattggctttgctaggtccctcaacatcaacctccagataagtgtaattgacaagtctcaactcattgatacctcacctcatgagaccttcatcgtctgtgctcagtttaggctgtaccaactgaagaatagcatcactgatgagagaagcgaggctttgaaagcacttagaagtttgaatcctaaaggagttgttctttgtgaatacaatggagaaggcagtagcagtgtggactttgcagcagacttctcaaggaaactggactatctatggaagtttttggactcaacaagctatggattcaaagaagagaacagcgaagagaggaatctaatggaaggagaggcaacaaaggtgttgatgagctcaggtgatactaatgaagggaaagagatatggtatgagaagatgagaaaagctggcttctcagcagaagcatttggggaagatgcaatagatggagccaaatctttactcagaaagtatgataacaattgggaaataaagatggaagatgaagatacctttgctggtttactatggaaaggagaagaagtttccttttgttccttgtggaagtag

SEQ ID NO: 122 - DNA, 1281 bp, Hordeum vulgare
  misc_feature 1..1281: note = ORF of protein Nsp1
  source 1..1281: mol_type = unassigned DNA
cttcgtcgtcgcctccgcgtccaactccccctccccgtccgcctggcgctgtctggctccggtcatggtagcagcaaccagcgccgccgaatcgagcaggacaggccgggaggcggagcggggtcggaccgggacgcgaggtgggcggagcagctgccgggtccctgcgccgtcgccgtcgaggcgcgcaacctgtcccgcgtgcagcacctcttctacgtgctcggcgagctcgcggccttctccggtgacgccaaccacaggcttgccgcgtacgggctgcgggcgctctcgcttcgcctccctgccgcggttggccccgcggccgccgcggccgtgagggtgccggccgcatgcgagtgcctgacgccgtcgttcgccggcgcggagccacggctgttccgcgcgtccctcatcaagttccacgaggtcagctcgtggttcatgctccccaacacgttggcgaacgccgccatctcgcagctggcctccccggcgctccacctcatcgacattggcgtctcgcacggggtgcggtggccgaccttgctggagtccctcacccgcatggccgccggacgggcgccgccgtccgtccggctgacggtcgtggccaccccgccggtgcccttctcggcctcacctccggggtacgacttctctccgcacctcctccggtacgccaagtccattaagctggacctcgagatcggccgcgccgcctcactagactccgcgcacggcttctgcaccgctcccgacgaggcgctcgtggtgtgcctccagttcctgctgggccatgtcaccgccgaggagaggacggacatcatccgcagggtcaaagacctcaagccgcagctggtggtgctatcagagctcgacgtcgggggcaacggcagcgcggcgggcgagttcacggcgaggctggagctgctgtggcagttcttggagtcgacgcacgcgacgttcaaggggagggacgggaaggacaggcggttcctggaggccgaggcagggaccgccgtcgcggtggccgcggccagggaggccgtggggcgagaggggtggcgggaccgcatgacggcgacggggttcgagcaggtggcgttcgggggcgaggcggtggagtcggccaggtcgctgctgaggaagtacgacaacgggtgggagagtccacggcggcggcggcgacgggaggcggcggcgccgtggcgctgcggtggaaagggcagccggtgtccttctgctcgctgtggccgccggcgtagcagcggtagctga

SEQ ID NO: 123 - DNA, 1446 bp, Hordeum vulgare
  misc_feature 1..1446: note = ORF of protein Nsp1
  misc_feature 1..26: note = n is a, c, g, or t
  misc_feature 1183..1187: note = n is a, c, g, or t
  source 1..1446: mol_type = unassigned DNA
nnnnnnnnnnnnnnnnnnnnnnnnnncaccgcagtagagccccaccgcccccacaccatagcagagccccaccgccccgcagcccagcccaaccccatcgcgccgccgctcgcgttggcccacgcccacccctcgccaagcattgcatcgcccgcggcgtccgagccgtcgcccgggaagcgcaagtctccggtgcgtgtggggtctggctccggtcagggtagcagcaaccagcgccgccgaactgagcaggacaggccgggaggcggcaagaagggcggcagagcggggtcggaccgggacgcgaggtgggcggagcagctgctgggtccctgcgccgtcgccttcgaggcgcgcaacctgtcccgcgtgcagcacctcttctacgtgctcggtgagctcgcggccttctccagacaccccaagcacaggcttgccgcgtacgggctgcgggcgctatcgcttcgcctccctaccgcggttggccccgcggccgccgcagctgtgagggtgccggccgcatgcgagtgcccgacgccgtcgatcgccggcgcggagccacgactgttccgcgcgtccctcgtcaagttccacgaggtcagcccgtggttcacgctcctcaacacgctggcgaacgccgccatctcacagctggcctctgcggcgccccgtccgctccacctcgtcgacattggcgtctcgcacggggtgcagttgccgaccttgctggagtccctcacatgcatggccgccggacgggcgccgccgtccgtccggctgacggtcgtggccaccccgccggtgcccttctcggcctcgcctccggggtacgacttttcttcgcacctcctccgatacgccaagtccattaagctggacctcgagatcggccgggccacatcactagactccgcgcacggcttctacaccgctcccaacaaggcgcgcgtggtgtgcctccagttccggctgggccatgtcaccgccgaggagaggatggacatcatccgcagggtgaaagacctcaagccgcagcgggtggtgctatcagggctcgacgtcggtggcaacggcagcgtggcgggcgagtccacggcgaggctggagctgctgtggcggttcttggagtcgacgcacgaggcgttcaaggggagagacgggaaggacaggcggctcctggagnnnnnggcggggaccgccgtcgcggtggccgcggccagggaggccacggggagaaaggggtggcgagaccgcatgacggcggcggggttcgaccaggtggtgttcgggggcgaggtggtggagtcggccaggtcgctgctgaggaagtacgacaacgggtgggagatgtccacggtggcggcggcgaagggaggcggcggcgccgtggcgctgcggtggaaagggcagccggtgtccttctgctcgctgtggcggccggcgtag

SEQ ID NO: 124 - DNA, 429 bp, Hordeum vulgare
  misc_feature 1..429: note = ORF of protein Nsp1
  misc_feature 1: note = n is a, c, g, or t
  source 1..429: mol_type = unassigned DNA
naagacctgaagccgcagctggtggtcctatcagagctcgacgtcgggggcaacggcagcgcggcgggcgagttcacagcgaggctggagctgctgtggcggttcttggagtcgacgcacgcggcgttcaaggggagggacggggaggacaggcggctcctggaggcggaggcgggaaccggcgtcgcggtggccgcggccagggaggccatggggcgagaggggtggcgagaccgcatgacggcggcggggttcgaccaggtggcgttcgggggcgaggcggtggagtcggccaggtcgctgctgaggaagtacgacaacgggtgggagatgtccacggcggcggcggcgacgggaggcggcggcgccgtggcgctgcggtggaaagggcagccggtgtccttctgctcactgtggcggccggcgtag

SEQ ID NO: 125 - DNA, 1356 bp, Hordeum vulgare
  misc_feature 1..1356: note = ORF of protein Nsp1
  source 1..1356: mol_type = unassigned DNA
acaaactgcagatacgtatataccagtcccctctttctccgtcggggtcggacgtgcctaccggagtgtcggaggtgcaaccgcgcatgcgcatgcgcagcaatgcaagcaggcggccgaggcaaggcgacgacgaacaagcagggatggatcgatgccttctcctcttccgaaatgggcccttgggcgtcgcggagcggggtcggaccgggacgtgaggtgggcggagcagctgctgggtccctgcgtcgtcgccgtcgaggcgcgcaacctgtcccgcgtgcaacacctcttctacgtgctcggcgagctcgcggccttctccggcgacgccaaccacaggcttgccgcgtacgggctgcgagcgctctcgcttcgcctccctgccgcagttggccccgcggccgccgcggccgtgagggtgccggccgcatgcgagtgcccgacgccgtcgctcgccgggcggagccacggatgttccgcgcgtccctcatcaagttccacgaggtcagcccgtggttcacgctccccaacacgctggcgaatgccgccatctcgcagctggcctctgcggcgccccggccgctccacctcgtcgacattgacgtctcgcacgaggtgcagtggccgaccttgctggagtccctcacccgcatggccgccggacgggcgccgccgttcgtccggctgacggtcgtggccaccccgccggtgcccttctcggcctcgcctccggggtacgacttctctccgcacctcctccggtacgccaagtccattaagctggacttcgagatcggtcgcgccgcctcactagactccgcgcacggcttctgcaccgctcccgacgaggcgctcgtggtgtgcctccagttccggctgggccatgtcaccgccgaggagaggacgtacatcatccgcagggtcaaagacctcaagccgcagctgatggtgctatcagagctcgacgtctggggcaacggcagcacggcgggcgagttcacggcgaggctggagctgctgtggcggttcttggagtcgacgcatgcggcgttcaaggggagggacggggaggacaggcggctcctggaggccgaggcggggagcgccgtcgcggtggccgcggccagggaggccgtggggcgagaggggtggcgggaccgcatgacggcggcggggttcgaccaggtggcgttcgggggcgaggcggtggagtcggccaggtcgctgctgaggaagtacgacaacgggtgggagatgtccacgacggcggcggcgacgggaggtggcggcgccgtggcgctgcggtggaaagggcagccggtgtccttctgctcgttgtggtggcgggcgtag

SEQ ID NO: 126 - DNA, 1134 bp, Hordeum vulgare
  misc_feature 1..1134: note = ORF of protein Nsp1
  source 1..1134: mol_type = unassigned DNA
gcaccaccaccgtgtgcatttaccaaattaactaagcgccctagcctgcaccttcgccgtcgcctccgcgtccaactccccctccccgtccgcctggcggtgtctggctccggtcatggtagccgcaaccagcgccgccgaaccgagcaggacaggccgggaggcggcaagaagggcggcggagcggggtcggaccgggacgcgaggtgggcggagcagctgctgggtccctgcgccgttgccgtcgaggcccgcaacttgtcccacgtgcagcacctcttctacgtgctcggtgagctcgcggccttctccggcgacgccaaccacaggcttgccgcgtacgggctgcgggcgctctcgcttcgcctccctgccgcggttggccccgctgccgccgcggccgtgagggtgccggccgcatgcgagtgcccgacgccgtcattcgccggcacggagccacggctgttccacgcgtccctcatcaaattccacgaggtcagcccgtggttcacgctccccaacacgctggcgaatgccgccatctcgcagctggcctctgcggcgccccggccgctccacctcatcgacattggcgtctcacacggggtgcagtggccgaccttgctcgagtccctcacccgcatggccgccggacgggcaccgctgtccgtccggctgacggtcgtggccaccccgccggtgcccttctcggcctcgcctccggggtacgacttctctccgcacctcctcccgtacgccaagtccattaagctggacctcgagatcggccgcgccgcctcactagactctgcgcacggcttctgcaccgctcccgacgaggcgctcgtggtgtgcctccagttccggctgggccatgtcaccgccgaggagaggacggacatcatccgcagggtcaaagacctcaagccgcagctggtggtgctatcagagctcgacgtcgggggcaacgacagcgcggcgggcgagttcactgcgaggctggagctgctatggcggttcttggagtcgacgcacgcggcgttcaaggggagggacggggaggacaggcggctcttggaggccgaggcggggaccgccgtcgcggtggccgcggccagggaggccgtggggtga

SEQ ID NO: 127 - DNA, 789 bp, Hordeum vulgare
  misc_feature 1..789: note = ORF of protein Nsp1
  source 1..789: mol_type = unassigned DNA
agcggcaaccagcgccgccgaaccgagcaggacaggccgggcggcggcaagaagggcggcggagcggggtcggaccgggacgcgaggtgggcggagcagctgctgggtccctgcgccgtcgccgtcgaggcgcgcaacctgtcgcgcgtgcagcacctcttctacgtgctcggcgagctcgcggccttctccggcgacgccaaccacaggctggccgcgtacgggctgcgtgcgctctcgcttcgcctccctgctgcggttggccccgcggcagccgcggccgtgagggtgccggccgcatgcgagtgcccgacgccgtcgttcgccggcgcggagccacggctgttccgcgcgtccctcatcaagttccacgaggtcagcccgtggttcacgctccccaacacgctggcgaacgccgccatctcgcagctggcctctgcggcgccccggccgctccacctcgtcgacattggcgtctcgcacggggtgcagtggccgaccttgctggagtccctcacccgcatggccgccggacgggcgccgccgtccgtccggctgacggtcgtggccaccccgccggtgcccttctcggcctcgcctccggggtacgacttctccccgcacctcctccggtacgccaagtccattaagctggacctcgagatcggccgcgccgcctcactagactccgcgcatggcttctgcaccgctcccgacgaggcgctcgtggtgtgcctccagttccggctgggccatgtcaccgccgaggagaggacggacatcatccgc

SEQ ID NO: 128 - DNA, 693 bp, Hordeum vulgare
  misc_feature 1..693: note = ORF of protein Nsp1
  source 1..693: mol_type = unassigned DNA
atgctccacctcctcctacaacgaaaaactgttacacctccacgaaaaaaagagccaatcaagtacagaaatttacgaacaatgcaccttcgccgtggcctccgcgtccaactccccctccccgtccgcctggcggtgtctggctccggtcatggtagcagcaaccagcgccaccgaaccgagcaggacaggccgggaggcggcaagaagggcggcggagcggggtcggaccgggacgcgaggtgggcggagcagctgctgggtccctgcgctgtcgccgtcgaggcgcgcaacctgtcccgcgtgcagcacctcttctacgtgggcgagctcgcggccttctccggcgacgccaaccacaggcttgccgcgtacgggctgcgggcgctctcgcttcgcctccctgccgcggttggccccgcggccgtgagggtgccggccgcatgcgagtgcccgacgccgtcgttcgccgccgcggagccacggctgttccgcgcatccctcatcaagttccacgaggtctgctcgtggttcacgctccccaacacgttggcgaacgccgccatctcgcagctagcctccccggcgctccacctcatcgacattggcgtatcgcacggggtgcagtggccgaccttgctggagccctcacccgcatggccgccggacgggcgccgccgtccgtccggctga

SEQ ID NO: 129 - DNA, 1629 bp, Solanum tuberosum
  misc_feature 1..1629: note = ORF of protein Nsp1
  source 1..1629: mol_type = unassigned DNA
atgaccattgaagaacctgagcctaattccaactccgaccccatatcagagtggttagctagtactttatccgatgttccatccttcctcgatgaaccttacagttacaccgatgatttaaatttctacgcggactcatggtgggttcccgatcaagatgatcagatcgttaatcacaacatcatcatcgacaatacttgtaactcattcaacattagctccccagtcaacacagctattagcaatatccctttggagcccatcattttggatcagccacagccggtggatctgtctaagaaaaggaaaatatctgatcaaaatcctaagacgtcaaagaaaaatcataaccgtcagattaatgaggcagaaaatgctcatactataggtgatcaaggaggagtgcaagtaagaagaccaaagagagtaacaacaaaatcaaccggaaataacagtaacaacaaagaaggaagatgggcagaacatttgctcaacccttgtgctgctgcaatcactgtcggtaacatgaaccgcgtgcaacatctgctgtacgttctccatgagttggcgtcgttcacaggtgacgccaaccacaggctagctgctcatggactccgcgccttgacacatcatttgtcttcccctggctcatcatcagcatcctctggaactattggagttactaatttctcttctgcgaatcataaattcttcagggactcgttgatcaatttcatcgacattagtccctggttccgaatccccaataacattgcgaattcatctgttctccaaataattggagaacaggatcggctaaagaaccttcatatccttgatattggagtttctcatggttttcaatggccaactcttcttgaagaattgactaggcggtcaggtggaccgccaccactggttcgcctgaccgttattacacccacaacagaaaacggagaattgagaggcactccatttgtgatcgggccacctggttacgatttttcctcgcaactgctagcctatgctaaggctatcaacattaatctacagatcaacagactggacaattttcctcttcaaaatctcaattcccaaatcataaattctacatctgatgaaaccttagtcatctgtgctcaatttagactgcataacttgaatcatactatcccggatgatcgaacagatttgttgaagatattgaagagtttggacccgaaaggattagttctcagcgagaacaacacagagtgtagctgcaacagctgtggggactttgcgacaatgttctcaaggcgagtggagtacttatggaggtttttggattccacaagtgtggcctacaaagggcgggagagcgaagaaaggaagatgatggaaggagaagcagcaaaggcgttgacgaacatgggagaaatgaatgaaagaaaggagaaatggtgtgaaagaatgaggaacgttggctttgtaaaagaggtgtttggtgaagatgccattgacggagctcgggctttgctgaggaagtatgacagcaattgggagatgagggtggaggagaaagatggctgtgtggatctatggtggaaagggcagccaatttcattctgctcattatggaagatttag

SEQ ID NO: 130 - DNA, 1662 bp, Solanum tuberosum
  misc_feature 1..1662: note = ORF of protein Nsp1
  source 1..1662: mol_type = unassigned DNA
atgaacattgatgaacctacttctgatcccatctcagagtggctggatagtcccttatcatattttccgtcgttactcgatgagtcatatagcatcgatgatttattcgatgagtcatatagcctcgatgatttattcgatgagtcatatagcctcgatgatatttacgaggactcgtggtgggctcctagtgagagtataaaccaagaaatacatatcaataacaataacaataacagtttaacgtgcttcgacactagcttccctgtcacagccgcgagcactgtctctttggaaccggtcatttcaagtcacccttcgccattggattcatccaagaaaaggaaagggaatgattccggttacaatcccaaggcgtcccggaagaatcagaaccgtcggatcaatgatgcagataaaagaagatcaacaggacacaagaaagcaacacacaagtccacaggaaataatggtaacaacagagaaggaagatgggcagagcagttacttaacccttgtgctgctgcaattactgcaggaaatctgaaccgtgtgcagcacttgttgtacgttctgcgcgagctctcctcactaaacggagatgccaaccatcggttggctgctcgtggactccaggcgctaaagtattatctttctactccatgcccaacttcactatcttctgtttctgctggtggtgttgctacttttgcttcaacaaatccaaagttcttcaagaactcactgatcaacttcaatgagatcagtccctggttccgcatacccaatagcattgcaaactcctcaatactccaaattcttgggaaacacgattcatcccagaacctacacattcttgatattggagtttctcatggtgtccaatggccaacgttgcttgaggagttgacccatagagcaggaggtccgccaccattggttcgtctaacagtaataacacctaccatagaagatgatcaacaaagaaatagtccatttgtagttggtccaggtggttacaacttttcggtgcaactccttgcctttgccaaggctattaacatcaatctacaaatcaacagactggaaaattacccacttcagaatcttgattcccaagtgataaattcatccccagatgaaatcttggttatttgtgcacagttcagactccataacttgaaacacaacaatccagacgagaggacagagttgttgaaaagactaaagagtttggagccaaaaggactggttttcagtgagaacaacatggattgcagctgcaacagttgcggggacttcacaacaagattctcaaggcgagtggagtacttgtggaagttcttggactccaccagtgtagcatacaaaggacgtgagagcgatggaaggaggatgatggaaggagaagcagcaaaggctttgacaaacatgggagagatgaatgagacaaaggagaaatggtgtgaaagaatgaggactgttggtttcgtatggaagatgtttggagatgatgccattgatagagctcgtgcattgttgaagaagtatgacaacaactgggagatgagagttgatgagaaggatggctgtgttggactgtggtggaaaggagatcctgtttcattttgttcattatggaagatagatcccaacacatga

SEQ ID NO: 131 - DNA, 1251 bp, Triticum aestivum
  misc_feature 1..1251: note = ORF of protein Nsp1
  source 1..1251: mol_type = unassigned DNA
atggtgtccggctccggccatagcagcagcaacaaccagcgtcgccgaactgagcaggataggccgggcggcggcaagaagggcggcggcgcggggtcggacagggacgcgaggtgggcggagcagctgctgggcccgtgcgccgtcgccgtcgaggcgcgcaacctgtcgcgcgtgcagcacctcttctacgtgctcggagaactcgcggccttctccggcgacgccaaccacaggctagccgcgtacgggctgcgggcgctctcgcttcgcctcccagccgcggtgggccccgcggccgccgcggccgtgagggtgccggccgcctgcgagtccccgacgccgtcgttcgctggcgccgagccacggctgttccgcgcgtccctcatcaagttccacgaggtcagcccgtggttcacgctccccaacacgctggcgaacgccgccatctcgcagctggcttcggcagcgccccggccgctacacctcgtcgacattggcatctcacacggggtgcagtggccgactttgctggaatctctcacccgcatggccgctgggcgagcaccgccatccgtccggctgactgttgtggccaccccgccggtgcccttctcggcctcgcctccggggtacgacttctcgccgcatctcctccggtacgccaagtccattaagctggacctcgagatcagccgcgccgcttcactagaccccgcgcatggcttgtgcaccactcccgacgaggcgctcgtggtgtgcctccagttccggctgggccatgtcaccgccgaggagaggacggacatcatccgcagggtcaaaggcctcaagccgcacctggtggtcctatcggagctcgacgtcgggggcaacggcagcgcggcaggcgagttcacggcgaggctggagctgctgtggcggttcttggagtccacgcacgcggcgttcaaggggagggacggggaggacaggcggctcctggaggccgaggcgggcaccgccgtggcggtggccgcggccagggaggccatggggcgggagggctggcgggaccgcatgacggcggcggggttcgagcaggtggcgttcgggggcgaggcggtggagtcggccaggtcgctgctgaggaagtacgacaacgggtgggagatgtccacggcggcggcggccacgggaggcggcggcgccgtggcgctgcggtggaaagggcagccggtgtccttctgctcgctgtggcggccggcgtag

SEQ ID NO: 132 - DNA, 213 bp, Triticum aestivum
  misc_feature 1..213: note = ORF of protein Nsp1
  source 1..213: mol_type = unassigned DNA
ccgccttccgtccggctgacggtcgtggccaccccgccggtgcccttctcggcctcgcctcccggatatgacttcgcgccgcatctcctccggtacgccaagtccattaagctggacctcgagatcggccgcgccgcttcactagaccccgcgcatggcttgtgcaccactcccgacgaggcgctcgtggtgtgcctccagttccggctcggc

SEQ ID NO: 133 - DNA, 1605 bp, Lolium perenne
  misc_feature 1..1605: note = ORF of protein Nsp1
  source 1..1605: mol_type = unassigned DNA
atgaaggccatgggctgccatggacaagaggaccaaaagctcgccctcgtcgcagccagcgacgatggcttcgactggctggaggactccatctccttgctcgctgcggacatcgatctcggtggcagctacgggtggtggtgtgatccattgccggcgcagcagcaagatgacattgggagcgtcgtggcgcagacgctgtcgcctccagcgccacagacgacaagctcgcccctggcccacgcctcaccgagcattgcatcgccagcggtgtcatccccgtccacggagccgtcgtccaagaagcgcaagtctccgacgcacagggtgtccggccatagcagcagcaaccagcgtcgccgcgccgaacaggagaggccaagtggcggcggcagcaagaagggcagcagcaagggcggcggagctggttcggacagggacgcgaggtgggcggagcagctgctgagtccgtgcgccgttgccgtcgaggcacgcaacctgtcgcgcgtgcagcacctgttctacgtgctcggcgagctcgcgtccttcgccggcgatgccaatcaccggctggccgcgtatgggctgcgagcgctctcgcttcgcctacctgcagcagtgggcacagctgccgcagctgcggtgaaagtgccggactgcgagtgcccgacgccgtcgttcgctggcgcggagccccggctgttccgcgcgtccctcatcaagttcaacgaggttagcccgtggttcgcgctgcccaacgctcttgcgaacgccgccatcgcgcagctggcggcctcgacgcgcggcgccgccgccgcgccccggccgctccacctcgtcgacatcggcgtttcgcacggagtgcagtggccgactttgctggagtccctgacccgcgtgcccgccggacgagcaccgccgtctgtccggctcacggtcgtaggcatcccaccggtgacgacgccattctcggcgtcgcctccggggtacgacttctcaccgcatctcctccggtacgccaagtccattaacctggacctcgagatcagccgcgccgcctcgctagactccgcgcatgccttgtacggtccggacgaggcgctcgttgtgtgcctccagttccggctcggccacgtcacggccgaggagcgaacggacatcctccggagggtcaaagacctcaagccggatctggtggtactctcggagctcgacgtcggcggaggcaacgtcagcaacgcggcgtgcgagttcacggcgaggctagagctgctgtggcggttcttggagtcgacgtcggcggcgttcaaggggagggacggggaggacaggcggctgatggaggccgaggcggggactgccgtggcggttaccgcggcaggggatcatggcagggagggctggcgggagcgcatgacggcggcggggttcgaggaggcgccgttcgacggcgaggcggtggagtccgccaggtcgctgctgaggaagtacgacaacgggtgggagatgtccacggcgcccgcggcgacaggaggcggcggcgcggtggcgctgcggtggaaggggcagccggtgtccttctgctcgctgtggcggccggcgtag

SEQ ID NO: 134 - DNA, 1605 bp, Lolium perenne; identical to SEQ ID NO: 133
  misc_feature 1..1605: note = ORF of protein Nsp1
  source 1..1605: mol_type = unassigned DNA

SEQ ID NO: 135 - DNA, 1701 bp, Arabidopsis thaliana
  misc_feature 1..1701: note = ORF of protein Pds
  source 1..1701: mol_type = unassigned DNA
atggttgtgtttgggaatgtttctgcggcgaatttgccttatcaaaacgggtttttggaggcactttcatctggaggttgtgaactaatgggacatagctttagggttcccacttctcaagcgcttaagacaagaacaaggaggaggagtactgctggtcctttgcaggtagtttgtgtggatattccaaggccagagctagagaacactgtcaatttcttggaagctgctagtttatctgcatccttccgtagtgctcctcgtcctgctaagcctttgaaagttgtaattgctggtgctggattggctggattgtcaactgcaaagtacctggctgatgcaggccacaaacctctgttgcttgaagcaagagatgttcttggtggaaagatagctgcatggaaggatgaagatggggactggtatgagactggtttacatattttcttcggtgcttatccgaatgtgcagaatttatttggagaacttgggatcaatgatcggttgcagtggaaggaacactccatgatttttgctatgccaagtaaacctggagaatttagtagatttgacttcccagatgtcctaccagcacccttaaatggtatttgggctattttgcggaacaacgagatgctgacatggccagagaaaataaagtttgctattggacttttgccagccatggtcggcggtcaggcttatgttgaggcccaagatggtttatcagtcaaagaatggatggaaaagcagggagtacctgagcgcgtgaccgacgaggtgtttattgccatgtcaaaggcgctaaactttataaaccctgatgaactgtcaatgcaatgcattttgatagctttgaaccggtttcttcaggaaaaacatggttccaagatggcattcttggatggtaatcctccggaaaggctttgtatgccagtagtggatcatattcgatcactaggtggggaagtgcaacttaattctaggataaagaaaattgagctcaatgacgatggcacggttaagagtttcttactcactaatggaagcactgtcgaaggagacgcttatgtgtttgccgctccagtcgatatcctgaagctccttttaccagatccctggaaagaaataccgtacttcaagaaattggataaattagttggagtaccagttattaatgttcatatatggtttgatcgaaaactgaagaacacatatgatcacctactctttagcagaagtaaccttctgagcgtgtatgccgacatgtccttaacttgtaaggaatattacgatcctaaccggtcaatgctggagctagtatttgcaccagcagaggaatggatatcacggactgattctgacatcatagatgcaacaatgaaagaacttgagaaactcttccctgatgaaatctcagctgaccaaagcaaagctaaaattctgaagtaccatgtcgttaagactccaagatctgtgtacaagaccatcccaaactgtgaaccatgtcgtcctctacaaagatcacctattgaaggattctacttagctggagattacacaaaacagaagtacttagcttccatggaaggcgctgtcctctctggcaaattctgctctcagtctattgttcaggattacgagctactggctgcgtctggaccaagaaagttgtcggaggcaacagtatcatcatcatga

SEQ ID NO: 136 - DNA, 1899 bp, Arabidopsis thaliana
  misc_feature 1..1899: note = ORF of protein Pen3
  source 1..1899: mol_type = unassigned DNA
atggaaactgatcctgctaactcttcatcgaagtctccagctgtagtatctgagaaagatgtattgattcctgaaccttctgaaaatactgtgggcgttgtacaagatcctgtgagcgctgaaagagaagctcatgaagactctattagtacagaagcttctgttgcaaaggttgatgatactcaaatgcctgctagttctaccggctctgaacctttaagtaaaacagatgatatagttccatgcccaccaggttcctccccacgcgaatcaccgccgtctatattttcttcttctggtctttcttcttgggccaaaagttttaaattccagcagcaagatccaaatcgtacagattctgggatgtcagctttcacaagattcacctctgaacttggtctgcatttgccaacgaaagggtctgaggaggttggtgattctagatcatcaaacacacaagttggtggtgcttttgagtccctcacaaaagctgtggttgattcatctcgtggtgccgttaaagcaatgcaggtgaaagcacgccacattgtctctcagaacaaaagaagataccaggagggggaattcgatttggacatgacatatataactgagaatataatcgcaatggggttcccagctggtgacataagctctggtcttttcggatttttcgagggactatatcgtaatcacatggaagaggtgataaagttttttgaaactcatcataaggataaatataaggtctacaacctttgttcagagagattgtacgatgcttcaagatttgaggggaaggttgcatctttcccatttgatgatcataattgtcctcctatccaattgataccatcgttttgtcaaagtgcatacacatggctgaaggaagatatccaaaatgtagtggtagttcattgtaaagctggaatggcaagaaccggattaatgatttgctgtcttcttctatatctcaagttctttcctacagctgaagaggctattgattactataaccaaaagagatgtcttgatgggaaagctcttgttctcccaagtcagattcgatatgtcaagtactacgaacgtgttcaaaaccaatttgacgggaaagttccacccgagcgaagatgcatgctcagaggatttcgcctaatcaactgcccttactggattagacctgccattacgatctccaaccacactgatatacttttctcaacaaagaaacatcagaaaaccaaggatttggggcctgaagatttttggataaaggcacccaaaaagggagtggttgtctttgcaatacctggtgaagctggtctaacagaattagccggcgacttcaaaatccactttcaagatagcgatggagatttctactgttggctaaatactacactgactgataacagaacaatgctgaaaggcagtgactttgatggctttgacaagagaaaactgcctgcaccgggtttccatgtcgagatcgtaatgatagaacccgacaattcacaaccaacaaagtccaaatctgattccacccaacaacaatctcaaagcagctccagtgctgattccagcaaacttaagtctaatgagaaagacgacgatgtattctctgacagtgatggggaagaggaaggaaactctcagtcttattcaactaacgagaaaaccgcaagctcaatgcacacaactagtaaacctcatcagatcaatgagccaccaaagagagatgacccttccgcaaacagaagtgtcacaagttcatcgtcctcgggtcattataatccaataccaaacaactcattagcagttagtgatatcaaagctattgctgcagatgcatcggtcttctcctttggagatgaggaagaagactatgaaagcgactga

SEQ ID NO: 137 - DNA, 3519 bp, Arabidopsis thaliana
  misc_feature 1..3519: note = ORF of protein Phyb
  source 1..3519: mol_type = unassigned DNA
atggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgcagttgcaagagagatggttaggctcattgagactgcaactgttcctatattcgctgtggatgccggaggctgcatcaatggatggaacgctaagattgcagagttgacaggtctctcagttgaagaagctatggggaagtctctggtttctgatttaatatacaaagagaatgaagcaactgtcaataagcttctttctcgtgctttgagaggggacgaggaaaagaatgtggaggttaagctgaaaactttcagccccgaactacaagggaaagcagtttttgtggttgtgaatgcttgttccagcaaggactacttgaacaacattgtcggcgtttgttttgttggacaagacgttactagtcagaaaatcgtaatggataagttcatcaacatacaaggagattacaaggctattgtacatagcccaaaccctctaatcccgccaatttttgctgctgacgagaacacgtgctgcctggaatggaacatggcgatggaaaagcttacgggttggtctcgcagtgaagtgattgggaaaatgattgtcggggaagtgtttgggagctgttgcatgctaaagggtcctgatgctttaaccaagttcatgattgtattgcataatgcgattggtggccaagatacggataagttccctttcccattctttgaccgcaatgggaagtttgttcaggctctattgactgcaaacaagcgggttagcctcgagggaaaggttattggggctttctgtttcttgcaaatcccgagccctgagctgcagcaagctttagcagtccaacggaggcaggacacagagtgtttcacgaaggcaaaagagttggcttatatttgtcaggtgataaagaatcctttgagcggtatgcgtttcgcaaactcattgttggaggccacagacttgaacgaggaccagaagcagttacttgaaacaagtgtttcttgcgagaaacagatctcaaggatcgtcggggacatggatcttgaaagcattgaagacggttcatttgtgctaaagagggaagagtttttccttggaagtgtcataaacgcgattgtaagtcaagcgatgttcttattaagggacagaggtcttcagctgatccgtgacattcccgaagagatcaaatcaatagaggtttttggagaccagataaggattcaacagctcctggctgagtttctgctgagtataatccggtatgcaccatctcaagagtgggtggagatccatttaagccaactttcaaagcaaatggctgatggattcgccgccatccgcacagaattcagaatggcgtgtccaggtgaaggtctgcctccagagctagtccgagacatgttccatagcagcaggtggacaagccctgaaggtttaggtctaagcgtatgtcgaaagattttaaagctaatgaacggtgaggttcaatacatccgagaatcagaacggtcctatttcctcatcattctggaactccctgtacctcgaaagcgaccattgtcaactgctagtggaagtggtgacatgatgctgatgatgccatattag

SEQ ID NO: 138 - DNA, 1575 bp, Arabidopsis thaliana
  misc_feature 1..1575: note = ORF of protein Pif3
  source 1..1575: mol_type = unassigned DNA
atgcctctgtttgagcttttcaggctcaccaaagctaagcttgaatctgctcaagacaggaacccttctccacctgtagatgaagttgtggagctggtgtgggaaaatggtcagatatcaactcaaagtcagtcaagtagatcgaggaacattcctccaccacaagcaaactcttctagagctagagagattggaaatggctcaaagacgactatggtggacgagatccctatgtcagtgccatcactaatgacgggtttgagtcaagacgatgactttgttccatggttgaatcatcatccctcccttgatggatattgctctgatttcttgcgtgatgtgtcgtctcctgttactgtcaacgagcaagagagtgatatggcggtaaaccaaactgctttcccgttgtttcagagaagaaaggatggcaatgaatcagctcctgctgcttcttcgtcgcagtataacggtttccaatcgcattctctgtatggaagtgatagagctagagatcttcctagccaacaaaccaatccggatcggtttactcagacgcaggaaccactaattactagtaacaagcctagtttggtcaacttttcacatttcttacgccctgcaacttttgcgaagactactaataataaccttcatgacactaaagaaaagagtcctcaaagcccgccaaatgtgtttcagaccagagttcttggagctaaagactctgaagataaggttcttaacgagtctgttgcttctgctacgcctaaagataaccaaaaggcttgcctaatatcagaggactcatgtagaaaagaccaagagagtgaaaaagcagttgtatgttcttctgttggctcgggtaatagtctcgatggcccatccgaaagtccttcactttctttaaagagaaagcattcgaatattcaagacattgactgtcatagtgaagatgtggaagaagaatcaggagatggaagaaaggaagcaggtccatctcgaacgggtttgggttcaaagagaagccgctctgcagaagtgcataatctgtctgaaaggagacggcgtgataggatcaacgagaagatgcgtgccctgcaagaactcattccaaactgtaacaaggtggacaaagcttcgatgctagatgaagccatcgagtatctcaagtcactccaacttcaagtgcagatcatgtcaatggcgtctggttactatctgccaccggcggttatgttcccaccgggtatggggcattacccggcagcagctgctgcaatggcaatgggtatgggaatgccttatgcaatgggcttgcctgatttgagccgtggtggttcatcggttaaccacggaccacagttccaagtctcggggatgcaacaacaaccagtggcgatgggtattccacgtgtctctggtggtggtatctttgccggttcttcgacgattggcaatggctcgactagagatttatctggttctaaagatcaaacaacgacgaataacaacagtaacttgaaaccaataaagagaaaacaggggtcttctgatcagttttgtggatcgtcgtga

SEQ ID NO: 139 - DNA, 1377 bp, Arabidopsis thaliana
  misc_feature 1..1377: note = ORF of protein Pizza
  source 1..1377: mol_type = unassigned DNA
atgcccatgttaatggcgacacgtatcgatataatccaaaagcttaatgtatatccaaggtttcaaaaccatgacaagaagaaactaatcactctctccaatttggaccgtcagtgtcctttactcatgtactctgtcttcttctacaagaataccacaactcgtgactttgactccgtcttctccaacctgaagctcgggctggaggagactatgtctgtgtggtatcccgcggcagggagactgggtttggacggaggtggctgcaagctcaacatccggtgtaacgatggtggcgcagtcatggtggaggcggtggcgacaggtgtcaagttgtccgagcttggtgatttgactcagtacaatgagttttatgagaatttagtttacaagccttccttggatggtgatttctctgtgatgcctcttgttgttgctcaggtgacaagatttgcatgtggaggttactcaattggaatcggtacaagccactctctatttgatggaatctcagcttacgaattcattcacgcgtgggcctccaactctcacattcacaacaaatccaacagcaagattactaataaaaaggaagatgtggtcatcaaaccggttcatgatcgacgaaatctactggttaaccgggatgctgtccgagaaaccaatgctgcagccatttgtcatctgtaccagttgatcaaacaggcgatgatgacctatcaggagcaaaaccgtaacttagagttaccagactctggttttgtgatcaaaacgttcgagcttaatggcgatgcgatagaaagcatgaagaagaaatcactagaagggttcatgtgctcctcctttgagtttcttgctgctcatttgtggaaggcaagaacaagggctttagggttgaggagagacgccatggtgtgtttacaattcgcagtggacataaggaaaagaacggagacaccgctgccagaagggttttccggcaacgcatacgtgcttgcctcggtggcatccaccgccagagaattacttgaagagctaacactcgagtcaatagtcaacaagatcagagaagccaagaaatcaattgaccaaggttacataaactcttacatggaagcactcggaggtagtaatgacggaaatctccctcctctcaaagagctaaccctaatctccgactggacaaaaatgccatttcacaatgttggctttggcaacggcggcgagccagcggattacatggccccactgtgtccaccggtgccacaagttgcttatttcatgaagaaccctaaagatgccaaaggtgttcttgtgaggattggcttggacccacgagatgttaatggcttttcaaatcatttccttgattgctaa

SEQ ID NO: 140 - DNA, 1521 bp, Arabidopsis thaliana
  misc_feature 1..1521: note = ORF of protein Ppox1
  source 1..1521: mol_type = unassigned DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctctcaggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaactacattggcgggtctacaaacaccggaattctgtccaagtctgaaggtgagttagtggaagcatttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 141 - DNA, 1527 bp, Arabidopsis thaliana
  misc_feature 1..1527: note = ORF of protein Ppox2
  source 1..1527: mol_type = unassigned DNA
atggcgtctggagcagtagcagatcatcaaattgaagcggtttcaggaaaaagagtcgcagtcgtaggtgcaggtgtaagtggacttgcggcggcttacaagttgaaatcgaggggtttgaatgtgactgtgtttgaagctgatggaagagtaggtgggaagttgagaagtgttatgcaaaatggtttgatttgggatgaaggagcaaacaccatgactgaggctgagccagaagttgggagtttacttgatgatcttgggcttcgtgagaaacaacaatttccaatttcacagaaaaagcggtatattgtgcggaatggtgtacctgtgatgctacctaccaatcccatagagctggtcacaagtagtgtgctctctacccaatctaagtttcaaatcttgttggaaccatttttatggaagaaaaagtcctcaaaagtctcagatgcatctgctgaagaaagtgtaagcgagttctttcaacgccattttggacaagaggttgttgactatctcatcgacccttttgttggtggaacaagtgctgcggaccctgattccctttcaatgaagcattctttcccagatctctggaatgtagagaaaagttttggctctattatagtcggtgcaatcagaacaaagtttgctgctaaaggtggtaaaagtagagacacaaagagttctcctggcacaaaaaagggttcgcgtgggtcattctcttttaaggggggaatgcagattcttcctgatacgttgtgcaaaagtctctcacatgatgagatcaatttagactccaaggtactctctttgtcttacaattctggatcaagacaggagaactggtcattatcttgtgtttcgcataatgaaacgcagagacaaaacccccattatgatgctgtaattatgacggctcctctgtgcaatgtgaaggagatgaaggttatgaaaggaggacaaccctttcagctaaactttctccccgagattaattacatgcccctctcggttttaatcaccacattcacaaaggagaaagtaaagagacctcttgaaggctttggggtactcattccatctaaggagcaaaagcatggtttcaaaactctaggtacacttttttcatcaatgatgtttccagatcgttcccctagtgacgttcatctatatacaacttttattggtgggagtaggaaccaggaactagccaaagcttccactgacgaattaaaacaagttgtgacttctgaccttcagcgactgttgggggttgaaggtgaacccgtgtctgtcaaccattactattggaggaaagcattcccgttgtatgacagcagctatgactcagtcatggaagcaattgacaagatggagaatgatctacctgggttcttctatgcaggtaatcatcgaggggggctctctgttgggaaatcaatagcatcaggttgcaaagcagctgaccttgtgatctcatacctggagtcttgctcaaatgacaagaaaccaaatgacagcttataa

SEQ ID NO: 142 - DNA, 2472 bp, Arabidopsis thaliana
  misc_feature 1..2472: note = ORF of protein Prp39
  source 1..2472: mol_type = unassigned DNA
atgggtgacagcgaggccatggtttccgagggttatacttctgctccatatggagactataatgcttctgctgctactgtggaatcgaccgggcaagagactgcaccaattgttgatgcatcacactcggtcaacaatgattctttggtcaatggtactgcgccagttgagaacggaagtgcaacagataatgtggctgtgactgctccagcagcggagcatggagacaatactggctctacactctcaacggaagaggagcgcttgtggaatattgtaagggcaaattctttagagtttaatgcttggactgccttgattgatgagacggagaggatagcgcaggacaatatagcaaaaatccggaaggtctatgatgctttcttagctgaatttcctctgtgttatggctattggaaaaagtttgccgatcatgaggctcgggtgggggcaatggacaaagtcgtggaggtttatgaaagagcagtgctgggagtgacatattcagtggatatctggttgcattattgcacttttgccatcaatacatatggagatccagaaacgatcagaaggctttttgaacgagctttggtttacgttggaactgattttctttcctctccgttgtgggacaaatacattgagtatgagtacatgcagcaggactggagccgagttgccttgatttacaccagaatattggagaatccaattcaaaatctggatagatatttcagcagttttaaggagctagctgaaacacggcctctgtcggaactaaggagtgctgaggaatccgcagctgctgctgttgctgttgctggtgatgcttctgaaagtgcagcatctgagtccggtgaaaaggcagatgaaggacgatctcaagttgatggttccaccgaacaatcccctaaattggaaagtgctagttcaactgaacctgaggagttgaagaagtatgtaggcatcagggaagccatgtacataaaatcgaaagagtttgaatctaaaatcattggttatgaaatggctataaggaggccctatttccatgtgcgtcctctgaatgtcgcagaactggagaattggcacaactatctggatttcattgagagggatggagacttcaataagttaagtagtatctggtgcatcatatgcttgattgggtttccattggatcaagcaacttttaaatgggaaataactgagaccaaagcatgtgcctcgatttgttccaatgttattaatgcaggtgtcttcctaacattctgtctgtcaggcaaggagggtccctctgtggtcaagctgtatgaaagatgtgtggttacctgtgcaaattacccagaatactggattcgttatgtgacaaacatggaagcaagtggaagtgcggaccttgcagaaaatgcccttgctcgagcaactcaagtctttgtcaagaaacaaccagagattcacctatttgctgctcgattaaaagagcagaatggagatatagctggtgctagagctgcataccaattagtgcactctgaaatttctcctggacttcttgaagcagtaatcaagcatgcaaatatggaataccgactaggtaatctggatgatgctttctctttgtatgagcaagtgattgctgttgaaaaggggaaagaacattccacaatactgccactgctgtatgcgcagtattcaaggttttcatacttggtctccagggatgctgagaaagctaggaggattattgtcgaagcacttgaccatgtacaaccgtcaaaacctctcatggaagcactgattcattttgaggcgattcagccaccaccaagagagattgattaccttgagccacttgtagagaaagttataaagccagatgcagatgcccaaaacattgcaagttccactgagagggaagagctatccttaatatatatagagttcctgggtatttttggagatgtgaagtccattaaaaaagcggaagatcaacatgttaaactgttttatcctcatcggagcacgtcggagctgaaaaagcgtagcgcagatgattttctcgcatcagataggacgaaaatggcaaaaacttacaacggcactccacctgctcagccagtatccaatgcatatccaaatgctcaggctcaatggtctggtggttatgctgcgcagcctcagacttggccaccagcacaagctgctcctgctcaaccacagcaatggaaccctgcctacggtcaacaggctgcttatggtgcatatgggggatatcctgctggctataccgctccacaagcaccaacacctgtgccacaggccgcagcttatggagcgtatcctgctcagacatacccaacgcagagttatgcacctccagttgcagcagcagcaccagcggctgcaccggtgcagcaaccggctgctgctgttgctcctcaagcgtactacaacacgtactactga

SEQ ID NO: 143 - DNA, 1062 bp, Arabidopsis thaliana
  misc_feature 1..1062: note = ORF of protein PsbA
  source 1..1062: mol_type = unassigned DNA
atgactgcaattttagagagacgcgaaagcgaaagcctatggggtcgcttctgtaactggataactagcactgaaaaccgtctttacattggatggtttggtgttttgatgatccctaccttattgaccgcaacttctgtttttattatcgcattcattgctgctcctccagtagatattgatggtattcgtgaacctgtttctggatctcttctttacggaaacaatattatttccggtgccattattcctacttctgcagctattggattgcatttttacccaatctgggaagctgcatccgttgatgaatggctatacaacggcggtccttatgaactaattgttctacactttttacttggtgtagcttgttatatgggtcgtgagtgggaacttagtttccgtctgggtatgcgtccttggattgctgttgcatattcagctcctgttgcagctgcgactgctgttttcttgatctatccaattggtcagggaagtttttctgatggtatgcctctaggaatctctggtactttcaactttatgattgtattccaggctgagcacaacattcttatgcacccatttcacatgttaggtgtagctggtgtattcggcggctccctttttagtgctatgcatggttccttggtaacttctagtttgatcagggaaaccacagaaaatgaatctgctaatgaaggttacagattcgggcaagaagaagaaacttacaacattgtagctgctcacggttattttggccgattgattttccaatatgctagtttcaacaattctcgttctttacatttcttcttagcggcttggccggtagtaggtatttggtttactgctttaggtattagtactatggctttcaacctaaatggtttcaatttcaaccaatcagtagttgatagtcaaggacgtgttattaatacttgggctgatattattaaccgtgctaaccttggtatggaagttatgcatgaacgtaatgctcacaacttccctctagacctagctgctgttgaggctccatctacaaatggataa

SEQ ID NO: 144 - DNA, 3027 bp, Arabidopsis thaliana
  misc_feature 1..3027: note = ORF of protein Pskr1
  source 1..3027: mol_type = unassigned DNA
atgcgtgttcatcgtttttgtgtgatcgtcatcttcctcacagagttactatgtttcttctattcctcggaatctcagaccacctccaggtgccatccacatgacctcgaagccttacgtgacttcatagcacatctcgaaccaaaaccagatggttggatcaattcttcttcttctacagactgctgcaactggaccggaatcacctgcaattcaaacaacaccggaagagttattagattggagcttgggaacaaaaagctgtcggggaagttgtctgaatctctcgggaagctagatgagattagggttcttaatctctctcgaaacttcatcaaagattcgatccctctttcgattttcaacttgaagaatctacaaactcttgatttgagctctaatgatctctccggcggaatcccaacaagtataaatctcccagctctgcaaagttttgatctttcttcaaataaattcaatgggtcgcttccgtctcatatctgccataactctactcaaattagggttgtgaaacttgcggtgaactacttcgccggaaacttcacttccgggtttgggaaatgtgtcttgcttgagcatctctgtcttggtatgaacgatcttactggtaacatccctgaggatttgtttcatctcaaaagattgaatcttttagggattcaagagaatcgtctctctggttcgttgagtcgtgagattaggaatctctcaagtcttgttcgtcttgatgtttcttggaatttgttttccggtgaaatccctgatgtgttcgacgaattgcctcagttaaagtttttcttaggtcagaccaatggattcattggaggaatacctaaatcgttggcgaattcaccgagtttgaatctgcttaacttgaggaacaattctttatcgggtcgtttgatgttgaattgtacggcgatgattgctttgaactctcttgatttaggtaccaatagattcaatgggaggttacctgagaatctaccggattgcaagcggttaaagaacgttaacctcgcgaggaacaccttccatggacaagtaccagagagtttcaagaacttcgagagcttatcttacttctcgttatcgaattcgagtttggctaatatctcttcagcgcttgggatacttcagcattgcaagaacttgacgactttggttcttacattgaatttccatggagaggctttacccgatgattcaagtcttcatttcgagaagcttaaggtgcttgtagtggcgaattgtaggcttactggttcgatgccgaggtggttaagctcgagtaatgaacttcagttgttggatctttcttggaaccgtttaaccggcgctatcccgagctggattggtgacttcaaggctctgttctacttggatttatctaacaactcgtttacaggagagatccctaagagcttaactaagttagagagtctcactagccgtaatatctcagtcaatgagccatctcctgatttcccgttctttatgaaaagaaacgagagcgcgagagcgttgcaatacaatcagattttcgggttcccgccaacgattgagcttggtcataacaatctctctggacctatttgggaggagtttggtaatctgaagaagcttcatgtgtttgatttgaaatggaatgcattatctggatcaatacctagctcgctttctggtatgacgagcttggaagctcttgatctctctaataaccgtctttcgggttcgatcccggtttctctgcaacagctctcgtttctgtcgaagttcagtgttgcttataacaatctctcgggagtaataccttccggtggtcagtttcagacgtttccaaactcgagctttgagagtaaccatctctgcggggaacacagattcccctgttctgaaggtactgagagtgcattgatcaaacggtcaagaagaagcagaggaggtgacattggaatggcgattgggatagcgtttggttcggtttttcttttgactcttctctcgttgattgtgttgcgtgctcgtagacggtcaggagaagttgatccggagatagaagaatccgagagcatgaatcgtaaagaactcggagagattggatctaagcttgtggttttgtttcagagcaatgataaagagctctcttatgatgaccttttggactcaacaaatagttttgatcaagctaacatcattggctgtggcgggtttggtatggtttacaaagcaacgttaccagacggtaagaaagttgcgatcaagaagttatccggtgattgcggtcaaatcgaaagagaattcgaagcagaagttgaaacactctcaagagcacagcatccaaatcttgttcttctccgaggattctgtttctacaaaaacgaccggcttttaatctactcgtatatggaaaacggaagcttagactattggctacacgagcgtaacgacggtccagcgttgttgaagtggaaaacacgtcttagaatcgctcaaggtgctgcaaaagggttactttacttgcatgaagggtgtgatcctcatatcttacaccgcgatattaaatcgagtaatattcttctcgacgagaatttcaactctcatttagcggatttcggactcgcaaggctgatgagtccttacgagacgcatgtaagtactgatttggttggaactttaggttacattcctccggaatacgggcaagcttcggttgctacttacaaaggcgatgtgtatagtttcggagttgtgcttctcgagcttttaaccgataaaagaccggtggatatgtgtaaaccgaaagggtgtagggatctgatctcgtgggtcgtcaagatgaagcatgagagtcgagcaagcgaggttttcgatccgttaatatacagtaaagagaatgataaagagatgtttcgggttctcgagattgcttgtttatgtttaagcgaaaacccgaaacagaggccaacgactcaacagttagtctcttggcttgatgatgtctag

SEQ ID NO: 145 - DNA, 1389 bp, Arabidopsis thaliana
  misc_feature 1..1389: note = ORF of protein Rd21
  source 1..1389: mol_type = unassigned DNA
atggggttccttaagccaaccatggcgattctttttctagcgatggtcgccgtttcatcagccgtggacatgtcaatcatctcctacgacgagaaacatggcgtctccaccaccggtggccgtagcgaagccgaggttatgagtatctacgaggcatggttggtgaaacacggcaaggctcagagccagaactctcttgttgagaaagatcgacggttcgagatctttaaagacaatcttcgtttcgtcgatgaacataacgagaagaatcttagttatagattgggtttgactcgttttgcggatcttactaacgatgagtatagatccaagtatcttggagctaagatggagaagaaaggtgagagaaggactagcctacggtacgaggctcgtgtcggtgatgagctaccggagtctattgactggaggaagaaaggagccgtggctgaggtcaaagatcagggtggttgcgggagttgttgggcgttttcaaccattggagcagtggagggaataaaccagatcgtaaccggagacctaataaccttgtctgaacaagagttggtcgattgtgacacttcatacaacgaaggttgtaacggaggtcttatggactatgcttttgagttcattatcaagaatggtggaatcgatacagacaaagattatccttacaagggtgttgatggaacttgtgaccagatcaggaaaaacgctaaagttgtcactatcgattcatatgaggatgttccaacttacagcgaggaatcgttgaagaaagcagttgctcatcaacccattagcattgccattgaggctggtggtcgtgcgttccagctctatgattcgggtatatttgatggaagttgtggaacacaactagaccacggagttgtggcggttggatacggaactgagaacggcaaagattactggattgtgagaaactcatggggtaaaagctggggagagagtggatacctaaggatggcgcgtaacattgcgtcttcatcaggaaaatgtggaatcgcgattgaaccttcatacccgataaagaatggcgaaaacccgccaaacccgggaccttcacctccatctcccatcaagcctccaacccaatgtgacagttactacacttgtcctgagagcaacacttgttgttgtctgtttgagtatggcaagtattgctttgcttggggatgttgcccactagaagcagccacttgctgtgatgacaactatagttgctgccctcacgagtacccggtttgtgaccttgatcaaggaacctgtttattgagcaagaacagtccatttagtgttaaggccttaaagcgtaaacccgcaacgccattctggtcacaaggcagaaagaacattgcctaa

SEQ ID NO: 146 - DNA, 906 bp, Arabidopsis thaliana
  misc_feature 1..906: note = ORF of protein Ring1
  source 1..906: mol_type = unassigned DNA
atggcgtttaatcacagaaaaatgcttctttcatgtctacagtttaaagatttacgcttttgtttccggcaataccctcccccgccaccaccaccaccgccaccgagggagctctctctcctactccccacctcgatctgcgtcgtcggctcaatcatactctttctatttctagtcttcttcctctacctccacatcacgcaacaacggcggatttcagccgccagcgtaactcccggtgacacaaaccaacaggaagatgaggacgaaacagaggagagagatttctccgactttcatcacgtttggcaaatcccaaccgtcggtctccaccgttccgcgatcaactcgatcacagtcgttggattcaaaaagggagaagggataatcgacggcacagaatgctctgtctgcttaaacgagtttgaagaagacgagtcgctgagactgttaccaaagtgtagccatgcctttcatctaaattgcatcgacacttggttgctctctcacaagaactgtcctttatgtcgtgcccctgtacttcttatcactgaaccacctcatcaagaaacagagacgaatcatcaaccagattctgaatcgagcaatgacctccgtggaagacaagacagttccagatctagaagaaaccataacatttttcttccaagggcacaaagtgatttggcaaactactgtggctcaggaagagtagaaaacgtgagaagatcgttttcaattggaggaagcttatcactttgcgatggtattaacaatgctactagaagtggaagacagttctataccagcttcagtgctaacttgttctcatcatcaagaagagtaaggaacgaacaaccgattccacaaaaccaaatgccatccgtaactggtaacaccagttaa

SEQ ID NO: 147 - DNA, 4182 bp, Arabidopsis thaliana
  misc_feature 1..4182: note = ORF of protein Ros1
  source 1..4182: mol_type = unassigned DNA
atggagaaacagaggagagaagaaagcagctttcaacaacctccatggattcctcagacacccatgaagccattttcaccgatctgcccatacacggtggaggatcaatatcatagcagtcaattggaggaaaggagatttgttgggaacaaggatatgagtggtcttgatcacttgtcttttggggatttgcttgctctagctaacactgcatccctcatattctctggtcagactccaatacctacaagaaacacagaggttatgcaaaaaggtactgaagaagtggagagtttgagctcagtgagtaacaatgttgctgaacagatcctcaagactcctgaaaaacctaagaggaagaagcatcggccaaaggttcgtagagaagctaaacccaagagggagcctaaaccacgagctccgaggaagtctgttgtcaccgatggtcaagaaagcaaaacaccaaagaggaaatatgtgcggaagaaggttgaagtcagtaaggatcaagacgctactccggttgaatcatcagcagctgttgaaacttcaactcgtcctaagaggctctgtagacgagtcttggattttgaagccgaaaatggagaaaaccagaccaacggtgacattagagaagcaggtgagatggaatcagctcttcaagagaagcagttagattctgggaatcaagagttaaaagattgccttctttcggctcctagcacgcccaagagaaagcgcagccaaggtaaaagaaagggagttcaaccaaagaaaaatggcagtaatctagaagaagtcgatatttcgatggcgcaagctgcaaagagaagacaaggaccaacttgttgcgacatgaatctatcagggattcagtatgatgagcaatgtgactaccagaaaatgcattggttgtattccccaaacttgcaacagggagggatgagatatgatgccatttgcagcaaagtattctctggacaacagcacaattatgtttctgcctttcacgctacgtgctacagttccacatctcagctcagtgctaatagagtcctaaccgttgaagaaagacgagaaggtatctttcaaggaaggcaagagtctgagctaaatgttctctcggataagatagacacgccgatcaagaagaaaacaacaggccatgctcgattccggaatttgtcttcaatgaataaacttgtggaagttcctgagcatttaacctcaggatattgtagcaagccacagcaaaataataagattcttgttgatacgcgggtgactgtgagcaaaaagaagccaaccaagtctgagaaatcacaaaccaaacagaaaaatcttcttccgaatctttgccgttttccaccttcatttactggtctttctccagatgaactttggaaacgacgtaactcgatcgaaacaatcagtgagctattgcgtctattagacatcaacagggagcattctgaaactgctctcgttccttacacaatgaatagccagattgtactctttggtggtggcgctggagcaattgtgcctgtaactcctgttaaaaaaccacgcccacgaccaaaggttgatctagacgatgagacagacagagtgtggaaactgctattggagaatattaatagcgaaggtgttgacggatcagacgagcagaaggcgaaatggtgggaggaagaacgtaatgtgtttcgaggacgagctgactcatttattgcaaggatgcaccttgtacaaggggatcgacgttttacgccttggaagggatccgtcgtggattctgttgttggagtatttctcactcaaaatgtttcagaccatctctcaagttcggctttcatgtcgttggcttcccagttccctgtcccttttgtaccgagcagtaactttgacgctggaacaagctcgatgccttctattcaaataacgtacttggactcagaggaaacgatgtcaagcccacccgatcacaatcacagttctgttactttgaaaaatacacagcctgatgaggagaaggattatgtacctagcaatgaaacctccagaagcagtagtgagattgccatctcagcccatgaatcagttgacaaaaccacggattcaaaggagtatgttgattcagatcgaaaaggctcaagtgtagaggttgataagacggatgagaagtgtcgtgtcctgaacctgtttccatctgaagattctgcacttacatgtcaacattcgatggtgtctgatgctcctcaaaatacagagagagcaggatcaagctcagagatcgacttagaaggagagtatcgtacttcctttatgaagctcctacagggggtacaagtctctctagaagattccaatcaagtatcaccaaatatgtctccgggtgattgtagctcagaaattaagggtttccagtcaatgaaagagcccacaaaatcctctgttgatagtagtgaacctggttgttgctctcagcaagatggggatgttttgagttgtcagaaacctaccttaaaagaaaaagggaaaaaggttttgaaggaggaaaaaaaagcgtttgactgggattgtttaagaagagaagcccaagctagagcaggaattagagaaaaaacaagaagtacaatggacaccgtggattggaaggcaatacgagcagcagatgttaaggaagttgctgaaacaatcaagagtcgcgggatgaaccataaacttgcagaacgtatacagggcttccttgatcgactggtaaatgaccatggaagtatcgatcttgaatggttgagagatgttccaccagataaagcaaaagaatatcttctgagctttaacggattgggactgaaaagtgtggagtgtgtgcggcttctaacacttcaccatcttgcctttccagttgatacaaatgttgggcgcatagccgtcagacttggatgggtgccccttcagccgctcccagagtcacttcagttgcatcttctggaaatgtatcctatgcttgaatctattcaaaagtatctttggccccgtctctgcaaactcgaccaaaaaacattgtatgagttgcactaccagatgattacttttggaaaggtcttttgcacaaagagcaaacctaattgcaatgcatgtccgatgaaaggagaatgcagacattttgccagtgcgtttgcaagtgcaaggcttgctttaccaagtacagagaaaggtatggggacacctgataaaaaccctttgcctctacacctgccagagccattccagagagagcaagggtctgaagtagtacagcactcagaaccagcaaaaaaggtcacatgttgtgaaccaatcatcgaagagcctgcttcaccggagccagaaaccgcagaagtatcaatagctgacatagaggaggcgttttttgaggatccagaagaaattcctaccatcaggctaaacatggatgcatttaccagtaacttgaagaagataatggaacacaacaaggaacttcaagacggaaacatgtccagcgctttagttgcacttactgctgaaactgcttctcttccaatgcctaagctcaagaatatcagccagttaaggacagaacaccgagtttacgaacttccagacgagcatcctcttctagctcagttggaaaagagagaacctgatgatccatgttcttatttgcttgctatatggacgccaggtgagacggctgattctattcaaccgtctgttagtacgtgcatattccaagcaaatggtatgctttgtgacgaggagacttgtttctcctgcaacagcatcaaggagactagatctcaaattgtgagagggacaattttgattccttgtagaacagcgatgaggggtagttttcctctaaatggaacgtactttcaagtaaatgaggtgtttgcggatcatgcatccagcctaaacccaatcaatgtcccaagggaattgatatgggaattacctcgaagaacggtctattttggtacctctgttcctacgatattcaaaggtttatcaactgagaagatacaggcttgcttttggaaagggtacgtatgtgtacgtggatttgatcgaaagacgaggggaccgaagcctttgattgcaagattgcacttcccggcgagcaaactgaagggacaacaagctaacctcgcctaa

SEQ ID NO: 148 - DNA, 1200 bp, Arabidopsis thaliana
  misc_feature 1..1200: note = ORF of protein Rpt4a
  source 1..1200: mol_type = unassigned DNA
atgaccgacgtcgatgaatccgttagacgccgtacagccgccgtttccgagtaccggaaaaagctcctccaacataaggaactcgaatcccgtgttcgaacagctagggagaacttaagaggagctaaaaaggaattcaacaaaaccgaggatgatctcaagtctcttcaaagtgttgggcagattattggagaagtgcttcgacccctggataatgaaagattgattgtgaaagcgagcagtggtccacgttatgtggtgggttgccgaagcaaagtggataaggaaaagctaacctccggaactcgagttgttctcgatatgacaacactaacaattatgcgagcccttccccgagaagttgatcctgttgtttataacatgcttcatgaagaccctggcaacattagctactccgctgtcggagggttaggtgatcagatcagagaactcagggaatctattgagctccctctcatgaatcctgagcttttcctcagagtagggatcaaacctccaaagggtgtccttctgtatggacctccaggtactggaaaaactctactagccagggctattgccagcaacatcgatgctaacttcttgaaggttgtatccagtgcaatcatagacaagtacattggagaaagtgctaggctgattcgggagatgtttaactatgcccgtgaacatcagccttgtatcatattcatggatgaaatcgatgctattggtgggcgtcgttttagtgagggaactagtgctgatcgtgaaattcaaagaacgcttatggagttactcaaccagcttgatgggtttgacaacctcggaaaggtgaaaatgataatggccacaaacaggcctgatgtgcttgatcctgcgcttctccgtccgggaaggttagatagaaagattgagatccctttgccaaacgagcagtcaagaatggatattcttaagatccatgcagctggtatcgccaaacatggtgaaatcgattacgaggctattgtcaaacttgcagagggattcaatggtgctgatctgcgtaacatatgcacggaagctggaatgtttgcaatccgagcagagcgggattatgtgatccatgaagatttcatgaaggcggtgagaaagctgagtgaggccaagaaactggagtccagttcgcactataacgctgattttggaaaagagtga

SEQ ID NO: 149 - DNA, 3804 bp, Arabidopsis thaliana
  misc_feature 1..3804: note = ORF of protein Sfr6
  source 1..3804: mol_type = unassigned DNA
atgaatcagcaaaacccagaagaagaagtttctttggttaataatagcggtggtggaggaatcatcgaagctccagctatagtggaggagaaagaggaagaaggattacagcagaagcaggaagagactattgagtctaccgatccgattctcgtcgtcgttgaggagaaattgttagagaaatcagtagacggagagaaagaagacgataatagtagtagtagtaacatggagattgatcctgtgagtccggctacagttttctgtgttaagcttaagcagcccaattccaatttgcttcataagatgagtgttcctgaattgtgccgtaacttcagtgctgttgcgtggtgtggcaaattgaatgctattgcttgtgcttccgagacctgtgccaggattccaagctccaaggcaaatacacctttttggataccaatacatatcttgatacctgagcgccctactgagtgtgcggtgtttaatgttgtggcagactctcctcgtgattctgtccaatttatcgaatggtctcccacttcttgtcctcgtgcgttactcattgctaattttcatggacgtataactatctggacgcagcctactcagggttcggctaatttagtgcacgacgctacctcctggcagtgtgagcatgaatggcgtcaggacattgctgttgttacaaagtggctgacaggggcttccccatataggtggttgtcctccaagccaagttctggtacaaatgcaaagtcaactttcgaggagaaatttctctcgcagagctctgaaagctcagctcggtggcccaactttctctgtgtatgctctgttttctcatccggctctgttcaaattcattggtcccagtggccttctaaccagggaagcactgcaccaaagtggtttagtacaaagaaaggtcttttaggtgcagggccaagtggaattatggctgctgatgctattataacagacagtggtgccatgcatgtagcaggagttccaattgtaaacccttcaacaattgtagtatgggaggtgacccctggccctggaaatggactccaggcgactccaaaaatctctacaggcagtcgtgtgccaccatcccttagttcttcttcttggacaggttttgctcctttagctgcgtacttgtttagctggcaagaatacttaatatccgagataaagcaagggaagaagccctcagatcaagattccagtgatgctatatcgctaagttgctcaccggtttccaatttttctgcttatgtaagtccagaggctgcagctcagtctgcagcaaccacaacatggggatctggtgttaccgctgttgcttttgatccaactcgtggtggttcagtgatagcagttgttatagttgaagggcagtacatgtctccgtatgatccagatgaaggtccttcaatcacaggctggagagtacagcgctgggaatcaagtgttcaacctgttgtactgcatcagatatttggaaacccaacttcaaattttggaggacaggtccccacgcaaactgtctgggtatccagagtggatatgagcataccacctactaaagattttaagaatcatcaagtagctgcagcaggaccaagtgtggatgcaccaaaggagcctgattctggtgatgagaaggctaacaaggttgtatttgatccttttgatttgccaagtgatattcggacacttgcacggattgtctattctgctcatggtggtgaaattgcgattgcttttcttcgtggtggagttcatatcttttctggtccaactttttcacctgttgaaaactatcaaataaatgttggatctgcaattgctgcacccgcattttcaccaacaagctgttgttcggcttctgtatggcatgatgctgctaaggactgcgcaatgttgaaaatcatccgtgttcttcctcctgctcttccgcgtaaccaatcaaaggttgatcaatcaacatgggagcgggcgatcgctgagagattctggtggagtcttttggtcggagttgattggtgggatgcagttggctgcacacagagtgctgcagaggatgggatagtttcattgaacagcgtgattgctgtcatggatgctgattttcactcccttccttcaacacagcacagacaacaatatggccctaacctagataggatcaaatgtcggttacttgaaggaaccaatgctcaagaggttcgtgccatggttttagatatgcaagcaaggttgttgttggacatgcttggaaaaggtattgaatcagctcttgtgaatccttcggcgttggtttttgagccatggcgagtagatggggagacaataacaggcatcaatccggaggcaatggctgttgatcctgctcttgtttccagtattcaggcttatgtggatgctgttcttgatcttgcttctcatttcatcacacgtttaaggcgttatgcgagtttttgtcggactcttgcaagccatgctgcttctgctggaactggcagtaatcgcaacaatgttaccagtcccacacaaaatgcatcatctcctgcaacacctcagggtcaacctactactactactactactactgctacgacaaactccagcggaagctcacatgtgcaagcttggatgcagggggccatagcgaaaattagtagctcgaatgatggatccaactctactgcaagcccaatcagtggttcacctaccttcatgccaataagcatcaatacaggaacatttccaggaacacctgctgttcggctcattggggattgtcatttccttcatcggttatgccagctgttgctcttctgttttctccagcggtcttcacgatttccacagcgaaatgctgatgttagttcacaaaaacttcaaacgggggctaccagcaaattggaagaagtcaactctgctaaaccaacccctgccttgaacaggatagaggacgcccagggattccggggtgcccagttgggtactggagtgaaagggattgatgaaaattctgctcgtacaacaaagatgggttctgggaatgccggtcaaggatatacttatgaggaggtgagagttcttttccatatactaatggatctctgcaagcgaacatctggtcttgcgcatcccttacctggctctcaggtaggtagtggaaacattcaagttcgactgcattatattgatggaaattacactgtgttacccgaggtggtagaagcggctcttggaccacatatgcagaacatgcctcgcccaagaggagctgatgctgctggtcttctacttcgggagttagagcttcatccgccttccgaagaatggcatagaagaaatttatttggtggtcccgggtcagagcctgaggatatgatcttgacagacgatgtttccaagctgagtaattccttagatctgcctgatacaaacttttccggaatatgtgatggatacaacagagtccatagtctttggccaagaaaacgcaggatgtctgaaagagatgcagcttttggttcaaatacttctgtgggtttgggtgcatatcttgggatcatgggttctcgtagggatgttgtgaccgcgacatggaaaactggtcttgaaggagtttggtacaagtgcataagatgcctaaggcagacatctgcatttgcttcaccaggtgccactaagcagccaaatccgaatgaacgagaaacctggtggacaagtcgttgggtttattgctgccccatgtgtggtggaacgtgggtccgtgttgtatag

SEQ ID NO: 150 - DNA, 1596 bp, Arabidopsis thaliana
  misc_feature 1..1596: note = ORF of protein Shr
  source 1..1596: mol_type = unassigned DNA
atggatactctctttagactagtcagtctccaacaacaacaacaatccgatagtatcattacaaatcaatcttcgttaagcagaacttccaccaccactactggctctccacaaactgcttatcactacaactttccacaaaacgacgtcgtcgaagaatgcttcaactttttcatggatgaagaagacctttcctcttcttcttctcaccacaaccatcacaaccacaacaatcctaatacttactactctcctttcactactcccacccaataccatcccgccacatcatcaaccccttcctccaccgccgcagccgcagctttagcctcgccttactcctcctccggccaccataatgacccttccgcgttctccatacctcaaactcctccgtccttcgacttctcagccaatgccaagtgggcagactcggtccttcttgaagcggcacgtgccttctccgacaaagacactgcacgtgcgcaacaaatcctatggacgctcaacgagctctcttctccgtacggagacaccgagcaaaaactggcttcttacttcctccaagctctcttcaaccgcatgaccggttcaggcgaacgatgctaccgaaccatggtaacagctgcagccacagagaagacttgctccttcgagtcaacgcgaaaaactgtactaaagttccaagaagttagcccctgggccacgtttggacacgtggcggcaaacggagcaatcttggaagcagtagacggagaggcaaagatccacatcgttgacataagctccacgttttgcactcaatggccgactcttctagaagctttagccacaagatcagacgacacgcctcacctaaggctaaccacagttgtcgtggccaacaagtttgtcaacgatcaaacggcgtcgcatcggatgatgaaagagatcggaaaccgaatggagaaattcgctaggcttatgggagttcctttcaaatttaacattattcatcacgttggagatttatctgagtttgatctcaacgaactcgacgttaaaccagacgaagtcttggccattaactgcgtaggcgcgatgcatgggatcgcttcacgtggaagccctagagacgctgtgatatcgagtttccgacggttaagaccgaggattgtgacggtcgtagaagaagaagctgatcttgtcggagaagaagaaggtggctttgatgatgagttcttgagagggtttggagaatgtttacgatggtttagggtttgcttcgagtcatgggaagagagttttccaaggacgagcaacgagaggttgatgctagagcgtgcagcgggacgtgcgatcgttgatcttgtggcttgtgagccgtcggattccacggagaggcgagagacagcgaggaagtggtcgaggaggatgaggaatagtgggtttggagcggtggggtatagtgatgaggtggcggatgatgtcagagctttgttgaggagatataaagaaggtgtttggtcgatggtacagtgtcctgatgccgccggaatattcctttgttggagagatcagccggtggtttgggctagtgcgtggcggccaacgtaa

SEQ ID NO: 151 - DNA, 870 bp, Arabidopsis thaliana
  misc_feature 1..870: note = ORF of protein Shy2
  source 1..870: mol_type = unassigned DNA
atgggagaaaaagaaagtgtccccacaaatgtccccaagaagatgggacacacttttgcctcaaagttgctgttactgctaccgacaagcttagctttttttcctttgtccttaattcagaaaacagtttcttctctctctaccagtatctatctttattctcctttaacttgtataaaacactcagcttcctcgaagcctctcatcttcatcatcagcagcttctctatatctctcctctctttcaaggataataaccaaaagcttttctttttatcttctcctgcaattcttgaagaaatggatgagtttgttaacctcaaggaaacagagctgaggctgggattaccgggaacagataatgtatgtgaagcaaaagagagagtttcttgctgtaataacaacaataagagagtactatcaactgatactgagaaggagattgaatcatcatcaaggaaaactgaaacatcccctcctcgaaaggctcagattgttggatggccaccagttagatcttacaggaagaacaacattcagagtaagaagaatgaatctgagcatgagggtcaaggaatctatgtgaaagtaagtatggatggtgcaccatacttgaggaaaatagatctgagttgttacaaaggatactcagagttgcttaaagctttagaagtgatgttcaaattctctgtgggagagtactttgagagagatggatataaaggttcagactttgtgcctacttatgaagacaaagatggtgattggatgctcattggtgatgttccatgggagatgttcatatgtacgtgcaagagactaaggatcatgaaaggatcagaagccaaaggtttaggctgtggtgtatga

SEQ ID NO: 152 - DNA, 3930 bp, Medicago truncatula
  misc_feature 1..3930: note = ORF of protein Skl
  source 1..3930: mol_type = unassigned DNA
atggaaacagaggcattgagttctgaacagacgaagagtaaaatggaggcagagacattgagtactaatccccctcctggttttcttattcgcgcacttccggctgttatacccgcgcttttgatttcgataggatatgttgaccctggaaagtgggtggcaagtatagaaggtggtgcgaggtttggttttgatctggtggcttttgcgctaattttcaattttgccgctatcttctgtcagtacttatccgcaagggttggtgtaatcactggacgagatcttgctcagatttgcagtgatgaatatgatacatggacatgccttctccttggaattcaagcagaactgtccgtgattatgctagatcttaacatgatcttgggcatggcacaaggattaaatcttatttttgggtgggacttgttcacctgtgtctttttaactgccactggtgctgtttttcatattcttcttgctatcctcttggacattgagaagacaaaattcctaggacagtttgtggcaggctttgtattactttcttttatacttggagtattcatccaatccgaagttccagtttccatgaatggaatactaataaatttaagcggggagagtacatttatgctgatgagtcttctcggagcaactcttgtgcctcacaacttttaccttcattcttctattgtacagtggcatcagggaccggcaaacatttctaaagatgcactgtgtcataaccattttttggccctcttatgtgtcttcagtggcctttatttggtaaattatattctgatgaccaccttagcaaatgagttctacagtacaggtcctgttttgcttatggaacaggtgttacacagcccaatagccctgattggctttgtactcattctgtttcttgccaatcaaactgcagcattaacttggagtttaggcggagaagttgtagttaatggtttcttaaaactagacataccaggttggcttcattatgcgacaattagagtgattgctgtgctgcccgccctttattgtgtctggagttcaggagctgaagggatctatcaactactaatattcactcaggttttagtagctttgcagcttccgtcgtctgtgattcctctttttcgagttgccttgtctagatcaataatgggtgcacacaaggtctcccagtcaatggagcttttggcgttgaccatattccttggggtgcttggcatgaatatcatgtttttgggagagatgatatttggcagtagtgattgggcgtgtgatttgaggtggaatttggggaatggcgtgtctgtcttgttttcagttctcctcattgctggttttttatcaatatgtttgatgcttcggttagctaccacacctttaagatctgccagcattcaattaaatgctcaggtcttaaactgggatatgccagaggctgtactaaatccgccggttgatggtgaagaatcacatgtaactgaaacagtgggtcatgaagatgcgtctttccaagctgacgaaccaaaaccagctttggcaaggtcattagagtaccctgaagtatcacttgctagttttcgtcctgatctacatctacctgaaactgtaatggaacctgatccccaggtgaatgctttaaaggaaaatcattctgttgcaccatcagtttccacatctgattcagggacagtatcaaaaactgtagctaatgatacttctagttctgattctaaacttaaagacacgaaaactatcattgaagcaaatgccccaattgagaagaccgtggaaattgaagatgattctaatgttgaaagggatgatgacgatgtagactcatgggaaactgaagaatcatccagagcagtcttggcaaatgccccgtcatcaacatcagaaggtcctccatcattcaggagtattagtgggaaaagtgatgacggtggttgtagctttggaagtctatcgagaatagaaggcttaggacgtgctgcaagacgtcagctagctgctactcttgatgaattttggggacagctttatgatttccatggtcaagcaacccaggcagcgaaggccaagaaaattgatgttttgctggggatgggagtagattcaaaacccaccgcttccttgcagaaaatggatgcatgtggaaaggactattctgaatacttggtatctgtagggggtagagcttccgacaatttaatcaacgctggcccgtatgactattccaatcagcctaggatgcaaagtaattcagagtctgcatatggtcttcaaaggagttcttcctcagtgcgggcaagtcccattcagttattggatgcatatgtacaaagttccaaccgcaatctcaatgattctggtgagaggcgctattccagtgttcgcaatcttcattcatctgaggcctgggattatcaaccagctacaattcatggttatcagaccgcatcctatcttagccggggtgtgaaagatagaagttctgaaaacataaatggttcaatgccattgacatcactgaagtccccttccacaggtaatccaaactatagggattcccttgcattcgttttgggaaaaaagttgcataatggttcaggtgtaggccatccccctgggtttgaaaatgtagctgtctctagaaatcgccaactacaaactgagaggtccaattatgattctagctcccctggagctgcagctaatacagtcagttcagttaacactaagaaataccacagcttgccggacatttcaggatactccattccccacagagctggctatgtgtctgataagaatgcaccgtgggatggttctgttggatatggttcttttgctggcaggatgggttatgaaccatctatgtattcaaattctggatcaagagctggaggagctcatttggcctttgatgaagtctctccatacagagaggcactctcttcgcagtttagttctggttttgacactggatccctctggtctagacagccttttgagcagtttggtgttgctggtaaaattcataatgttgcaatggaaggtgcgggaagcaggcctaatgcaattgttcaagaaattacatttgaggatatagaggggaaacttcttcagtctgttagacttaccattatgaagctcttaaaactagagggatctgattggttgtttaaacagaatgatggaattgatgaggatctgattgatcgtgtagcagcaagggataagtttgtttatgaaattgaagctagggaaacgaatcagggtattcatatgggtgacactcgttattttccttctgataggaaatctgtttcttcgatgaaagtcaacgaggcaaatgcctctagtctttcggtttcctcagttcccaactgcggggagggatgtgtatggagagcagatttaataataagctttggggtgtggtgtatccaccgtattcttgacctgtcacttttggaaagccggccagagctatggggaaaatacacctacgtgctcaatcgcctccagggcatcatcgaaccagctttttcgaagccccgtactccttcggccccttgtttttgcattcaagttccaaccacgcaccagcaaaagtcaagcccccctctttctaacgggatgctacccccaacggtaaaaccaggccggggaaaatacactactgcatcatcgttgcttgagctgattaaagatgtggagatcgctatctctagcaggaaaggacgtacaggtactgcagctggtgaggtggctttcccaaaggggaaggaaaatttggcatccgttcttaaacggtacaagcgaaggttatcatccaacaagcttgttggcaatcaggaggggactagttctcgcaagatcccgtcatcaggaccatacaaccaatag

SEQ ID NO: 153 - DNA, 3144 bp, Arabidopsis thaliana
  misc_feature 1..3144: note = ORF of protein Sps1
  source 1..3144: mol_type = unassigned DNA
atggtgggaaacgactgggtgaatagttacctggaggcgattctcgcagccgaacctggaatcgccaattccaaaccgcctggaaccggcgattccaaatcgtctcttctgctgagagagcgtggccatttcagtcccactcgttatttcgttgaagaggttatcacaggctttgatgagactgatcttcaccgttcctgggttcaggctgctgcaaccaggagtccgcaagagcgtaacacaaggttggagaatttgtgctggaggatctggaatttggctcgccagaagaagcaggttgaagggaagaatgctaagcgtgaagctaaaagggagcgtgagcgtgagaaagcaaggagggaagtaactgctgaaatgtctgaagatttctctgaaggagagaaggcagatttgcctggtgaaatcccaactcctagtgataacaacaccaaaggaagaatgtctcggattagttctgttgatgtctttgagaattggtttgctcaacacaaagagaaaaagctttacatcgtgttaataagtcttcatggtttgatacgtggtgaaaacatggagcttggtcgcgattctgatactggtggccaggtgaagtatgttgtggaacttgcaagggcattaggctcaatgccaggagtctatcgtgttgatttgttgaccagacaggtcaccgcgccagatgtggattccagctactctgaaccgtctgagatgttaaatcctatagacacagatatagagcaggagaatggagagagtagtggcgcttatataatccgtattccttttggcccgaaagataaatatgtaccaaaagagctcctctggcctcatatccctgaatttgttgatagggcacttagccatattatgcagatttcaaaggttcttggtgagcaaattggtggtggacaacaagtatggccagtttctattcacgggcattatgcagatgctggtgattccactgctcttttatctggggctctgaatgtaccaatggtttttactgggcattctcttggtcgtgataagcttgagcaactcctgaaacaaggacggccaaaagaagaaatcaattccaactacaagatatggagacggatagaggcagaggagttatgtcttgatgcttctgagattgtcataactagtacaaggcaagaggtagacgagcaatggcgcctttatgatggttttgatccggttttggagcgaaaactcagagcaagaatgaaaaggggtgtgagttgtctaggcagatttatgcctcgcatggttgttattcctccaggaatggaatttcatcacattgtaccacatgatgtggatgcagatggagatgatgaaaatccacaaactgctgatccaccaatttggtctgagattatgcgcttcttctctaacccgcgcaagcctatgattctcgctcttgctcggccagaccctaaaaagaacttggttactctagtcaaagcctttggagaatgccgtccattacgggagcttgcgaaccttacattgataatgggaaatcgaaatgatattgatgagttgtcctccacgaattcctctgtgctgctctcaattctgaagctaatagacaagtacgacttatacggtcaagtggcaatgcctaagcatcaccaacaatctgatgtgcctgaaatttaccgtttggcagccaaaacaaagggagtatttataaatccagcgttcatcgaaccatttggactgactctaatcgaggcaggagctcatgggctccccactgttgcaacaattaatggaggacctgtcgatatacatcgggttcttgacaatggtcttctagttgatcctcatgatcagcaagctatagctgatgcgctcctgaaactagtttctgacagacagctatggggaagatgcagacaaaatggcctaaataacattcacctgttttcatggccagagcactgcaaaacatatttggctcggatagcttcgtgcaagcaaagacatcctaaatggcagagagttgagtttgagaactcagactccgattcacccagcgactctcttagagatataaatgacatctccttgaatctaaaactttccttagatggcgaaaagagtggaagtaacaatggtgtagatactaacttagatgctgaagacagagctgctgaacgtaaagctgaggtagagaaagctgtttcaaccttggcacaaaagagcaaaccaacagagaaatttgattccaagatgcctactttgaaaagacggaaaaatatatttgtcatttctgtggactgtagcgcaacctcagatcttcttgcagtggtgaaaacagttattgatgctgcaggaagaggcagttcaacaggattcattctctcaacctcaatgacaatatctgagactcacacagcactgctttcaggagggttgaaaccacaagattttgatgctgttatttgcagcagtggaagtgaactctacttcacgtcctctggttcagaggataagaccgcacttccttatacactcgatgcagattaccattcccatatagaattccgctggggtggagagagcttgaggaagactttgatccgttggattagttctgttgaagagaagaaaaagacgaaaaaaggagagattttagttgaagacgagtcttcttctaccaactattgcctatccttcaaagtgaaagatcccgctttgatgcctccaatgaaagagctgaggaaattgatgagaaatcaagcattacgttgcaatgcagtttactgccaaaatggagctaggcttaatgtaatccctgttcttgcatcacgatctcaggcccttaggtatttgctagtgcgatggggcattgacttatcgaacatggtggtgttcgtgggagattcaggtgacacggattacgaaggcttgcttggaggaattcacaaaacagtgatacttaaaggcttagccagtgacttgcgtgagcaacccggcaacagaagctacccaatggaggatgtgactccactgaacagtcccaacatcaccgaggctaaagaatgtggtcgtgacgccattaaggtcgctctagagaagcttgggatcagtctcctcaagccttga

SEQ ID NO: 154 - DNA, 1122 bp, Arabidopsis thaliana
  misc_feature 1..1122: note = ORF of protein Spt
  source 1..1122: mol_type = unassigned DNA
atgatatcacagagagaagaaagagaagagaagaagcagagagtgatgggagataagaaattgatttcatcttcttcttcttcctcggtttacgatactcgtatcaatcatcatcttcatcatcctccgtcttcttccgacgaaatctctcagtttctccggcatattttcgaccgttcttctcctttaccttcttactactccccggcgacgactacaacgacggcgtctttgattggtgtgcacgggagcggtgacccacatgcagataactcgagaagtctcgtttctcatcatccaccgtcagattctgtgcttatgtcgaaacgtgtcggagatttctctgaggttttaatcggcggaggatcaggctcagccgccgcgtgttttggtttctccggtggtggtaataataacaacgttcaaggaaatagctctgggactcgagtatcgtcttcttccgttggagctagtggcaacgagacagatgagtatgactgtgaaagcgaggaaggaggagaagctgtagttgatgaagctccctcttccaagtcaggtccttcttctcgtagttcatctaaaagatgcagagctgctgaagttcataatctctctgagaagaggaggagaagtagaattaatgaaaaaatgaaagctttacaaagtctcatccctaattcaaataagacggataaggcttcaatgcttgatgaagccattgagtatctgaaacagcttcagctccaagttcagatgttgactatgagaaatggaataaacttgcatcctttgtgtttacctggaactacattacacccattgcaactctctcagattcgaccccctgaagcaaccaatgatcctctgcttaatcataccaatcagtttgcttcgacttctaatgcaccggaaatgatcaatactgtggcttcttcatacgctttggaaccttctattcgcagtcactttggacctttccctctccttacttcacccgtggagatgagtcgggaaggtgggttaactcatccaaggttgaacattggtcattccaacgcaaacataaccggggaacaagctctgtttgatggacaacctgacctaaaagatcgaattacttga

SEQ ID NO: 155 - DNA, 1488 bp, Arabidopsis thaliana
  misc_feature 1..1488: note = ORF of protein Stn8
  source 1..1488: mol_type = unassigned DNA
atggcctctcttctctctcccgccacccccaccgccacctccgccgcatttcattcctgctccaccgccggattctccactcccactcacatttcatcccagaacagcagcctgtccctcctcagccgccggggttgtatgatgagatgcagtttttctccgcaggacattcctgttgattccctctctcatctccctcctttccttgactttcagaactccctcgccacattttcggatacccagaaatgggggttctttgtctccgccggaatcgtctggttttacctcacggcgaggcctggtgttctcatcggcgccattgacgcctaccttctcgctcccctccagcttggcctcgacactctgattggaaggaggttgaagaggtccgatttcttggtcactgagaaactcggcgaaggctctttcggcgtcgtctatgccggcgtcctactccccaagaattctactcttgttgatgacgttagggttagcaaagcaagagctaaagcgatggactttaccggcgaattcaagcagagagtcatcctcaagaaggtcaaggtgggagtacgaggggccgaggaatttggcgagtacgaagagtggttcaactaccgactctcaagggccgctcctgacacctgcgctgagtttctcggaagctttgtcgccgacaagaccaatactatgtttactaaaggcggcaaatggcttgtctggaggttcgagggagaccgtgatctcgctgattacatgaaagatcggagctttccttctaatttggagtccatcatgtttgggcgcgttctccaaggagtcgagtctgtgaaacgtcgtgcactgatcattaagcagattatgcgccagatcattacatctctcaggaaaatccatggcacgggcattgttcaccgggacgtgaagcctgccaacttggtggttacaaagaagggccagattaagctcatcgactttggtgccgctgctgatcttcgcattggcaagaattacatcccggaacgcactttactcgatcctgactactgtcctcctgagctctatgtcctcccggaggaaacgccaagccctcctccggagcctatagccgctttgctttccccgattctctggcagttgaacagtccggatctgtttgatatgtattctgctgggatagtgcttctccaaatggcggttccgaccttaagatctactgccggtctgaagaattttaatctggagatcaagtctgttgaatatgatcttaataggtggcgggaaaggactcggacgaggccagacttgagcatactagatctggactcgggcagagggtgggacctcgtcactaaactcatatccgagagaggttctctgagacgtggacgcctttcagcagctgctgctctcagacacccttattttttgttgggcggtgatcaagcggccgctgttctttcaaagctcagtttcagcaagtga

SEQ ID NO: 156 - DNA, 1218 bp, Arabidopsis thaliana
  misc_feature 1..1218: note = ORF of protein Tap46
  source 1..1218: mol_type = unassigned DNA
atgggtggtttggctatggaggaaatgcctttatcggtgctattcgagcaagctaggaaaattcatcttgctgcttcagagtctggtgttgatcaggatgttgtgaagaaaggatgtgagatgtttcagaagtgcgaagatatgatcgggaagctggcgctgttttcgtctaacgagactaaagaagatattagcaccaacaatctcaaatatcttttggtgccttattatcttgcggagttgacagagaaaatcatacaggaggaccggattcagattgtcaaagcctcgtatgctaagttgaaggagttcttttcgttctgtgaggcaatggagcttgttccggatgaggagttggaggcttcttcacgaggaggttctggtgcacctgctgatcgaagggctctcaagatagctcggttcaaacgtcaaaaggctgcagaggcaaagcttcttgaaattaaagagaggaaggagcgacgtggacgttccactaaagcatcagctttgtcgactccggttgaatctggagaagatgatattccagatgatgacagcgaagaagagagagaggcctggctctcctcaattaacttggctatttgtaaggctattgatctgttggaaatgctaaagagagaagaggaaatgctctctgcaataaaggaaagacagttgaaggatggagagggtggattttctcgggatgctcttgatgatcgtacaaagaaagctgaaacctggcacagagacgctgctgcaaggatacagtactctaaaccggcacaaccaatcacttgtgccacatttgcacaagatgtgttagaaggaagagcttcggtatcacaaggtcacgaacacaagaaccaacctcttatattcggtccagcaagcatcgtgggtggaccgctctctaccgagagagaaagaatgatagctcaggtcttccaaccaagtcacaggatgccaacaatgtgcatagaggatgctgggttaacagagatgaacataatgaatgattggcaagagcagacgaaaaaagccattgaagaagcaaccacctcatggtacaatgataaacctctaagaagaaaagaagaagacgaggaagatgatgatgaagacgaagaagctgtgatgaaagctagagctttcgatgattggaaggatgataatcctcgcggtgcaggtaacaagaaactcacaccttgtggctga

SEQ ID NO: 157 - DNA, 972 bp, Arabidopsis thaliana
  misc_feature 1..972: note = ORF of protein Topp6
  source 1..972: mol_type = unassigned DNA
atggatcctggtactttaaactcggtgatcaataggttgcttgaagctagagaaaaaccaggaaagattgttcagttgtctgaaacagagatcaaacagctctgtttcgtctctagagatatcttcttgagacaaccaaatctcttggaacttgaagctcctgttaaaatatgtggggacattcatggacaatatccggatctcttgagactattcgaacatggcggataccctcctaattcaaactacttgtttcttggagattatgtcgatcgcggcaagcaaagcctcgaaacgatttgtcttttacttgcttacaagattaagttccctgaaaacttcttccttctcagaggaaaccatgaaagtgcatcaatcaatcgtatttacggcttctatgacgagtgtaaacgtagattcagtgtcaagatttggcgaatcttcactgattgcttcaactgtctccccgtcgctgcactcatcgatgagcggattttttgtatgcatggtgggctctccccggagctgctaagcttgaggcagattagggatattcgtcgtccaacggatattcctgatcgtggtttactctgtgatctcttgtggtctgatcctgataaagatgttagaggttgggggcctaacgatcgcggagtttcttacacttttggatcagatatagtttctggatttcttaaaagactcgatcttgacctcatttgtagggctcaccaggttgttgaagatggattcgagttctttgcgaataagcagctcgtaacgatattctctgcgccgaattactgtggggaatttgacaatgcaggtgcgatgatgagtgtgtctgaggatttgacctgctcttttcagatcttaaaatctaatgacaagaaatcaaagttcagtttcggaagcagaggtggtgctaaaactagcttcccttatcctaaagtgaaggattgtaattggtag

SEQ ID NO: 158 - DNA, 1350 bp, Arabidopsis thaliana
  misc_feature 1..1350: note = ORF of protein TubB6
  source 1..1350: mol_type = unassigned DNA
atgagagaaatccttcacattcaaggtggtcaatgtgggaaccagattggttccaagttctgggaagttgtatgtgatgagcatggtattgatcccactggtcgttacgttggaaactctgatctgcagttggagcgtgtcaatgtttactataacgaggcatcctgcggaagatatgttccccgtgcaattctcatggatcttgagcctggtactatggacagtgtcagaactggaccttatggtcaaatcttcaggcctgacaactttgttttcgggcaatctggtgctggaaacaactgggctaaagggcattacactgaaggagctgagcttattgatgctgtactcgatgttgtacgcaaagaggctgagaattgcgactgtcttcaaggtttccaagtatgtcactcacttggtggaggcaccgggtctggaatgggaactctgctcatatctaagatcagggaagagtatcctgatagaatgatgcttacattttctgtcttcccatcaccaaaggtctctgacacagtggttgagccatacaatgcgacactttcagttcatcagctggtggaaaatgctgatgagtgtatggttttggacaatgaagccctttacgacatctgttttagaacacttaagcttaccactcctagctttggtgatctgaatcatcttatctctgcaaccatgagtggagttacatgctgtcttaggttcccgggtcagctcaactctgatctgaggaagctcgcagtgaacctcattcctttccctcgtctccactttttcatggttggttttgcccctctcacctcccgtgggtctcagcagtaccgtgcactcactgtccctgagctgacccaacagatgtgggattcaaagaacatgatgtgcgcagcagacccgcgccacgggcggtacctaactgcctcggccatgttccgtggcaagatgagcacaaaagaagtggacgagcagatgataaacgtacagaacaaaaactcttcctactttgtggaatggattccaaacaacgtgaagtcaagcgtctgtgacatagctccccgaggtctctcaatggcatcgacatttattgggaattccacatcgatccaagagatgtttaggcgggtgagcgagcagttcactgctatgttcaggaggaaagctttcttgcattggtacacaggtgaaggaatggacgagatggagtttactgaagctgagagcaacatgaacgatctagtctcagagtaccagcaataccaagacgcaactgcagatgacgaaggcgagtatgaagaagacgaggatgaagaagagatattggatcatgagtga

SEQ ID NO: 159 - DNA, 2700 bp, Arabidopsis thaliana
  misc_feature 1..2700: note = ORF of protein TubB8
  source 1..2700: mol_type = unassigned DNA
atgcgagagattcttcacatacaaggtggccaatgcggaaaccagatcggagctaagttctgggaagtagtttgcgccgagcacgggatcgattcgacgggaagataccaaggagagaacgatttgcaactcgaacgagtcaatgtctactacaacgaagcgagttgcggaagattcgtcccacgcgccgtcctcatggatctagagcctggtactatggatagtgtcagatccggtccgtacggtcagatcttccgtcctgataacttcgtatttggtcaatccggtgccggtaacaattgggctaaaggacactacactgaaggtgctgagcttatcgattccgttctcgatgttgttcgtaaggaagctgagaattgtgactgcttgcaaggattccaagtgtgtcactcattaggaggaggaactggatctggaatgggaacgttgttgatttcaaagattagggaagagtatccagataggatgatgctgacgttttctgtattcccatcaccgaaagtgtcagacactgttgttgagccttataatgcaactctctctgttcatcagcttgtggagaatgcagatgagtgtatggttcttgacaatgaagctctctatgacatttgcttcagaactcttaagctcaccactccaagctttggagacttgaatcacctcatctctgcaaccatgagtggtgtcacttgctgccttcgtttcccgggtcagctaaactctgacctgcgcaagcttgcagtgaatttaatcccattccctcgtctccacttcttcatggttgggtttgcaccactcacatctcgtggatcacagcaatacagagccttaaccgtccctgagctcactcaacagatgtgggatgccaagaatatgatgtgtgcagctgatccacgccacggcaggtacttgacagcctcagccatgttcagaggcaaaatgagcacgaaagaagttgacgaacagatgatcaacgttcagaacaagaactcgtcatactttgtggaatggatcccaaacaacgtgaaatcaaccgtgtgtgacattccaccaacaggtctgaaaatggcttcgactttcatcgggaactcgacatcgatccaggagatgtttaggcgtgtgagcgaacagttcacagcgatgttcagaagaaaagctttcttgcattggtacacaggtgaaggaatggacgagatggagttcacagaggcagagagcaacatgaatgatttggtatcagagtatcaacagtaccaagacgcaacggctgatgaggaagaagggtatgagtatgaagaagacgaagtggaagtgcaggaggagcaataaatgcgagagattcttcacatacaaggtggccaatgcggaaaccagatcggagctaagttctgggaagtagtttgcgccgagcacgggatcgattcgacgggaagataccaaggagagaacgatttgcaactcgaacgagtcaatgtctactacaacgaagcgagttgcggaagattcgtcccacgcgccgtcctcatggatctagagcctggtactatggatagtgtcagatccggtccgtacggtcagatcttccgtcctgataacttcgtatttggtcaatccggtgccggtaacaattgggctaaaggacactacactgaaggtgctgagcttatcgattccgttctcgatgttgttcgtaaggaagctgagaattgtgactgcttgcaaggattccaagtgtgtcactcattaggaggaggaactggatctggaatgggaacgttgttgatttcaaagattagggaagagtatccagataggatgatgctgacgttttctgtattcccatcaccgaaagtgtcagacactgttgttgagccttataatgcaactctctctgttcatcagcttgtggagaatgcagatgagtgtatggttcttgacaatgaagctctctatgacatttgcttcagaactcttaagctcaccactccaagctttggagacttgaatcacctcatctctgcaaccatgagtggtgtcacttgctgccttcgtttcccgggtcagctaaactctgacctgcgcaagcttgcagtgaatttaatcccattccctcgtctccacttcttcatggttgggtttgcaccactcacatctcgtggatcacagcaatacagagccttaaccgtccctgagctcactcaacagatgtgggatgccaagaatatgatgtgtgcagctgatccacgccacggcaggtacttgacagcctcagccatgttcagaggcaaaatgagcacgaaagaagttgacgaacagatgatcaacgttcagaacaagaactcgtcatactttgtggaatggatcccaaacaacgtgaaatcaaccgtgtgtgacattccaccaacaggtctgaaaatggcttcgactttcatcgggaactcgacatcgatccaggagatgtttaggcgtgtgagcgaacagttcacagcgatgttcagaagaaaagctttcttgcattggtacacaggtgaaggaatggacgagatggagttcacagaggcagagagcaacatgaatgatttggtatcagagtatcaacagtaccaagacgcaacggctgatgaggaagaagggtatgagtatgaagaagacgaagtggaagtgcaggaggagcaataa

SEQ ID NO: 160 - DNA, 1044 bp, Arabidopsis thaliana
  misc_feature 1..1044: note = ORF of protein Uba1a
  source 1..1044: mol_type = unassigned DNA
atggcgaaaaccctagacaaatctaagaagcgcaaactcgtgaaatccaaatccacgaatttcgataagaaacagaagattaacaaacaacaacaacaaccagaatcctcaacgccatattcaagttcaagctccagctctgattctagcgattccgaatcagacaacgaattcgacccagaagagctccgtgagcttctacaaccttattccaaggaccagcttgtcgatctagtttgttccgcttctcgaatcggatcttcgatctattccgccgtcgtagaagccgcagatcgtgatgttacacacaggaaaatcttcgtttatggtcttccatgggagacgactcgcgagactcttgttggagtctttgaaggttatggtgagattgaagagtgtaccgttgttatagataaggctactggtaaggctaaagggtttggatttgttatgttcaagactaggaaaggagctaaagaagctcttaaagaaccgaagaagaggattcttaatagaactgccacttgccagcttgcttctatgggtcctgcggcttcaggaaaggggcatgatcaacctggtccggtgaagattagtatgggttcgatggctaatcatggacagcctcagcaacagcaggtacaaggtcaacatgtgtttaacggtggtggaatggctgcttcgccgtttatgttagggaaccaataccatccgctttatggagctgggatgttgggaaatccagctttagcagcagcagcagcagctgggggaggttacatgtatccgatgttagcgggtgctctagcacatggcgggttgggttctgatatggtacagtctagtcaaatgggtgggattggtgttgatccaagcgttggtgctgctggtctttcagctctaggttcttactttagaggtcagagtctgcctagtacttatcctgattcggatgctggtggaaaaagagggacgggtaaagattcggatgctggtggctcctcctttcatggctactcaaactattcatggtacgttcactga

SEQ ID NO: 161 - DNA, 1854 bp, Arabidopsis thaliana
  misc_feature 1..1854: note = ORF of protein Vim3
  source 1..1854: mol_type = unassigned DNA
atggcgattgaaactcagcttccttgcgacggtgacggtgtgtgtatgcggtgtcaggtgaatcctccgtcagaagagactctcacttgtggcacgtgcgtcactccatggcacgtgccgtgtctcctccccgaatcactcgcttcttccactggagagtgggagtgtcccgattgctccggcgttgtcgttccctccgccgctccgggtaccggaaacgctcgacctgaatcttccggttcagttctcgttgctgcgatccgtgcgattcaggctgatgagactttaaccgaagctgagaaagccaaaaaaaggcagaaactgatgagtgggggtggtgacgatggtgtcgatgaagaagagaagaagaagttagaaatcttttgttctatttgcattcaattgccagaaagacctatcacgacaccgtgtgggcacaatttctgtttgaaatgtttcgagaaatgggcagtaggtcaagggaagctaacttgtatgatatgccgaagcaaaattccgagacatgtggcaaaaaatcctcgcatcaacttagctctagtttctgctattcgtttagcaaatgttaccaaatgttctgttgaggcaactgcagccaaggttcatcatattatccgcaaccaagaccgtcctgagaaagcatttactaccgagcgggcagtaaaaactgggaaagctaatgctgctagcggtaagttttttgtgacaatacctcgtgatcattttggtcccataccagctgagaatgatgtcactagaaagcaaggtgttttggttggagaatcttgggaggacaggcaagagtgtaggcagtggggagctcatttcccgcatattgctggcattgccgggcaatcagcggttggagctcagtctgtggccctctctggaggttatgacgatgatgaggatcatggtgaatggtttctctacacaggaagtggtggaagggatctcagtggaaacaaaagaattaacaagaaacagtcgtctgaccaggcgtttaaaaacatgaatgaatctctaagacttagttgcaaaatgggctatcctgtccgagttgtcaggtcttggaaggagaagcgttctgcatatgcccctgctgaaggtgtgagatatgatggggtctatcgaattgagaagtgctggagtaatgttggagtacagggttcttttaaggtctgtcgttacctgtttgttagatgtgacaatgagccagctccatggaccagtgatgagcatggcgatcgtccaagaccgttgcctaatgttccggagcttgagactgctgctgacctgtttgtgagaaaggagagtccatcatgggatttcgatgaagctgagggtcgttggaaatggatgaagtctcctcctgttagcagaatggctttggatcctgaggagaggaagaagaataagagagcaaaaaatactatgaaggccagacttctgaaagaatttagttgccaaatctgtcgggaagtgctgagtcttccagtgacgacgccttgtgcacacaacttctgcaaagcatgcttagaagcgaagtttgctgggataactcaactgagagagagaagcaatggcggacgtaaactacgtgcaaagaagaacatcatgacctgcccttgctgcacgacggatctctccgagtttctccaaaacccgcaggtgaacagagagatgatggagataatagagaattttaagaagagtgaggaagaggctgatgcatccatttctgaagaagaagaagaagaatccgaacctccaactaagaagattaagatggataacaactctgttggtggtagtggtacaagtctctcagcttaa

SEQ ID NO: 162 - protein, 11 aa, synthetic construct
  REGION 1..11: note = NSP1-5UTR-11
KPFKFSSSISH

SEQ ID NO: 163 - protein, 5 aa, synthetic construct
  REGION 1..5: note = NSP1-5UTR-5
KPFKF

SEQ ID NO: 164 - protein, 10 aa, synthetic construct
  REGION 1..10: note = NSP1-3UTR
CHHDEVISPQ

SEQ ID NO: 165 - RNA, 22 nt, synthetic construct
  misc_feature 1..22: note = Fig. 1
  source 1..22: mol_type = other RNA
aggccgacaagattcggtagaa

SEQ ID NO: 166 - RNA, 12 nt, synthetic construct
  misc_feature 1..12: note = Fig. 1
  source 1..12: mol_type = other RNA
ccgacaagattc

SEQ ID NO: 167 - RNA, 12 nt, synthetic construct
  misc_feature 1..12: note = Fig. 1
  source 1..12: mol_type = other RNA
cgacaagattcg

SEQ ID NO: 168 - RNA, 12 nt, synthetic construct
  misc_feature 1..12: note = Fig. 1
  source 1..12: mol_type = other RNA
gccgacaagatt

SEQ ID NO: 169 - DNA, 6813 bp, Medicago truncatula
  misc_feature 1..6813: note = NSP1
  source 1..6813: mol_type = unassigned DNA
aagcttatcccccaaagctatccttaaccctaatcttttccccctaatttttccctccacttttctaagccattggatcatgaacatttaggcgccatagaacatcagccgttgatctatctacacttattgctcatcctaatcagcattccattatgtattcatattttccctccctcaatcaactcctccatttaattgtttggttccagtgcaaaaatattattccaatgcaattgagcatttaaatgctcattctctttaccatgcatatatttattgtacttcatgttgttccctttttctctcatcacttgatttgatcactctctcctttcctaatcatgttcttcttctcctctttccacgaattacttcatctccgctttcactatgcagatcaaccatgttcttttttccacactcttttgtctccattgcctctcaccatcgtctcaaccctttcactatgttgcgcttttaatttgttttattgatgatgcagagttctttgatccagatctgggtcatttgaagatgtgatacaacatgtacttggttatggtatattaagagaaggtatttggttatggtaaatatttggtatttggttgtgcaatcatgttataaatttatggtttgttaacagctccctttgcctttttgctctcctatcattgcatcactggtttgtatattctttccttatctcccataggttttttcgattcagctatataacctgctgtgtcgttttatttttctaaccaactgttttaatactttcttttgtagcaccattggtgcggaatccagttacggtaatggatcgactaatcaataagcagcattaaacaaaaaatgatccggaagaagaatgattgatgcacgaaagactaattgtttatcgattttgtgttttgtcattagacatatcttttttgattcaagtagcgtttcacaatgtatccgtcagaactaattgtttttgtttattaagtaatattaaaacttcaatccgaagtattctttatattttttcattaccgaaacatatgcctttatcatttttggggttagtacttgcatgtcgattacaatcatcgaagttgaacagaacttgattatgctatataatggtttagcacagtaatcttcatatgcgttaatttttacttatacacatgcgttttttttttttaataaaaggaaggtatgcaactgcattcaaaatctaattaacattttcatacatttttgagtaatattcaaaattgtcaacgatgaagaggaagctacctttgttaaattgacattacaattgaataagtattgatgcaaaactagaaaagatgttaaatgaaaaaaattggctagatgtaaattgaaatcaaaagagaattaccattgctacaagattaatttctccaaatgaacaattggtgtcaagaacgagtcaacctatccattttcatggtaattatcacacgagttttaacatttttaacttaaatgctttttactgttgttttaaaactagagtcaaccatccacctttatattttatgtttgatatgctattttaacatgaaccattactttggaaataccaacctcatgatccaacttatattgaactcctatggcttaagtggcgtagtcgaatttcatcctgaagactttatcagtagttctatattccacataatcgattagttttaagctaaaatttatattttatcaacaattctattttacaattggagtcccaatccatttcttttattttacaattctaatttttattttctttatagcttcatactacaaaataaatgttgtcattagaaaaacccataaaattgtttcaaacaaaaaaccataaaataaatttggtccttatatgctttttttaaaaagaaaataaatttaaggctgtcttttcttttaatataaatggattaatagggtaaacaatgaacctggaaatccataagtttcgggggtgcaacatttagagtggtacatcgttagaagaaaataaatttaatgctgctttttcgcgaaaactgattttatctatactataaaataactgttgacattatatgcttttccacgaaaacttatatgaatttatttttaagaagaaagtttatttacatagaagttagaagaaagttatccatgaagtataataaaataaatgttgtcgttagatgcttttccacgaaaacttatttgaatctatttttaagaagaaagtttatttacatagaagctagaaaaaagttatccatgaattaatatatatgacatcaatatcaagaatattatggtagaatttagcaataacaaatttaactccaaaaatttagatagcatgaacaaattcacggattaaccatgaacactctttacttttgtagtatgaagttataaaaaaaataaaaataaaaataaaaataaaaaataaaaaattgaatgtttaataggatatagaaaattatcattttttaagaaaaggataatcaatattaataatgtgagacaattggcattttttgttttcttaacatgttttttaagtgtttatggtgtattaatgctacaatctagtatgttagcaggatgtatacattgagggaaaattgtttgataaggaggttggatatgatttagtataccgttacatcaagatgatcaatcagacattaaattagtatgaggtcgtggttgaccaaaagcatggcaagatatgtttcacacatggtttgagtgagttggacaaagaatatactccctcccatcactattataagtaaaaaacaatttttagcttcattgaataaatgatatatttgtctatattgttagtcaaatacatcatttaataaatgaacctaaaagtttgtttgtgcttataacagtgataggagggagtatttataccgagattatcagttacactatcactttcaaatttcaatcaagacaatttccgttaattgtgttatttgcaggggcggatccagacatttgatattggtgtggcaaaatatttaaatcaactatatttattaacaaagtaaataattgataccttgaacaatatagcgaaatggtatagatggaagcattttaccattgtgttgcaggttcgattccctgcaacaccctttcaactgccacaccatgtttcatattagatccgcccatggtcatttgtaatgactattagcacgggacatatatctagtttataaataatttgtcttgcttttttacgtttatatgattagtttttttttttagggaaagtttgtatgattagttatttccatcataatatttattgatatatacaaacaaacaaaattgtacaataaatacggatattttggaggaaaaaaattatgtagtaaaaatttgaaatcgaacgttataaaaagctacataaatttttgggttaaataagttttttatcctcaaaaaattaaacaaacatattttaagatagtggtagtacatattttcaacacattatctcctgttaatttagattaaaatcagtccctctaaattgggaaggtccaaaacttttgacaaaatctatgtacagtaaattttaatcaattaaattaaatatagtacattgagttgcatatcatttttgtcttcacaatttaaaaatacaaatttatgacctattaacaacaaaatacagtacaaaacccatccagacataaactttcaagaattatgaaattagtcaaatattggttgccggctagagttttgtatataacctatcacattcttcaacacataaattcaatcttaaacagtatatatgaatctcctatataatacaaaaagaacaagttaaccacccaacataaacaagagacaacctcgtctgctagttgcctatgtaaaccattaaaaaacatcaataaaacatcacatcttccaaaccaaacagcacacaaacaattagaaaattccctcatttgtcactactaggtaattatcaatcctctatgctttattttataagtacatgctagaaactattgaactcaaaagtctttggttctatgatctaatacctatcctcctcttgtttttggtcaatgatccaacactattaacctcttcaaacccttcaaattttcttcttcaatatctcatagtttttttctgttttttcattacaatgactatggaaccaaatccaacatcagatcacattcttgattggcttgaaggttctgtctctttctttccatcatttttggatgatccatataacaatggttacatccatgagtatgagatatggaatcagaatcaagatataagtaaccagtaccaaattgatgccaatactaattcttctaatgccacaaatagcacaaccaacattgttgctgctagtactaccacaagtacaacatcccttgagcctaatagctttaataacatacccttttcagatttgccgaagaaacgaaatgccgaagatgaactaagtctcaaaaaacaacctcaaaaccaaaagaacaagagacttaagagtcgtccgatgaatgaaagtgataatggagatgcagctcttgaagggacagtggttagaaaatctggtgggaacaagaaaggtgcagctaaggccaatggaagtaacagtaacaatggaaacaataaggatggtaggtgggctgagcagttgctcaacccttgtgctgtagccataaccggtggaaatctgaatcgtgtgcaacatctcttatatgttctccatgagctagcctcaactaccggtgatgccaaccaccggcttgcagcacatggtcttcgagcattgacacaccatttatcttcatcttcgtcatctaccccatcagggactattacttttgcatctacagaaccgcgattcttccaaaagtcattactgaagttctatgagtttagcccttggttttcctttcctaataacatagcaaatgcttccattcttcaagttctagctgaagagccgaacaatttgcgcacccttcacatccttgacattggagtctctcatggtgtgcaatggccaacttttctagaggccttgagtcgtcgacctggtggacctcctcctcttgttcgccttactgtcgttaatgcctcttcatccactgaaaatgaccaaaacatggagaccccattttcaataggtccatgtggtgatactttctcttctggactccttggttatgctcagtccttaaatgtcaatctgcagataaaaaagcttgataatcatccattgcagacattgaatgctaaaagtgttgacacatcctctgatgaaacccttattgtctgtgctcagttcaggttgcatcacttgaatcataacaatcctgatgaaagaagtgagtttctgaaggtgttgagaggcatggagcctaaaggggtgatattaagtgagaacaacatggagtgttgctgcagtagttgtggcgatttcgccactggattctcacgaagagtggagtacttatggaggtttttagattcaaccagttcggcattcaaaaaccgtgacagtgatgaaaggaaaatgatggaaggcgaggctgcaaaagcattgacaaaccagcgtgaaatgaacgaaaggagagaaaaatggtgtgaaagaatgaaagaagcagggtttgcaggggaagtatttggagaggatgcaattgatggaggtcgagctttgttaaggaagtatgataataattgggagatgaaagtagaagaaaatagtacaagtgtggaactatggtggaagagccaacctgtttctttctgttctttgtggaaactggataaacaaccagagtagttcgagctcaacattgacagcaaggctataagcaaggtaccttggttttggagttctgtatagaagtagcagttgccaccatgatgaagttatttcaccacaatgtagttcttccttatgctaaacttacaactacaagcatatttgtatgatattgagttaagaattatttaatttgatgtatgttttgtaatgattgatagctattcagaaggtagtattgattcaaaattgatagctattcagaatgtagtattgattcatagttaggcacttgttgtttttctgctacaaatctgcaaattagatcatctaagctaacaaatgtggcattaaaaatcaaaaggttcggccactactctattttgttaacacatctattaaatatgtatgctttgaaagttccattacaggtcaagcaataaaactcatgttcccttaactggtatgtaggaccgtctgacaaaaatataaagctgtatttcgaaagtggcatttacaagcacacttgtataaacagatatatctttaaactgggagtagagtaatacaggtaacaaggaacatttcagggttcactctatagttgtctagtgaaattaaatgtgtacactgcaaccgggaaaatcggaaatctttcaatattacgcaataaggatggatcatagatgtatctgttacaaaggtaaatgcaatatgaatctcagtcaacgcctaaattgctcaatttagttacagaatcagaatgtttaattaacgtcaaacgcctcaatattcaagtggtatatttgtcagttttggattacacgttctcgttcttcacctcttcaaacatgcacattcttactagttaagcttaggcttcttctaattcgcattcttattgcaaaattcatatttgaaactaagaatgagcttctcactctaag

SEQ ID NO: 170 - protein, 11 aa, synthetic construct
  REGION 1..11: note = TAT peptide
YGRKKRRQRRR

SEQ ID NO: 171 - DNA, 807 bp, Arabidopsis thaliana
  source 1..807: mol_type = other DNA; note = Magnesium dechelatase
atgtgtagtttgtcggcgattatgttgttaccaacgaagctgaaaccagcttattcagacaaacggagtaacagtagcagcagcagctcactcttcttcaacaatagaagatccaagaagaagaaccaatcgattgttcccgttgcaaggttgtttggaccggcgattttcgaatcatccaaattgaaagtactcttcttaggggttgatgagaagaagcatccttcaacgctccctaggacttacacactcactcacagtgacattacagctaaactaaccttagctatttctcaatccataaacaactctcagttgcaaggatgggcaaataggctataccgggatgaagttgtggcagaatggaagaaagtgaaagggaaaatgtcgcttcacgttcattgtcacataagcggtggccatttccttttagatctctttgcaaagtttcgatatttcatcttttgcaaagaactacctgtggtgttgaaggcttttgtgcatggagatgggaacttgttgaacaactatcctgagctacaagaagctcttgtttgggtctatttccattctaatgtcaatgagttcaacaaagtcgagtgttggggtccgctttgggaagctgtttcgcctgatggtcacaagactgagactcttcccgaggctcggtgtgcggacgagtgtagttgttgttttccaaccgttagctcgattccatggtctcatagtcttagtaatgaaggtgtaaatggttactctgggactcagactgagggaattgctactccaaatccggagaaactctag

SEQ ID NO: 172 - DNA, 1329 bp, Arabidopsis thaliana
  source 1..1329: mol_type = other DNA; note = Abscisic acid insensitive
atggtaactagagaaacgaagttgacgtcagagcgagaagtagagtcgtccatggcgcaagcgagacataatggaggaggtggtggtgagaatcatccgtttacttctttgggaagacaatcctctatctactcattgacccttgacgagttccaacatgctttatgtgagaacggcaagaactttgggtccatgaacatggacgagtttcttgtctctatttggaacgcagaggagaataataacaatcaacaacaagcagcagcagctgcaggttcacattctgttccggctaatcacaatggtttcaacaacaacaataacaatggaggcgagggtggtgttggtgtctttagtggtggttctagaggcaacgaagatgctaacaataagagagggatagcgaacgagtctagtcttcctcgacaaggctctttgacacttccagctccgctttgtaggaagactgttgatgaggtttggtctgagatacatagaggtggtggtagcggtaatggaggagacagcaatggacgtagtagtagtagtaatggacagaacaatgctcagaacggcggtgagactgcggctagacaaccgacttttggagagatgacacttgaggatttcttggtgaaggctggtgtggttagagaacatcccactaatcctaaacctaatccaaacccgaaccaaaaccaaaacccgtctagtgtaatacccgcagctgcacagcaacagctttatggtgtgtttcaaggaaccggtgatccttcattcccgggtcaagctatgggtgtgggtgacccatcaggttatgctaaaaggacaggaggaggagggtatcagcaggcgccaccagttcaggcaggtgtttgctatggaggtggcgttgggtttggagcgggtggacagcaaatgggaatggttggaccgttaagcccggtgtcttcagatggattaggacatggacaagtggataacataggaggtcagtatggagtagatatgggagggctaaggggaaggaaaagagtagtggatggtccagtggagaaagtagtggagagaagacagaggaggatgatcaagaaccgcgagtctgctgctagatctagagcaagaaaacaagcatatacagtggaattggaagctgaacttaaccagttgaaagaagagaatgcgcagctaaaacatgcattggcggagttggagaggaagaggaagcaacagtattttgagagtttgaagtcaagggcacaaccgaaattgccgaaatcgaacgggagattgcggacattgatgaggaacccgagttgtccactctaa

SEQ ID NO: 173 - DNA, 3284 bp, Arabidopsis thaliana
  source 1..3284: mol_type = other DNA; note = Heat shock protein 101
caaaaagagtggaattgacatttgtttctctcacactctctctcgaattctctggtagcttctagttctatgcaaaaaaacgacgatagttctctatctttccagatgaatctccttccatatacaaaagcagtcatgcctcctcgctctctcgcaattcacaaagtatccaaacatctaaagttatcaattttacaacattaccgctataatctgcttgattctctgcaaaaagagaagactttttaccgagaagaagtcctctggctcattgaagaaactcaacgaaacaaacccagttctcatatatcgttttaagggattacaaaagctaatcgaagatgaatccagagaaattcacacacaagacaaacgagacaattgctacagctcatgagctagctgtgaatgcaggacatgctcaattcactcctttgcatttagctggtgctttgatctctgatcccaccggtatatttcctcaagcaatctctagtgccggtggcgagaacgcagctcaatctgctgaaagagtgatcaatcaagccttgaagaagcttccttcacaatctcctccacctgatgatattccagcgagttctagtcttattaaggtcattcgtcgtgctcaagctgctcagaagtcacgaggtgatactcatttggctgttgaccagttgattatgggtcttcttgaagattctcaaatcagggatttgttgaacgaagtcggtgtagcgacggcgagggtaaagtctgaggttgagaagcttcgtgggaaagaagggaagaaagttgagagtgcttcaggggacacaaattttcaagctttaaagacttatggaagagatttggttgagcaagcagggaagcttgatcctgtgattggtcgtgatgaggagattagaagagtcgtgaggattctttcgaggagaacgaagaacaatcctgtgcttattggagagccaggagttggtaaaacagctgtggttgaaggtttagcacaaaggattgtgaaaggagatgtgcccaacagtcttactgatgtgagattaatttcgttggacatgggtgcgttagttgctggtgctaaataccgaggagagtttgaagaaaggttgaaatctgttttgaaagaagttgaggacgctgaaggcaaagtgattctctttattgatgagattcatttggttcttggtgctggcaaaactgaagggtcgatggatgcagctaatctgttcaagcccatgttagctagagggcagcttcgatgcattggtgctacaacgcttgaagaatacaggaaatatgttgagaaagatgctgcctttgagaggaggttccaacaagtctatgttgcggagccaagtgtgcctgacaccattagtatccttagaggactcaaggagaagtatgagggacatcatggtgtgcgaatccaagacagagctcttataaatgctgctcagctgtctgctcgttacataactggtcggcatttaccggataaagcaattgatttggttgatgaggcttgtgcgaatgtgagagtccagcttgatagtcaacctgaagagattgataaccttgaaaggaagaggatgcagctggaaattgaacttcacgccttggaaagggagaaggataaagccagcaaagctcgacttatagaggtgcggaaagagcttgatgacctgagagacaagcttcagcctctcacgatgaaatacagaaaggagaaagagagaattgatgagattcgaaggcttaaacagaaaagagaagagctcatgttttctttgcaggaggcagaacgaagatatgaccttgcaagagctgctgatctaagatatggcgcaattcaagaagtggaatctgcaattgcccaacttgaaggaacttcttctgaagagaatgtgatgctcacagaaaacgttgggcctgaacacattgctgaggttgtgagccgttggacagggattccagtgacgagacttggccaaaatgagaaggagaggttgattggtcttgctgataggttgcataagcgggttgtgggacagaatcaagcggtaaatgcagtttctgaggcaattctaaggtcaagggcaggacttggaaggccacaacagccaactggatcattcttattccttggaccaactggtgttggcaaaactgagctcgccaaggctcttgctgagcagctgtttgatgatgaaaacctcttagttcggattgatatgtcggaatatatggaacaacactctgtctctcgcctcattggggcaccaccagggtatgttggtcacgaggaaggtggacaactaactgaggctgtgaggaggcgaccttattgtgtcatactctttgatgaagtggagaaggctcatgttgctgtcttcaacactctgctccaagttttggatgatggtcgattgacagacgggcaaggcaggacagtcgatttcaggaactcggtgataatcatgacatcaaaccttggtgctgaacacctccttgcagggctaactgggaaagtaacaatggaagtggcccgggactgtgtgatgcgggaggtgaggaaacacttcagaccagagctcttgaacaggcttgacgagattgtggtgttcgaccccctttcacatgaccagttgaggaaagtagctcggcttcaaatgaaagacgttgctgtccggcttgctgaaagaggagttgctttggcagtcactgatgctgctttggactatatcttggcagagagttatgacccggtgtatggtgctaggcctataaggagatggatggagaagaaggtggtgacagaactgtcaaagatggttgtgcgtgaggaaatcgatgaaaactccactgtttacatagatgcaggcgctggtgatcttgtgtaccgggtagaaagtggaggtctagtggacgcttcaacaggcaagaagtcagatgtgctgattcatattgctaacgggccaaagagaagtgatgcagctcaggcggtgaagaagatgaggatcgaggaaatagaagatgacgataatgaggaaatgatcgaggattaaagtcttgacctccaacgtttgagttcttttgaggtgttgtgattgtatgacttgagctttcatgctctgttttggttttgactcttttggtagactataatggtgtggaactgtaaaaaagtctactttattatgttttcgtatatgtaaatcattttacataagaaatccattgtacgtcccataatcttttggggttttttcata

SEQ ID NO: 174 - DNA, 1254 bp, Medicago truncatula
  source 1..1254: mol_type = other DNA; note = ATP dependent RNA helicase
atgaaatcatttacggatttgggcttatctgttcaattggtggaagcttgtgaaaaaatgcgttggtatagtccacttaagattcagacagaagtgattccccttgctctacaaggaaaagatgatgtgatcggaatttcaccaccgcgttctggtaaggccggggcttttgttcttcctatattgcaggccctcttagaagccggacccaacctaaacacgtcctttgcttgtgtactgtctccctcaagggaccttgtttttcgtattgctgagtattttcaagttctaggttctcaatttggtgtcaagtgtgctacccttgttgaagcgaacgacatcatagatcaaaccaaccagattttacagcaacctcatcttattgttggaacccttcgacaagtcttttatcacttaagactaacccaaggattttctcttgctagattaaaatacctggtcatacatgaggcagatctgctgttgaatgaccagttcgaggaacaacaactaaatgatattttatcaataattcctagtgagcggagaacttttcttttttcatctacaatgacagagaaggtccatatgatccaaagattgtctttaaggaaccctctaaagattgatgtatcttccaaatattctactgtggtcacacagctgcagcagagttgctttatgccagcaatgcttaaggattgctaccttgtatacattctcactgaaatgactggaagaaaatcaacggtattcactcagacttgtggttcagcgtttcttctggctttaattcttaagaatcttgatttcagagccatcccaattattagttatatgagccaggcaaagaaactaggagccctgaatgcgttcaagtctggaaagttcaatattctcctttgtagtgaagcagctaggaggggactagatattccagcggtagatatggtgattaactacaacatacctagagatcccaatgattatatgcatcgtgtgggatggactggtcatgttaatgctgccatttcttttgtgaacccatatgaagcgggacaacttgaaatgatagagagacatacaggcaagaagctaccggtgtatccggcaccatgtgagaatgttttgctttggagagggactgttagggaggccgaaagattagcacgaaaggaaatcaaagaatctggttggaagagtggaggatatttaggagacgaggaggtcaaaaagatctcaagtctgcgttga

SEQ ID NO: 175 - DNA, 909 bp, Arabidopsis thaliana
  source 1..909: mol_type = other DNA; note = WUSCHEL
ctttgtgaacaaaagtcgaatcaaacacacatggagccgccacagcatcagcatcatcatcatcaagccgaccaagaaagcggcaacaacaacaacaacaagtccggctctggtggttacacgtgtcgccagaccagcacgaggtggacaccgacgacggagcaaatcaaaatcctcaaagaactttactacaacaatgcaatccggtcaccaacagccgatcagatccagaagatcactgcaaggctgagacagttcggaaagattgagggcaagaacgtcttttactggttccagaaccataaggctcgtgagcgtcagaagaagagattcaacggaacaaacatgaccacaccatcttcatcacccaactcggttatgatggcggctaacgatcattatcatcctctacttcaccatcatcacggtgttcccatgcagagacctgctaattccgtcaacgttaaacttaaccaagaccatcatctctatcatcataacaagccatatcccagcttcaataacgggaatttaaatcatgcaagctcaggtactgaatgtggtgttgttaatgcttctaatggctacatgagtagccatgtctatggatctatggaacaagactgttctatgaattacaacaacgtaggtggaggatgggcaaacatggatcatcattactcatctgcaccttacaacttcttcgatagagcaaagcctctgtttggtctagaaggtcatcaagaagaagaagaatgtggtggcgatgcttatctggaacatcgacgtacgcttcctctcttccctatgcacggtgaagatcacatcaacggtggtagtggtgccatctggaagtatggccaatcggaagttcgcccttgcgcttctcttgagctacgtctgaactag